(19) United States
(12) Patent Application Publication
(10) Pub. No.: US 2016/0032395 A1
(43) Pub. Date: Feb. 4, 2016

(54) CANCER BIOMARKERS AND CLASSIFIERS AND USES THEREOF

(71) Applicants: Elai DAVICIONI, La Jolla, CA (US); Nicholas George ERHO, (US); Lucia LAM, (US); GenomeDx Biosciences, Inc., Vancouver (CA)

(72) Inventors: Elai Davicioni, La Jolla, CA (US); Nicholas George Erho, Vancouver (CA); Lucia Lam, Burnaby (CA)

(21) Appl. No.: 14/772,348

(22) PCT Filed: Mar. 11, 2014
(86) PCT No.: PCT/US2014/023693

§ 371 Date: Sep. 2, 2015

Related U.S. Application Data
(60) Provisional application No. 61/783,628, filed on Mar. 14, 2013.

(51) Int. Cl. C12Q 1/68; G06F 19/20

(52) U.S. Cl. CPC C12Q 1/6886; G06F 19/20; C12Q 2600/158; C12Q 2600/118; C12Q 2600/106

(57) ABSTRACT

Disclosed herein, in certain instances, are methods, systems and kits for the diagnosis, prognosis and determination of cancer progression of a cancer in a subject. Further disclosed herein, in certain instances, are methods, systems and kits for determining the treatment modality of a cancer in a subject. The methods, systems and kits comprise expression-based analysis of biomarkers. Further disclosed herein, in certain instances, are probe sets for use in assessing a cancer status in a subject. Further disclosed herein are classifiers for analyzing a cancer.

### BACKGROUND OF THE INVENTION

Cancer is the uncontrolled growth of abnormal cells anywhere in a body. The abnormal cells are termed cancer cells, malignant cells, or tumor cells. Many cancers and the abnormal cells that compose the cancer tissue are further identified by the name of the tissue that the abnormal cells originated from (for example, breast cancer, lung cancer, colon cancer, prostate cancer, pancreatic cancer, thyroid cancer). Cancer is not confined to humans; animals and other living organisms can get cancer. Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if breast cancer cells spread to a bone (or anywhere else), it can mean that the individual has metastatic breast cancer.

Standard clinical parameters such as tumor size, grade, lymph node involvement and tumor-node-metastasis (TNM) staging (American Joint Committee on Cancer http://www.cancerstaging.org) may correlate with outcome and serve to stratify patients with respect to (neo)adjuvant chemotherapy, immunotherapy, antibody therapy and/or radiotherapy regimens. Incorporation of molecular markers in clinical practice may define tumor subtypes that are more likely to respond to targeted therapy. However, stage-matched tumors grouped by histological or molecular subtypes may respond differently to the same treatment regimen. Additional key genetic and epigenetic alterations may exist with important etiological contributions. A more detailed understanding of the molecular mechanisms and regulatory pathways at work in cancer cells and the tumor microenvironment (TME) could dramatically improve the design of novel anti-tumor drugs and inform the selection of optimal therapeutic strategies. The development and implementation of diagnostic, prognostic and therapeutic biomarkers to characterize the biology of each tumor may assist clinicians in making important decisions with regard to individual patient care and treatment. Thus, disclosed herein are methods, compositions and systems for the analysis of coding and non-coding targets for the diagnosis, prognosis, and monitoring of a cancer.

This background information is provided for the purpose of making known information believed by the applicant to be of possible relevance to the present invention. No admission is necessarily intended, nor should be construed, that any of the preceding information constitutes prior art against the present invention.

### REFERENCE TO A SEQUENCE LISTING

This application contains references to nucleic acid sequences which have been submitted concurrently herewith as the sequence listing text file “GBX1210_IWO_ST25_Sequence_Listing.txt”, file size 283 kilobytes (kb), created on Mar. 5, 2014. The aforementioned sequence listing is hereby incorporated by reference in its entirety pursuant to 37 C.F.R. §1.52(e)(iii)(5).

### SUMMARY OF THE INVENTION

Disclosed herein in some embodiments is a method of diagnosing, prognosing, determining progression the cancer, or predicting benefit from therapy in a subject, comprising (a) assaying an expression level in a sample from the subject for a plurality of targets, wherein the plurality of targets comprises one or more targets selected from Table 1; and (b) diagnosing, prognosing, determining progression the cancer, or predicting benefit from therapy in a subject based on the expression levels of the plurality of targets. In some embodiments, the cancer is selected from the group consisting of a carcinoma, sarcoma, leukemia, lymphoma, myeloma, and a CNS tumor. In some embodiments, cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a thyroid cancer. In some embodiments, the plurality of targets comprises a coding target. In some embodiments, the coding target is an exonic sequence. In some embodiments, the plurality of targets comprises a non-coding target. In some embodiments, the non-coding target comprises an intronic sequence or partially overlaps an intronic sequence. In some embodiments, the non-coding target comprises a sequence within the UTR or partially overlaps with a UTR sequence. In some embodiments, the target comprises a nucleic acid sequence. In some embodiments, the nucleic acid sequence is a DNA sequence. In some embodiments, the nucleic acid sequence is an RNA sequence. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the diagnosing, prognosing, determining progression the cancer, or predicting benefit from therapy includes determining the malignancy of the cancer. In some embodiments, the diagnosing, prognosing, determining progression the cancer, or predicting benefit from therapy includes determining the stage of the cancer. In some embodiments, the diagnosing, prognosing, determining progression the cancer, or predicting benefit from therapy includes assessing the risk of cancer recurrence. In some embodiments, determining the treatment for the cancer includes determining the efficacy of treatment. In some embodiments, the method further comprises sequencing the plurality of targets. In some embodiments, the method further comprises hybridizing the plurality of targets to a solid support. In some embodiments, the solid support is a bead or array. In some embodiments, assaying the expression level of a plurality of targets may comprise the use of a probe set. In some embodiments, assaying the expression level may comprise the use of a classifier. The classifier may comprise a probe selection region (PSR). In some embodiments, the classifier may comprise the use of an algorithm. The algorithm may comprise a machine learning algorithm. In some embodiments, assaying the expression level may also comprise sequencing the plurality of targets.

Disclosed herein in some embodiments is a method of determining a treatment for a cancer in a subject, comprising (a) assaying an expression level in a sample from the subject for a plurality of targets, wherein the plurality of targets comprises one or more targets selected from Table 1; and (b) determining the treatment for the cancer based on the expression level of the plurality of targets. In some embodiments, the cancer is selected from the group consisting of a carcinoma, sarcoma, leukemia, lymphoma, myeloma, and a CNS tumor. In some embodiments, cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a thyroid cancer. In some embodiments, the plurality of targets comprises a coding target. In some embodiments, the coding target is an exonic sequence. In some embodiments, the plurality of targets comprises a non-coding target. In some embodiments, the non-coding target comprises an intronic sequence or partially overlaps an intronic sequence. In some embodiments, the non-coding target comprises a sequence within the UTR or partially overlaps with a UTR sequence. In some embodiments, the target comprises a nucleic acid sequence. In some embodiments, the nucleic acid sequence is a DNA sequence. In some embodiments, the nucleic acid sequence is an RNA sequence. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the diagnosing, prognosing, determining progression the cancer, or predicting benefit from therapy includes determining the malignancy of the cancer. In some embodiments, the diagnosing, prognosing, determining progression the cancer, or predicting benefit from therapy includes determining the stage of the cancer. In some embodiments, the diagnosing, prognosing, determining progression the cancer, or predicting benefit from therapy includes assessing the risk of cancer recurrence. In some embodiments, determining the treatment for the cancer includes determining the efficacy of treatment. In some embodiments, the method further comprises sequencing the plurality of targets. In some embodiments, the method further comprises hybridizing the plurality of targets to a solid support. In some embodiments, the solid support is a bead or array. In some embodiments, assaying the expression level of a plurality of targets may comprise the use of a probe set. In some embodiments, assaying the expression level may comprise the use of a classifier. The classifier may comprise a probe selection region (PSR). In some embodiments, the classifier may comprise the use of an algorithm. The algorithm may comprise a machine learning algorithm. In some embodiments, assaying the expression level may also comprise amplifying the plurality of targets. In some embodiments, assaying the expression level may also comprise quantifying the plurality of targets.

Further disclosed herein in some embodiments is a probe set for assessing a cancer status of a subject comprising a plurality of probes, wherein the probes in the set are capable of detecting an expression level of one or more targets selected from Table 1, wherein the expression level determines the cancer status of the subject with at least 40% specificity. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a thyroid cancer. In some embodiments, the probe set further comprises a probe capable of detecting an expression level of at least one coding target. In some embodiments, the coding target is an exonic sequence. In some embodiments, the probe set further comprises a probe capable of detecting an expression level of at least one non-coding target. In some embodiments, the non-coding target is an intronic sequence or partially overlaps with an intronic sequence. In some embodiments, the non-coding target is a UTR sequence or partially overlaps with a UTR sequence. In some embodiments, assessing the cancer status includes assessing cancer recurrence risk. In some embodiments, assessing the cancer status includes determining a treatment modality. In some embodiments, assessing the cancer status includes determining the efficacy of treatment. In some embodiments, the target is a nucleic acid sequence. In some embodiments, the nucleic acid sequence is a DNA sequence. In some embodiments, the nucleic acid sequence is an RNA sequence. In some embodiments, the probes are between about 15 nucleotides and about 500 nucleotides in length. In some embodiments, the probes are between about 15 nucleotides and about 450 nucleotides in length. In some embodiments, the probes are between about 15 nucleotides and about 400 nucleotides in length. In some embodiments, the probes are between about 15 nucleotides and about 350 nucleotides in length. In some embodiments, the probes are between about 15 nucleotides and about 300 nucleotides in length. In some embodiments, the probes are between about 15 nucleotides and about 250 nucleotides in length. In some embodiments, the probes are between about 15 nucleotides and about 200 nucleotides in length. In some embodiments, the probes are at least 15 nucleotides in length. In some embodiments, the probes are at least 25 nucleotides in length. In some embodiments, the expression level determines the cancer status of the subject with at least 50% specificity. In some embodiments, the expression level determines the cancer status of the subject with at least 60% specificity. In some embodiments, the expression level determines the cancer status of the subject with at least 65% specificity. In some embodiments, the expression level determines the cancer status of the subject with at least 70% specificity. In some embodiments, the expression level determines the cancer status of the subject with at least 75% specificity. In some embodiments, the expression level determines the cancer status of the subject with at least 80% specificity. In some embodiments, the expression level determines the cancer status of the subject with at least 85% specificity. In some embodiments, the non-coding target is a non-coding RNA transcript and the non-coding RNA transcript is non-polyadenylated.

Further disclosed herein in some embodiments is a system for analyzing a cancer, comprising: (a) a probe set comprising a plurality of target sequences, wherein (i) the plurality of target sequences hybridizes to one or more targets selected from Table 1; or (ii) the plurality of target sequences comprises one or more target sequences selected from Table 1; and (b) a computer model or algorithm for analyzing an expression level and/or expression profile of the target hybridized to the probe in a sample from a subject suffering from a cancer. In some embodiments, the system further comprises an electronic memory for capturing and storing an expression profile. In some embodiments, the system further comprises a computer-processing device, optionally connected to a computer network. In some embodiments, the system further comprises a software module executed by the computer-processing device to analyze an expression profile. In some embodiments, the system further comprises a software module executed by the computer-processing device to compare the expression profile to a standard or control. In some embodiments, the system further comprises a software module executed by the computer-processing device to determine the expression level of the target. In some embodiments, the system further comprises a machine to isolate the target or the probe from the sample. In some embodiments, the system further comprises a machine to sequence the target or the probe. In some embodiments, the system further comprises a machine to amplify the target or the probe. In some embodiments, the system further comprises a label that specifically binds to the target, the probe, or a combination thereof. In some embodiments, the system further comprises a software module executed by the computer-processing device to transmit an analysis of the expression profile to the individual or a medical professional treating the individual. In some embodiments, the system further comprises a software module executed by the computer-processing device to transmit a diagnosis or prognosis to the individual or a medical professional treating the individual. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the system further comprises a sequence for sequencing the plurality of targets. In some embodiments, the system further comprises an instrument for amplifying the plurality of targets. In some embodiments, the system further comprises a label for labeling the plurality of targets.

Further disclosed herein in some embodiments is a method of analyzing a cancer in an individual in need thereof, comprising: (a) obtaining an expression profile from a sample obtained from the individual, wherein the expression profile comprises one or more targets selected from Table 1; and (b) comparing the expression profile from the sample to an expression profile of a control or standard. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the method further comprises providing diagnostic or prognostic information to the individual about the cardiovascular disorder based on the comparison. In some embodiments, the method further comprises diagnosing the individual with a cancer if the expression profile of the sample (a) deviates from the control or standard from a healthy individual or population of healthy individuals, or (b) matches the control or standard from an individual or population of individuals who have or have had the cancer. In some embodiments, the method further comprises predicting the susceptibility of the individual for developing a cancer based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises prescribing a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises altering a treatment regimen prescribed or administered to the individual based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, the method further comprises quantifying the expression level of the plurality of targets. In some embodiments, the method further comprises labeling the plurality of targets. In some embodiments, assaying the expression level of a plurality of targets may comprise the use of a probe set. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets.

Disclosed herein in some embodiments is a method of diagnosing cancer in an individual in need thereof, comprising (a) obtaining an expression profile from a sample obtained from the individual, wherein the expression profile comprises one or more targets selected from Table 1; (b) comparing the expression profile from the sample to an expression profile of a control or standard; and (c) diagnosing a cancer in the individual if the expression profile of the sample (i) deviates from the control or standard from a healthy individual or population of healthy individuals, or (ii) matches the control or standard from an individual or population of individuals who have or have had the cancer. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, the method further comprises quantifying the expression level of the plurality of targets. In some embodiments, the method further comprises labeling the plurality of targets. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets.

Further disclosed herein in some embodiments is a method of predicting whether an individual is susceptible to developing a cancer, comprising (a) obtaining an expression profile from a sample obtained from the individual, wherein the expression profile comprises one or more targets selected from Table 1; (b) comparing the expression profile from the sample to an expression profile of a control or standard; and (c) predicting the susceptibility of the individual for developing a cancer based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets. In some embodiments, obtaining the expression level may also comprise amplifying the plurality of targets. In some embodiments, obtaining the expression level may also comprise quantifying the plurality of targets.

Further disclosed herein in some embodiments is a method of predicting an individual's response to a treatment regimen for a cancer, comprising (a) obtaining an expression profile from a sample obtained from the individual, wherein the expression profile comprises one or more targets selected from Table 1; (b) comparing the expression profile from the sample to an expression profile of a control or standard; and (c) predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, the method further comprises quantifying the target, the probe, or any combination thereof. In some embodiments, the method further comprises labeling the target, the probe, or any combination thereof. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets. In some embodiments, obtaining the expression level may also comprise amplifying the plurality of targets. In some embodiments, obtaining the expression level may also comprise quantifying the plurality of targets.

Disclosed herein in some embodiments is a method of prescribing a treatment regimen for a cancer to an individual in need thereof, comprising (a) obtaining an expression profile from a sample obtained from the individual, wherein the expression profile comprises one or more targets selected from Table 1; (b) comparing the expression profile from the sample to an expression profile of a control or standard; and (c) prescribing a treatment regimen based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the plurality of targets comprises at least 30 targets selected from Table 1. In some embodiments, the plurality of targets comprises at least 35 targets selected from Table 1. In some embodiments, the plurality of targets comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the method further comprises quantifying the expression level of the plurality of targets. In some embodiments, the method further comprises labeling the plurality of targets. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets. In some embodiments, obtaining the expression level may also comprise amplifying the plurality of targets. In some embodiments, obtaining the expression level may also comprise quantifying the plurality of targets.

Further disclosed herein is a classifier for analyzing a cancer, wherein the classifier has an AUC value of at least about 0.60. The AUC of the classifier may be at least about 0.60, 0.61, 0.62, 0.63, 0.64, 0.65, 0.66, 0.67, 0.68, 0.69, 0.70 or more. The AUC of the classifier may be at least about 0.71, 0.72, 0.73, 0.74, 0.75, 0.76, 0.77, 0.78, 0.79, 0.80 or more. The AUC of the classifier may be at least about 0.81, 0.82, 0.83, 0.84, 0.85, 0.86, 0.87, 0.88, 0.89, 0.90 or more. The AUC of the classifier may be at least about 0.91, 0.92, 0.93, 0.94, 0.95, 0.96, 0.97, 0.98, 0.99 or more. The 95% CI of a classifier or biomarker may be between about 1.10 to 1.70. In some instances, the difference in the range of the 95% CI for a biomarker or classifier is between about 0.25 to about 0.50, between about 0.27 to about 0.47, or between about 0.30 to about 0.45.

Further disclosed herein is a classifier for analyzing a cancer, wherein the classifier has an AUC value of at least about 0.60. The AUC of the classifier may be at least about 0.60, 0.61, 0.62, 0.63, 0.64, 0.65, 0.66, 0.67, 0.68, 0.69, 0.70 or more. The AUC of the classifier may be at least about 0.71, 0.72, 0.73, 0.74, 0.75, 0.76, 0.77, 0.78, 0.79, 0.80 or more. The AUC of the classifier may be at least about 0.81, 0.82, 0.83, 0.84, 0.85, 0.86, 0.87, 0.88, 0.89, 0.90 or more. The AUC of the classifier may be at least about 0.91, 0.92, 0.93, 0.94, 0.95, 0.96, 0.97, 0.98, 0.99 or more. The 95% CI of a classifier or biomarker may be between about 1.10 to 1.70. In some instances, the difference in the range of the 95% CI for a biomarker or classifier is between about 0.25 to about 0.50, between about 0.27 to about 0.47, or between about 0.30 to about 0.45.

Further disclosed herein is a method for analyzing a cancer, comprising use of one or more classifiers, wherein the significance of the one or more classifiers is based on one or more metrics selected from the group comprising AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the one or more classifiers may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) and Multivariable Analysis Hazard Ratio P-value (mvaHRPval). The significance of the one or more classifiers may be based on three or more metrics selected from the group comprising AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval).

The one or more metrics may comprise AUC. The one or more metrics may comprise AUC and AUC P-value. The one or more metrics may comprise AUC P-value and Wilcoxon Test P-value. The one or more metrics may comprise Wilcoxon Test P-value. The one or more metrics may comprise AUC and Univariable Analysis Odds Ratio P-value (uvaORPval). The one or more metrics may comprise multivariable analysis Odds Ratio P-value (mvaORPval) and Multivariable Analysis Hazard Ratio P-value (mvaHRPval). The one or more metrics may comprise AUC and Multivariable Analysis Hazard Ratio P-value (mvaHRPval). The one or more metrics may comprise Wilcoxon Test P-value and Multivariable Analysis Hazard Ratio P-value (mvaHRPval).

The clinical significance of the classifier may be based on the AUC value. The AUC of the classifier may be at least about about 0.60, 0.61, 0.62, 0.63, 0.64, 0.65, 0.66, 0.67, 0.68, 0.69, 0.70 or more. The AUC of the classifier may be at least about 0.71, 0.72, 0.73, 0.74, 0.75, 0.76, 0.77, 0.78, 0.79, 0.80 or more. The AUC of the classifier may be at least about 0.81, 0.82, 0.83, 0.84, 0.85, 0.86, 0.87, 0.88, 0.89, 0.90 or more. The AUC of the classifier may be at least about 0.91, 0.92, 0.93, 0.94, 0.95, 0.96, 0.97, 0.98, 0.99 or more. The 95% CI of a classifier or biomarker may be between about 1.10 to 1.70. In some instances, the difference in the range of the 95% CI for a biomarker or classifier is between about 0.25 to about 0.50, between about 0.27 to about 0.47, or between about 0.30 to about 0.45.

The clinical significance of the classifier may be based on Univariable Analysis Odds Ratio P-value (uvaORPval). The Univariable Analysis Odds Ratio P-value (uvaORPval) of the classifier may be between about 0-0.4. The Univariable Analysis Odds Ratio P-value (uvaORPval)) of the classifier may be between about 0-0.3. The Univariable Analysis Odds Ratio P-value (uvaORPval) of the classifier may be between about 0-0.2. The Univariable Analysis Odds Ratio P-value (uvaORPval) of the classifier may be less than or equal to 0.25, 0.22, 0.21, 0.20, 0.19, 0.18, 0.17, 0.16, 0.15, 0.14, 0.13, 0.12, 0.11. The Univariable Analysis Odds Ratio P-value (uvaORPval) of the classifier may be less than or equal to 0.10, 0.09, 0.08, 0.07, 0.06, 0.05, 0.04, 0.03, 0.02, 0.01. The Univariable Analysis Odds Ratio P-value (uvaORPval) of the classifier may be less than or equal to 0.009, 0.008, 0.007, 0.006, 0.005, 0.004, 0.003, 0.002, 0.001.

The clinical significance of the classifier may be based on multivariable analysis Odds Ratio P-value (mvaORPval). The multivariable analysis Odds Ratio P-value (mvaORPval) of the classifier may be between about 0-1. The multivariable analysis Odds Ratio P-value (mvaORPval) of the classifier may be between about 0-0.9. The multivariable analysis Odds Ratio P-value (mvaORPval)) of the classifier may be between about 0-0.8. The multivariable analysis Odds Ratio P-value (mvaORPval) of the classifier may be less than or equal to 0.90, 0.88, 0.86, 0.84, 0.82, 0.80. The multivariable analysis Odds Ratio P-value (mvaORPval)) of the classifier may be less than or equal to 0.78, 0.76, 0.74, 0.72, 0.70, 0.68, 0.66, 0.64, 0.62, 0.60, 0.58, 0.56, 0.54, 0.52, 0.50. The multivariable analysis Odds Ratio P-value (mvaORPval) of the classifier may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The multivariable analysis Odds Ratio P-value (mvaORPval) of the classifier may be less than or equal to 0.10, 0.09, 0.08, 0.07, 0.06, 0.05, 0.04, 0.03, 0.02, 0.01. The multivariable analysis Odds Ratio P-value (mvaORPval) of the classifier may be less than or equal to 0.009, 0.008, 0.007, 0.006, 0.005, 0.004, 0.003, 0.002, 0.001.

The clinical significance of the classifier may be based on the Kaplan Meier P-value (KM P-value). The Kaplan Meier P-value (KM P-value) of the classifier may be between about 0-0.8. The Kaplan Meier P-value (KM P-value) of the classifier may be between about 0-0.7. The Kaplan Meier P-value (KM P-value) of the classifier may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Kaplan Meier P-value (KM P-value) of the classifier may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Kaplan Meier P-value (KM P-value) of the classifier may be less than or equal to 0.10, 0.09, 0.08, 0.07, 0.06, 0.05, 0.04, 0.03, 0.02, 0.01. The Kaplan Meier P-value (KM P-value) of the classifier may be less than or equal to 0.009, 0.008, 0.007, 0.006, 0.005, 0.004, 0.003, 0.002, 0.001.

The clinical significance of the classifier may be based on the survival AUC value (survAUC). The survival AUC value (survAUC) of the classifier may be between about 0-1. The survival AUC value (survAUC) of the classifier may be between about 0-0.9. The survival AUC value (survAUC) of the classifier may be less than or equal to 1, 0.98, 0.96, 0.94, 0.92, 0.90, 0.88, 0.86, 0.84, 0.82, 0.80. The survival AUC value (survAUC) of the classifier may be less than or equal to 0.80, 0.78, 0.76, 0.74, 0.72, 0.70, 0.68, 0.66, 0.64, 0.62, 0.60, 0.58, 0.56, 0.54, 0.52, 0.50. The survival AUC value (survAUC) of the classifier may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The survival AUC value (survAUC) of the classifier may be less than or equal to 0.10, 0.09, 0.08, 0.07, 0.06, 0.05, 0.04, 0.03, 0.02, 0.01. The survival AUC value (survAUC) of the classifier may be less than or equal to 0.009, 0.008, 0.007, 0.006, 0.005, 0.004, 0.003, 0.002, 0.001.

The clinical significance of the classifier may be based on the Univariable Analysis Hazard Ratio P-value (uvaHRPval). The Univariable Analysis Hazard Ratio P-value (uvaHRPval) of the classifier may be between about 0-0.4. The Univariable Analysis Hazard Ratio P-value (uvaHRPval) of the classifier may be between about 0-0.3. The Univariable Analysis Hazard Ratio P-value (uvaHRPval) of the classifier may be less than or equal to 0.40, 0.38, 0.36, 0.34, 0.32. The Univariable Analysis Hazard Ratio P-value (uvaHRPval) of the classifier may be less than or equal to 0.30, 0.29, 0.28, 0.27, 0.26, 0.25, 0.24, 0.23, 0.22, 0.21, 0.20. The Univariable Analysis Hazard Ratio P-value (uvaHRPval) of the classifier may be less than or equal to 0.19, 0.18, 0.17, 0.16, 0.15, 0.14, 0.13, 0.12, 0.11. The Univariable Analysis Hazard Ratio P-value (uvaHRPval) of the classifier may be less than or equal to 0.10, 0.09, 0.08, 0.07, 0.06, 0.05, 0.04, 0.03, 0.02, 0.01. The Univariable Analysis Hazard Ratio P-value (uvaHRPval) of the classifier may be less than or equal to 0.009, 0.008, 0.007, 0.006, 0.005, 0.004, 0.003, 0.002, 0.001.

The clinical significance of the classifier may be based on the Multivariable Analysis Hazard Ratio P-value (mvaHRPval)mva HRPval. The Multivariable Analysis Hazard Ratio P-value (mvaHRPval)mva HRPval of the classifier may be between about 0-1. The Multivariable Analysis Hazard Ratio P-value (mvaHRPval)mva HRPval of the classifier may be between about 0-0.9. The Multivariable Analysis Hazard Ratio P-value (mvaHRPval)mva HRPval of the classifier may be less than or equal to 1, 0.98, 0.96, 0.94, 0.92, 0.90, 0.88, 0.86, 0.84, 0.82, 0.80. The Multivariable Analysis Hazard Ratio P-value (mvaHRPval)mva HRPval of the classifier may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Multivariable Analysis Hazard Ratio P-value (mvaHRPval)mva HRPval of the classifier may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Multivariable Analysis Hazard Ratio P-value (mvaHRPval)mva HRPval of the classifier may be less than or equal to 0.10, 0.09, 0.08, 0.07, 0.06, 0.05, 0.04, 0.03, 0.02, 0.01. The Multivariable Analysis Hazard Ratio P-value (mvaHRPval)mva HRPval of the classifier may be less than or equal to 0.009, 0.008, 0.007, 0.006, 0.005, 0.004, 0.003, 0.002, 0.001.

The clinical significance of the classifier may be based on the Multivariable Analysis Hazard Ratio P-value (mvaHRPval). The Multivariable Analysis Hazard Ratio P-value (mvaHRPval) of the classifier may be between about 0 to about 0.60. significance of the classifier may be based on the Multivariable Analysis Hazard Ratio P-value (mvaHRPval). The Multivariable Analysis Hazard Ratio P-value (mvaHRPval) of the classifier may be between about 0 to about 0.50. significance of the classifier may be based on the Multivariable Analysis Hazard Ratio P-value (mvaHRPval). The Multivariable Analysis Hazard Ratio P-value (mvaHRPval) of the classifier may be less than or equal to 0.50, 0.47, 0.45, 0.43, 0.40, 0.38, 0.35, 0.33, 0.30, 0.28, 0.25, 0.22, 0.20, 0.18, 0.16, 0.15, 0.14, 0.13, 0.12, 0.11, 0.10. The Multivariable Analysis Hazard Ratio P-value (mvaHRPval) of the classifier may be less than or equal to 0.10, 0.09, 0.08, 0.07, 0.06, 0.05, 0.04, 0.03, 0.02, 0.01. The Multivariable Analysis Hazard Ratio P-value (mvaHRPval) of the classifier may be less than or equal to 0.01, 0.009, 0.008, 0.007, 0.006, 0.005, 0.004, 0.003, 0.002, 0.001.

The method may further comprise determining an expression profile based on the one or more classifiers. The method may further comprise providing a sample from a subject. The subject may be a healthy subject. The subject may be suffering from a cancer or suspected of suffering from a cancer. The method may further comprise diagnosing a cancer in a subject based on the expression profile or classifier. The method may further comprise treating a cancer in a subject in need thereof based on the expression profile or classifier. The method may further comprise determining a treatment regimen for a cancer in a subject in need thereof based on the expression profile or classifier. The method may further comprise prognosing a cancer in a subject based on the expression profile or classifier.

Further disclosed herein is a kit for analyzing a cancer, comprising (a) a probe set comprising a plurality of target sequences, wherein the plurality of target sequences comprises at least one target sequence listed in Table 1; and (b) a computer model or algorithm for analyzing an expression level and/or expression profile of the target sequences in a sample. In some embodiments, the kit further comprises a computer model or algorithm for correlating the expression level or expression profile with disease state or outcome. In some embodiments, the kit further comprises a computer model or algorithm for designating a treatment modality for the individual. In some embodiments, the kit further comprises a computer model or algorithm for normalizing expression level or expression profile of the target sequences. In some embodiments, the kit further comprises a computer model or algorithm comprising a robust multichip average (RMA), probe logarithmic intensity error estimation (PLIER), non-linear fit (NLFIT) quantile-based, nonlinear normalization, or a combination thereof. In some embodiments, the plurality of target sequences comprises at least 5 target sequences selected from Table 1. In some embodiments, the plurality of target sequences comprises at least 10 target sequences selected from Table 1. In some embodiments, the plurality of target sequences comprises at least 15 target sequences selected from Table 1. In some embodiments, the plurality of target sequences comprises at least 20 target sequences selected from Table 1. In some embodiments, the plurality of target sequences comprises at least 30 target sequences selected from Table 1. In some embodiments, the plurality of target sequences comprises at least 35 target sequences selected from Table 1. In some embodiments, the plurality of targets comprises at least 40 target sequences selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer.

Further disclosed herein is a kit for analyzing a cancer, comprising (a) a probe set comprising a plurality of target sequences, wherein the plurality of target sequences hybridizes to one or more targets selected from Table 1; and (b) a computer model or algorithm for analyzing an expression level and/or expression profile of the target sequences in a sample. In some embodiments, the kit further comprises a computer model or algorithm for correlating the expression level or expression profile with disease state or outcome. In some embodiments, the kit further comprises a computer model or algorithm for designating a treatment modality for the individual. In some embodiments, the kit further comprises a computer model or algorithm for normalizing expression level or expression profile of the target sequences. In some embodiments, the kit further comprises a computer model or algorithm comprising a robust multichip average (RMA), probe logarithmic intensity error estimation (PLIER), non-linear fit (NLFIT) quantile-based, nonlinear normalization, or a combination thereof. In some embodiments, the targets comprise at least 5 targets selected from Table 1. In some embodiments, the targets comprise at least 10 targets selected from Table 1. In some embodiments, the targets comprise at least 15 targets selected from Table 1. In some embodiments, the targets comprise at least 20 targets selected from Table 1. In some embodiments, the targets comprise at least 30 targets selected from Table 1. In some embodiments, the targets comprise at least 35 targets selected from Table 1. In some embodiments, the targets comprise comprises at least 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 shows the Score Distribution for patients with and without BCR in the MSKCC Dataset.

FIG. 2A-C show the Score Distribution for patients with and without BCR the Mayo Datasets. FIG. 2A shows the Mayo Training Dataset. FIG. 2B shows the Mayo Testing Dataset. FIG. 2C shows the Mayo Validation Dataset.

FIG. 3A-C show the Score Distribution for patients with PSADT <9 months and PSADT >9 months in the Mayo Datasets. FIG. 3A shows the Mayo Training Dataset. FIG. 3B shows the Mayo Testing Dataset. FIG. 3C shows the Mayo Validation Dataset.

FIG. 4A-B shows the Discrimination Plots for patients with and without ADT Failure in the Mayo Datasets, FIG. 4A shows the Mayo Validation Dataset. FIG. 4B shows the Mayo Testing+Testing Datasets.

FIG. 5A shows the Boxplots of KNN392 GC scores for predicting presence of Gleason Grade 4 (GG4+) compared to Gleason Grade 3 (GG3) in Mayo training cohort.

FIG. 5B shows the ROC Curve of KNN392 GC scores for predicting presence of Gleason Grade 4 (GG4+) compared to Gleason Grade 3 (GG3) in Mayo training cohort.

FIG. 6A shows the Boxplots of KNN392 GC scores for predicting presence of Gleason Grade 4 (GG4+) compared to Gleason Grade 3 (GG3) in MSKCC testing cohort.

FIG. 6B shows the ROC Curve of KNN392 GC scores for predicting presence of Gleason Grade 4 (GG4+) compared to Gleason Grade 3 (GG3) in MSKCC testing cohort.

FIG. 7A shows the Boxplots of KNN104 GC scores for predicting presence of Gleason Grade 4 (GG4+) compared to Gleason Grade 3 (GG3) in Mayo discovery dataset.

FIG. 7B shows the ROC Curve of KNN104 GC scores for predicting presence of Gleason Grade 4 (GG4+) compared to Gleason Grade 3 (GG3) in Mayo discovery dataset.

FIG. 8A shows the Boxplots of KNN104 GC scores for predicting presence of Gleason Grade 4 (GG4+) compared to Gleason Grade 3 (GG3) in Mayo validation dataset.

FIG. 8B shows the ROC Curve of KNN104 GC scores for predicting presence of Gleason Grade 4 (GG4+) compared to Gleason Grade 3 (GG3) in Mayo validation dataset.

FIG. 9A shows the Boxplots of KNN41 GC scores for predicting non-malignant versus tumor samples in MSKCC, DKFZ and ICR training cohort.

FIG. 9B shows the ROC Curve of KNN41 GC scores for predicting non-malignant versus tumor samples in MSKCC, DKFZ and ICR training cohort

FIG. 10A shows the Boxplots for the prediction of MET (AIX=0.82 [071-0.93, p=1.60e-05]). MET endpoint acts as surrogate of Hormone Treatment Failure.

FIG. 10B shows the receiver operating characteristic curve for the prediction of MET (AUC=0.82 [0.71-0.93, p 1.60e-05]). MET endpoint acts as surrogate of Hormone Treatment Failure.

FIG. 11 shows the MVA Forest Plot. Multivariable analysis odds ratios with 95% confidence intervals for the MET endpoint. The nuiltivariable analysis included the genomic signature, pre-operative PSA, Gleason Score, seminal vesicic. invasion (SA/4 surgical margin status (SMS), and extra capillary extension (ECE).

FIG. 12 shows the Kaplan Meier curve showing differences in the MET-free survival from the time of initiation of salvage hormone treatment of patience with high and low prediction scores (P-Value=4.82e-04). MET endpoint acts as surrogate of Hormone Treatment Failure.

FIG. 13A shows the Boxplots for the prediction of MET in patients which received salvage or adjuvant radiation (AUC=0.65 [0.49-0.80]). MET endpoint acts as surrogate of Radiation Treatment Failure.

FIG. 13B shows receiver operating characteristic curve for the prediction of MET in patients which received salvage or adjuvant radiation (AUC=0.65 [0.49-0.80]). MET endpoint acts as surrogate of Radiation Treatment Failure.

FIG. 14A shows the Boxplots off KNN34 scores in the DFKZ validation dataset along with the selected model ca point (shown by the dashed line).

FIG. 14B shows the Boxplots off KNN34 scores in the MSKCC validation dataset along with the selected model cutpoint (shown by the dashed line),

FIG. 14C shows the Boxplots off KNN34 scores in the ICR validation dataset along with the selected model cutpoint (shown by the dashed line).

FIG. 14D shows the Boxplots off KNN34 scores in the Mayo validation dataset along with the selected model cutpoint (shown by the dashed line).

FIG. 15A shows a Boxplot of RF72 GC scores for predicting presence of Gleason Grade 4 (GG4+) compared to Gleason Grade 3 (GG3) in Mayo training and DKFZ cohort.

FIG. 15B shows ROC Curve of RF72 GC scores for predicting presence of Gleason Grade 4 (GG4+) compared to Gleason Grade 3 (GG3) in Mayo training and DKFZ cohort.

FIG. 16A shows the Boxplots of RF72 GC scores for predicting presence of Gleason Grade 4 (GG4+) compared to Gleason Grade 3 (GG3) in the independent Mayo validation set.

FIG. 16B shows ROC Curve of RF72 GC scores for predicting presence of Gleason Grade 4 (GG4+) compared to Gleason Grade 3 (GG3) in the independent Mayo validation set.

FIG. 17A shows the Boxplots of RF132 GC scores for predicting presence of Gleason Grade 4 (GG4+) compared to Gleason Grade 3 (GG3) in Mayo training and DKFZ cohort.

FIG. 17B shows ROC Curve of RF132 GC scores for predicting presence of Gleason Grade 4 (GG4+) compared to Gleason Grade 3 (GG3) in Mayo training and DKFZ cohort.

FIG. 18A shows the Boxplots of RF132 GC scores for predicting presence of Gleason Grade 4 (GG4+) compared to Gleason Grade 3 (GG3) in Mayo independent validation dataset.

FIG. 18B shows ROC Curve of RF132 GC scores for predicting presence of Gleason Grade 4 (GG4+) compared to Gleason Grade 3 (GG3) in Mayo independent validation dataset.

### DETAILED DESCRIPTION OF THE INVENTION

The present invention discloses systems and methods for diagnosing, predicting, and/or monitoring the status or outcome of a cancer in a subject using expression-based analysis of a plurality of targets. Generally, the method comprises (a) optionally providing a sample from a subject; (b) assaying the expression level for a plurality of targets in the sample; and (c) diagnosing, predicting and/or monitoring the status or outcome of a cancer based on the expression level of the plurality of targets.

Assaying the expression level for a plurality of targets in the sample may comprise applying the sample to a microarray. In some instances, assaying the expression level may comprise the use of an algorithm. The algorithm may be used to produce a classifier. Alternatively, the classifier may comprise a probe selection region. In some instances, assaying the expression level for a plurality of targets comprises detecting and/or quantifying the plurality of targets. In some embodiments, assaying the expression level for a plurality of targets comprises sequencing the plurality of targets. In some embodiments, assaying the expression level for a plurality of targets comprises amplifying the plurality of targets. In some embodiments, assaying the expression level for a plurality of targets comprises quantifying the plurality of targets. In some embodiments, assaying the expression level for a plurality of targets comprises conducting a multiplexed reaction on the plurality of targets.

In some instances, the plurality of targets comprises one or more targets selected from Table 1. In some instances, the plurality of targets comprises at least about 2, at least about 3, at least about 4, at least about 5, at least about 6, at least about 7, at least about 8, at least about 9, or at least about 10 targets selected from Table 1. In other instances, the plurality of targets comprises at least about 12, at least about 15, at least about 17, at least about 20, at least about 22, at least about 25, at least about 27, at least about 30, at least about 32, at least about 35, at least about 37, or at least about 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some instances, the plurality of targets comprises a coding target, non-coding target, or any combination thereof. In some instances, the coding target comprises an exonic sequence. In other instances, the non-coding target comprises a non-exonic sequence. In some instances, the non-exonic sequence comprises an untranslated region (e.g., UTR), intronic region, intergenic region, or any combination thereof. Alternatively, the plurality of targets comprises an anti-sense sequence. In other instances, the plurality of targets comprises a non-coding RNA transcript.

Further disclosed herein, is a probe set for diagnosing, predicting, and/or monitoring a cancer in a subject. In some instances, the probe set comprises a plurality of probes capable of detecting an expression level of one or more targets selected from Table 1, wherein the expression level determines the cancer status of the subject with at least about 45% specificity. In some instances, detecting an expression level comprise detecting gene expression, protein expression, or any combination thereof. In some instances, the plurality of targets comprises one or more targets selected from Table 1. In some instances, the plurality of targets comprises at least about 2, at least about 3, at least about 4, at least about 5, at least about 6, at least about 7, at least about 8, at least about 9, or at least about 10 targets selected from Table 1. In other instances, the plurality of targets comprises at least about 12, at least about 15, at least about 17, at least about 20, at least about 22, at least about 25, at least about 27, at least about 30, at least about 32, at least about 35, at least about 37, or at least about 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some instances, the plurality of targets comprises a coding target, non-coding target, or any combination thereof. In some instances, the coding target comprises an exonic sequence. In other instances, the non-coding target comprises a non-exonic sequence. In some instances, the non-exonic sequence comprises an untranslated region (e.g., UTR), intronic region, intergenic region, or any combination thereof. Alternatively, the plurality of targets comprises an anti-sense sequence. In other instances, the plurality of targets comprises a non-coding RNA transcript.

Further disclosed herein are methods for characterizing a patient population. Generally, the method comprises: (a) providing a sample from a subject; (b) assaying the expression level for a plurality of targets in the sample; and (c) characterizing the subject based on the expression level of the plurality of targets. In some instances, the plurality of targets comprises one or more targets selected from Table 1. In some instances, the plurality of targets comprises at least about 2, at least about 3, at least about 4, at least about 5, at least about 6, at least about 7, at least about 8, at least about 9, or at least about 10 targets selected from Table 1. In other instances, the plurality of targets comprises at least about 12, at least about 15, at least about 17, at least about 20, at least about 22, at least about 25, at least about 27, at least about 30, at least about 32, at least about 35, at least about 37, or at least about 40 targets selected from Table 1. In some embodiments, the plurality of targets comprises at least 50 targets selected from Table 1. In some embodiments, the plurality of targets comprises at least 60 targets selected from Table 1. In some embodiments, the plurality of targets comprises at least 100 targets selected from Table 1. In some embodiments, the plurality of targets comprises at least 125 targets selected from Table 1. In some embodiments, the plurality of targets comprises at least 150 targets selected from Table 1. In some embodiments, the plurality of targets comprises at least 175 targets selected from Table 1. In some embodiments, the plurality of targets comprises at least 200 targets selected from Table 1. In some embodiments, the plurality of targets comprises at least 225 targets selected from Table 1. In some embodiments, the plurality of targets comprises at least 250 targets selected from Table 1. In some embodiments, the plurality of targets comprises at least 275 targets selected from Table 1. In some embodiments, the plurality of targets comprises at least 300 targets selected from Table 1. In some embodiments, the plurality of targets comprises at least 350 targets selected from Table 1. In some embodiments, the plurality of targets comprises at least 400 targets selected from Table 1. In some embodiments, the plurality of targets comprises at least 450 targets selected from Table 1. In some embodiments, the plurality of targets comprises at least 500 targets selected from Table 1. In some embodiments, the plurality of targets comprises at least 550 targets selected from Table 1. In some embodiments, the plurality of targets comprises at least 600 targets selected from Table 1. In some embodiments, the plurality of targets comprises at least 650 targets selected from Table 1. In some embodiments, the plurality of targets comprises at least 700 targets selected from Table 1. In some embodiments, the plurality of targets comprises at least 750 targets selected from Table 1. In some embodiments, the plurality of targets comprises at least 800 targets selected from Table 1. In some instances, the plurality of targets comprises a coding target, non-coding target, or any combination thereof. In some instances, the coding target comprises an exonic sequence. In other instances, the non-coding target comprises a non-exonic sequence. In some instances, the non-exonic sequence comprises an untranslated region (e.g., UTR), intronic region, intergenic region, or any combination thereof. Alternatively, the plurality of targets comprises an anti-sense sequence. In other instances, the plurality of targets comprises a non-coding RNA transcript.

In some instances, characterizing the subject comprises determining whether the subject would respond to an anti-cancer therapy. Alternatively, characterizing the subject comprises identifying the subject as a non-responder to an anti-cancer therapy. Optionally, characterizing the subject comprises identifying the subject as a responder to an anti-cancer therapy.

Before the present invention is described in further detail, it is to be understood that this invention is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present invention.

### DEFINITIONS

Unless defined otherwise or the context clearly dictates otherwise, all technical and scientific terms used herein have the same meaning as commonly understood by one of ordinary skill in the art to which this invention belongs. In describing the present invention, the following terms may be employed, and are intended to be defined as indicated below.

The term “polynucleotide” as used herein refers to a polymer of greater than one nucleotide in length of ribonucleic acid (RNA), deoxyribonucleic acid (DNA), hybrid RNA/DNA, modified RNA or DNA, or RNA or DNA mimetics, including peptide nucleic acids (PNAs). The polynucleotides may be single- or double-stranded. The term includes polynucleotides composed of naturally-occurring nucleobases, sugars and covalent internucleoside (backbone) linkages as well as polynucleotides having non-naturally-occurring portions which function similarly. Such modified or substituted polynucleotides are well known in the art and for the purposes of the present invention, are referred to as “analogues.”

“Complementary” or “substantially complementary” refers to the ability to hybridize or base pair between nucleotides or nucleic acids, such as, for instance, between a sensor peptide nucleic acid or polynucleotide and a target polynucleotide. Complementary nucleotides are, generally, A and T (or A and U), or C and G. Two single-stranded polynucleotides or PNAs are said to be substantially complementary when the bases of one strand, optimally aligned and compared and with appropriate insertions or deletions, pair with at least about 80% of the bases of the other strand, usually at least about 90% to 95%, and more preferably from about 98 to 100%.

Alternatively, substantial complementarity exists when a polynucleotide may hybridize under selective hybridization conditions to its complement. Typically, selective hybridization may occur when there is at least about 65% complementarity over a stretch of at least 14 to 25 bases, for example at least about 75%, or at least about 90% complementarity.

“Preferential binding” or “preferential hybridization” refers to the increased propensity of one polynucleotide to bind to its complement in a sample as compared to a noncomplementary polymer in the sample.

Hybridization conditions may typically include salt concentrations of less than about 1M, more usually less than about 500 mM, for example less than about 200 mM. In the case of hybridization between a peptide nucleic acid and a polynucleotide, the hybridization can be done in solutions containing little or no salt. Hybridization temperatures can be as low as 5° C., but are typically greater than 22° C., and more typically greater than about 30° C., for example in excess of about 37° C. Longer fragments may require higher hybridization temperatures for specific hybridization as is known in the art. Other factors may affect the stringency of hybridization, including base composition and length of the complementary strands, presence of organic solvents and extent of base mismatching, and the combination of parameters used is more important than the absolute measure of any one alone. Other hybridization conditions which may be controlled include buffer type and concentration, solution pH, presence and concentration of blocking reagents to decrease background binding such as repeat sequences or blocking protein solutions, detergent type(s) and concentrations, molecules such as polymers which increase the relative concentration of the polynucleotides, metal ion(s) and their concentration(s), chelator(s) and their concentrations, and other conditions known in the art.

“Multiplexing” herein refers to an assay or other analytical method in which multiple analytes are assayed. In some instances, the multiple analytes are from the same sample. In some instances, the multiple analytes are assayed simultaneously. Alternatively, the multiple analytes are assayed sequentially. In some instances, assaying the multiple analytes occurs in the same reaction volume. Alternatively, assaying the multiple analytes occurs in separate or multiple reaction volumes.

A “target sequence” as used herein (also occasionally referred to as a “PSR” or “probe selection region”) refers to a region of the genome against which one or more probes can be designed. A “target sequence” may be a coding target or a non-coding target. A “target sequence” may comprise exonic and/or non-exonic sequences. Alternatively, a “target sequence” may comprise an ultraconserved region. An ultraconserved region is generally a sequence that is at least 200 base pairs and is conserved across multiple species. An ultraconserved region may be exonic or non-exonic. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof.

As used herein, a probe is any polynucleotide capable of selectively hybridizing to a target sequence or its complement, or to an RNA version of either. A probe may comprise ribonucleotides, deoxyribonucleotides, peptide nucleic acids, and combinations thereof. A probe may optionally comprise one or more labels. In some embodiments, a probe may be used to amplify one or both strands of a target sequence or an RNA form thereof, acting as a sole primer in an amplification reaction or as a member of a set of primers.

As used herein, a non-coding target may comprise a nucleotide sequence. The nucleotide sequence is a DNA or RNA sequence. A non-coding target may include a UTR sequence, an intronic sequence, or a non-coding RNA transcript. A non-coding target also includes sequences which partially overlap with a UTR sequence or an intronic sequence. A non-coding target also includes non-exonic transcripts.

As used herein, a coding target includes nucleotide sequences that encode for a protein and peptide sequences. The nucleotide sequence is a DNA or RNA sequence. The coding target includes protein-coding sequence. Protein-coding sequences include exon-coding sequences (e.g., exonic sequences).

As used herein, diagnosis of cancer may include the identification of cancer in a subject, determining the malignancy of the cancer, or determining the stage of the cancer.

As used herein, prognosis of cancer may include predicting the clinical outcome of the patient, assessing the risk of cancer recurrence, determining treatment modality, or determining treatment efficacy.

“Having” is an open-ended phrase like “comprising” and “including,” and includes circumstances where additional elements are included and circumstances where they are not.

“Optional” or “optionally” means that the subsequently described event or circumstance may or may not occur, and that the description includes instances where the event or circumstance occurs and instances in which it does not.

As used herein ‘NED’ describes a clinically distinct disease state in which patients show no evidence of disease (NED′) at least 5 years after surgery, ‘PSA’ describes a clinically distinct disease state in which patients show biochemical relapse only (two successive increases in prostate-specific antigen levels but no other symptoms of disease with at least 5 years follow up after surgery; ‘PSA’) and ‘SYS’ describes a clinically distinct disease state in which patients develop biochemical relapse and present with systemic cancer disease or metastases (‘SYS’) within five years after the initial treatment with radical prostatectomy.

The terms “METS”, “SYS”, “systemic event”, “Systemic progression”, “CR” or “Clinical Recurrence” may be used interchangeably and generally refer to patients that experience BCR (biochemical reccurrence) and that develop metastases (confirmed by bone or CT scan). The patients may experience BCR within 5 years of RP (radial prostectomy). The patients may develop metastases within 5 years of BCR. In some cases, patients regarded as METS may experience BCR after 5 years of RP.

As used herein, the term “about” refers to approximately a +/−10% variation from a given value. It is to be understood that such a variation is always included in any given value provided herein, whether or not it is specifically referred to.

Use of the singular forms “a,” “an,” and “the” include plural references unless the context clearly dictates otherwise. Thus, for example, reference to “a polynucleotide” includes a plurality of polynucleotides, reference to “a target” includes a plurality of such targets, reference to “a normalization method” includes a plurality of such methods, and the like. Additionally, use of specific plural references, such as “two,” “three,” etc., read on larger numbers of the same subject, unless the context clearly dictates otherwise.

Terms such as “connected,” “attached,” “linked” and “conjugated” are used interchangeably herein and encompass direct as well as indirect connection, attachment, linkage or conjugation unless the context clearly dictates otherwise.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values, which are about the same quantity or amount as the recited value, are also within the scope of the invention, as are ranges based thereon. Where a combination is disclosed, each sub-combination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

### Coding and Non-Coding Targets

The methods disclosed herein often comprise assaying the expression level of a plurality of targets. The plurality of targets may comprise coding targets and/or non-coding targets of a protein-coding gene or a non protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non protein-coding gene structure may comprise an exon and intron. Usually, the exon region of a non protein-coding gene primarily contains a UTR. The non protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets may be differentially expressed. In some instances, a plurality of probe selection regions (PSRs) is differentially expressed.

In some instances, the plurality of targets comprises one or more targets selected from Table 1. In some instances, the plurality of targets comprises at least about 2, at least about 3, at least about 4, at least about 5, at least about 6, at least about 7, at least about 8, at least about 9, or at least about 10 targets selected from Table 1. In other instances, the plurality of targets comprises at least about 12, at least about 15, at least about 17, at least about 20, at least about 22, at least about 25, at least about 27, at least about 30, at least about 32, at least about 35, at least about 37, or at least about 40 targets selected from Table 1. The plurality of targets may comprise about 40, 45, 50, 55, 60, 65, 70, 75, 80, 85, 90, 95, 100 or more targets selected from Table 1. The plurality of targets may comprise about 110, 120, 130, 140, 150, 160, 170, 180, 190, 200, 225, 250, 275, 300, 325, 350, 375, 400, 425, 450, 475, 500 or more targets selected from Table 1. The plurality of targets may comprise about 500, 525, 550, 575, 600, 625, 650, 675, 700, 725, 750, 775, 800, 810, 820, 830, 840, 850 or more targets selected from Table 1. In some instances, the plurality of targets comprises a coding target, non-coding target, or any combination thereof. In some instances, the coding target comprises an exonic sequence. In other instances, the non-coding target comprises a non-exonic sequence. Alternatively, a non-coding target comprises a UTR sequence, an intronic sequence, or a non-coding RNA transcript. In some instances, a non-coding target comprises sequences which partially overlap with a UTR sequence or an intronic sequence. A non-coding target also includes non-exonic transcripts. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

In some instances, the plurality of targets is at least about 70% identical to a sequence selected from SEQ ID NOs 1-853. Alternatively, the plurality of targets is at least about 80% identical to a sequence selected from SEQ ID NOS 1-853. In some instances, the plurality of targets is at least about 85% identical to a sequence selected from SEQ ID NOS 1-853. In some instances, the plurality of targets is at least about 90% identical to a sequence selected from SEQ ID NOS 1-853. Alternatively, the plurality of targets is at least about 95% identical to a sequence selected from SEQ ID NOS 1-853.

The plurality of targets may comprise one or more targets selected from a classifier disclosed herein. The classifier may be generated from one or more models or algorithms. The one or more models or algorithms may be random forest, support vector machine (SVM), k-nearest neighbor (KNN), high dimensional discriminate analysis (i-IDDA), or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a Random Forest (RF) classifier. The plurality of targets may comprise two or more targets selected from a Random Forest (RF) classifier. The plurality of targets may comprise three or more targets selected from a Random Forest (RF) classifier. The plurality of targets may comprise 5, 6, 7, 8, 9, 10 or more targets selected from a Random Forest (RF) classifier. The RF classifier may be an RF13 classifier. The RF classifier may be an RF72 classifier. The RF classifier may be an RF132 classifier.

In some instances, the plurality of targets is at least about 70% identical to a sequence selected from a target selected from a RF classifier. Alternatively, the plurality of targets is at least about 80% identical to a sequence selected from a target selected from a RF classifier. In some instances, the plurality of targets is at least about 85% identical to a sequence selected from a target selected from a RF classifier. In some instances, the plurality of targets is at least about 90% identical to a sequence selected from a target selected from a RF classifier. Alternatively, the plurality of targets is at least about 95% identical to a sequence selected from a target selected from a RF classifier. The RF classifier may be an RF13 classifier. The RF classifier may be an RF72 classifier. The RF classifier may be an RF132 classifier.

The RF13 classifier may comprise SEQ ID NO. 380, SEQ ID NO. 111, SEQ ID NO. 318, SEQ ID NO. 338, SEQ ID NO. 559, SEQ ID NO. 610, SEQ ID NO. 614, SEQ ID NO. 712, SEQ ID NO. 750, SEQ ID NO. 751, SEQ ID NO. 752, SEQ ID NO. 753, SEQ ID NO. 818, or a combination thereof. Alternatively, or additionally, the RF13 classifier may comprise SEQ ID NO. 123, SEQ ID NO. 807, SEQ ID NO. 247, SEQ ID NO. 100, SEQ ID NO. 6, SEQ ID NO. 213, SEQ ID NO. 169, SEQ ID NO. 42, SEQ ID NO. 78, SEQ ID NO. 159, SEQ ID NO. 32, SEQ ID NO. 398, SEQ ID NO. 108, or a combination thereof.

The RF72 classifier may comprise SEQ ID NO. 646, SEQ ID NO. 373, SEQ ID NO. 674, SEQ ID NO. 602, SEQ ID NO. 372, SEQ ID NO. 375, SEQ ID NO. 377, SEQ ID NO. 512, SEQ ID NO. 32, SEQ ID NO. 307, SEQ ID NO. 487, SEQ ID NO. 594, SEQ ID NO. 306, SEQ ID NO. 295, SEQ ID NO. 374, SEQ ID NO. 610, SEQ ID NO. 329, SEQ ID NO. 599, SEQ ID NO. 784, SEQ ID NO. 554, SEQ ID NO. 489, SEQ ID NO. 376, SEQ ID NO. 311, SEQ ID NO. 738, SEQ ID NO. 553, SEQ ID NO. 64, SEQ ID NO. 332, SEQ ID NO. 556, SEQ ID NO. 309, SEQ ID NO. 513, SEQ ID NO. 837, SEQ ID NO. 611, SEQ ID NO. 496, SEQ ID NO. 590, SEQ ID NO. 187, SEQ ID NO. 119, SEQ ID NO. 813, SEQ ID NO. 313, SEQ ID NO. 649, SEQ ID NO. 609, SEQ ID NO. 439, SEQ ID NO. 491, SEQ ID NO. 836, SEQ ID NO. 613, SEQ ID NO. 240, SEQ ID NO. 81, SEQ ID NO. 515, SEQ ID NO. 449, SEQ ID NO. 123, SEQ ID NO. 312, SEQ ID NO. 61, SEQ ID NO. 314, SEQ ID NO. 338, SEQ ID NO. 121, SEQ ID NO. 600, SEQ ID NO. 330, SEQ ID NO. 305, SEQ ID NO. 343, SEQ ID NO. 694, SEQ ID NO. 657, SEQ ID NO. 122, SEQ ID NO. 829, SEQ ID NO. 571, SEQ ID NO. 71, SEQ ID NO. 28, SEQ ID NO. 785, SEQ ID NO. 700, SEQ ID NO. 82, SEQ ID NO. 636, SEQ ID NO. 378, SEQ ID NO. 344, SEQ ID NO. 555, or a combination thereof.

The RF132 classifier may comprise SEQ ID NO. 373, SEQ ID NO. 646, SEQ ID NO. 602, SEQ ID NO. 372, SEQ ID NO. 307, SEQ ID NO. 375, SEQ ID NO. 377, SEQ ID NO. 487, SEQ ID NO. 32, SEQ ID NO. 374, SEQ ID NO. 306, SEQ ID NO. 784, SEQ ID NO. 295, SEQ ID NO. 311, SEQ ID NO. 594, SEQ ID NO. 376, SEQ ID NO. 496, SEQ ID NO. 489, SEQ ID NO. 64, SEQ ID NO. 567, SEQ ID NO. 309, SEQ ID NO. 332, SEQ ID NO. 553, SEQ ID NO. 31, SEQ ID NO. 554, SEQ ID NO. 513, SEQ ID NO. 119, SEQ ID NO. 314, SEQ ID NO. 512, SEQ ID NO. 611, SEQ ID NO. 610, SEQ ID NO. 63, SEQ ID NO. 813, SEQ ID NO. 338, SEQ ID NO. 836, SEQ ID NO. 305, SEQ ID NO. 609, SEQ ID NO. 556, SEQ ID NO. 652, SEQ ID NO. 240, SEQ ID NO. 187, SEQ ID NO. 121, SEQ ID NO. 66, SEQ ID NO. 829, SEQ ID NO. 515, SEQ ID NO. 658, SEQ ID NO. 803, SEQ ID NO. 199, SEQ ID NO. 491, SEQ ID NO. 81, SEQ ID NO. 378, SEQ ID NO. 703, SEQ ID NO. 573, SEQ ID NO. 648, SEQ ID NO. 700, SEQ ID NO. 312, SEQ ID NO. 71, SEQ ID NO. 123, SEQ ID NO. 649, SEQ ID NO. 590, SEQ ID NO. 804, SEQ ID NO. 122, SEQ ID NO. 330, SEQ ID NO. 128, SEQ ID NO. 516, SEQ ID NO. 593, SEQ ID NO. 599, SEQ ID NO. 57, SEQ ID NO. 636, SEQ ID NO. 777, SEQ ID NO. 647, SEQ ID NO. 343, SEQ ID NO. 308, SEQ ID NO. 161, SEQ ID NO. 94, SEQ ID NO. 837, SEQ ID NO. 105, SEQ ID NO. 695, SEQ ID NO. 785, SEQ ID NO. 99, SEQ ID NO. 367, SEQ ID NO. 20, SEQ ID NO. 238, SEQ ID NO. 168, SEQ ID NO. 527, SEQ ID NO. 442, SEQ ID NO. 672, SEQ ID NO. 682, SEQ ID NO. 239, SEQ ID NO. 156, SEQ ID NO. 705, SEQ ID NO. 186, SEQ ID NO. 334, SEQ ID NO. 278, SEQ ID NO. 379, SEQ ID NO. 4, SEQ ID NO. 541, SEQ ID NO. 160, SEQ ID NO. 761, SEQ ID NO. 706, SEQ ID NO. 25, SEQ ID NO. 577, SEQ ID NO. 297, SEQ ID NO. 555, SEQ ID NO. 248, SEQ ID NO. 825, SEQ ID NO. 67, SEQ ID NO. 637, SEQ ID NO. 612, SEQ ID NO. 540, SEQ ID NO. 313, SEQ ID NO. 745, SEQ ID NO. 588, SEQ ID NO. 273, SEQ ID NO. 514, SEQ ID NO. 449, SEQ ID NO. 645, SEQ ID NO. 207, SEQ ID NO. 490, SEQ ID NO. 591, SEQ ID NO. 805, SEQ ID NO. 760, SEQ ID NO. 23, SEQ ID NO. 576, SEQ ID NO. 244, SEQ ID NO. 310, SEQ ID NO. 846, SEQ ID NO. 759, SEQ ID NO. 131, SEQ ID NO. 120, SEQ ID NO. 109, SEQ ID NO. 237, or a combination thereof.

The plurality of targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25, 27, 30 or more targets selected from an SVM classifier. The plurality of targets may comprise 32, 35, 37, 40, 43, 45, 47, 50, 53, 55, 57, 60 or more targets selected from an SVM classifier. The SVM classifier may be an SVM58 classifier.

In some instances, the plurality of targets is at least about 70% identical to a sequence selected from a target selected from a SVM classifier. Alternatively, the plurality of targets is at least about 80% identical to a sequence selected from a target selected from a SVM classifier. In some instances, the plurality of targets is at least about 85% identical to a sequence selected from a target selected from a SVM classifier. In some instances, the plurality of targets is at least about 90% identical to a sequence selected from a target selected from a SVM classifier. Alternatively, the plurality of targets is at least about 95% identical to a sequence selected from a target selected from a SVM classifier. The SVM classifier may be an SVM58 classifier.

The SVM58 classifier may comprise SEQ ID NO. 421, SEQ ID NO. 277, SEQ ID NO. 634, SEQ ID NO. 250, SEQ ID NO. 530, SEQ ID NO. 336, SEQ ID NO. 136, SEQ ID NO. 826, SEQ ID NO. 534, SEQ ID NO. 710, SEQ ID NO. 495, SEQ ID NO. 714, SEQ ID NO. 679, SEQ ID NO. 770, SEQ ID NO. 727, SEQ ID NO. 815, SEQ ID NO. 624, SEQ ID NO. 754, SEQ ID NO. 678, SEQ ID NO. 385, SEQ ID NO. 320, SEQ ID NO. 655, SEQ ID NO. 396, SEQ ID NO. 234, SEQ ID NO. 558, SEQ ID NO. 266, SEQ ID NO. 48, SEQ ID NO. 83, SEQ ID NO. 834, SEQ ID NO. 816, SEQ ID NO. 414, SEQ ID NO. 2, SEQ ID NO. 392, SEQ ID NO. 617, SEQ ID NO. 693, SEQ ID NO. 355, SEQ ID NO. 87, SEQ ID NO. 755, SEQ ID NO. 697, SEQ ID NO. 482, SEQ ID NO. 519, SEQ ID NO. 69, SEQ ID NO. 817, SEQ ID NO. 607, SEQ ID NO. 395, SEQ ID NO. 627, SEQ ID NO. 89, SEQ ID NO. 9, SEQ ID NO. 303, SEQ ID NO. 500, SEQ ID NO. 604, SEQ ID NO. 223, SEQ ID NO. 598, SEQ ID NO. 98, SEQ ID NO. 668, SEQ ID NO. 523, SEQ ID NO. 782, SEQ ID NO. 68, or a combination thereof.

The plurality of targets may comprise one or more targets selected from an KNN classifier. The plurality of targets may comprise 2, 3, 4, 5, 6, 7, 8, 9, 10 or more targets selected from an KNN classifier. The plurality of targets may comprise 12, 13, 14, 15, 17, 20, 22, 25, 27, 30 or more targets selected from an KNN classifier. The plurality of targets may comprise 32, 35, 37, 40, 43, 45, 47, 50, 53, 55, 57, 60 or more targets selected from an KNN classifier. The plurality of targets may comprise 65, 70, 75, 80, 85, 90, 95, 100 or more targets selected from an KNN classifier. The plurality of targets may comprise 125, 150, 175, 200, 225, 250, 275, 300, 325, 350, 375, 390 or more targets selected from an KNN classifier. The KNN classifier may be a KNN392 classifier. The KNN classifier may be a KNN104 classifier. The KNN classifier may be a KNN41 classifier. The KNN classifier may be a KNN22 classifier. The KNN classifier may be a KNN34 classifier.

In some instances, the plurality of targets is at least about 70% identical to a sequence selected from a target selected from a KNN classifier. Alternatively, the plurality of targets is at least about 80% identical to a sequence selected from a target selected from a KNN classifier. In some instances, the plurality of targets is at least about 85% identical to a sequence selected from a target selected from a KNN classifier. In some instances, the plurality of targets is at least about 90% identical to a sequence selected from a target selected from a KNN classifier. Alternatively, the plurality of targets is at least about 95% identical to a sequence selected from a target selected from a KNN classifier. The KNN classifier may be a KNN392 classifier. The KNN classifier may be a KNN104 classifier. The KNN classifier may be a KNN41 classifier. The KNN classifier may be a KNN22 classifier. The KNN classifier may be a KNN34 classifier.

The KNN392 classifier may comprise SEQ ID NO. 1, SEQ ID NO. 3, SEQ ID NO. 4, SEQ ID NO. 5, SEQ ID NO. 7, SEQ ID NO. 15, SEQ ID NO. 17, SEQ ID NO. 18, SEQ ID NO. 19, SEQ ID NO. 21, SEQ ID NO. 22, SEQ ID NO. 26, SEQ ID NO. 27, SEQ ID NO. 30, SEQ ID NO. 31, SEQ ID NO. 32, SEQ ID NO. 33, SEQ ID NO. 34, SEQ ID NO. 35, SEQ ID NO. 40, SEQ ID NO. 41, SEQ ID NO. 43, SEQ ID NO. 45, SEQ ID NO. 50, SEQ ID NO. 51, SEQ ID NO. 52, SEQ ID NO. 53, SEQ ID NO. 54, SEQ ID NO. 56, SEQ ID NO. 58, SEQ ID NO. 61, SEQ ID NO. 62, SEQ ID NO. 70, SEQ ID NO. 72, SEQ ID NO. 75, SEQ ID NO. 76, SEQ ID NO. 77, SEQ ID NO. 79, SEQ ID NO. 80, SEQ ID NO. 85, SEQ ID NO. 88, SEQ ID NO. 91, SEQ ID NO. 92, SEQ ID NO. 93, SEQ ID NO. 96, SEQ ID NO. 101, SEQ ID NO. 102, SEQ ID NO. 103, SEQ ID NO. 104, SEQ ID NO. 107, SEQ ID NO. 110, SEQ ID NO. 112, SEQ ID NO. 113, SEQ ID NO. 114, SEQ ID NO. 126, SEQ ID NO. 127, SEQ ID NO. 132, SEQ ID NO. 134, SEQ ID NO. 135, SEQ ID NO. 138, SEQ ID NO. 139, SEQ ID NO. 140, SEQ ID NO. 141, SEQ ID NO. 142, SEQ ID NO. 144, SEQ ID NO. 145, SEQ ID NO. 147, SEQ ID NO. 148, SEQ ID NO. 149, SEQ ID NO. 150, SEQ ID NO. 151, SEQ ID NO. 152, SEQ ID NO. 153, SEQ ID NO. 154, SEQ ID NO. 157, SEQ ID NO. 162, SEQ ID NO. 171, SEQ ID NO. 172, SEQ ID NO. 173, SEQ ID NO. 174, SEQ ID NO. 176, SEQ ID NO. 178, SEQ ID NO. 180, SEQ ID NO. 181, SEQ ID NO. 182, SEQ ID NO. 183, SEQ ID NO. 185, SEQ ID NO. 188, SEQ ID NO. 192, SEQ ID NO. 193, SEQ ID NO. 194, SEQ ID NO. 200, SEQ ID NO. 201, SEQ ID NO. 202, SEQ ID NO. 203, SEQ ID NO. 205, SEQ ID NO. 206, SEQ ID NO. 208, SEQ ID NO. 210, SEQ ID NO. 211, SEQ ID NO. 214, SEQ ID NO. 215, SEQ ID NO. 216, SEQ ID NO. 218, SEQ ID NO. 221, SEQ ID NO. 222, SEQ ID NO. 226, SEQ ID NO. 227, SEQ ID NO. 228, SEQ ID NO. 230, SEQ ID NO. 231, SEQ ID NO. 235, SEQ ID NO. 236, SEQ ID NO. 240, SEQ ID NO. 242, SEQ ID NO. 243, SEQ ID NO. 245, SEQ ID NO. 246, SEQ ID NO. 249, SEQ ID NO. 261, SEQ ID NO. 263, SEQ ID NO. 264, SEQ ID NO. 265, SEQ ID NO. 267, SEQ ID NO. 268, SEQ ID NO. 269, SEQ ID NO. 270, SEQ ID NO. 271, SEQ ID NO. 275, SEQ ID NO. 276, SEQ ID NO. 279, SEQ ID NO. 280, SEQ ID NO. 281, SEQ ID NO. 282, SEQ ID NO. 284, SEQ ID NO. 285, SEQ ID NO. 286, SEQ ID NO. 287, SEQ ID NO. 288, SEQ ID NO. 289, SEQ ID NO. 290, SEQ ID NO. 291, SEQ ID NO. 292, SEQ ID NO. 293, SEQ ID NO. 295, SEQ ID NO. 298, SEQ ID NO. 300, SEQ ID NO. 301, SEQ ID NO. 302, SEQ ID NO. 304, SEQ ID NO. 305, SEQ ID NO. 306, SEQ ID NO. 307, SEQ ID NO. 309, SEQ ID NO. 311, SEQ ID NO. 312, SEQ ID NO. 315, SEQ ID NO. 316, SEQ ID NO. 317, SEQ ID NO. 319, SEQ ID NO. 321, SEQ ID NO. 322, SEQ ID NO. 324, SEQ ID NO. 328, SEQ ID NO. 329, SEQ ID NO. 330, SEQ ID NO. 331, SEQ ID NO. 332, SEQ ID NO. 333, SEQ ID NO. 335, SEQ ID NO. 337, SEQ ID NO. 338, SEQ ID NO. 339, SEQ ID NO. 340, SEQ ID NO. 341, SEQ ID NO. 345, SEQ ID NO. 346, SEQ ID NO. 347, SEQ ID NO. 348, SEQ ID NO. 351, SEQ ID NO. 352, SEQ ID NO. 354, SEQ ID NO. 356, SEQ ID NO. 357, SEQ ID NO. 360, SEQ ID NO. 361, SEQ ID NO. 363, SEQ ID NO. 364, SEQ ID NO. 366, SEQ ID NO. 367, SEQ ID NO. 368, SEQ ID NO. 369, SEQ ID NO. 370, SEQ ID NO. 371, SEQ ID NO. 372, SEQ ID NO. 373, SEQ ID NO. 374, SEQ ID NO. 375, SEQ ID NO. 376, SEQ ID NO. 377, SEQ ID NO. 381, SEQ ID NO. 382, SEQ ID NO. 384, SEQ ID NO. 386, SEQ ID NO. 387, SEQ ID NO. 388, SEQ ID NO. 389, SEQ ID NO. 397, SEQ ID NO. 400, SEQ ID NO. 401, SEQ ID NO. 402, SEQ ID NO. 403, SEQ ID NO. 404, SEQ ID NO. 405, SEQ ID NO. 408, SEQ ID NO. 410, SEQ ID NO. 413, SEQ ID NO. 415, SEQ ID NO. 416, SEQ ID NO. 418, SEQ ID NO. 426, SEQ ID NO. 429, SEQ ID NO. 430, SEQ ID NO. 431, SEQ ID NO. 440, SEQ ID NO. 441, SEQ ID NO. 444, SEQ ID NO. 445, SEQ ID NO. 446, SEQ ID NO. 448, SEQ ID NO. 450, SEQ ID NO. 451, SEQ ID NO. 453, SEQ ID NO. 454, SEQ ID NO. 455, SEQ ID NO. 456, SEQ ID NO. 457, SEQ ID NO. 459, SEQ ID NO. 460, SEQ ID NO. 461, SEQ ID NO. 462, SEQ ID NO. 463, SEQ ID NO. 464, SEQ ID NO. 465, SEQ ID NO. 468, SEQ ID NO. 474, SEQ ID NO. 476, SEQ ID NO. 477, SEQ ID NO. 478, SEQ ID NO. 480, SEQ ID NO. 483, SEQ ID NO. 484, SEQ ID NO. 485, SEQ ID NO. 486, SEQ ID NO. 487, SEQ ID NO. 488, SEQ ID NO. 489, SEQ ID NO. 490, SEQ ID NO. 491, SEQ ID NO. 493, SEQ ID NO. 494, SEQ ID NO. 496, SEQ ID NO. 497, SEQ ID NO. 512, SEQ ID NO. 517, SEQ ID NO. 539, SEQ ID NO. 542, SEQ ID NO. 544, SEQ ID NO. 545, SEQ ID NO. 546, SEQ ID NO. 547, SEQ ID NO. 548, SEQ ID NO. 550, SEQ ID NO. 551, SEQ ID NO. 552, SEQ ID NO. 554, SEQ ID NO. 560, SEQ ID NO. 561, SEQ ID NO. 562, SEQ ID NO. 563, SEQ ID NO. 564, SEQ ID NO. 565, SEQ ID NO. 566, SEQ ID NO. 567, SEQ ID NO. 568, SEQ ID NO. 569, SEQ ID NO. 570, SEQ ID NO. 572, SEQ ID NO. 573, SEQ ID NO. 574, SEQ ID NO. 575, SEQ ID NO. 578, SEQ ID NO. 579, SEQ ID NO. 581, SEQ ID NO. 582, SEQ ID NO. 583, SEQ ID NO. 584, SEQ ID NO. 590, SEQ ID NO. 592, SEQ ID NO. 596, SEQ ID NO. 597, SEQ ID NO. 601, SEQ ID NO. 602, SEQ ID NO. 603, SEQ ID NO. 606, SEQ ID NO. 609, SEQ ID NO. 610, SEQ ID NO. 618, SEQ ID NO. 619, SEQ ID NO. 620, SEQ ID NO. 625, SEQ ID NO. 628, SEQ ID NO. 629, SEQ ID NO. 630, SEQ ID NO. 631, SEQ ID NO. 632, SEQ ID NO. 638, SEQ ID NO. 642, SEQ ID NO. 643, SEQ ID NO. 652, SEQ ID NO. 653, SEQ ID NO. 657, SEQ ID NO. 661, SEQ ID NO. 662, SEQ ID NO. 666, SEQ ID NO. 669, SEQ ID NO. 674, SEQ ID NO. 692, SEQ ID NO. 699, SEQ ID NO. 707, SEQ ID NO. 708, SEQ ID NO. 715, SEQ ID NO. 717, SEQ ID NO. 718, SEQ ID NO. 719, SEQ ID NO. 720, SEQ ID NO. 721, SEQ ID NO. 722, SEQ ID NO. 725, SEQ ID NO. 728, SEQ ID NO. 729, SEQ ID NO. 731, SEQ ID NO. 732, SEQ ID NO. 733, SEQ ID NO. 734, SEQ ID NO. 736, SEQ ID NO. 737, SEQ ID NO. 738, SEQ ID NO. 740, SEQ ID NO. 743, SEQ ID NO. 744, SEQ ID NO. 746, SEQ ID NO. 748, SEQ ID NO. 749, SEQ ID NO. 756, SEQ ID NO. 757, SEQ ID NO. 758, SEQ ID NO. 771, SEQ ID NO. 772, SEQ ID NO. 775, SEQ ID NO. 778, SEQ ID NO. 779, SEQ ID NO. 780, SEQ ID NO. 781, SEQ ID NO. 784, SEQ ID NO. 787, SEQ ID NO. 789, SEQ ID NO. 793, SEQ ID NO. 794, SEQ ID NO. 796, SEQ ID NO. 798, SEQ ID NO. 801, SEQ ID NO. 807, SEQ ID NO. 811, SEQ ID NO. 814, SEQ ID NO. 820, SEQ ID NO. 828, SEQ ID NO. 833, SEQ ID NO. 835, SEQ ID NO. 836, SEQ ID NO. 837, SEQ ID NO. 838, SEQ ID NO. 842, SEQ ID NO. 843, SEQ ID NO. 844, SEQ ID NO. 847, SEQ ID NO. 848, SEQ ID NO. 849, SEQ ID NO. 850, SEQ ID NO. 851, SEQ ID NO. 852, SEQ ID NO. 853, or a combination thereof.

The KNN104 classifier may comprise SEQ ID NO. 222, SEQ ID NO. 646, SEQ ID NO. 807, SEQ ID NO. 674, SEQ ID NO. 821, SEQ ID NO. 316, SEQ ID NO. 443, SEQ ID NO. 294, SEQ ID NO. 575, SEQ ID NO. 358, SEQ ID NO. 783, SEQ ID NO. 798, SEQ ID NO. 582, SEQ ID NO. 602, SEQ ID NO. 702, SEQ ID NO. 126, SEQ ID NO. 34, SEQ ID NO. 364, SEQ ID NO. 795, SEQ ID NO. 8, SEQ ID NO. 459, SEQ ID NO. 383, SEQ ID NO. 628, SEQ ID NO. 365, SEQ ID NO. 768, SEQ ID NO. 307, SEQ ID NO. 477, SEQ ID NO. 618, SEQ ID NO. 341, SEQ ID NO. 258, SEQ ID NO. 236, SEQ ID NO. 580, SEQ ID NO. 663, SEQ ID NO. 653, SEQ ID NO. 327, SEQ ID NO. 46, SEQ ID NO. 622, SEQ ID NO. 411, SEQ ID NO. 373, SEQ ID NO. 95, SEQ ID NO. 542, SEQ ID NO. 390, SEQ ID NO. 261, SEQ ID NO. 549, SEQ ID NO. 326, SEQ ID NO. 651, SEQ ID NO. 726, SEQ ID NO. 493, SEQ ID NO. 650, SEQ ID NO. 375, SEQ ID NO. 843, SEQ ID NO. 445, SEQ ID NO. 190, SEQ ID NO. 758, SEQ ID NO. 717, SEQ ID NO. 179, SEQ ID NO. 626, SEQ ID NO. 406, SEQ ID NO. 664, SEQ ID NO. 479, SEQ ID NO. 205, SEQ ID NO. 225, SEQ ID NO. 174, SEQ ID NO. 381, SEQ ID NO. 492, SEQ ID NO. 229, SEQ ID NO. 299, SEQ ID NO. 665, SEQ ID NO. 170, SEQ ID NO. 306, SEQ ID NO. 830, SEQ ID NO. 432, SEQ ID NO. 184, SEQ ID NO. 730, SEQ ID NO. 584, SEQ ID NO. 374, SEQ ID NO. 407, SEQ ID NO. 788, SEQ ID NO. 842, SEQ ID NO. 453, SEQ ID NO. 461, SEQ ID NO. 350, SEQ ID NO. 276, SEQ ID NO. 424, SEQ ID NO. 535, SEQ ID NO. 595, SEQ ID NO. 33, SEQ ID NO. 427, SEQ ID NO. 831, SEQ ID NO. 399, SEQ ID NO. 691, SEQ ID NO. 819, SEQ ID NO. 356, SEQ ID NO. 65, SEQ ID NO. 409, SEQ ID NO. 538, SEQ ID NO. 735, SEQ ID NO. 452, SEQ ID NO. 771, SEQ ID NO. 608, SEQ ID NO. 391, SEQ ID NO. 44, SEQ ID NO. 447, SEQ ID NO. 799, or a combination thereof.

The KNN41 classifier may comprise: SEQ ID NO. 255, SEQ ID NO. 167, SEQ ID NO. 501, SEQ ID NO. 504, SEQ ID NO. 254, SEQ ID NO. 503, SEQ ID NO. 224, SEQ ID NO. 502, SEQ ID NO. 509, SEQ ID NO. 507, SEQ ID NO. 557, SEQ ID NO. 506, SEQ ID NO. 251, SEQ ID NO. 644, SEQ ID NO. 90, SEQ ID NO. 260, SEQ ID NO. 766, SEQ ID NO. 510, SEQ ID NO. 166, SEQ ID NO. 241, SEQ ID NO. 436, SEQ ID NO. 256, SEQ ID NO. 118, SEQ ID NO. 257, SEQ ID NO. 676, SEQ ID NO. 283, SEQ ID NO. 508, SEQ ID NO. 253, SEQ ID NO. 252, SEQ ID NO. 840, SEQ ID NO. 196, SEQ ID NO. 765, SEQ ID NO. 165, SEQ ID NO. 10, SEQ ID NO. 212, SEQ ID NO. 827, SEQ ID NO. 434, SEQ ID NO. 769, SEQ ID NO. 505, SEQ ID NO. 742, SEQ ID NO. 704, or a combination thereof.

The KNN22 classifier may comprise SEQ ID NO. 677, SEQ ID NO. 687, SEQ ID NO. 522, SEQ ID NO. 438, SEQ ID NO. 690, SEQ ID NO. 435, SEQ ID NO. 533, SEQ ID NO. 688, SEQ ID NO. 129, SEQ ID NO. 686, SEQ ID NO. 130, SEQ ID NO. 832, SEQ ID NO. 615, SEQ ID NO. 531, SEQ ID NO. 543, SEQ ID NO. 524, SEQ ID NO. 323, SEQ ID NO. 433, SEQ ID NO. 616, SEQ ID NO. 437, SEQ ID NO. 84, SEQ ID NO. 723, or a combination thereof.

The KNN34 classifier may comprise SEQ ID NO. 677, SEQ ID NO. 687, SEQ ID NO. 522, SEQ ID NO. 438, SEQ ID NO. 690, SEQ ID NO. 435, SEQ ID NO. 533, SEQ ID NO. 688, SEQ ID NO. 129, SEQ ID NO. 686, SEQ ID NO. 130, SEQ ID NO. 832, SEQ ID NO. 615, SEQ ID NO. 531, SEQ ID NO. 543, SEQ ID NO. 524, SEQ ID NO. 323, SEQ ID NO. 433, SEQ ID NO. 616, SEQ ID NO. 437, SEQ ID NO. 84, SEQ ID NO. 723, SEQ ID NO. 684, SEQ ID NO. 724, SEQ ID NO. 764, SEQ ID NO. 525, SEQ ID NO. 537, SEQ ID NO. 763, SEQ ID NO. 685, SEQ ID NO. 471, SEQ ID NO. 532, SEQ ID NO. 526, SEQ ID NO. 472, SEQ ID NO. 673, or a combination thereof.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10 or more targets selected from a high dimensional discriminate analysis (HDDA) classifier. The HDDA classifier may be an HDDA150 classifier.

In some instances, the plurality of targets is at least about 70% identical to a sequence selected from a target selected from a HDDA classifier. Alternatively, the plurality of targets is at least about 80% identical to a sequence selected from a target selected from a HDDA classifier. In some instances, the plurality of targets is at least about 85% identical to a sequence selected from a target selected from a HDDA classifier. In some instances, the plurality of targets is at least about 90% identical to a sequence selected from a target selected from a HDDA classifier. Alternatively, the plurality of targets is at least about 95% identical to a sequence selected from a target selected from a HDDA classifier. The HDDA classifier may be an HDDA150 classifier.

The HDDA150 classifier may comprise SEQ ID NO. 739, SEQ ID NO. 797, SEQ ID NO. 86, SEQ ID NO. 209, SEQ ID NO. 175, SEQ ID NO. 711, SEQ ID NO. 518, SEQ ID NO. 101, SEQ ID NO. 670, SEQ ID NO. 29, SEQ ID NO. 713, SEQ ID NO. 425, SEQ ID NO. 498, SEQ ID NO. 792, SEQ ID NO. 585, SEQ ID NO. 362, SEQ ID NO. 467, SEQ ID NO. 49, SEQ ID NO. 36, SEQ ID NO. 37, SEQ ID NO. 656, SEQ ID NO. 791, SEQ ID NO. 353, SEQ ID NO. 641, SEQ ID NO. 359, SEQ ID NO. 233, SEQ ID NO. 47, SEQ ID NO. 475, SEQ ID NO. 38, SEQ ID NO. 14, SEQ ID NO. 473, SEQ ID NO. 117, SEQ ID NO. 680, SEQ ID NO. 56, SEQ ID NO. 107, SEQ ID NO. 499, SEQ ID NO. 125, SEQ ID NO. 274, SEQ ID NO. 39, SEQ ID NO. 146, SEQ ID NO. 824, SEQ ID NO. 639, SEQ ID NO. 623, SEQ ID NO. 394, SEQ ID NO. 822, SEQ ID NO. 12, SEQ ID NO. 155, SEQ ID NO. 587, SEQ ID NO. 716, SEQ ID NO. 469, SEQ ID NO. 589, SEQ ID NO. 810, SEQ ID NO. 747, SEQ ID NO. 823, SEQ ID NO. 800, SEQ ID NO. 807, SEQ ID NO. 640, SEQ ID NO. 659, SEQ ID NO. 511, SEQ ID NO. 108, SEQ ID NO. 189, SEQ ID NO. 773, SEQ ID NO. 654, SEQ ID NO. 505, SEQ ID NO. 272, SEQ ID NO. 417, SEQ ID NO. 349, SEQ ID NO. 536, SEQ ID NO. 59, SEQ ID NO. 325, SEQ ID NO. 419, SEQ ID NO. 839, SEQ ID NO. 137, SEQ ID NO. 671, SEQ ID NO. 802, SEQ ID NO. 633, SEQ ID NO. 262, SEQ ID NO. 24, SEQ ID NO. 259, SEQ ID NO. 790, SEQ ID NO. 16, SEQ ID NO. 158, SEQ ID NO. 423, SEQ ID NO. 164, SEQ ID NO. 786, SEQ ID NO. 470, SEQ ID NO. 219, SEQ ID NO. 635, SEQ ID NO. 60, SEQ ID NO. 521, SEQ ID NO. 841, SEQ ID NO. 809, SEQ ID NO. 683, SEQ ID NO. 698, SEQ ID NO. 466, SEQ ID NO. 232, SEQ ID NO. 528, SEQ ID NO. 145, SEQ ID NO. 97, SEQ ID NO. 13, SEQ ID NO. 696, SEQ ID NO. 675, SEQ ID NO. 621, SEQ ID NO. 133, SEQ ID NO. 605, SEQ ID NO. 116, SEQ ID NO. 296, SEQ ID NO. 204, SEQ ID NO. 689, SEQ ID NO. 342, SEQ ID NO. 198, SEQ ID NO. 806, SEQ ID NO. 163, SEQ ID NO. 774, SEQ ID NO. 808, SEQ ID NO. 660, SEQ ID NO. 762, SEQ ID NO. 586, SEQ ID NO. 11, SEQ ID NO. 177, SEQ ID NO. 701, SEQ ID NO. 220, SEQ ID NO. 393, SEQ ID NO. 458, SEQ ID NO. 191, SEQ ID NO. 195, SEQ ID NO. 767, SEQ ID NO. 776, SEQ ID NO. 520, SEQ ID NO. 709, SEQ ID NO. 55, SEQ ID NO. 143, SEQ ID NO. 420, SEQ ID NO. 422, SEQ ID NO. 481, SEQ ID NO. 529, SEQ ID NO. 845, SEQ ID NO. 412, SEQ ID NO. 667, SEQ ID NO. 681, SEQ ID NO. 812, SEQ ID NO. 197, SEQ ID NO. 73, SEQ ID NO. 115, SEQ ID NO. 74, SEQ ID NO. 217, SEQ ID NO. 428, SEQ ID NO. 106, SEQ ID NO. 741, SEQ ID NO. 124, or a combination thereof.

### Probes/Primers

The present invention provides for a probe set for diagnosing, monitoring and/or predicting a status or outcome of a cancer in a subject comprising a plurality of probes, wherein (i) the probes in the set are capable of detecting an expression level of at least one non-coding target; and (ii) the expression level determines the cancer status of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from prostate cancer tissue samples and amplified, may yield a dataset that is then summarized and normalized by the fRMA technique. After removal (or filtration) of cross-hybridizing PSRs, highly variable PSRs (variance above the 90th percentile), and PSRs containing more than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

One skilled in the art understands that the nucleotide sequence of the polynucleotide probe need not be identical to its target sequence in order to specifically hybridize thereto. The polynucleotide probes of the present invention, therefore, comprise a nucleotide sequence that is at least about 65% identical to a region of the coding target or non-coding target selected from Table 1. In another embodiment, the nucleotide sequence of the polynucleotide probe is at least about 70% identical a region of the coding target or non-coding target from Table 1. In another embodiment, the nucleotide sequence of the polynucleotide probe is at least about 75% identical a region of the coding target or non-coding target from Table 1. In another embodiment, the nucleotide sequence of the polynucleotide probe is at least about 80% identical a region of the coding target or non-coding target from Table 1. In another embodiment, the nucleotide sequence of the polynucleotide probe is at least about 85% identical a region of the coding target or non-coding target from Table 1. In another embodiment, the nucleotide sequence of the polynucleotide probe is at least about 90% identical a region of the coding target or non-coding target from Table 1. In a further embodiment, the nucleotide sequence of the polynucleotide probe is at least about 95% identical to a region of the coding target or non-coding target from Table 1.

Methods of determining sequence identity are known in the art and can be determined, for example, by using the BLASTN program of the University of Wisconsin Computer Group (GCG) software or provided on the NCBI website. The nucleotide sequence of the polynucleotide probes of the present invention may exhibit variability by differing (e.g. by nucleotide substitution, including transition or transversion) at one, two, three, four or more nucleotides from the sequence of the coding target or non-coding target.

Other criteria known in the art may be employed in the design of the polynucleotide probes of the present invention. For example, the probes can be designed to have <50% G content. The probes can be designed to have between about 25% and about 70% G+C content. Strategies to optimize probe hybridization to the target nucleic acid sequence can also be included in the process of probe selection.

Hybridization under particular pH, salt, and temperature conditions can be optimized by taking into account melting temperatures and by using empirical rules that correlate with desired hybridization behaviors. Computer models may be used for predicting the intensity and concentration-dependence of probe hybridization.

The polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In some embodiments, the probe set comprise a plurality of target sequences that hybridize to at least about 5 coding targets and/or non-coding targets selected from Table 1. Alternatively, the probe set comprise a plurality of target sequences that hybridize to at least about 10 coding targets and/or non-coding targets selected from Table 1. In some embodiments, the probe set comprise a plurality of target sequences that hybridize to at least about 15 coding targets and/or non-coding targets selected from Table 1. In some embodiments, the probe set comprise a plurality of target sequences that hybridize to at least about 20 coding targets and/or non-coding targets selected from Table 1. In some embodiments, the probe set comprise a plurality of target sequences that hybridize to at least about 30 coding targets and/or non-coding targets selected from Table 1. The probe set can comprise a plurality of targets that hybridize to at least about 40, 50, 60, 70, 80, 90, 100 or more coding targetns and/or non-coding targets selected from Table 1. The probe set can comprise a plurality of targets that hybridize to at least about 100, 125, 150, 175, 200, 225, 250, 275, 300 or more coding targetns and/or non-coding targets selected from Table 1. The probe set can comprise a plurality of targets that hybridize to at least about 300, 325, 350, 375, 400, 425, 450, 475, 500, 525, 550, 575, 600 or more coding targetns and/or non-coding targets selected from Table 1. The probe set can comprise a plurality of targets that hybridize to at least about 600, 625, 650, 675, 700, 725, 750, 775, 800, 825, 850 or more coding targetns and/or non-coding targets selected from Table 1.

In some embodiments, the probe set comprises a plurality of target sequences that hybridize to a plurality of targets, wherein the at least about 20% of the plurality of targets are targets selected from Table 1. In some embodiments, the probe set comprises a plurality of target sequences that hybridize to a plurality of targets, wherein the at least about 25% of the plurality of targets are targets selected from Table 1. In some embodiments, the probe set comprise a plurality of target sequences that hybridize to a plurality of targets, wherein the at least about 30% of the plurality of targets are targets selected from Table 1. In some embodiments, the probe set comprise a plurality of target sequences that hybridize to a plurality of targets, wherein the at least about 35% of the plurality of targets are targets selected from Table 1. In some embodiments, the probe set comprise a plurality of target sequences that hybridize to a plurality of targets, wherein the at least about 40% of the plurality of targets are targets selected from Table 1. In some embodiments, the probe set comprise a plurality of target sequences that hybridize to a plurality of targets, wherein the at least about 45% of the plurality of targets are targets selected from Table 1. In some embodiments, the probe set comprise a plurality of target sequences that hybridize to a plurality of targets, wherein the at least about 50% of the plurality of targets are targets selected from Table 1. In some embodiments, the probe set comprise a plurality of target sequences that hybridize to a plurality of targets, wherein the at least about 60% of the plurality of targets are targets selected from Table 1. In some embodiments, the probe set comprise a plurality of target sequences that hybridize to a plurality of targets, wherein the at least about 70% of the plurality of targets are targets selected from Table 1.

The system of the present invention further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In one embodiment, the primers or primer pairs, when used in an amplification reaction, specifically amplify at least a portion of a nucleic acid sequence of a target selected from Table 1 (or subgroups thereof as set forth herein), an RNA form thereof, or a complement to either thereof.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of polynucleotide probes or primers useful in this invention include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary polynucleotide probes or primers having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkyl-phosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulfide, sulfoxide and sulphone backbones; formacetyl and thioformacetyl backbones; methylene formacetyl and thioformacetyl backbones; alkene containing backbones; sulphamate backbones; methyleneimino and methylenehydrazino backbones; sulphonate and sulfonamide backbones; amide backbones; and others having mixed N, O, S and CH₂ component parts.

The present invention also contemplates oligonucleotide mimetics in which both the sugar and the internucleoside linkage of the nucleotide units are replaced with novel groups. The base units are maintained for hybridization with an appropriate nucleic acid target compound. An example of such an oligonucleotide mimetic, which has been shown to have excellent hybridization properties, is a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

The present invention also contemplates polynucleotide probes or primers comprising “locked nucleic acids” (LNAs), which may be novel conformationally restricted oligonucleotide analogues containing a methylene bridge that connects the 2′-O of ribose with the 4′-C. LNA and LNA analogues may display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described. Studies of mismatched sequences show that LNA obey the Watson-Crick base pairing rules with generally improved selectivity compared to the corresponding unmodified reference strands.

LNAs may form duplexes with complementary DNA or RNA or with complementary LNA, with high thermal affinities. The universality of LNA-mediated hybridization has been emphasized by the formation of exceedingly stable LNA:LNA duplexes. LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA and 2′-methylamino-LNA has been described and thermal stability of their duplexes with complementary RNA and DNA strands reported. Preparation of phosphorothioate-LNA and 2′-thio-LNA have also been described.

Modified polynucleotide probes or primers may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙO]ₘCH₃, O(CH₂)ₙOCH₃, O(CH₂)ₙNH₂, O(CH₂)ₙCH₃ONH₂, and O(CH₂)ₙON[((CH₂)ₙCH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂CH₂OCH₃, also known as 2′-O-(2-methoxyethyl) or 2′-MOE), 2′-dimethylaminooxyethoxy (O(CH2)2ON(CH₃)₂ group, also known as 2′-DMA0E), 2′-methoxy (2′-O—CH₃), 2′-aminopropoxy (2′-OCH₂CH₂CH₂NH₂) and 2′-fluoro (2′-F).

Similar modifications may also be made at other positions on the polynucleotide probes or primers, particularly the 3′ position of the sugar on the 3′ terminal nucleotide or in 2′-5′ linked oligonucleotides and the 5′ position of 5′ terminal nucleotide. Polynucleotide probes or primers may also have sugar mimetics such as cyclobutyl moieties in place of the pentofuranosyl sugar.

Polynucleotide probes or primers may also include modifications or substitutions to the nucleobase. As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the polynucleotide probes of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C.

One skilled in the art recognizes that it is not necessary for all positions in a given polynucleotide probe or primer to be uniformly modified. The present invention, therefore, contemplates the incorporation of more than one of the aforementioned modifications into a single polynucleotide probe or even at a single nucleoside within the probe or primer.

One skilled in the art also appreciates that the nucleotide sequence of the entire length of the polynucleotide probe or primer does not need to be derived from the target sequence. Thus, for example, the polynucleotide probe may comprise nucleotide sequences at the 5′ and/or 3′ termini that are not derived from the target sequences. Nucleotide sequences which are not derived from the nucleotide sequence of the target sequence may provide additional functionality to the polynucleotide probe. For example, they may provide a restriction enzyme recognition sequence or a “tag” that facilitates detection, isolation, purification or immobilization onto a solid support. Alternatively, the additional nucleotides may provide a self-complementary sequence that allows the primer/probe to adopt a hairpin configuration. Such configurations are necessary for certain probes, for example, molecular beacon and Scorpion probes, which can be used in solution hybridization techniques.

The polynucleotide probes or primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target sequence is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Coding schemes may optionally be used, comprising encoded particles and/or encoded tags associated with different polynucleotides of the invention. A variety of different coding schemes are known in the art, including fluorophores, including SCNCs, deposited metals, and RF tags.

Polynucleotides from the described target sequences may be employed as probes for detecting target sequences expression, for ligation amplification schemes, or may be used as primers for amplification schemes of all or a portion of a target sequences. When amplified, either strand produced by amplification may be provided in purified and/or isolated form.

In one embodiment, polynucleotides of the invention include (a) a nucleic acid depicted in Table 1; (b) an RNA form of any one of the nucleic acids depicted in Table 1; (c) a peptide nucleic acid form of any of the nucleic acids depicted in Table 1; (d) a nucleic acid comprising at least 20 consecutive bases of any of (a-c); (e) a nucleic acid comprising at least 25 bases having at least 90% sequenced identity to any of (a-c); and (f) a complement to any of (a-e).

Complements may take any polymeric form capable of base pairing to the species recited in (a)-(e), including nucleic acid such as RNA or DNA, or may be a neutral polymer such as a peptide nucleic acid. Polynucleotides of the invention can be selected from the subsets of the recited nucleic acids described herein, as well as their complements.

In some embodiments, polynucleotides of the invention comprise at least 20 consecutive bases of the nucleic acid sequence of a target selected from Table 1 or a complement thereto. The polynucleotides may comprise at least 21, 22, 23, 24, 25, 27, 30, 32, 35 or more consecutive bases of the nucleic acids sequence of a target selected from Table 1, as applicable.

The polynucleotides may be provided in a variety of formats, including as solids, in solution, or in an array. The polynucleotides may optionally comprise one or more labels, which may be chemically and/or enzymatically incorporated into the polynucleotide.

In one embodiment, solutions comprising polynucleotide and a solvent are also provided. In some embodiments, the solvent may be water or may be predominantly aqueous. In some embodiments, the solution may comprise at least two, three, four, five, six, seven, eight, nine, ten, twelve, fifteen, seventeen, twenty or more different polynucleotides, including primers and primer pairs, of the invention. Additional substances may be included in the solution, alone or in combination, including one or more labels, additional solvents, buffers, biomolecules, polynucleotides, and one or more enzymes useful for performing methods described herein, including polymerases and ligases. The solution may further comprise a primer or primer pair capable of amplifying a polynucleotide of the invention present in the solution.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

Substrates can be planar crystalline substrates such as silica based substrates (e.g. glass, quartz, or the like), or crystalline substrates used in, e.g., the semiconductor and microprocessor industries, such as silicon, gallium arsenide, indium doped GaN and the like, and include semiconductor nanocrystals.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

Silica aerogels can also be used as substrates, and can be prepared by methods known in the art. Aerogel substrates may be used as free standing substrates or as a surface coating for another substrate material.

The substrate can take any form and typically is a plate, slide, bead, pellet, disk, particle, microparticle, nanoparticle, strand, precipitate, optionally porous gel, sheets, tube, sphere, container, capillary, pad, slice, film, chip, multiwell plate or dish, optical fiber, etc. The substrate can be any form that is rigid or semi-rigid. The substrate may contain raised or depressed regions on which an assay component is located. The surface of the substrate can be etched using known techniques to provide for desired surface features, for example trenches, v-grooves, mesa structures, or the like.

Surfaces on the substrate can be composed of the same material as the substrate or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate materials. The surface can be optically transparent and can have surface Si-OH functionalities, such as those found on silica surfaces.

The substrate and/or its optional surface can be chosen to provide appropriate characteristics for the synthetic and/or detection methods used. The substrate and/or surface can be transparent to allow the exposure of the substrate by light applied from multiple directions. The substrate and/or surface may be provided with reflective “mirror” structures to increase the recovery of light.

The substrate and/or its surface is generally resistant to, or is treated to resist, the conditions to which it is to be exposed in use, and can be optionally treated to remove any resistant material after exposure to such conditions.

The substrate or a region thereof may be encoded so that the identity of the sensor located in the substrate or region being queried may be determined. Any suitable coding scheme can be used, for example optical codes, RFID tags, magnetic codes, physical codes, fluorescent codes, and combinations of codes.

### Preparation of Probes and Primers

The polynucleotide probes or primers of the present invention can be prepared by conventional techniques well-known to those skilled in the art. For example, the polynucleotide probes can be prepared using solid-phase synthesis using commercially available equipment. As is well-known in the art, modified oligonucleotides can also be readily prepared by similar methods. The polynucleotide probes can also be synthesized directly on a solid support according to methods standard in the art. This method of synthesizing polynucleotides is particularly useful when the polynucleotide probes are part of a nucleic acid array.

Polynucleotide probes or primers can be fabricated on or attached to the substrate by any suitable method, for example the methods described in U.S. Pat. No. 5,143,854, PCT Publ. No. WO 92/10092, U.S. patent application Ser. No. 07/624,120, filed Dec. 6, 1990 (now abandoned), Fodor et al., Science, 251: 767-777 (1991), and PCT Publ. No. WO 90/15070). Techniques for the synthesis of these arrays using mechanical synthesis strategies are described in, e.g., PCT Publication No. WO 93/09668 and U.S. Pat. No. 5,384,261. Still further techniques include bead based techniques such as those described in PCT Appl. No. PCT/US93/04145 and pin based methods such as those described in U.S. Pat. No. 5,288,514. Additional flow channel or spotting methods applicable to attachment of sensor polynucleotides to a substrate are described in U.S. patent application Ser. No. 07/980,523, filed Nov. 20, 1992, and U.S. Pat. No. 5,384,261.

Alternatively, the polynucleotide probes of the present invention can be prepared by enzymatic digestion of the naturally occurring target gene, or mRNA or cDNA derived therefrom, by methods known in the art.

### Diagnostic Samples

Diagnostic samples for use with the systems and in the methods of the present invention comprise nucleic acids suitable for providing RNAs expression information. In principle, the biological sample from which the expressed RNA is obtained and analyzed for target sequence expression can be any material suspected of comprising cancer tissue or cells. The diagnostic sample can be a biological sample used directly in a method of the invention. Alternatively, the diagnostic sample can be a sample prepared from a biological sample.

In one embodiment, the sample or portion of the sample comprising or suspected of comprising cancer tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine. Alternatively, the sample is from blood, plasma or serum. In some embodiments, the sample is from saliva.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, Pa.). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, Del.). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100 or more primers or pairs of primers suitable for selectively amplifying one or more targets. The kit may comprise at least 100, 125, 150, 175, 200, 250, 300, 350, 400, 450, 500 or more primers or pairs of primers suitable for selectively amplifying one or more targets. The kit may comprise at least 500, 550, 600, 650, 700, 750, 800, 850 or more primers or pairs of primers suitable for selectively amplifying one or more targets.

In some embodiments, the primers or primer pairs of the kit, when used in an amplification reaction, specifically amplify a non-coding target, coding target, or non-exonic target described herein, at least a portion of a nucleic acid sequence depicted in one of SEQ ID NOs: 1-853, a nucleic acid sequence corresponding to a target selected from Table 1, an RNA form thereof, or a complement to either thereof. The kit may include a plurality of such primers or primer pairs which can specifically amplify a corresponding plurality of different amplify a non-coding target, coding target, or non-exonic transcript described herein, nucleic acids depicted in one of SEQ ID NOs: 1-853, a nucleic acid sequence corresponding to a target selected from Table 1, RNA forms thereof, or complements thereto. At least two, three, four or five primers or pairs of primers suitable for selectively amplifying the one or ore targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Devices

Devices useful for performing methods of the invention are also provided. The devices can comprise means for characterizing the expression level of a target sequence of the invention, for example components for performing one or more methods of nucleic acid extraction, amplification, and/or detection. Such components may include one or more of an amplification chamber (for example a thermal cycler), a plate reader, a spectrophotometer, capillary electrophoresis apparatus, a chip reader, and or robotic sample handling components. These components ultimately can obtain data that reflects the expression level of the target sequences used in the assay being employed.

The devices may include an excitation and/or a detection means. Any instrument that provides a wavelength that can excite a species of interest and is shorter than the emission wavelength(s) to be detected can be used for excitation. Commercially available devices can provide suitable excitation wavelengths as well as suitable detection component.

Exemplary excitation sources include a broadband UV light source such as a deuterium lamp with an appropriate filter, the output of a white light source such as a xenon lamp or a deuterium lamp after passing through a monochromator to extract out the desired wavelength(s), a continuous wave (cw) gas laser, a solid state diode laser, or any of the pulsed lasers. Emitted light can be detected through any suitable device or technique; many suitable approaches are known in the art. For example, a fluorimeter or spectrophotometer may be used to detect whether the test sample emits light of a wavelength characteristic of a label used in an assay.

The devices typically comprise a means for identifying a given sample, and of linking the results obtained to that sample. Such means can include manual labels, barcodes, and other indicators which can be linked to a sample vessel, and/or may optionally be included in the sample itself, for example where an encoded particle is added to the sample. The results may be linked to the sample, for example in a computer memory that contains a sample designation and a record of expression levels obtained from the sample. Linkage of the results to the sample can also include a linkage to a particular sample receptacle in the device, which is also linked to the sample identity.

In some instances, the devices also comprise a means for correlating the expression levels of the target sequences being studied with a prognosis of disease outcome. In some instances, such means comprises one or more of a variety of correlative techniques, including lookup tables, algorithms, multivariate models, and linear or nonlinear combinations of expression models or algorithms. The expression levels may be converted to one or more likelihood scores, reflecting likelihood that the patient providing the sample may exhibit a particular disease outcome. The models and/or algorithms can be provided in machine readable format and can optionally further designate a treatment modality for a patient or class of patients.

The device also comprises output means for outputting the disease status, prognosis and/or a treatment modality. Such output means can take any form which transmits the results to a patient and/or a healthcare provider, and may include a monitor, a printed format, or both. The device may use a computer system for performing one or more of the steps provided.

In some embodiments, the method, systems, and kits disclosed herein further comprise the transmission of data/information. For example, data/information derived from the detection and/or quantification of the target may be transmitted to another device and/or instrument. In some instances, the information obtained from an algorithm is transmitted to another device and/or instrument. Transmission of the data/information may comprise the transfer of data/information from a first source to a second source. The first and second sources may be in the same approximate location (e.g., within the same room, building, block, campus). Alternatively, first and second sources may be in multiple locations (e.g., multiple cities, states, countries, continents, etc).

In some instances, transmission of the data/information comprises digital transmission or analog transmission. Digital transmission may comprise the physical transfer of data (a digital bit stream) over a point-to-point or point-to-multipoint communication channel. Examples of such channels are copper wires, optical fibers, wireless communication channels, and storage media. In some embodiments, the data is represented as an electromagnetic signal, such as an electrical voltage, radiowave, microwave, or infrared signal.

Analog transmission may comprise the transfer of a continuously varying analog signal. The messages can either be represented by a sequence of pulses by means of a line code (baseband transmission), or by a limited set of continuously varying wave forms (passband transmission), using a digital modulation method. The passband modulation and corresponding demodulation (also known as detection) can be carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation′m FFPE Exon Module, WT-Ovation™ FFPE Exon Module RiboAmp and RiboAmp^{Plus} RNA Amplification Kits (MDS Analytical Technologies (formerly Arcturus) (Mountain View, Calif.), Genisphere, Inc. (Hatfield, Pa.), including the RampUp Plus™ and SenseAmp™ RNA Amplification kits, alone or in combination. Amplified nucleic acids may be subjected to one or more purification reactions after amplification and labeling, for example using magnetic beads (e.g., RNAC 1 can magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

Detection and/or Quantification of Target Sequences

Any method of detecting and/or quantitating the expression of the encoded target sequences can in principle be used in the invention. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for QRT-PCR Analysis

Reverse transcription can be performed by any method known in the art. For example, reverse transcription may be performed using the Omniscript kit (Qiagen, Valencia, Calif.), Superscript III kit (Invitrogen, Carlsbad, Calif.), for RT-PCR. Target-specific priming can be performed in order to increase the sensitivity of detection of target sequences and generate target-specific cDNA.

### TaqMan® Gene Expression Analysis

TaqMan® RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present invention contemplates that a probe set or probes derived therefrom may be provided in an array format. In the context of the present invention, an “array” is a spatially or logically organized collection of polynucleotide probes. An array comprising probes specific for a coding target, non-coding target, or a combination thereof may be used. Alternatively, an array comprising probes specific for two or more of transcripts of a target selected from Table 1 or a product derived thereof can be used. Desirably, an array may be specific for 5, 10, 15, 20, 25, 30, 50, 75, 100, 150, 200 or more of transcripts of a target selected from Table 1. The array may be specific for 200, 225, 250, 275, 300, 325, 350, 375, 400 or more of the transcripts of a target selected from Table 1. The array may be specific for 400, 425, 450, 475, 500, 525, 550, 575, 600 or more of the transcripts of a target selected from Table 1. The array may be specific for 600, 625, 650, 675, 700, 725, 750, 775, 800, 825, 850 or more of the transcripts of a target selected from Table 1. Expression of these sequences may be detected alone or in combination with other transcripts. In some embodiments, an array is used which comprises a wide range of sensor probes for prostate-specific expression products, along with appropriate control sequences. In some instances, the array may comprise the Human Exon 1.0 ST Array (HuEx 1.0 ST, Affymetrix, Inc., Santa Clara, Calif.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present invention, the Cancer Prognosticarray is a chip.

### Data Analysis

In some embodiments, one or more pattern recognition methods can be used in analyzing the expression level of target sequences. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Assaying the expression level for a plurality of targets may comprise the use of an algorithm or classifier. Array data can be managed, classified, and analyzed using techniques known in the art. Assaying the expression level for a plurality of targets may comprise probe set modeling and data pre-processing. Probe set modeling and data pre-processing can be derived using the Robust Multi-Array (RMA) algorithm or variants GC-RMA, JRMA, Probe Logarithmic Intensity Error (PLIER) algorithm or variant iterPLIER. Variance or intensity filters can be applied to pre-process data using the RMA algorithm, for example by removing target sequences with a standard deviation of <10 or a mean intensity of <100 intensity units of a normalized data range, respectively.

Alternatively, assaying the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Additional Techniques and Tests

Factors known in the art for diagnosing and/or suggesting, selecting, designating, recommending or otherwise determining a course of treatment for a patient or class of patients suspected of having cancer can be employed in combination with measurements of the target sequence expression. The methods disclosed herein may include additional techniques such as cytology, histology, ultrasound analysis, MRI results, CT scan results, and measurements of PSA levels.

Certified tests for classifying disease status and/or designating treatment modalities may also be used in diagnosing, predicting, and/or monitoring the status or outcome of a cancer in a subject. A certified test may comprise a means for characterizing the expression levels of one or more of the target sequences of interest, and a certification from a government regulatory agency endorsing use of the test for classifying the disease status of a biological sample.

In some embodiments, the certified test may comprise reagents for amplification reactions used to detect and/or quantitate expression of the target sequences to be characterized in the test. An array of probe nucleic acids can be used, with or without prior target amplification, for use in measuring target sequence expression.

The test is submitted to an agency having authority to certify the test for use in distinguishing disease status and/or outcome. Results of detection of expression levels of the target sequences used in the test and correlation with disease status and/or outcome are submitted to the agency. A certification authorizing the diagnostic and/or prognostic use of the test is obtained.

Also provided are portfolios of expression levels comprising a plurality of normalized expression levels of the target selected from Table 1. Such portfolios may be provided by performing the methods described herein to obtain expression levels from an individual patient or from a group of patients. The expression levels can be normalized by any method known in the art; exemplary normalization methods that can be used in various embodiments include Robust Multichip Average (RMA), probe logarithmic intensity error estimation (PLIER), non-linear fit (NLFIT) quantile-based and nonlinear normalization, and combinations thereof. Background correction can also be performed on the expression data; exemplary techniques useful for background correction include mode of intensities, normalized using median polish probe modeling and sketch-normalization.

In some embodiments, portfolios are established such that the combination of genes in the portfolio exhibit improved sensitivity and specificity relative to known methods. In considering a group of genes for inclusion in a portfolio, a small standard deviation in expression measurements correlates with greater specificity. Other measurements of variation such as correlation coefficients can also be used in this capacity. The invention also encompasses the above methods where the expression level determines the status or outcome of a cancer in the subject with at least about 45% specificity. In some embodiments, the expression level determines the status or outcome of a cancer in the subject with at least about 50% specificity. In some embodiments, the expression level determines the status or outcome of a cancer in the subject with at least about 55% specificity. In some embodiments, the expression level determines the status or outcome of a cancer in the subject with at least about 60% specificity. In some embodiments, the expression level determines the status or outcome of a cancer in the subject with at least about 65% specificity. In some embodiments, the expression level determines the status or outcome of a cancer in the subject with at least about 70% specificity. In some embodiments, the expression level determines the status or outcome of a cancer in the subject with at least about 75% specificity. In some embodiments, the expression level determines the status or outcome of a cancer in the subject with at least about 80% specificity. In some embodiments, t the expression level determines the status or outcome of a cancer in the subject with at least about 85% specificity. In some embodiments, the expression level determines the status or outcome of a cancer in the subject with at least about 90% specificity. In some embodiments, the expression level determines the status or outcome of a cancer in the subject with at least about 95% specificity.

The invention also encompasses the any of the methods disclosed herein where the accuracy of diagnosing, monitoring, and/or predicting a status or outcome of a cancer is at least about 45%. In some embodiments, the accuracy of diagnosing, monitoring, and/or predicting a status or outcome of a cancer is at least about 50%. In some embodiments, the accuracy of diagnosing, monitoring, and/or predicting a status or outcome of a cancer is at least about 55%. In some embodiments, the accuracy of diagnosing, monitoring, and/or predicting a status or outcome of a cancer is at least about 60%. In some embodiments, the accuracy of diagnosing, monitoring, and/or predicting a status or outcome of a cancer is at least about 65%. In some embodiments, the accuracy of diagnosing, monitoring, and/or predicting a status or outcome of a cancer is at least about 70%. In some embodiments, the accuracy of diagnosing, monitoring, and/or predicting a status or outcome of a cancer is at least about 75%. In some embodiments, the accuracy of diagnosing, monitoring, and/or predicting a status or outcome of a cancer is at least about 80%. In some embodiments, the accuracy of diagnosing, monitoring, and/or predicting a status or outcome of a cancer is at least about 85%. In some embodiments, the accuracy of diagnosing, monitoring, and/or predicting a status or outcome of a cancer is at least about 90%. In some embodiments, the accuracy of diagnosing, monitoring, and/or predicting a status or outcome of a cancer is at least about 95%.

The accuracy of a classifier or biomarker may be determined by the 95% confidence interval (CI). Generally, a classifier or biomarker is considered to have good accuracy if the 95% CI does not overlap 1. In some instances, the 95% CI of a classifier or biomarker is at least about 1.08, 1.10, 1.12, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, or 1.35 or more. The 95% CI of a classifier or biomarker may be at least about 1.14, 1.15, 1.16, 1.20, 1.21, 1.26, or 1.28. The 95% CI of a classifier or biomarker may be less than about 1.75, 1.74, 1.73, 1.72, 1.71, 1.70, 1.69, 1.68, 1.67, 1.66, 1.65, 1.64, 1.63, 1.62, 1.61, 1.60, 1.59, 1.58, 1.57, 1.56, 1.55, 1.54, 1.53, 1.52, 1.51, 1.50 or less. The 95% CI of a classifier or biomarker may be less than about 1.61, 1.60, 1.59, 1.58, 1.56, 1.55, or 1.53. The 95% CI of a classifier or biomarker may be between about 1.10 to 1.70, between about 1.12 to about 1.68, between about 1.14 to about 1.62, between about 1.15 to about 1.61, between about 1.15 to about 1.59, between about 1.16 to about 1.160, between about 1.19 to about 1.55, between about 1.20 to about 1.54, between about 1.21 to about 1.53, between about 1.26 to about 1.63, between about 1.27 to about 1.61, or between about 1.28 to about 1.60.

In some instances, the accuracy of a biomarker or classifier is dependent on the difference in range of the 95% CI (e.g., difference in the high value and low value of the 95% CI interval). Generally, biomarkers or classifiers with large differences in the range of the 95% CI interval have greater variability and are considered less accurate than biomarkers or classifiers with small differences in the range of the 95% CI intervals. In some instances, a biomarker or classifier is considered more accurate if the difference in the range of the 95% CI is less than about 0.60, 0.55, 0.50, 0.49, 0.48, 0.47, 0.46, 0.45, 0.44, 0.43, 0.42, 0.41, 0.40, 0.39, 0.38, 0.37, 0.36, 0.35, 0.34, 0.33, 0.32, 0.31, 0.30, 0.29, 0.28, 0.27, 0.26, 0.25 or less. The difference in the range of the 95% CI of a biomarker or classifier may be less than about 0.48, 0.45, 0.44, 0.42, 0.40, 0.37, 0.35, 0.33, or 0.32. In some instances, the difference in the range of the 95% CI for a biomarker or classifier is between about 0.25 to about 0.50, between about 0.27 to about 0.47, or between about 0.30 to about 0.45.

The invention also encompasses the any of the methods disclosed herein where the sensitivity is at least about 45%. In some embodiments, the sensitivity is at least about 50%. In some embodiments, the sensitivity is at least about 55%. In some embodiments, the sensitivity is at least about 60%. In some embodiments, the sensitivity is at least about 65%. In some embodiments, the sensitivity is at least about 70%. In some embodiments, the sensitivity is at least about 75%. In some embodiments, the sensitivity is at least about 80%. In some embodiments, the sensitivity is at least about 85%. In some embodiments, the sensitivity is at least about 90%. In some embodiments, the sensitivity is at least about 95%.

In some instances, the classifiers or biomarkers disclosed herein are clinically significant. In some instances, the clinical significance of the classifiers or biomarkers is determined by the AUC value. In order to be clinically significant, the AUC value is at least about 0.5, 0.55, 0.6, 0.65, 0.7, 0.75, 0.8, 0.85, 0.9, or 0.95. The clinical significance of the classifiers or biomarkers can be determined by the percent accuracy. For example, a classifier or biomarker is determined to be clinically significant if the accuracy of the classifier or biomarker is at least about 50%, 55%, 60%, 65%, 70%, 72%, 75%, 77%, 80%, 82%, 84%, 86%, 88%, 90%, 92%, 94%, 96%, or 98%. In other instances, the clinical significance of the classifiers or biomarkers is determined by the median fold difference (MDF) value. In order to be clinically significant, the MDF value is at least about 0.8, 0.9, 1.0, 1.1, 1.2, 1.3, 1.4, 1.5, 1.6, 1.7, 1.9, or 2.0. In some instances, the MDF value is greater than or equal to 1.1. In other instances, the MDF value is greater than or equal to 1.2. Alternatively, or additionally, the clinical significance of the classifiers or biomarkers is determined by the t-test P-value. In some instances, in order to be clinically significant, the t-test P-value is less than about 0.070, 0.065, 0.060, 0.055, 0.050, 0.045, 0.040, 0.035, 0.030, 0.025, 0.020, 0.015, 0.010, 0.005, 0.004, or 0.003. The t-test P-value can be less than about 0.050. Alternatively, the t-test P-value is less than about 0.010. In some instances, the clinical significance of the classifiers or biomarkers is determined by the clinical outcome. For example, different clinical outcomes can have different minimum or maximum thresholds for AUC values, MDF values, t-test P-values, and accuracy values that would determine whether the classifier or biomarker is clinically significant. In another example, a classifier or biomarker is considered clinically significant if the P-value of the t-test was less than about 0.08, 0.07, 0.06, 0.05, 0.04, 0.03, 0.02, 0.01, 0.005, 0.004, 0.003, 0.002, or 0.001. In some instances, the P-value may be based on any of the following comparisons: BCR vs non-BCR, CP vs non-CP, PCSM vs non-PCSM. For example, a classifier or biomarker is determined to be clinically significant if the P-values of the differences between the KM curves for BCR vs non-BCR, CP vs non-CP, PCSM vs non-PCSM is lower than about 0.08, 0.07, 0.06, 0.05, 0.04, 0.03, 0.02, 0.01, 0.005, 0.004, 0.003, 0.002, or 0.001.

In some instances, the performance of the classifier or biomarker is based on the odds ratio. A classifier or biomarker may be considered to have good performance if the odds ratio is at least about 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.52, 1.55, 1.57, 1.60, 1.62, 1.65, 1.67, 1.70 or more. In some instances, the odds ratio of a classifier or biomarker is at least about 1.33.

The clinical significance of the classifiers and/or biomarkers may be based on Univariable Analysis Odds Ratio P-value (uvaORPval). The Univariable Analysis Odds Ratio P-value (uvaORPval)) of the classifier and/or biomarker may be between about 0-0.4. The Univariable Analysis Odds Ratio P-value (uvaORPval) of the classifier and/or biomarker may be between about 0-0.3. The Univariable Analysis Odds Ratio P-value (uvaORPval) of the classifier and/or biomarker may be between about 0-0.2. The Univariable Analysis Odds Ratio P-value (uvaORPval) of the classifier and/or biomarker may be less than or equal to 0.25, 0.22, 0.21, 0.20, 0.19, 0.18, 0.17, 0.16, 0.15, 0.14, 0.13, 0.12, 0.11. The Univariable Analysis Odds Ratio P-value (uvaORPval) of the classifier and/or biomarker may be less than or equal to 0.10, 0.09, 0.08, 0.07, 0.06, 0.05, 0.04, 0.03, 0.02, 0.01. The Univariable Analysis Odds Ratio P-value (uvaORPval) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on multivariable analysis Odds Ratio P-value (mvaORPval). The multivariable analysis Odds Ratio P-value (mvaORPval) of the classifier and/or biomarker may be between about 0-1. The multivariable analysis Odds Ratio P-value (mvaORPval) of the classifier and/or biomarker may be between about 0-0.9. The multivariable analysis Odds Ratio P-value (mvaORPval) of the classifier and/or biomarker may be between about 0-0.8. The multivariable analysis Odds Ratio P-value (mvaORPval)) of the classifier and/or biomarker may be less than or equal to 0.90, 0.88, 0.86, 0.84, 0.82, 0.80. The multivariable analysis Odds Ratio P-value (mvaORPval) of the classifier and/or biomarker may be less than or equal to 0.78, 0.76, 0.74, 0.72, 0.70, 0.68, 0.66, 0.64, 0.62, 0.60, 0.58, 0.56, 0.54, 0.52, 0.50. The multivariable analysis Odds Ratio P-value (mvaORPval) of the classifier and/or biomarker may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The multivariable analysis Odds Ratio P-value (mvaORPval)) of the classifier and/or biomarker may be less than or equal to 0.10, 0.09, 0.08, 0.07, 0.06, 0.05, 0.04, 0.03, 0.02, 0.01. The multivariable analysis Odds Ratio P-value (mvaORPval)) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the Kaplan Meier P-value (KM P-value). The Kaplan Meier P-value (KM P-value) of the classifier and/or biomarker may be between about 0-0.8. The Kaplan Meier P-value (KM P-value) of the classifier and/or biomarker may be between about 0-0.7. The Kaplan Meier P-value (KM P-value) of the classifier and/or biomarker may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Kaplan Meier P-value (KM P-value) of the classifier and/or biomarker may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Kaplan Meier P-value (KM P-value) of the classifier and/or biomarker may be less than or equal to 0.10, 0.09, 0.08, 0.07, 0.06, 0.05, 0.04, 0.03, 0.02, 0.01. The Kaplan Meier P-value (KM P-value) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the survival AUC value (survAUC). The survival AUC value (survAUC) of the classifier and/or biomarker may be between about 0-1. The survival AUC value (survAUC) of the classifier and/or biomarker may be between about 0-0.9. The survival AUC value (survAUC) of the classifier and/or biomarker may be less than or equal to 1, 0.98, 0.96, 0.94, 0.92, 0.90, 0.88, 0.86, 0.84, 0.82, 0.80. The survival AUC value (survAUC) of the classifier and/or biomarker may be less than or equal to 0.80, 0.78, 0.76, 0.74, 0.72, 0.70, 0.68, 0.66, 0.64, 0.62, 0.60, 0.58, 0.56, 0.54, 0.52, 0.50. The survival AUC value (survAUC) of the classifier and/or biomarker may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The survival AUC value (survAUC) of the classifier and/or biomarker may be less than or equal to 0.10, 0.09, 0.08, 0.07, 0.06, 0.05, 0.04, 0.03, 0.02, 0.01. The survival AUC value (survAUC) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the Univariable Analysis Hazard Ratio P-value (uvaHRPval). The Univariable Analysis Hazard Ratio P-value (uvaHRPval) of the classifier and/or biomarker may be between about 0-0.4. The Univariable Analysis Hazard Ratio P-value (uvaHRPval) of the classifier and/or biomarker may be between about 0-0.3. The Univariable Analysis Hazard Ratio P-value (uvaHRPval) of the classifier and/or biomarker may be less than or equal to 0.40, 0.38, 0.36, 0.34, 0.32. The Univariable Analysis Hazard Ratio P-value (uvaHRPval) of the classifier and/or biomarker may be less than or equal to 0.30, 0.29, 0.28, 0.27, 0.26, 0.25, 0.24, 0.23, 0.22, 0.21, 0.20. The Univariable Analysis Hazard Ratio P-value (uvaHRPval) of the classifier and/or biomarker may be less than or equal to 0.19, 0.18, 0.17, 0.16, 0.15, 0.14, 0.13, 0.12, 0.11. The Univariable Analysis Hazard Ratio P-value (uvaHRPval) of the classifier and/or biomarker may be less than or equal to 0.10, 0.09, 0.08, 0.07, 0.06, 0.05, 0.04, 0.03, 0.02, 0.01. The Univariable Analysis Hazard Ratio P-value (uvaHRPval) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the Multivariable Analysis Hazard Ratio P-value (mvaHRPval)mva HRPval. The Multivariable Analysis Hazard Ratio P-value (mvaHRPval)mva HRPval of the classifier and/or biomarker may be between about 0-1. The Multivariable Analysis Hazard Ratio P-value (mvaHRPval)mva HRPval of the classifier and/or biomarker may be between about 0-0.9. The Multivariable Analysis Hazard Ratio P-value (mvaHRPval)mva HRPval of the classifier and/or biomarker may be less than or equal to 1, 0.98, 0.96, 0.94, 0.92, 0.90, 0.88, 0.86, 0.84, 0.82, 0.80. The Multivariable Analysis Hazard Ratio P-value (mvaHRPval)mva HRPval of the classifier and/or biomarker may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Multivariable Analysis Hazard Ratio P-value (mvaHRPval)mva HRPval of the classifier and/or biomarker may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Multivariable Analysis Hazard Ratio P-value (mvaHRPval)mva HRPval of the classifier and/or biomarker may be less than or equal to 0.10, 0.09, 0.08, 0.07, 0.06, 0.05, 0.04, 0.03, 0.02, 0.01. The Multivariable Analysis Hazard Ratio P-value (mvaHRPval)mva HRPval of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the Multivariable Analysis Hazard Ratio P-value (mvaHRPval). The Multivariable Analysis Hazard Ratio P-value (mvaHRPval) of the classifier and/or biomarker may be between about 0 to about 0.60. significance of the classifier and/or biomarker may be based on the Multivariable Analysis Hazard Ratio P-value (mvaHRPval). The Multivariable Analysis Hazard Ratio P-value (mvaHRPval) of the classifier and/or biomarker may be between about 0 to about 0.50. significance of the classifier and/or biomarker may be based on the Multivariable Analysis Hazard Ratio P-value (mvaHRPval). The Multivariable Analysis Hazard Ratio P-value (mvaHRPval) of the classifier and/or biomarker may be less than or equal to 0.50, 0.47, 0.45, 0.43, 0.40, 0.38, 0.35, 0.33, 0.30, 0.28, 0.25, 0.22, 0.20, 0.18, 0.16, 0.15, 0.14, 0.13, 0.12, 0.11, 0.10. The Multivariable Analysis Hazard Ratio P-value (mvaHRPval) of the classifier and/or biomarker may be less than or equal to 0.10, 0.09, 0.08, 0.07, 0.06, 0.05, 0.04, 0.03, 0.02, 0.01. The Multivariable Analysis Hazard Ratio P-value (mvaHRPval) of the classifier and/or biomarker may be less than or equal to 0.01, 0.009, 0.008, 0.007, 0.006, 0.005, 0.004, 0.003, 0.002, 0.001.

The classifiers and/or biomarkers disclosed herein may outperform current classifiers or clinical variables in providing clinically relevant analysis of a sample from a subject. In some instances, the classifiers or biomarkers may more accurately predict a clinical outcome or status as compared to current classifiers or clinical variables. For example, a classifier or biomarker may more accurately predict metastatic disease. Alternatively, a classifier or biomarker may more accurately predict no evidence of disease. In some instances, the classifier or biomarker may more accurately predict death from a disease. The performance of a classifier or biomarker disclosed herein may be based on the AUC value, odds ratio, 95% CI, difference in range of the 95% CI, p-value or any combination thereof.

The performance of the classifiers and/or biomarkers disclosed herein may be determined by AUC values and an improvement in performance may be determined by the difference in the AUC value of the classifier or biomarker disclosed herein and the AUC value of current classifiers or clinical variables. In some instances, a classifier and/or biomarker disclosed herein outperforms current classifiers or clinical variables when the AUC value of the classifier and/or or biomarker disclosed herein is greater than the AUC value of the current classifiers or clinical variables by at least about 0.05, 0.06, 0.07, 0.08, 0.09, 0.10, 0.11, 0.12, 0.13, 0.14, 0.15, 0.16, 0.17, 0.18, 0.19, 0.20, 0.022, 0.25, 0.27, 0.30, 0.32, 0.35, 0.37, 0.40, 0.42, 0.45, 0.47, 0.50 or more. In some instances, the AUC value of the classifier and/or or biomarker disclosed herein is greater than the AUC value of the current classifiers or clinical variables by at least about 0.10. In some instances, the AUC value of the classifier and/or or biomarker disclosed herein is greater than the AUC value of the current classifiers or clinical variables by at least about 0.13. In some instances, the AUC value of the classifier and/or or biomarker disclosed herein is greater than the AUC value of the current classifiers or clinical variables by at least about 0.18.

The performance of the classifiers and/or biomarkers disclosed herein may be determined by the odds ratios and an improvement in performance may be determined by comparing the odds ratio of the classifier or biomarker disclosed herein and the odds ratio of current classifiers or clinical variables. Comparison of the performance of two or more classifiers, biomarkers, and/or clinical variables can be generally be based on the comparison of the absolute value of (1-odds ratio) of a first classifier, biomarker or clinical variable to the absolute value of (1-odds ratio) of a second classifier, biomarker or clinical variable. Generally, the classifier, biomarker or clinical variable with the greater absolute value of (1-odds ratio) can be considered to have better performance as compared to the classifier, biomarker or clinical variable with a smaller absolute value of (1-odds ratio).

In some instances, the performance of a classifier, biomarker or clinical variable is based on the comparison of the odds ratio and the 95% confidence interval (CI). For example, a first classifier, biomarker or clinical variable may have a greater absolute value of (1-odds ratio) than a second classifier, biomarker or clinical variable, however, the 95% CI of the first classifier, biomarker or clinical variable may overlap 1 (e.g., poor accuracy), whereas the 95% CI of the second classifier, biomarker or clinical variable does not overlap 1. In this instance, the second classifier, biomarker or clinical variable is considered to outperform the first classifier, biomarker or clinical variable because the accuracy of the first classifier, biomarker or clinical variable is less than the accuracy of the second classifier, biomarker or clinical variable. In another example, a first classifier, biomarker or clinical variable may outperform a second classifier, biomarker or clinical variable based on a comparison of the odds ratio; however, the difference in the 95% CI of the first classifier, biomarker or clinical variable is at least about 2 times greater than the 95% CI of the second classifier, biomarker or clinical variable. In this instance, the second classifier, biomarker or clinical variable is considered to outperform the first classifier.

In some instances, a classifier or biomarker disclosed herein more accurate than a current classifier or clinical variable. The classifier or biomarker disclosed herein is more accurate than a current classifier or clinical variable if the range of 95% CI of the classifier or biomarker disclosed herein does not span or overlap 1 and the range of the 95% CI of the current classifier or clinical variable spans or overlaps 1.

In some instances, a classifier or biomarker disclosed herein more accurate than a current classifier or clinical variable. The classifier or biomarker disclosed herein is more accurate than a current classifier or clinical variable when difference in range of the 95% CI of the classifier or biomarker disclosed herein is about 0.70, 0.60, 0.50, 0.40, 0.30, 0.20, 0.15, 0.14, 0.13, 0.12, 0.10, 0.09, 0.08, 0.07, 0.06, 0.05, 0.04, 0.03, 0.02 times less than the difference in range of the 95% CI of the current classifier or clinical variable. The classifier or biomarker disclosed herein is more accurate than a current classifier or clinical variable when difference in range of the 95% CI of the classifier or biomarker disclosed herein between about 0.20 to about 0.04 times less than the difference in range of the 95% CI of the current classifier or clinical variable.

In some instances, the methods disclosed herein may comprise the use of a genomic classifier (GC) model. A general method for developing a GC model may comprise (a) providing a sample from a subject suffering from a cancer; (b) assaying the expression level for a plurality of targets; (c) generating a model by using a machine learning algorithm. In some instances, the machine learning algorithm comprises Random Forests. In another example, a GC model may developed by using a machine learning algorithm to analyze and rank genomic features. Analyzing the genomic features may comprise classifying one or more genomic features. The method may further comprise validating the classifier and/or refining the classifier by using a machine learning algorithm.

The methods disclosed herein may comprise generating one or more clinical classifiers (CC). The clinical classifier can be developed using one or more clinicopathologic variables. The clinicopathologic variables may be selected from the group comprising Lymph node invasion status (LNI); Surgical Margin Status (SMS); Seminal Vesicle Invasion (SVI); Extra Capsular Extension (ECE); Pathological Gleason Score; and the pre-operative PSA. The method may comprise using one or more of the clinicopathologic variables as binary variables. Alternatively, or additionally, the one or more clinicopathologic variables may be converted to a logarithmic value (e.g., log 10). The method may further comprise assembling the variables in a logistic regression. In some instances, the CC is combined with the GC to produce a genomic clinical classifier (GCC).

In some instances, the methods disclosed herein may comprise the use of a genomic-clinical classifier (GCC) model. A general method for developing a GCC model may comprise (a) providing a sample from a subject suffering from a cancer; (b) assaying the expression level for a plurality of targets; (c) generating a model by using a machine learning algorithm. In some instances, the machine learning algorithm comprises Random Forests.

### Cancer

The systems, compositions and methods disclosed herein may be used to diagnosis, monitor and/or predict the status or outcome of a cancer. Generally, a cancer is characterized by the uncontrolled growth of abnormal cells anywhere in a body. The abnormal cells may be termed cancer cells, malignant cells, or tumor cells. Many cancers and the abnormal cells that compose the cancer tissue are further identified by the name of the tissue that the abnormal cells originated from (for example, breast cancer, lung cancer, colon cancer, prostate cancer, pancreatic cancer, thyroid cancer). Cancer is not confined to humans; animals and other living organisms can get cancer.

In some instances, the cancer may be malignant. Alternatively, the cancer may be benign. The cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Alternatively, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. Preferably, the cancer is a prostate cancer. Alternatively, the cancer may be a thyroid cancer, bladder cancer, or pancreatic cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavitity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma.

Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

### Cancer Staging

Diagnosing, predicting, or monitoring a status or outcome of a cancer may comprise determining the stage of the cancer. Generally, the stage of a cancer is a description (usually numbers I to IV with IV having more progression) of the extent the cancer has spread. The stage often takes into account the size of a tumor, how deeply it has penetrated, whether it has invaded adjacent organs, how many lymph nodes it has metastasized to (if any), and whether it has spread to distant organs. Staging of cancer can be used as a predictor of survival, and cancer treatment may be determined by staging. Determining the stage of the cancer may occur before, during, or after treatment. The stage of the cancer may also be determined at the time of diagnosis.

Cancer staging can be divided into a clinical stage and a pathologic stage. Cancer staging may comprise the TNM classification. Generally, the TNM Classification of Malignant Tumours (TNM) is a cancer staging system that describes the extent of cancer in a patient's body. T may describe the size of the tumor and whether it has invaded nearby tissue, N may describe regional lymph nodes that are involved, and M may describe distant metastasis (spread of cancer from one body part to another). In the TNM (Tumor, Node, Metastasis) system, clinical stage and pathologic stage are denoted by a small “c” or “p” before the stage (e.g., cT3N1M0 or pT2N0).

Often, clinical stage and pathologic stage may differ. Clinical stage may be based on all of the available information obtained before a surgery to remove the tumor. Thus, it may include information about the tumor obtained by physical examination, radiologic examination, and endoscopy. Pathologic stage can add additional information gained by examination of the tumor microscopically by a pathologist. Pathologic staging can allow direct examination of the tumor and its spread, contrasted with clinical staging which may be limited by the fact that the information is obtained by making indirect observations at a tumor which is still in the body. The TNM staging system can be used for most forms of cancer.

Alternatively, staging may comprise Ann Arbor staging. Generally, Ann Arbor staging is the staging system for lymphomas, both in Hodgkin's lymphoma (previously called Hodgkin's disease) and Non-Hodgkin lymphoma (abbreviated NHL). The stage may depend on both the place where the malignant tissue is located (as located with biopsy, CT scanning and increasingly positron emission tomography) and on systemic symptoms due to the lymphoma (“B symptoms”: night sweats, weight loss of >10% or fevers). The principal stage may be determined by location of the tumor. Stage I may indicate that the cancer is located in a single region, usually one lymph node and the surrounding area. Stage I often may not have outward symptoms. Stage II can indicate that the cancer is located in two separate regions, an affected lymph node or organ and a second affected area, and that both affected areas are confined to one side of the diaphragm—that is, both are above the diaphragm, or both are below the diaphragm. Stage III often indicates that the cancer has spread to both sides of the diaphragm, including one organ or area near the lymph nodes or the spleen. Stage IV may indicate diffuse or disseminated involvement of one or more extralymphatic organs, including any involvement of the liver, bone marrow, or nodular involvement of the lungs.

Modifiers may also be appended to some stages. For example, the letters A, B, E, X, or S can be appended to some stages. Generally, A or B may indicate the absence of constitutional (B-type) symptoms is denoted by adding an “A” to the stage; the presence is denoted by adding a “B” to the stage. E can be used if the disease is “extranodal” (not in the lymph nodes) or has spread from lymph nodes to adjacent tissue. X is often used if the largest deposit is >10 cm large (“bulky disease”), or whether the mediastinum is wider than 1/3 of the chest on a chest X-ray. S may be used if the disease has spread to the spleen.

The nature of the staging may be expressed with CS or PS. CS may denote that the clinical stage as obtained by doctor's examinations and tests. PS may denote that the pathological stage as obtained by exploratory laparotomy (surgery performed through an abdominal incision) with splenectomy (surgical removal of the spleen).

### Therapeutic Regimens

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting responders to an anti-cancer therapy. In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include surgery, chemotherapy, radiation therapy, immunotherapy/biological therapy, photodynamic therapy.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agens may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antiobiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning) In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therepy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a prostate tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat patients with breast cancer who have undergone breast-conserving surgery.

The placement of brachytherapy sources can be temporary or permanent. For permament brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO2) lasers, argon lasers, and neodymium:yttrium-aluminum-garnet (Nd:YAG) lasers. Each of these can shrink or destroy tumors and can be used with endoscopes. CO2 and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

For patients with high test scores consistent with systemic disease outcome after prostatectomy, additional treatment modalities such as adjuvant chemotherapy (e.g., docetaxel, mitoxantrone and prednisone), systemic radiation therapy (e.g., samarium or strontium) and/or anti-androgen therapy (e.g., surgical castration, finasteride, dutasteride) can be designated. Such patients would likely be treated immediately with anti-androgen therapy alone or in combination with radiation therapy in order to eliminate presumed micro-metastatic disease, which cannot be detected clinically but can be revealed by the target sequence expression signature.

Such patients can also be more closely monitored for signs of disease progression. For patients with intermediate test scores consistent with biochemical recurrence only (BCR-only or elevated PSA that does not rapidly become manifested as systemic disease only localized adjuvant therapy (e.g., radiation therapy of the prostate bed) or short course of anti-androgen therapy would likely be administered. For patients with low scores or scores consistent with no evidence of disease (NED) adjuvant therapy would not likely be recommended by their physicians in order to avoid treatment-related side effects such as metabolic syndrome (e.g., hypertension, diabetes and/or weight gain), osteoporosis, proctitis, incontinence or impotence. Patients with samples consistent with NED could be designated for watchful waiting, or for no treatment. Patients with test scores that do not correlate with systemic disease but who have successive PSA increases could be designated for watchful waiting, increased monitoring, or lower dose or shorter duration anti-androgen therapy.

Target sequences can be grouped so that information obtained about the set of target sequences in the group can be used to make or assist in making a clinically relevant judgment such as a diagnosis, prognosis, or treatment choice.

A patient report is also provided comprising a representation of measured expression levels of a plurality of target sequences in a biological sample from the patient, wherein the representation comprises expression levels of target sequences corresponding to any one, two, three, four, five, six, eight, ten, twenty, thirty, fifty or more of the target sequences corresponding to a target selected from Table 1, the subsets described herein, or a combination thereof. A patient report is also provided comprising a representation of measured expression levels of a plurality of target sequences in a biological sample from the patient, wherein the representation comprises expression levels of target sequences corresponding to 40, 50, 60, 70, 80, 90, 100 or more of the target sequences corresponding to a target selected from Table 1, the subsets described herein, or a combination thereof or more coding targets and/or non-coding targets selected from Table 1. A patient report is also provided comprising a representation of measured expression levels of a plurality of target sequences in a biological sample from the patient, wherein the representation comprises expression levels of target sequences corresponding to 100, 125, 150, 175, 200, 225, 250, 275, 300 or more of the target sequences corresponding to a target selected from Table 1, the subsets described herein, or a combination thereof. A patient report is also provided comprising a representation of measured expression levels of a plurality of target sequences in a biological sample from the patient, wherein the representation comprises expression levels of target sequences corresponding to 300, 325, 350, 375, 400, 425, 450, 475, 500, 525, 550, 575, 600 or more of the target sequences corresponding to a target selected from Table 1, the subsets described herein, or a combination thereof. A patient report is also provided comprising a representation of measured expression levels of a plurality of target sequences in a biological sample from the patient, wherein the representation comprises expression levels of target sequences corresponding to 600, 625, 650, 675, 700, 725, 750, 775, 800, 825, 850 or more of the target sequences corresponding to a target selected from Table 1, the subsets described herein, or a combination thereof. In some embodiments, the representation of the measured expression level(s) may take the form of a linear or nonlinear combination of expression levels of the target sequences of interest. The patient report may be provided in a machine (e.g., a computer) readable format and/or in a hard (paper) copy. The report can also include standard measurements of expression levels of said plurality of target sequences from one or more sets of patients with known disease status and/or outcome. The report can be used to inform the patient and/or treating physician of the expression levels of the expressed target sequences, the likely medical diagnosis and/or implications, and optionally may recommend a treatment modality for the patient.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms can assist in the visualization of such data.

### EXEMPLARY EMBODIMENTS

Disclosed herein, in some embodiments, is a method for diagnosing, predicting, and/or monitoring a status or outcome of a cancer a subject, comprising: (a) assaying an expression level of a plurality of targets in a sample from the subject, wherein at least one target of the plurality of targets is selected from the group consisting of targets identified in Table 1; and (b) for diagnosing, predicting, and/or monitoring a status or outcome of a cancer based on the expression levels of the plurality of targets. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a thyroid cancer. In some embodiments, the cancer is a bladder cancer. In some embodiments, the cancer is a lung cancer. In some embodiments, the method further comprises assaying an expression level of a coding target. In some instances, the coding target is selected from the group consisting of targets identified in Table 1. In some embodiments, the coding target is an exon-coding transcript. In some embodiments, the exon-coding transcript is an exonic sequence. In some embodiments, the method further comprises assaying an expression level of a non-coding target. In some instances, the non-coding target is selected from the group consisting of targets identified in Table 1. In some instances, the non-coding target is a non-coding transcript. In other instances, the non-coding target is an intronic sequence. In other instances, the non-coding target is an intergenic sequence. In some instances, the non-coding target is a UTR sequence. In other instances, the non-coding target is a non-coding RNA transcript. In some embodiments, the target comprises a nucleic acid sequence. In some embodiments, the nucleic acid sequence is a DNA sequence. In some embodiments, the nucleic acid sequence is an RNA sequence. In other instances, the target comprises a polypeptide sequence. In some instances, the plurality of targets comprises 2 or more targets selected from the group of targets identified in Table 1. In some instances, the plurality of targets comprises 5 or more targets selected from the group of targets identified in Table 1. In some instances, the plurality of targets comprises 10 or more targets selected from the group of targets identified in Table 1. In some instances, the plurality of targets comprises 15 or more targets selected from the group of targets identified in Table 1. In some instances, the plurality of targets comprises 20 or more targets selected from the group of targets identified in Table 1. In some instances, the plurality of targets comprises 25 or more targets selected from the group of targets identified in Table 1. In some instances, the plurality of targets comprises 30 or more targets selected from the group of targets identified in Table 1. In some instances, the plurality of targets comprises 35 or more targets selected from the group of targets identified in Table 1. In some instances, the plurality of targets comprises 40 or more targets selected from the group of targets identified in Table 1. In some embodiments, assaying the expression level comprises detecting and/or quantifying a nucleotide sequence of the plurality of targets. Alternatively, assaying the expression level comprises detecting and/or quantifying a polypeptide sequence of the plurality of targets. In some embodiments, assaying the expression level comprises detecting and/or quantifying the DNA levels of the plurality of targets. In some embodiments, assaying the expression level comprises detecting and/or quantifying the RNA or mRNA levels of the plurality of targets. In some embodiments, assaying the expression level comprises detecting and/or quantifying the protein level of the plurality of targets. In some embodiments, the diagnosing, predicting, and/or monitoring the status or outcome of a cancer comprises determining the malignancy of the cancer. In some embodiments, the diagnosing, predicting, and/or monitoring the status or outcome of a cancer includes determining the stage of the cancer. In some embodiments, the diagnosing, predicting, and/or monitoring the status or outcome of a cancer includes assessing the risk of cancer recurrence. In some embodiments, diagnosing, predicting, and/or monitoring the status or outcome of a cancer may comprise determining the efficacy of treatment. In some embodiments, diagnosing, predicting, and/or monitoring the status or outcome of a cancer may comprise determining a therapeutic regimen. Determining a therapeutic regimen may comprise administering an anti-cancer therapeutic. Alternatively, determining the treatment for the cancer may comprise modifying a therapeutic regimen. Modifying a therapeutic regimen may comprise increasing, decreasing, or terminating a therapeutic regimen.

Further disclosed, in some embodiments, is method for determining a treatment for a cancer in a subject, comprising: a) assaying an expression level of a plurality of targets in a sample from the subject, wherein at least one target of the plurality of targets is selected from the group consisting of targets identified in Table 1; and b) determining the treatment for a cancer based on the expression levels of the plurality of targets. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a bladder cancer. In some embodiments, the cancer is a thyroid cancer. In some embodiments, the cancer is a lung cancer. In some embodiments, the coding target is selected from a sequence listed in Table 1. In some embodiments, the method further comprises assaying an expression level of a coding target. In some instances, the coding target is selected from the group consisting of targets identified in Table 1. In some embodiments, the coding target is an exon-coding transcript. In some embodiments, the exon-coding transcript is an exonic sequence. In some embodiments, the method further comprises assaying an expression level of a non-coding target. In some instances, the non-coding target is selected from the group consisting of targets identified in Table 1. In some instances, the non-coding target is a non-coding transcript. In other instances, the non-coding target is an intronic sequence. In other instances, the non-coding target is an intergenic sequence. In some instances, the non-coding target is a UTR sequence. In other instances, the non-coding target is a non-coding RNA transcript. In some embodiments, the target comprises a nucleic acid sequence. In some embodiments, the nucleic acid sequence is a DNA sequence. In some embodiments, the nucleic acid sequence is an RNA sequence. In other instances, the target comprises a polypeptide sequence. In some instances, the plurality of targets comprises 2 or more targets selected from the group of targets identified in Table 1. In some instances, the plurality of targets comprises 5 or more targets selected from the group of targets identified in Table 1. In some instances, the plurality of targets comprises 10 or more targets selected from the group of targets identified in Table 1. In some instances, the plurality of targets comprises 15 or more targets selected from the group of targets identified in Table 1. In some instances, the plurality of targets comprises 20 or more targets selected from the group of targets identified in Table 1. In some instances, the plurality of targets comprises 25 or more targets selected from the group of targets identified in Table 1. In some instances, the plurality of targets comprises 30 or more targets selected from the group of targets identified in Table 1. In some instances, the plurality of targets comprises 35 or more targets selected from the group of targets identified in Table 1. In some instances, the plurality of targets comprises 40 or more targets selected from the group of targets identified in Table 1. In some embodiments, assaying the expression level comprises detecting and/or quantifying a nucleotide sequence of the plurality of targets. In some embodiments, determining the treatment for the cancer includes determining the efficacy of treatment. Determining the treatment for the cancer may comprise administering an anti-cancer therapeutic. Alternatively, determining the treatment for the cancer may comprise modifying a therapeutic regimen. Modifying a therapeutic regimen may comprise increasing, decreasing, or terminating a therapeutic regimen.

The methods use the probe sets, probes and primers described herein to provide expression signatures or profiles from a test sample derived from a subject having or suspected of having cancer. In some embodiments, such methods involve contacting a test sample with a probe set comprising a plurality of probes under conditions that permit hybridization of the probe(s) to any target nucleic acid(s) present in the test sample and then detecting any probe:target duplexes formed as an indication of the presence of the target nucleic acid in the sample. Expression patterns thus determined are then compared to one or more reference profiles or signatures. Optionally, the expression pattern can be normalized. The methods use the probe sets, probes and primers described herein to provide expression signatures or profiles from a test sample derived from a subject to classify the cancer as recurrent or non-recurrent.

In some embodiments, such methods involve the specific amplification of target sequences nucleic acid(s) present in the test sample using methods known in the art to generate an expression profile or signature which is then compared to a reference profile or signature.

In some embodiments, the invention further provides for prognosing patient outcome, predicting likelihood of recurrence after prostatectomy and/or for designating treatment modalities.

In one embodiment, the methods generate expression profiles or signatures detailing the expression of the target sequences having altered relative expression with different cancer outcomes.

In some embodiments, the methods detect combinations of expression levels of sequences exhibiting positive and negative correlation with a disease status. In one embodiment, the methods detect a minimal expression signature.

The gene expression profiles of each of the target sequences comprising the portfolio can fixed in a medium such as a computer readable medium. This can take a number of forms. For example, a table can be established into which the range of signals (e.g., intensity measurements) indicative of disease or outcome is input. Actual patient data can then be compared to the values in the table to determine the patient samples diagnosis or prognosis. In a more sophisticated embodiment, patterns of the expression signals (e.g., fluorescent intensity) are recorded digitally or graphically.

The expression profiles of the samples can be compared to a control portfolio. The expression profiles can be used to diagnose, predict, or monitor a status or outcome of a cancer. For example, diagnosing, predicting, or monitoring a status or outcome of a cancer may comprise diagnosing or detecting a cancer, cancer metastasis, or stage of a cancer. In other instances, diagnosing, predicting, or monitoring a status or outcome of a cancer may comprise predicting the risk of cancer recurrence. Alternatively, diagnosing, predicting, or monitoring a status or outcome of a cancer may comprise predicting mortality or morbidity.

Further disclosed herein are methods for characterizing a patient population. Generally, the method comprises: (a) providing a sample from a subject; (b) assaying the expression level for a plurality of targets in the sample; and (c) characterizing the subject based on the expression level of the plurality of targets. In some embodiments, the method further comprises assaying an expression level of a coding target. In some instances, the coding target is selected from the group consisting of targets identified in Table 1. In some embodiments, the coding target is an exon-coding transcript. In some embodiments, the exon-coding transcript is an exonic sequence. In some embodiments, the method further comprises assaying an expression level of a non-coding target. In some instances, the non-coding target is selected from the group consisting of targets identified in Table 1. In some instances, the non-coding target is a non-coding transcript. In other instances, the non-coding target is an intronic sequence. In other instances, the non-coding target is an intergenic sequence. In some instances, the non-coding target is a UTR sequence. In other instances, the non-coding target is a non-coding RNA transcript. In some embodiments, the target comprises a nucleic acid sequence. In some embodiments, the nucleic acid sequence is a DNA sequence. In some embodiments, the nucleic acid sequence is an RNA sequence. In other instances, the target comprises a polypeptide sequence. In some instances, the plurality of targets comprises 2 or more targets selected from the group of targets identified in Table 1. In some instances, the plurality of targets comprises 5 or more targets selected from the group of targets identified in Table 1. In some instances, the plurality of targets comprises 10 or more targets selected from the group of targets identified in Table 1. In some instances, the plurality of targets comprises 15 or more targets selected from the group of targets identified in Table 1. In some instances, the plurality of targets comprises 20 or more targets selected from the group of targets identified in Table 1. In some instances, the plurality of targets comprises 25 or more targets selected from the group of targets identified in Table 1. In some instances, the plurality of targets comprises 30 or more targets selected from the group of targets identified in Table 1.

In some instances, the plurality of targets comprises 35 or more targets selected from the group of targets identified in Table 1. In some instances, the plurality of targets comprises 40 or more targets selected from the group of targets identified in Table 1. In some embodiments, assaying the expression level comprises detecting and/or quantifying a nucleotide sequence of the plurality of targets. In some instances, the method may further comprise diagnosing a cancer in the subject. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a bladder cancer. In some embodiments, the cancer is a thyroid cancer. In some embodiments, the cancer is a lung cancer. In some instances, characterizing the subject comprises determining whether the subject would respond to an anti-cancer therapy. Alternatively, characterizing the subject comprises identifying the subject as a non-responder to an anti-cancer therapy. Optionally, characterizing the subject comprises identifying the subject as a responder to an anti-cancer therapy.

Further disclosed herein are methods for selecting a subject suffering from a cancer for enrollment into a clinical trial. Generally, the method comprises: (a) providing a sample from a subject; (b) assaying the expression level for a plurality of targets in the sample; and (c) characterizing the subject based on the expression level of the plurality of targets. In some embodiments, the method further comprises assaying an expression level of a coding target. In some instances, the coding target is selected from the group consisting of targets identified in Table 1. In some embodiments, the coding target is an exon-coding transcript. In some embodiments, the exon-coding transcript is an exonic sequence. In some embodiments, the method further comprises assaying an expression level of a non-coding target. In some instances, the non-coding target is selected from the group consisting of targets identified in Table 1. In some instances, the non-coding target is a non-coding transcript. In other instances, the non-coding target is an intronic sequence. In other instances, the non-coding target is an intergenic sequence. In some instances, the non-coding target is a UTR sequence. In other instances, the non-coding target is a non-coding RNA transcript. In some embodiments, the target comprises a nucleic acid sequence. In some embodiments, the nucleic acid sequence is a DNA sequence. In some embodiments, the nucleic acid sequence is an RNA sequence. In other instances, the target comprises a polypeptide sequence. In some instances, the plurality of targets comprises 2 or more targets selected from the group of targets identified in Table 1. In some instances, the plurality of targets comprises 5 or more targets selected from the group of targets identified in Table 1. In some instances, the plurality of targets comprises 10 or more targets selected from the group of targets identified in Table 1. In some instances, the plurality of targets comprises 15 or more targets selected from the group of targets identified in Table 1. In some instances, the plurality of targets comprises 20 or more targets selected from the group of targets identified in Table 1. In some instances, the plurality of targets comprises 25 or more targets selected from the group of targets identified in Table 1. In some instances, the plurality of targets comprises 30 or more targets selected from the group of targets identified in Table 1. In some instances, the plurality of targets comprises 35 or more targets selected from the group of targets identified in Table 1. In some instances, the plurality of targets comprises 40 or more targets selected from the group of targets identified in Table 1. In some embodiments, assaying the expression level comprises detecting and/or quantifying a nucleotide sequence of the plurality of targets. In some instances, the method may further comprise diagnosing a cancer in the subject. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a prostate cancer. In some embodiments, the cancer is a pancreatic cancer. In some embodiments, the cancer is a bladder cancer. In some embodiments, the cancer is a thyroid cancer. In some embodiments, the cancer is a lung cancer. In some instances, characterizing the subject comprises determining whether the subject would respond to an anti-cancer therapy. Alternatively, characterizing the subject comprises identifying the subject as a non-responder to an anti-cancer therapy. Optionally, characterizing the subject comprises identifying the subject as a responder to an anti-cancer therapy.

Further disclosed herein is a method of analyzing a cancer in an individual in need thereof, comprising (a) obtaining an expression profile from a sample obtained from the individual, wherein the expression profile comprises one or more targets selected from Table 1; and (b) comparing the expression profile from the sample to an expression profile of a control or standard. In some embodiments, the plurality of targets comprises at least 5 targets selected from Table 1. In some embodiments, wherein the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the method further comprises providing diagnostic or prognostic information to the individual about the cardiovascular disorder based on the comparison. In some embodiments, the method further comprises diagnosing the individual with a cancer if the expression profile of the sample (a) deviates from the control or standard from a healthy individual or population of healthy individuals, or (b) matches the control or standard from an individual or population of individuals who have or have had the cancer. In some embodiments, the method further comprises predicting the susceptibility of the individual for developing a cancer based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises prescribing a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises altering a treatment regimen prescribed or administered to the individual based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1 or a combination thereof. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

Also disclosed herein is a method of diagnosing cancer in an individual in need thereof, comprising (a) obtaining an expression profile from a sample obtained from the individual, wherein the expression profile comprises one or more targets selected from Table 1; (b) comparing the expression profile from the sample to an expression profile of a control or standard; and (c) diagnosing a cancer in the individual if the expression profile of the sample (i) deviates from the control or standard from a healthy individual or population of healthy individuals, or (ii) matches the control or standard from an individual or population of individuals who have or have had the cancer. In some embodiments, the plurality of targets comprises at least 5 targets selected from Table 1. In some embodiments, wherein the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

In some embodiments is a method of predicting whether an individual is susceptible to developing a cancer, comprising (a) obtaining an expression profile from a sample obtained from the individual, wherein the expression profile comprises one or more targets selected from Table 1; (b) comparing the expression profile from the sample to an expression profile of a control or standard; and (c) predicting the susceptibility of the individual for developing a cancer based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 1. In some embodiments, wherein the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

In some embodiments is a method of predicting an individual's response to a treatment regimen for a cancer, comprising: (a) obtaining an expression profile from a sample obtained from the individual, wherein the expression profile comprises one or more targets selected from Table 1; (b) comparing the expression profile from the sample to an expression profile of a control or standard; and (c) predicting the individual's response to a treatment regimen based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 1. In some embodiments, wherein the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

A method of prescribing a treatment regimen for a cancer to an individual in need thereof, comprising (a) obtaining an expression profile from a sample obtained from the individual, wherein the expression profile comprises one or more targets selected from Table 1; (b) comparing the expression profile from the sample to an expression profile of a control or standard; and (c) prescribing a treatment regimen based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 1. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

Further disclosed herein is a kit for analyzing a cancer, comprising (a) a probe set comprising a plurality of target sequences, wherein the plurality of target sequences comprises at least one target sequence listed in Table 1; and (b) a computer model or algorithm for analyzing an expression level and/or expression profile of the target sequences in a sample. In some embodiments, the kit further comprises a computer model or algorithm for correlating the expression level or expression profile with disease state or outcome. In some embodiments, the kit further comprises a computer model or algorithm for designating a treatment modality for the individual. In some embodiments, the kit further comprises a computer model or algorithm for normalizing expression level or expression profile of the target sequences. In some embodiments, the kit further comprises a computer model or algorithm comprising a robust multichip average (RMA), probe logarithmic intensity error estimation (PLIER), non-linear fit (NLFIT) quantile-based, nonlinear normalization, or a combination thereof. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

Further disclosed herein is a system for analyzing a cancer, comprising (a) a probe set comprising a plurality of target sequences, wherein (i) the plurality of target sequences hybridizes to one or more targets selected from Table 1; or (ii) the plurality of target sequences comprises one or more target sequences selected from Table 1; and (b) a computer model or algorithm for analyzing an expression level and/or expression profile of the target hybridized to the probe in a sample from a subject suffering from a cancer. In some embodiments, the system further comprises electronic memory for capturing and storing an expression profile. In some embodiments, the system further comprises a computer-processing device, optionally connected to a computer network. In some embodiments, the system further comprises a software module executed by the computer-processing device to analyze an expression profile. In some embodiments, the system further comprises a software module executed by the computer-processing device to compare the expression profile to a standard or control. In some embodiments, the system further comprises a software module executed by the computer-processing device to determine the expression level of the target. In some embodiments, the system further comprises a machine to isolate the target or the probe from the sample. In some embodiments, the system further comprises a machine to sequence the target or the probe. In some embodiments, the system further comprises a machine to amplify the target or the probe. In some embodiments, the system further comprises a label that specifically binds to the target, the probe, or a combination thereof. In some embodiments, the system further comprises a software module executed by the computer-processing device to transmit an analysis of the expression profile to the individual or a medical professional treating the individual. In some embodiments, the system further comprises a software module executed by the computer-processing device to transmit a diagnosis or prognosis to the individual or a medical professional treating the individual. In some embodiments, the plurality of targets comprises at least 5 targets selected from Table 1. In some embodiments, the plurality of targets comprises at least 10 targets selected from Table 1. In some embodiments, the plurality of targets comprises at least 15 targets selected from Table 1. In some embodiments, the plurality of targets comprises at least 20 targets selected from Table 1. In some embodiments, the cancer is selected from the group consisting of a carcinoma, sarcoma, leukemia, lymphoma, myeloma, and a CNS tumor. In some embodiments, the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

### EXAMPLES

### Example 1

### A 13 Biomarker Classifier to Predict Biochemical Recurrence in Prostate Cancer Samples

Methods

The publically available Memorial Sloan Kettering (MSKCC) Prostate Oncogenome project dataset (http://www.ncbi.nlm.nih.gov/geo/query/acc.cgi?acc=GSE21034) was used for this analysis, which consists of 131 primary tumor microarray samples (Affymetrix Human Exon 1.0 ST array) (Taylor et al 2010). Information on Tissue samples, RNA extraction, RNA amplification and hybridization can be found elsewhere (Taylor et al 2010). These samples were preprocessed using frozen Robust Multiarray Average (fRMA), with quantile normalization and robust weighted average summarization. Additional publicly available datasets used in the coming examples are the DKFZ (http://www.ncbi.nlm.nih.gov/geo/query/acc.cgi?acc=GSE29079) and the ICR dataset (http://www.ncbi.nlm.nih.gov/geo/query/acc.cgi?acc=GSE212378) and were pre-processed in the same manner as the MSKCC dataset. Further details can be found in the links provided.

The 1,411,399 expression features on the array were filtered to remove unreliable probesets using a cross hybridization and background filter. The cross hybridization filter removes any probesets which are defined by Affymetrix to have cross hybridization potential (class 1), which ensures that the probeset is measuring only the expression level of only a specific genomic location. Feature selection was performed in the MSKCC (n=131) datasets using a T-Test filter. Features found to have a significance less then p<0.001 (n=13) were included in the model. The 13 features were standardized using the percentile rank of the expression values across the patients before being modeled with a random forest (R package randomForest 4.6-7) classifier using the default parameters. The classifier generates a score between 0 and 1 where higher values indicate higher potential for Biochemical Recurrence.

This study used a previously described case-control study (Nakagawa et al 2008) and a case-cohort for independent validation.

RNA Extraction and Microarray Hybridization

Following pathological review of FFPE primary prostatic adenocarcinoma specimens from patients in the discovery and validation cohorts, tumor was macrodissected from surrounding stroma from 3-4 10 μm tissue sections. Total RNA was extracted, amplified using the Ovation FFPE kit (NuGEN, San Carlos, Calif.), and hybridized to Human Exon 1.0 ST GeneChips (Affymetrix, Santa Clara, Calif.) that profiles coding and non-coding regions of the transcriptome using approximately 1.4 million probe selection regions, hereinafter referred to as features.

For the discovery study, total RNA was prepared as described herein. For the independent validation study, total RNA was extracted and purified using a modified protocol for the commercially available RNeasy FFPE nucleic acid extraction kit (Qiagen Inc., Valencia, Calif.). RNA concentrations were determined using a Nanodrop ND-1000 spectrophotometer (Nanodrop Technologies, Rockland, Del.). Purified total RNA was subjected to whole-transcriptome amplification using the WT-Ovation FFPE system according to the manufacturer's recommendation with minor modifications (NuGen, San Carlos, Calif.). For the discovery study the WT-Ovation FFPE V2 kit was used together with the Exon Module while for the validation only the Ovation® FFPE WTA System was used. Amplified products were fragmented and labeled using the Encore™ Biotin Module (NuGen, San Carlos, Calif.) and hybridized to Affymetrix Human Exon (HuEx) 1.0 ST GeneChips following manufacturer's recommendations (Affymetrix, Santa Clara, Calif.). Only 604 out of a total 621 patients had specimens available for hybridization.

Microarray Quality Control

The Affymetrix Power Tools packages provide an index characterizing the quality of each chip, independently, named “pos_vs_neg_AUC”. This index compares signal values for positive and negative control probesets defined by the manufacturer. Values for the AUC are in [0, 1], arrays that fall under 0.6 were removed from analysis.

Only 545 unique samples, out of the total 604 with available specimens (inter- and intra-batch duplicates were run), were of sufficient quality for further analysis; 359 and 187 samples were available from the training (Mayo Training) and testing (Mayo Testing) sets respectively. We re-evaluated the variable balance between the training and testing sets and found there to be no statistically significant difference for any of the variables.

Microarray Normalization, Probeset filtering, and Batch Effect Correction

Probeset summarization and normalization was performed by fRMA, which is available through Bioconductor. The fRMA algorithm relates to RMA with the exception that it specifically attempts to consider batch effect during probeset summarization and is capable of storing the model parameters in so called ‘frozen vectors’. We generated a custom set of frozen vectors by randomly selecting 15 arrays from each of the 19 batches in the discovery study. The frozen vectors can be applied to novel data without having to renormalize the entire dataset. We furthermore filtered out unreliable PSRs by removing cross-hybridizing probes as well as high PSRs variability of expression values in a prostate cancer cell line and those with fewer than 4 probes. Following fRMA and filtration the data was decomposed into its principal components and an analysis of variance model was used to determine the extent to which a batch effect remains present in the first 10 principal components. We chose to remove the first two principal components, as they were highly correlated with the batch processing date.

The discovery study was a nested case-control described in detail in Nakagawa. Archived formalin-fixed paraffin embedded (FFPE) blocks of tumors were selected from 621 patients that had undergone a radical prostatectomy (RP) at the Mayo Clinic Comprehensive Cancer Centre between the years 1987-2001 providing a median of 18.16 years of follow-up. After chip quality control (http://www.affymetrix.com), 545 unique patients were available for biomarker validation. The study patients were further subdivided by random draw into training (n=359) and testing (n=186) subsets, balancing for the distribution of clinicopathologic variables. Subjects for the case-cohort group were identified from a population of 1,010 men prospectively enrolled in the Mayo Clinic tumor registry who underwent RP for prostatic adenocarcinoma from 2000-2006 and were at high risk for disease recurrence. High-risk for recurrence was defined by pre-operative PSA>20 ng/mL, or pathological Gleason score >8, or seminal vesicle invasion (SVI) or GPSM (Gleason, PSA, seminal vesicle and margin status) score >10. Data was collected using a case-cohort design over the follow-up period (median, 8.06 years), 71 patients developed metastatic disease (mets) as evidenced by positive bone and/or CT scans. Data was collected using a case-cohort design, which involved selection of all 73 cases combined with a random sample of 202 patients (˜20%) from the entire cohort. After exclusion for tissue unavailability and samples that failed microarray quality control, the independent validation cohort consisted of 219 (69 cases) unique patients.

Results

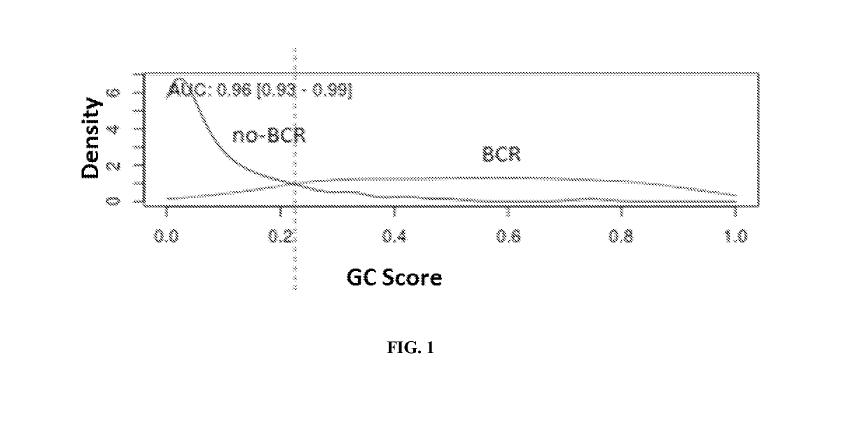

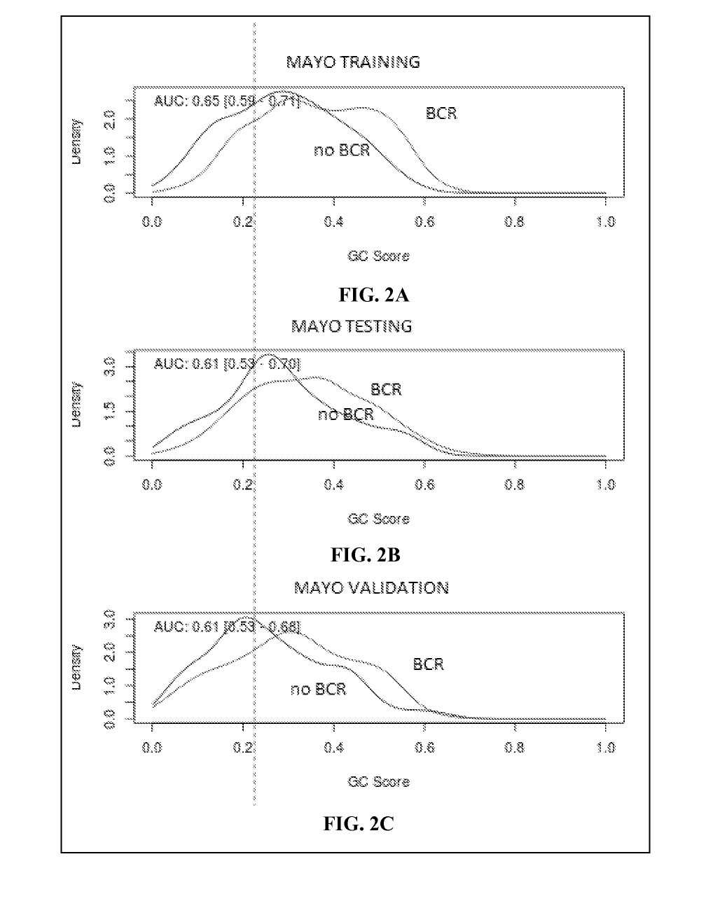

The 13 features that correspond to the generated Random Forest classifier are: SEQ ID NO. 380, SEQ ID NO. 111, SEQ ID NO. 318, SEQ ID NO. 338, SEQ ID NO. 559, SEQ ID NO. 610, SEQ ID NO. 614, SEQ ID NO. 712, SEQ ID NO. 750, SEQ ID NO. 751, SEQ ID NO. 752, SEQ ID NO. 753, SEQ ID NO. 818. Further details on these sequences are provided in Table 1. Performance of this classifier based on AUC on the MSKCC data reaches a value of 0.96 (FIG. 1; 95% Confidence Interval: [0.93-0.99]). The fact that the confidence interval doesn't overlap with the 0.5 threshold demonstrates the statistical significance of the result. AUC Performance on the Mayo Training, Mayo testing and Mayo Validation datasets is 0.65, 0.61 and 0.61 respectively, with all AUCs being statistically significant based on their 95% Confidence Interval (FIG. 2).

### Example 2

### A 13 Biomarker Classifier to Predict PSA Doubling Time in Prostate Cancer Samples

Methods

The Mayo discovery dataset described in Example 1 was used for feature selection and to train the model. Both the Mayo training, testing and validation datasets were used for performance assessment. The top 13 features were selected for modeling based on a t-test p-value ranking Standardization of the 13 features was performed via a percentile ranking of the features across patients. These standardized features were then modeled using a tuned cross validation) random forest model (mtry and node parameters, R package randomForest 4.6-7) to produce the classifier. PSADT event was defined by a threshold of 9 months after surgery. The classifier generates a score between 0 and 1 where higher values indicate higher potential for rapid PSADT.

Results

The 13 features that correspond to the generated Random Forest classifier are: SEQ ID NO. 123, SEQ ID NO. 807, SEQ ID NO. 247, SEQ ID NO. 100, SEQ ID NO. 6, SEQ ID NO. 213, SEQ ID NO. 169, SEQ ID NO. 42, SEQ ID NO. 78, SEQ ID NO. 159, SEQ ID NO. 32, SEQ ID NO. 398, SEQ ID NO. 108.

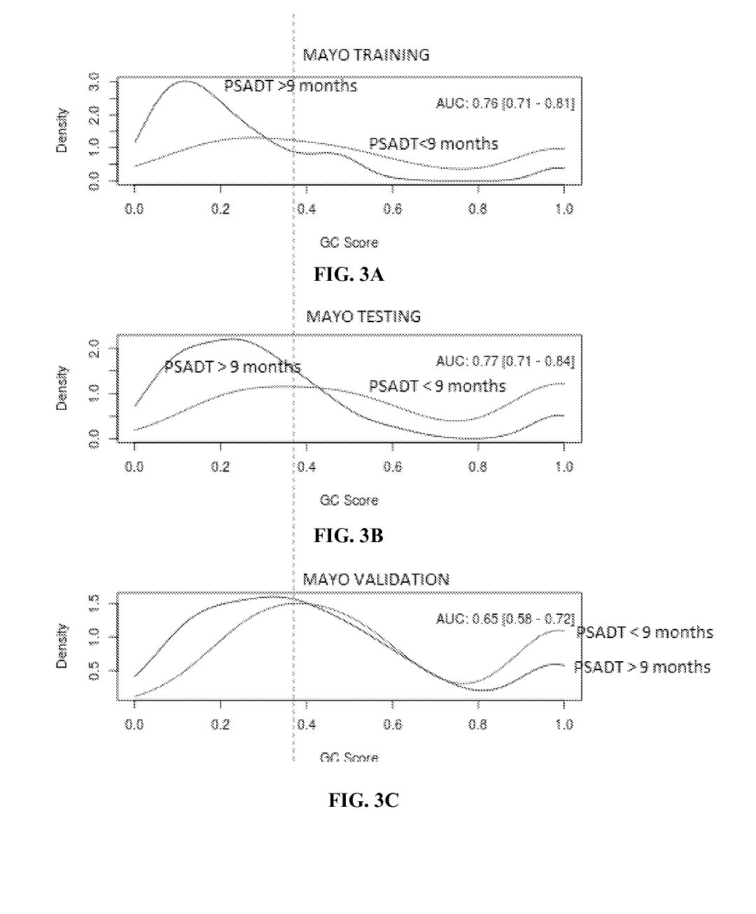

Further details on these sequences are provided in Table 1. Performance on the Mayo Training, Mayo testing and Mayo Validation datasets is 0.76, 0.77 and 0.65 respectively, with all AUCs being statistically significant based on their 95% Confidence Interval (FIG. 3). These results show the prognostic ability of the classifier to predict rapid PSADT after surgery.

### Example 3

### A 58 Biomarker Classifier to Predict Androgen Deprivation Therapy (ADT) Failure in Prostate Cancer Samples

Methods

The Mayo discovery dataset described in Example 1 was used for feature selection and to train the model. Performance of the model was further assessed in the validation dataset. Modeling is done using patients who received only hormone therapy and not radiation from the Mayo discovery set. Background and cross hybridization filtering (http://www.affymetrix.com) is performed, reducing the number of PSRs to 752,497. 58 features are selected which have the lowest t-test p-values of all the PSRs left. Modeling is performed with a tuned SVM (R package e1071 v1.6-1) after the 58 features are standardized using a percentile rank across the rows. Since SVM generates between −∞ and ∞, these scores are transformed to a probability score by logistic regression, where higher values indicate higher potential for ADT failure.

Results

The 58 features that correspond to the generated SVM classifier are: SEQ ID NO. 421, SEQ ID NO. 277, SEQ ID NO. 634, SEQ ID NO. 250, SEQ ID NO. 530, SEQ ID NO. 336, SEQ ID NO. 136, SEQ ID NO. 826, SEQ ID NO. 534, SEQ ID NO. 710, SEQ ID NO. 495, SEQ ID NO. 714, SEQ ID NO. 679, SEQ ID NO. 770, SEQ ID NO. 727, SEQ ID NO. 815, SEQ ID NO. 624, SEQ ID NO. 754, SEQ ID NO. 678, SEQ ID NO. 385, SEQ ID NO. 320, SEQ ID NO. 655, SEQ ID NO. 396, SEQ ID NO. 234, SEQ ID NO. 558, SEQ ID NO. 266, SEQ ID NO. 48, SEQ ID NO. 83, SEQ ID NO. 834, SEQ ID NO. 816, SEQ ID NO. 414, SEQ ID NO. 2, SEQ ID NO. 392, SEQ ID NO. 617, SEQ ID NO. 693, SEQ ID NO. 355, SEQ ID NO. 87, SEQ ID NO. 755, SEQ ID NO. 697, SEQ ID NO. 482, SEQ ID NO. 519, SEQ ID NO. 69, SEQ ID NO. 817, SEQ ID NO. 607, SEQ ID NO. 395, SEQ ID NO. 627, SEQ ID NO. 89, SEQ ID NO. 9, SEQ ID NO. 303, SEQ ID NO. 500, SEQ ID NO. 604, SEQ ID NO. 223, SEQ ID NO. 598, SEQ ID NO. 98, SEQ ID NO. 668, SEQ ID NO. 523, SEQ ID NO. 782, SEQ ID NO. 68. Further details on these sequences are provided in Table 1.

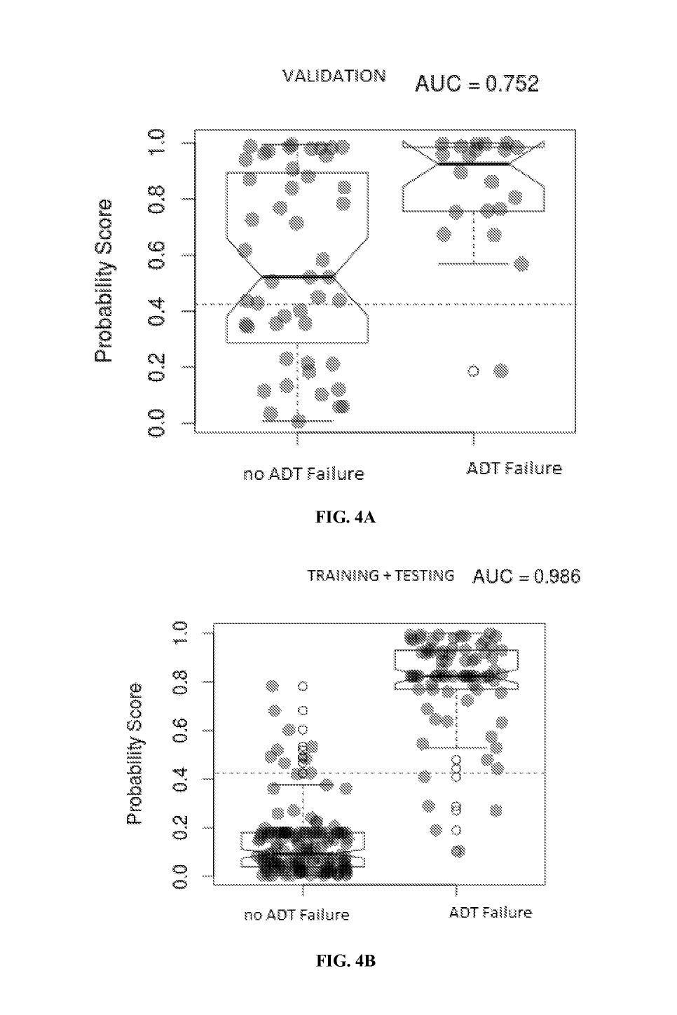

Discrimination plots for the groups of patients with and without ADT Failure based on Discovery and Validation datasets (see Example 1) show no overlap of the associated 95% Confidence Intervals, as demonstrated by the non-overlapping notches in FIG. 4. This suggests that the distribution of scores for both groups is significantly different. The AUC of this classifier is 0.986 and 0.752 for the Discovery (training+testing) and Validation

Datasets, respectively. These results demonstrate the predictive ability of the classifier for ADT Failure.

### Example 4

### A 392-Biomarker Signature that Discriminates Between Patients with High Grade Tumor from Patients with Low Grade Tumor

Methods

Classifier KNN392 is a signature that discriminates between patients with high grade tumor (Gleason Grade 4 or greater) from patients with low grade tumor (Gleason Grade 3 or lower). Features with significant expression difference between patients with low grade tumor and high grade tumor in the mayo discovery and validation datasets (n=400 patients, after excluding Gleason Score 7 patients), as denoted by a Bonferroni-adjusted t-test p-value <0.05 were selected. The 392 features were used after percentile ranking standardization to generate a classifier from the k-Nearest Neighbor algorithm with parameter k=11. Performance of the classifier is assessed in MSKCC cohort (http://www.ncbi.nlm.nih.gov/geo/query/acc.cgi?acc=GSE21034). The score of the classifier represent the probability an individual would be classified as having high grade tumor based on the expression values of the closest 11 patients in the training cohort of 400 prostate samples. The probabilities range from 0 to 1 where low probabilities represent a lower chance a patient would have high grade tumor while higher probabilities represent a higher chance a patient would have high grade tumor.

Results

The 392 features that compose KNN392 are: SEQ ID NO. 1, SEQ ID NO. 3, SEQ ID NO. 4, SEQ ID NO. 5, SEQ ID NO. 7, SEQ ID NO. 15, SEQ ID NO. 17, SEQ ID NO. 18, SEQ ID NO. 19, SEQ ID NO. 21, SEQ ID NO. 22, SEQ ID NO. 26, SEQ ID NO. 27, SEQ ID NO. 30, SEQ ID NO. 31, SEQ ID NO. 32, SEQ ID NO. 33, SEQ ID NO. 34, SEQ ID NO. 35, SEQ ID NO. 40, SEQ ID NO. 41, SEQ ID NO. 43, SEQ ID NO. 45, SEQ ID NO. 50, SEQ ID NO. 51, SEQ ID NO. 52, SEQ ID NO. 53, SEQ ID NO. 54, SEQ ID NO. 56, SEQ ID NO. 58, SEQ ID NO. 61, SEQ ID NO. 62, SEQ ID NO. 70, SEQ ID NO. 72, SEQ ID NO. 75, SEQ ID NO. 76, SEQ ID NO. 77, SEQ ID NO. 79, SEQ ID NO. 80, SEQ ID NO. 85, SEQ ID NO. 88, SEQ ID NO. 91, SEQ ID NO. 92, SEQ ID NO. 93, SEQ ID NO. 96, SEQ ID NO. 101, SEQ ID NO. 102, SEQ ID NO. 103, SEQ ID NO. 104, SEQ ID NO. 107, SEQ ID NO. 110, SEQ ID NO. 112, SEQ ID NO. 113, SEQ ID NO. 114, SEQ ID NO. 126, SEQ ID NO. 127, SEQ ID NO. 132, SEQ ID NO. 134, SEQ ID NO. 135, SEQ ID NO. 138, SEQ ID NO. 139, SEQ ID NO. 140, SEQ ID NO. 141, SEQ ID NO. 142, SEQ ID NO. 144, SEQ ID NO. 145, SEQ ID NO. 147, SEQ ID NO. 148, SEQ ID NO. 149, SEQ ID NO. 150, SEQ ID NO. 151, SEQ ID NO. 152, SEQ ID NO. 153, SEQ ID NO. 154, SEQ ID NO. 157, SEQ ID NO. 162, SEQ ID NO. 171, SEQ ID NO. 172, SEQ ID NO. 173, SEQ ID NO. 174, SEQ ID NO. 176, SEQ ID NO. 178, SEQ ID NO. 180, SEQ ID NO. 181, SEQ ID NO. 182, SEQ ID NO. 183, SEQ ID NO. 185, SEQ ID NO. 188, SEQ ID NO. 192, SEQ ID NO. 193, SEQ ID NO. 194, SEQ ID NO. 200, SEQ ID NO. 201, SEQ ID NO. 202, SEQ ID NO. 203, SEQ ID NO. 205, SEQ ID NO. 206, SEQ ID NO. 208, SEQ ID NO. 210, SEQ ID NO. 211, SEQ ID NO. 214, SEQ ID NO. 215, SEQ ID NO. 216, SEQ ID NO. 218, SEQ ID NO. 221, SEQ ID NO. 222, SEQ ID NO. 226, SEQ ID NO. 227, SEQ ID NO. 228, SEQ ID NO. 230, SEQ ID NO. 231, SEQ ID NO. 235, SEQ ID NO. 236, SEQ ID NO. 240, SEQ ID NO. 242, SEQ ID NO. 243, SEQ ID NO. 245, SEQ ID NO. 246, SEQ ID NO. 249, SEQ ID NO. 261, SEQ ID NO. 263, SEQ ID NO. 264, SEQ ID NO. 265, SEQ ID NO. 267, SEQ ID NO. 268, SEQ ID NO. 269, SEQ ID NO. 270, SEQ ID NO. 271, SEQ ID NO. 275, SEQ ID NO. 276, SEQ ID NO. 279, SEQ ID NO. 280, SEQ ID NO. 281, SEQ ID NO. 282, SEQ ID NO. 284, SEQ ID NO. 285, SEQ ID NO. 286, SEQ ID NO. 287, SEQ ID NO. 288, SEQ ID NO. 289, SEQ ID NO. 290, SEQ ID NO. 291, SEQ ID NO. 292, SEQ ID NO. 293, SEQ ID NO. 295, SEQ ID NO. 298, SEQ ID NO. 300, SEQ ID NO. 301, SEQ ID NO. 302, SEQ ID NO. 304, SEQ ID NO. 305, SEQ ID NO. 306, SEQ ID NO. 307, SEQ ID NO. 309, SEQ ID NO. 311, SEQ ID NO. 312, SEQ ID NO. 315, SEQ ID NO. 316, SEQ ID NO. 317, SEQ ID NO. 319, SEQ ID NO. 321, SEQ ID NO. 322, SEQ ID NO. 324, SEQ ID NO. 328, SEQ ID NO. 329, SEQ ID NO. 330, SEQ ID NO. 331, SEQ ID NO. 332, SEQ ID NO. 333, SEQ ID NO. 335, SEQ ID NO. 337, SEQ ID NO. 338, SEQ ID NO. 339, SEQ ID NO. 340, SEQ ID NO. 341, SEQ ID NO. 345, SEQ ID NO. 346, SEQ ID NO. 347, SEQ ID NO. 348, SEQ ID NO. 351, SEQ ID NO. 352, SEQ ID NO. 354, SEQ ID NO. 356, SEQ ID NO. 357, SEQ ID NO. 360, SEQ ID NO. 361, SEQ ID NO. 363, SEQ ID NO. 364, SEQ ID NO. 366, SEQ ID NO. 367, SEQ ID NO. 368, SEQ ID NO. 369, SEQ ID NO. 370, SEQ ID NO. 371, SEQ ID NO. 372, SEQ ID NO. 373, SEQ ID NO. 374, SEQ ID NO. 375, SEQ ID NO. 376, SEQ ID NO. 377, SEQ ID NO. 381, SEQ ID NO. 382, SEQ ID NO. 384, SEQ ID NO. 386, SEQ ID NO. 387, SEQ ID NO. 388, SEQ ID NO. 389, SEQ ID NO. 397, SEQ ID NO. 400, SEQ ID NO. 401, SEQ ID NO. 402, SEQ ID NO. 403, SEQ ID NO. 404, SEQ ID NO. 405, SEQ ID NO. 408, SEQ ID NO. 410, SEQ ID NO. 413, SEQ ID NO. 415, SEQ ID NO. 416, SEQ ID NO. 418, SEQ ID NO. 426, SEQ ID NO. 429, SEQ ID NO. 430, SEQ ID NO. 431, SEQ ID NO. 440, SEQ ID NO. 441, SEQ ID NO. 444, SEQ ID NO. 445, SEQ ID NO. 446, SEQ ID NO. 448, SEQ ID NO. 450, SEQ ID NO. 451, SEQ ID NO. 453, SEQ ID NO. 454, SEQ ID NO. 455, SEQ ID NO. 456, SEQ ID NO. 457, SEQ ID NO. 459, SEQ ID NO. 460, SEQ ID NO. 461, SEQ ID NO. 462, SEQ ID NO. 463, SEQ ID NO. 464, SEQ ID NO. 465, SEQ ID NO. 468, SEQ ID NO. 474, SEQ ID NO. 476, SEQ ID NO. 477, SEQ ID NO. 478, SEQ ID NO. 480, SEQ ID NO. 483, SEQ ID NO. 484, SEQ ID NO. 485, SEQ ID NO. 486, SEQ ID NO. 487, SEQ ID NO. 488, SEQ ID NO. 489, SEQ ID NO. 490, SEQ ID NO. 491, SEQ ID NO. 493, SEQ ID NO. 494, SEQ ID NO. 496, SEQ ID NO. 497, SEQ ID NO. 512, SEQ ID NO. 517, SEQ ID NO. 539, SEQ ID NO. 542, SEQ ID NO. 544, SEQ ID NO. 545, SEQ ID NO. 546, SEQ ID NO. 547, SEQ ID NO. 548, SEQ ID NO. 550, SEQ ID NO. 551, SEQ ID NO. 552, SEQ ID NO. 554, SEQ ID NO. 560, SEQ ID NO. 561, SEQ ID NO. 562, SEQ ID NO. 563, SEQ ID NO. 564, SEQ ID NO. 565, SEQ ID NO. 566, SEQ ID NO. 567, SEQ ID NO. 568, SEQ ID NO. 569, SEQ ID NO. 570, SEQ ID NO. 572, SEQ ID NO. 573, SEQ ID NO. 574, SEQ ID NO. 575, SEQ ID NO. 578, SEQ ID NO. 579, SEQ ID NO. 581, SEQ ID NO. 582, SEQ ID NO. 583, SEQ ID NO. 584, SEQ ID NO. 590, SEQ ID NO. 592, SEQ ID NO. 596, SEQ ID NO. 597, SEQ ID NO. 601, SEQ ID NO. 602, SEQ ID NO. 603, SEQ ID NO. 606, SEQ ID NO. 609, SEQ ID NO. 610, SEQ ID NO. 618, SEQ ID NO. 619, SEQ ID NO. 620, SEQ ID NO. 625, SEQ ID NO. 628, SEQ ID NO. 629, SEQ ID NO. 630, SEQ ID NO. 631, SEQ ID NO. 632, SEQ ID NO. 638, SEQ ID NO. 642, SEQ ID NO. 643, SEQ ID NO. 652, SEQ ID NO. 653, SEQ ID NO. 657, SEQ ID NO. 661, SEQ ID NO. 662, SEQ ID NO. 666, SEQ ID NO. 669, SEQ ID NO. 674, SEQ ID NO. 692, SEQ ID NO. 699, SEQ ID NO. 707, SEQ ID NO. 708, SEQ ID NO. 715, SEQ ID NO. 717, SEQ ID NO. 718, SEQ ID NO. 719, SEQ ID NO. 720, SEQ ID NO. 721, SEQ ID NO. 722, SEQ ID NO. 725, SEQ ID NO. 728, SEQ ID NO. 729, SEQ ID NO. 731, SEQ ID NO. 732, SEQ ID NO. 733, SEQ ID NO. 734, SEQ ID NO. 736, SEQ ID NO. 737, SEQ ID NO. 738, SEQ ID NO. 740, SEQ ID NO. 743, SEQ ID NO. 744, SEQ ID NO. 746, SEQ ID NO. 748, SEQ ID NO. 749, SEQ ID NO. 756, SEQ ID NO. 757, SEQ ID NO. 758, SEQ ID NO. 771, SEQ ID NO. 772, SEQ ID NO. 775, SEQ ID NO. 778, SEQ ID NO. 779, SEQ ID NO. 780, SEQ ID NO. 781, SEQ ID NO. 784, SEQ ID NO. 787, SEQ ID NO. 789, SEQ ID NO. 793, SEQ ID NO. 794, SEQ ID NO. 796, SEQ ID NO. 798, SEQ ID NO. 801, SEQ ID NO. 807, SEQ ID NO. 811, SEQ ID NO. 814, SEQ ID NO. 820, SEQ ID NO. 828, SEQ ID NO. 833, SEQ ID NO. 835, SEQ ID NO. 836, SEQ ID NO. 837, SEQ ID NO. 838, SEQ ID NO. 842, SEQ ID NO. 843, SEQ ID NO. 844, SEQ ID NO. 847, SEQ ID NO. 848, SEQ ID NO. 849, SEQ ID NO. 850, SEQ ID NO. 851, SEQ ID NO. 852, and SEQ ID NO. 853. Further details can be found in Table 1.

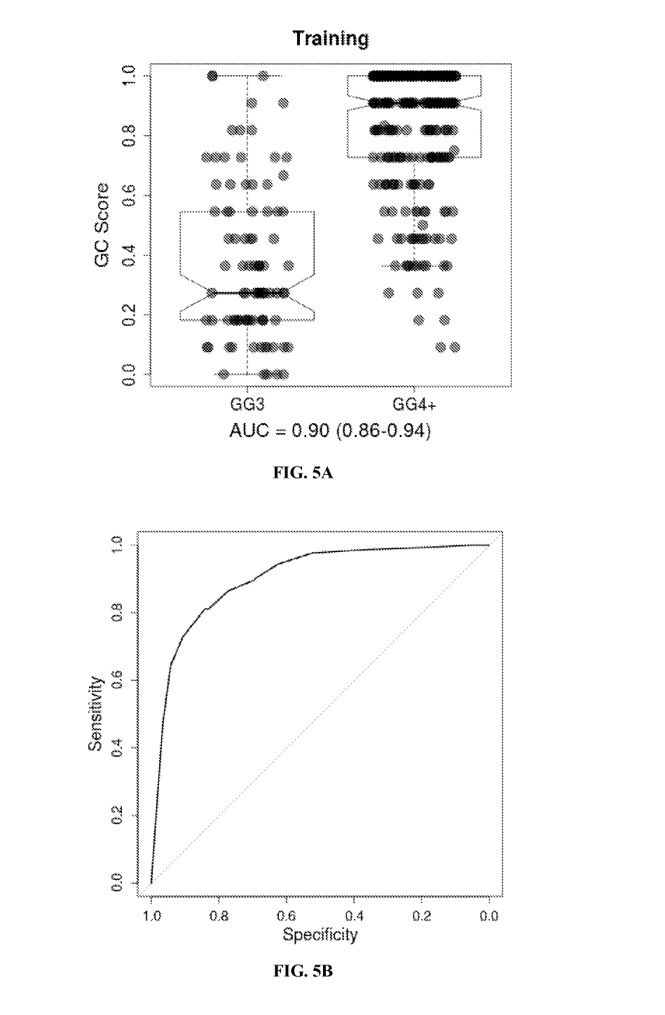

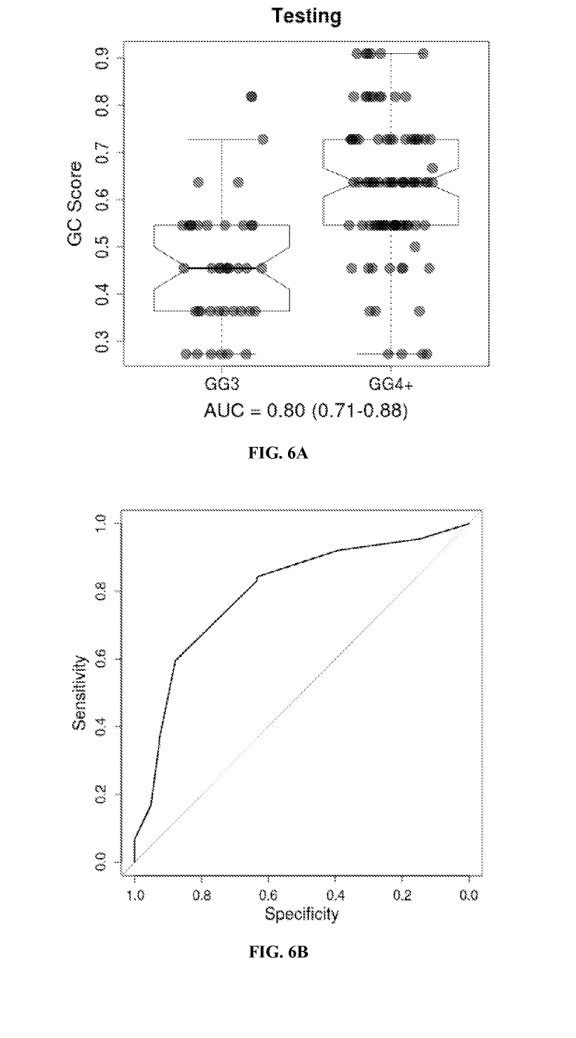

The good performance of classifier KNN392 is demonstrated by an AUC of 0.90 [95% CI 0.86-0.94] (FIG. 5) and an accuracy of 86% (p<0.01) in the Mayo Validation cohort (training) and an AUC of 0.74 [95% CI 0.68-0.91] (FIG. 6) and an accuracy of 78% (p<0.05) in the DKFZ dataset (testing). The fact that the confidence interval doesn't overlap with the 0.5 threshold demonstrates the statistical significance of the AUC values. Furthermore, as judged by a wilcoxon rank sum test, the classifier can significantly discriminate between non-malignant sample and tumor sample in both the training and testing datasets (p<0.001).

### Example 5

### A 104-Biomarker Signature that Discriminates Between Patients with High Grade Tumor from Patients with Low Grade Tumor

Methods

Classifier KNN104 is a signature that discriminates between patients with high grade tumor (Gleason Grade 4 or greater) from patients with low grade tumor (Gleason Grade 3 or lower). Feature selection was conducted using the Mayo training cohort described in example 1 (excluding patients with Gleason Score 7-n=167). The top 104 features ranked by AUC as highly differentially expressed between patients with low grade tumor and high grade tumor were used after z-score standardization to generate a classifier from the k-Nearest Neighbor algorithm. The model was further tuned in the Mayo testing cohort described in example 1 (n=57 after excluding patients with Gleason Score 7) to select a k-Nearest Neighbor algorithm parameter of k=27 using the tune function (R package e1071_{—}1.6-1). Performance of the classifier is assess in the Mayo Independent Validation dataset. The score of the classifier represent the probability an individual would be classified as having high grade tumor based on the expression values of the closest 27 patients in the training cohort of 167 prostate samples. The probabilities range from 0 to 1 where low probabilities represent a lower chance a patient would have high grade tumor while higher probabilities represent a higher chance a patient would have high grade tumor.

Results

The 104 features that compose KNN104 are: SEQ ID NO. 222, SEQ ID NO. 646, SEQ ID NO. 807, SEQ ID NO. 674, SEQ ID NO. 821, SEQ ID NO. 316, SEQ ID NO. 443, SEQ ID NO. 294, SEQ ID NO. 575, SEQ ID NO. 358, SEQ ID NO. 783, SEQ ID NO. 798, SEQ ID NO. 582, SEQ ID NO. 602, SEQ ID NO. 702, SEQ ID NO. 126, SEQ ID NO. 34, SEQ ID NO. 364, SEQ ID NO. 795, SEQ ID NO. 8, SEQ ID NO. 459, SEQ ID NO. 383, SEQ ID NO. 628, SEQ ID NO. 365, SEQ ID NO. 768, SEQ ID NO. 307, SEQ ID NO. 477, SEQ ID NO. 618, SEQ ID NO. 341, SEQ ID NO. 258, SEQ ID NO. 236, SEQ ID NO. 580, SEQ ID NO. 663, SEQ ID NO. 653, SEQ ID NO. 327, SEQ ID NO. 46, SEQ ID NO. 622, SEQ ID NO. 411, SEQ ID NO. 373, SEQ ID NO. 95, SEQ ID NO. 542, SEQ ID NO. 390, SEQ ID NO. 261, SEQ ID NO. 549, SEQ ID NO. 326, SEQ ID NO. 651, SEQ ID NO. 726, SEQ ID NO. 493, SEQ ID NO. 650, SEQ ID NO. 375, SEQ ID NO. 843, SEQ ID NO. 445, SEQ ID NO. 190, SEQ ID NO. 758, SEQ ID NO. 717, SEQ ID NO. 179, SEQ ID NO. 626, SEQ ID NO. 406, SEQ ID NO. 664, SEQ ID NO. 479, SEQ ID NO. 205, SEQ ID NO. 225, SEQ ID NO. 174, SEQ ID NO. 381, SEQ ID NO. 492, SEQ ID NO. 229, SEQ ID NO. 299, SEQ ID NO. 665, SEQ ID NO. 170, SEQ ID NO. 306, SEQ ID NO. 830, SEQ ID NO. 432, SEQ ID NO. 184, SEQ ID NO. 730, SEQ ID NO. 584, SEQ ID NO. 374, SEQ ID NO. 407, SEQ ID NO. 788, SEQ ID NO. 842, SEQ ID NO. 453, SEQ ID NO. 461, SEQ ID NO. 350, SEQ ID NO. 276, SEQ ID NO. 424, SEQ ID NO. 535, SEQ ID NO. 595, SEQ ID NO. 33, SEQ ID NO. 427, SEQ ID NO. 831, SEQ ID NO. 399, SEQ ID NO. 691, SEQ ID NO. 819, SEQ ID NO. 356, SEQ ID NO. 65, SEQ ID NO. 409, SEQ ID NO. 538, SEQ ID NO. 735, SEQ ID NO. 452, SEQ ID NO. 771, SEQ ID NO. 608, SEQ ID NO. 391, SEQ ID NO. 44, SEQ ID NO. 447, SEQ ID NO. 799. Further details on these sequences are provided in Table 1.

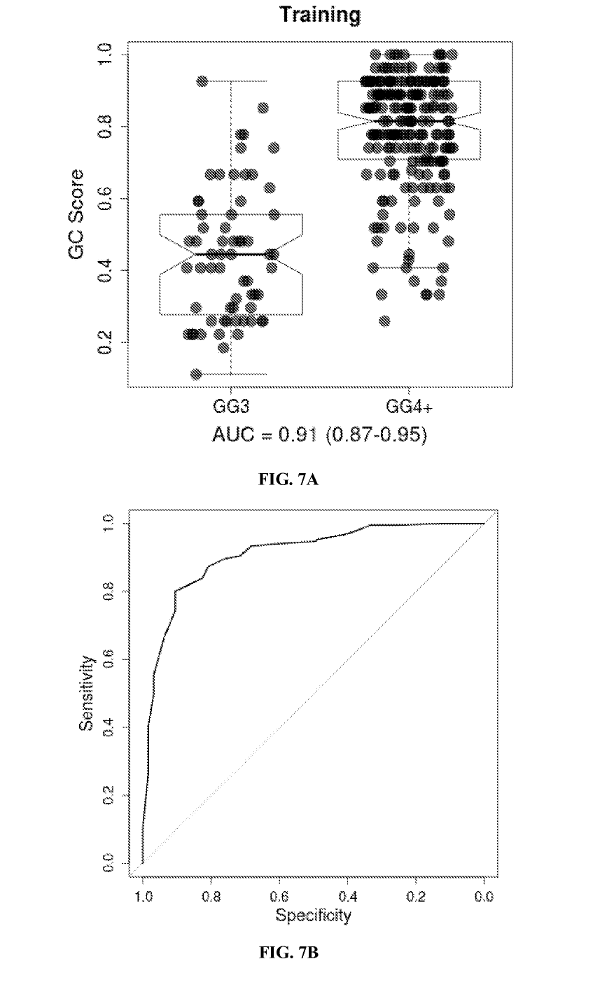

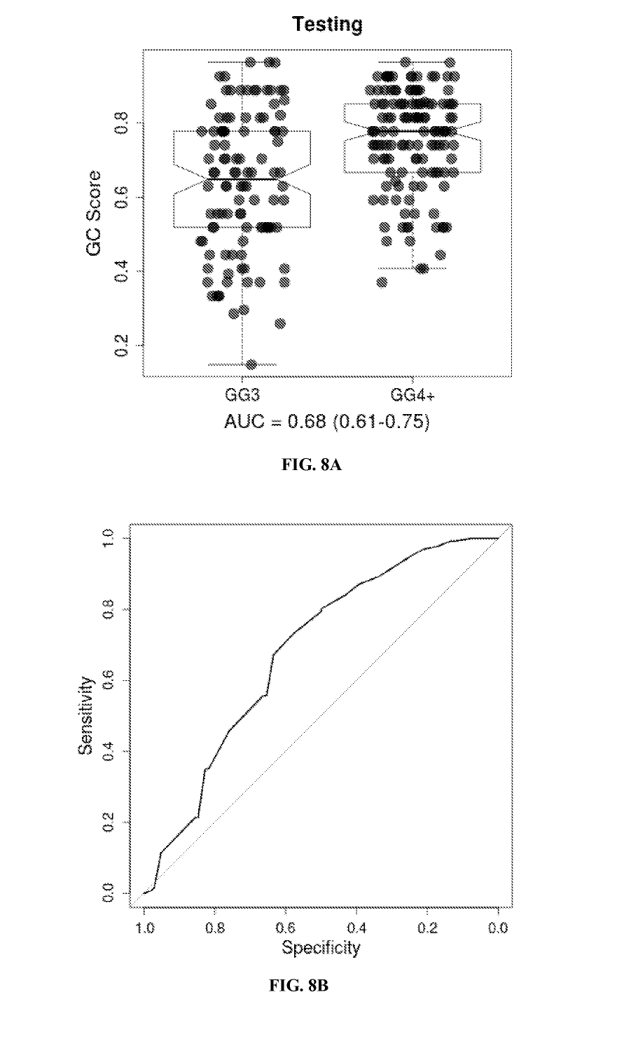

The good performance of classifier KNN104 is demonstrated by an AUC of 0.91 [95% CI 0.87-0.95] (FIG. 7) and an accuracy of 88% (p<0.01) in the Mayo discovery dataset (excluding Gleason 7 patients—training) and an AUC of 0.68 [95% CI 0.61-0.75] (FIG. 8) and an accuracy of 64% (p<0.01) in the Mayo independent validation dataset (testing). The fact that the confidence interval doesn't overlap with the 0.5 threshold demonstrates the statistical significance of the result. Furthermore, as judged by a wilcoxon rank sum test, the classifier can significantly discriminate between low grade tumor and high grade tumor in both the training and testing cohort (p<0.001). These results show the strong ability of KNN104 to predict whether a patient sample contains Gleason grade 3 or Gleason grade 4+.

### Example 6

### A 41-Biomarker Signature that Discriminates Between Prostate Tumor Samples from Non-Malignant Samples

Methods

Classifier KNN41 is a signature that discriminates between prostate tumor samples from non-malignant samples. Top 41 features ranked, by mean fold difference, as highly differentially expressed between tumor samples and non-malignant samples in MSKCC, DKFZ and ICR (accession number GSE12378) patient cohorts described in example 1 (n=294 patients) were percentile rank standardized and used to generate a classifier from the k-Nearest Neighbor algorithm with parameter k=23. The score of the classifier represent the probability a patient sample would be classified as tumor samples based on the expression values of the closest 13 patients in the training cohort of 294 prostate samples. The probabilities range from 0 to 1 where low probabilities represent a lower chance of the sample being a non-malignant sample while higher probabilities represent a higher chance of the sample being a tumor sample.

Results

The 41 features that compose KNN41 are: SEQ ID NO. 255, SEQ ID NO. 167, SEQ ID NO. 501, SEQ ID NO. 504, SEQ ID NO. 254, SEQ ID NO. 503, SEQ ID NO. 224, SEQ ID NO. 502, SEQ ID NO. 509, SEQ ID NO. 507, SEQ ID NO. 557, SEQ ID NO. 506, SEQ ID NO. 251, SEQ ID NO. 644, SEQ ID NO. 90, SEQ ID NO. 260, SEQ ID NO. 766, SEQ ID NO. 510, SEQ ID NO. 166, SEQ ID NO. 241, SEQ ID NO. 436, SEQ ID NO. 256, SEQ ID NO. 118, SEQ ID NO. 257, SEQ ID NO. 676, SEQ ID NO. 283, SEQ ID NO. 508, SEQ ID NO. 253, SEQ ID NO. 252, SEQ ID NO. 840, SEQ ID NO. 196, SEQ ID NO. 765, SEQ ID NO. 165, SEQ ID NO. 10, SEQ ID NO. 212, SEQ ID NO. 827, SEQ ID NO. 434, SEQ ID NO. 769, SEQ ID NO. 505, SEQ ID NO. 742, and SEQ ID NO. 704.

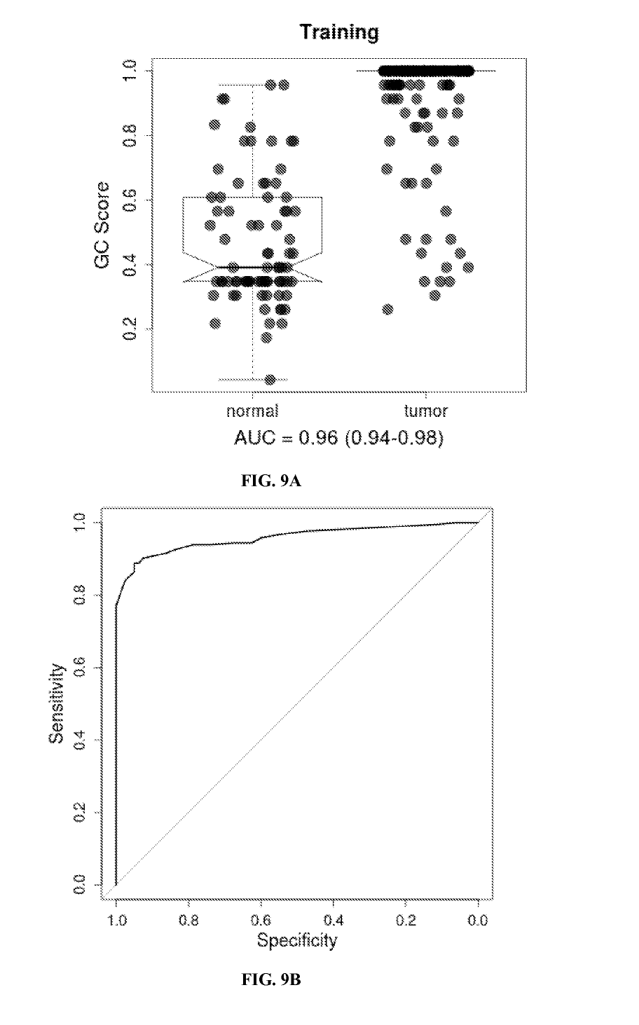

The good performance of classifier KNN41 is demonstrated by an AUC of 0.96 [95% CI 0.94-0.98] (FIG. 9) and an accuracy of 89% (p<0.01) in the MSKCC, DKFZ and ICR cohort. The significance is highlighted by a CI that does not span 0.5 which is the performance expected by random chance alone. Furthermore, as judged by a wilcoxon rank sum test, the classifier can significantly discriminate between non-malignant sample and tumor sample (p<0.001).

### Example 7

### A 150 Biomarker Classifier to Predict Androgen Deprivation Therapy (ADT) Failure in Prostate Cancer Samples

HDDA150 classifier was developed on a cohort of 780 radical prostatectomy samples from the Mayo clinic (pooled Discovery and Validation cohorts, described in Example 1).

In order to select biomarkers specific to hormone treatment failure, patients subjected to salvage hormone therapy were randomly divided into a training (n=119) and testing (n=57) set. In the testing set, background and cross hybridization filtering was performed to remove unreliable microarray features. The expression values of the 761,085 remaining genomic features were used to rank the features according to their differential expression between hormone treatment patients who failed the therapy, as defined by distant metastasis from those who remained metastasis free. The most differentially expressed features (n=150) were modeled using a high dimensional discriminate analysis classifier (HDDA150).

Results

The 150 features that compose HDDA150 are: SEQ ID NO. 739, SEQ ID NO. 797, SEQ ID NO. 86, SEQ ID NO. 209, SEQ ID NO. 175, SEQ ID NO. 711, SEQ ID NO. 518, SEQ ID NO. 101, SEQ ID NO. 670, SEQ ID NO. 29, SEQ ID NO. 713, SEQ ID NO. 425, SEQ ID NO. 498, SEQ ID NO. 792, SEQ ID NO. 585, SEQ ID NO. 362, SEQ ID NO. 467, SEQ ID NO. 49, SEQ ID NO. 36, SEQ ID NO. 37, SEQ ID NO. 656, SEQ ID NO. 791, SEQ ID NO. 353, SEQ ID NO. 641, SEQ ID NO. 359, SEQ ID NO. 233, SEQ ID NO. 47, SEQ ID NO. 475, SEQ ID NO. 38, SEQ ID NO. 14, SEQ ID NO. 473, SEQ ID NO. 117, SEQ ID NO. 680, SEQ ID NO. 56, SEQ ID NO. 107, SEQ ID NO. 499, SEQ ID NO. 125, SEQ ID NO. 274, SEQ ID NO. 39, SEQ ID NO. 146, SEQ ID NO. 824, SEQ ID NO. 639, SEQ ID NO. 623, SEQ ID NO. 394, SEQ ID NO. 822, SEQ ID NO. 12, SEQ ID NO. 155, SEQ ID NO. 587, SEQ ID NO. 716, SEQ ID NO. 469, SEQ ID NO. 589, SEQ ID NO. 810, SEQ ID NO. 747, SEQ ID NO. 823, SEQ ID NO. 800, SEQ ID NO. 807, SEQ ID NO. 640, SEQ ID NO. 659, SEQ ID NO. 511, SEQ ID NO. 108, SEQ ID NO. 189, SEQ ID NO. 773, SEQ ID NO. 654, SEQ ID NO. 505, SEQ ID NO. 272, SEQ ID NO. 417, SEQ ID NO. 349, SEQ ID NO. 536, SEQ ID NO. 59, SEQ ID NO. 325, SEQ ID NO. 419, SEQ ID NO. 839, SEQ ID NO. 137, SEQ ID NO. 671, SEQ ID NO. 802, SEQ ID NO. 633, SEQ ID NO. 262, SEQ ID NO. 24, SEQ ID NO. 259, SEQ ID NO. 790, SEQ ID NO. 16, SEQ ID NO. 158, SEQ ID NO. 423, SEQ ID NO. 164, SEQ ID NO. 786, SEQ ID NO. 470, SEQ ID NO. 219, SEQ ID NO. 635, SEQ ID NO. 60, SEQ ID NO. 521, SEQ ID NO. 841, SEQ ID NO. 809, SEQ ID NO. 683, SEQ ID NO. 698, SEQ ID NO. 466, SEQ ID NO. 232, SEQ ID NO. 528, SEQ ID NO. 145, SEQ ID NO. 97, SEQ ID NO. 13, SEQ ID NO. 696, SEQ ID NO. 675, SEQ ID NO. 621, SEQ ID NO. 133, SEQ ID NO. 605, SEQ ID NO. 116, SEQ ID NO. 296, SEQ ID NO. 204, SEQ ID NO. 689, SEQ ID NO. 342, SEQ ID NO. 198, SEQ ID NO. 806, SEQ ID NO. 163, SEQ ID NO. 774, SEQ ID NO. 808, SEQ ID NO. 660, SEQ ID NO. 762, SEQ ID NO. 586, SEQ ID NO. 11, SEQ ID NO. 177, SEQ ID NO. 701, SEQ ID NO. 220, SEQ ID NO. 393, SEQ ID NO. 458, SEQ ID NO. 191, SEQ ID NO. 195, SEQ ID NO. 767, SEQ ID NO. 776, SEQ ID NO. 520, SEQ ID NO. 709, SEQ ID NO. 55, SEQ ID NO. 143, SEQ ID NO. 420, SEQ ID NO. 422, SEQ ID NO. 481, SEQ ID NO. 529, SEQ ID NO. 845, SEQ ID NO. 412, SEQ ID NO. 667, SEQ ID NO. 681, SEQ ID NO. 812, SEQ ID NO. 197, SEQ ID NO. 73, SEQ ID NO. 115, SEQ ID NO. 74, SEQ ID NO. 217, SEQ ID NO. 428, SEQ ID NO. 106, SEQ ID NO. 741, SEQ ID NO. 124.

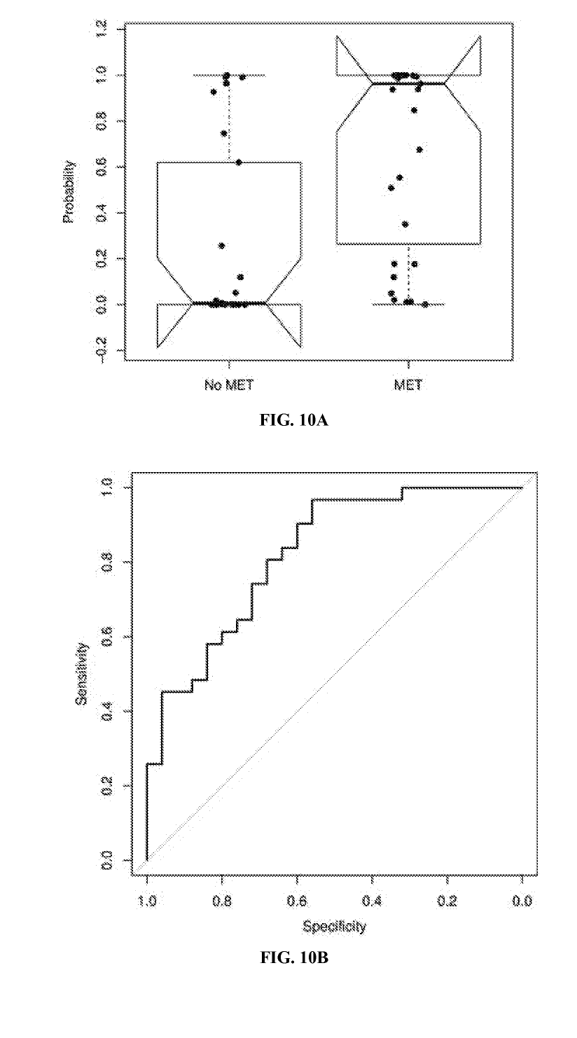

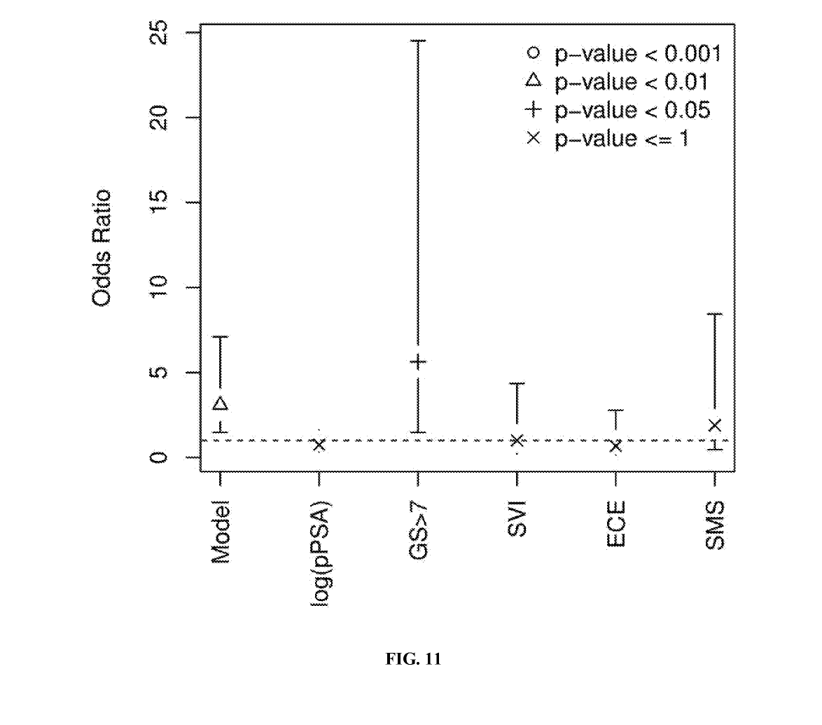

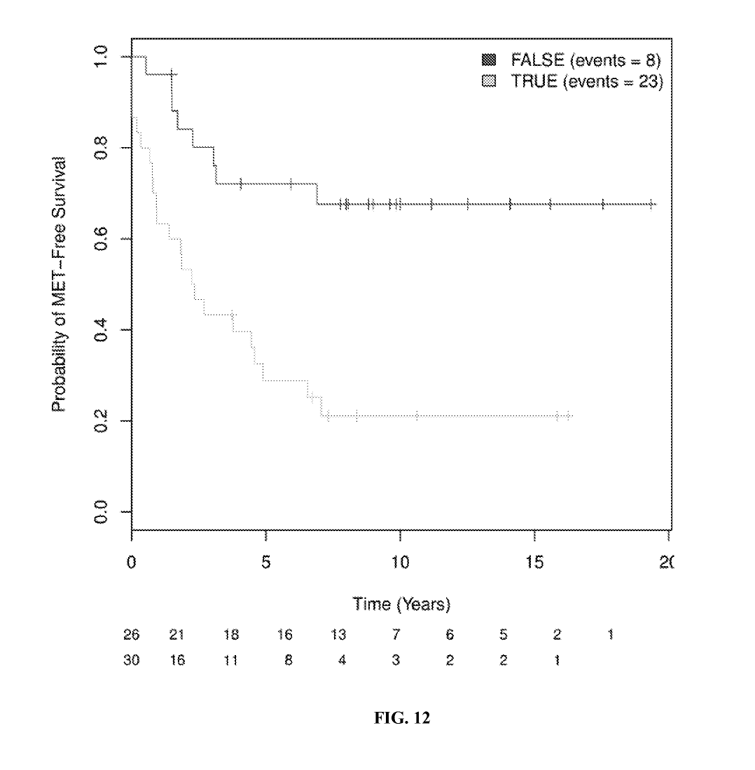

When HDDA150 was applied to the Mayo testing set it achieved an area under the curve (AUC) of 0.82 [95% ci=0.71-0.93] (FIG. 10) and an accuracy of 73% (p<0.01) over a null model accuracy of 55%. In multivariable analysis (FIG. 11, Table 2) adjusting the model for pre-operative PSA, Gleason score, seminal vesicle invasion, surgical margin status, and extra capillary extension HDDA150 was found to be significant (p<0.01) suggesting that the genomic markers add novel information over the clinicopathologic variables. The survival analysis, in FIG. 12, shows that there is a significant difference in metastasis-free survival for the patients classified as high risk by HDDA150.

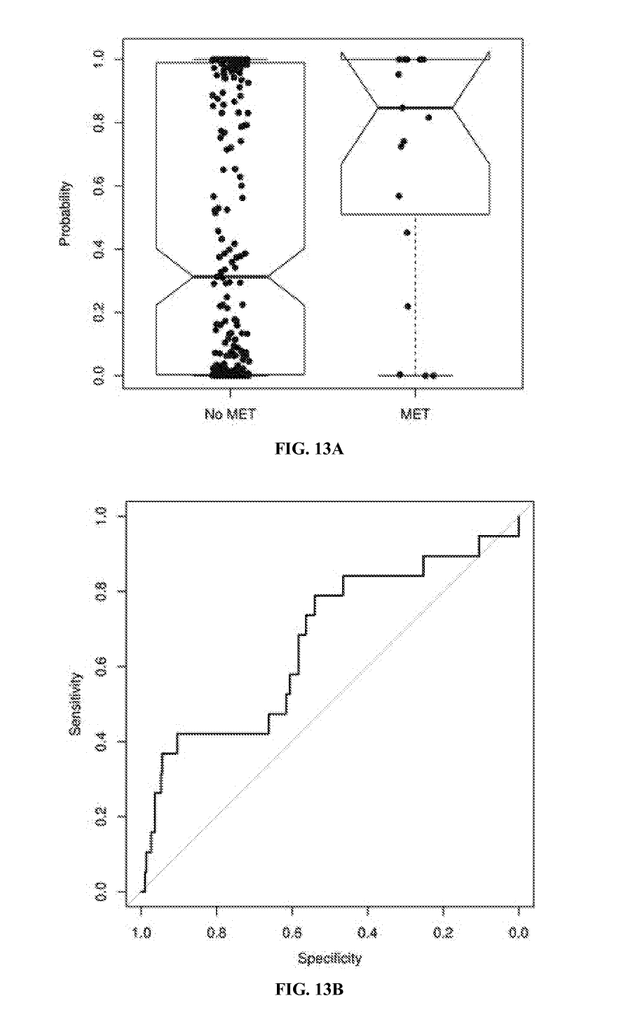

When HDDA150 was applied to patients who underwent either salvage or adjuvant radiation therapy (FIG. 13) the signature's accuracy and discrimination performance were found to be insignificant having a 95% confidence intervals which crosses the no discrimination point (=0.50). This difference in HDDA150 performance between treatment subsets provides evidence that the signature is composed of markers which are specific to predicting salvage hormone treatment failure and not failure to any treatment.

TABLE 2
MVA Odds Ratios for HDDA150 in comparison to clinical variables

| | OR | 2.5% | 97.5% | P-Value |
|---|---|---|---|---|
| ECE | 0.68 | 0.15 | 2.78 | 0.59 |
| HDDA150 | 3.09 | 1.49 | 7.10 | 0.00 |
| GS > 7 | 5.63 | 1.48 | 24.51 | 0.01 |
| log(pPSA) | 0.74 | 0.33 | 1.62 | 0.46 |
| SMS | 1.89 | 0.46 | 8.44 | 0.38 |
| SVI | 1.00 | 0.22 | 4.36 | 1.00 |

### Example 8

### A 22 Biomarker Classifier to Predict Whether a Prostate Sample is Tumurous

Methods

The MSKCC dataset described in Example 1 was used for feature selection and to train the model. This model is a signature that discriminates between prostate tumor samples from non-malignant samples. The top 22 features ranked as highly differentially expressed between tumor samples and non-malignant samples (n=160 patients) were percentile rank standardization and used to generate a classifier with the k-Nearest Neighbor algorithm using parameter k=21. The score of the classifier represents the probability that an individual sample would be classified as tumor samples based on the expression values of the closest 21 patients in the training cohort of 160 prostate samples. The probabilities range from 0 to 1 where low probabilities represent a lower chance of the sample being a non-malignant sample while higher probabilities represent a higher chance of the sample being a tumor sample.

Results

The 22 features that correspond to the generated KNN classifier are: SEQ ID NO. 677, SEQ ID NO. 687, SEQ ID NO. 522, SEQ ID NO. 438, SEQ ID NO. 690, SEQ ID NO. 435, SEQ ID NO. 533, SEQ ID NO. 688, SEQ ID NO. 129, SEQ ID NO. 686, SEQ ID NO. 130, SEQ ID NO. 832, SEQ ID NO. 615, SEQ ID NO. 531, SEQ ID NO. 543, SEQ ID NO. 524, SEQ ID NO. 323, SEQ ID NO. 433, SEQ ID NO. 616, SEQ ID NO. 437, SEQ ID NO. 84, SEQ ID NO. 723.

Further details on these sequences are provided in Table 1. Performance of KNN22 is shown in Table 3. In all the validation sets DKFZ and ICR the classifier achieved AUCs of 0.98 and 0.91 respectively. Likewise the model's accuracy in the validation sets DKFZ, ICR, and Mayo was 0.94, 0.92, 0.99 respectively, using a 0.5 classification threshold. These results show the strong ability of KNN22 to predict whether a sample comes from normal tissue or tumor tissue.

TABLE 3
The prediction accuracy (cutoff = 0.5) and discrimination of KNN22
in the DKFZ, MKSCC, ICR, and Mayo prostate datasets.

| | | MSKCC | | |
|---|---|---|---|---|
| | DKFZ | (Training) | ICR | Mayo |
| AUC | 0.98 | 0.99 | 0.91 | NA |
| Accuracy | 0.94 | 0.96 | 0.92 | 0.99 |

### Example 9

### A 34 Biomarker Classifier to Predict Whether a Prostate Sample is Tumurous

Methods

The MSKCC dataset described in Example 1 was used for feature selection and to train the model. Classifier KNN34 is a signature that discriminates between prostate tumor samples from non-malignant samples. Top 34 features ranked as highly differentially expressed between tumor samples and non-malignant samples (n=160 patients) were percentile rank standardization and used to generate a classifier from the k-Nearest Neighbor algorithm with parameter k=15. The 34 features, corresponding to Affymetrix Probe Set Ids and genomic regions specified in Table 4. The score of the classifier represent the probability an individual would be classified as tumor samples based on the expression values of the closest 15 patients in the training cohort of 160 prostate samples. The probabilities range from 0 to 1 where low probabilities represent a lower chance of the sample being a non-malignant sample while higher probabilities represent a higher chance of the sample being a tumor sample.

Results

The 34 features that correspond to the generated KNN classifier are: SEQ ID NO. 677, SEQ ID NO. 687, SEQ ID NO. 522, SEQ ID NO. 438, SEQ ID NO. 690, SEQ ID NO. 435, SEQ ID NO. 533, SEQ ID NO. 688, SEQ ID NO. 129, SEQ ID NO. 686, SEQ ID NO. 130, SEQ ID NO. 832, SEQ ID NO. 615, SEQ ID NO. 531, SEQ ID NO. 543, SEQ ID NO. 524, SEQ ID NO. 323, SEQ ID NO. 433, SEQ ID NO. 616, SEQ ID NO. 437, SEQ ID NO. 84, SEQ ID NO. 723, SEQ ID NO. 684, SEQ ID NO. 724, SEQ ID NO. 764, SEQ ID NO. 525, SEQ ID NO. 537, SEQ ID NO. 763, SEQ ID NO. 685, SEQ ID NO. 471, SEQ ID NO. 532, SEQ ID NO. 526, SEQ ID NO. 472, SEQ ID NO. 673.

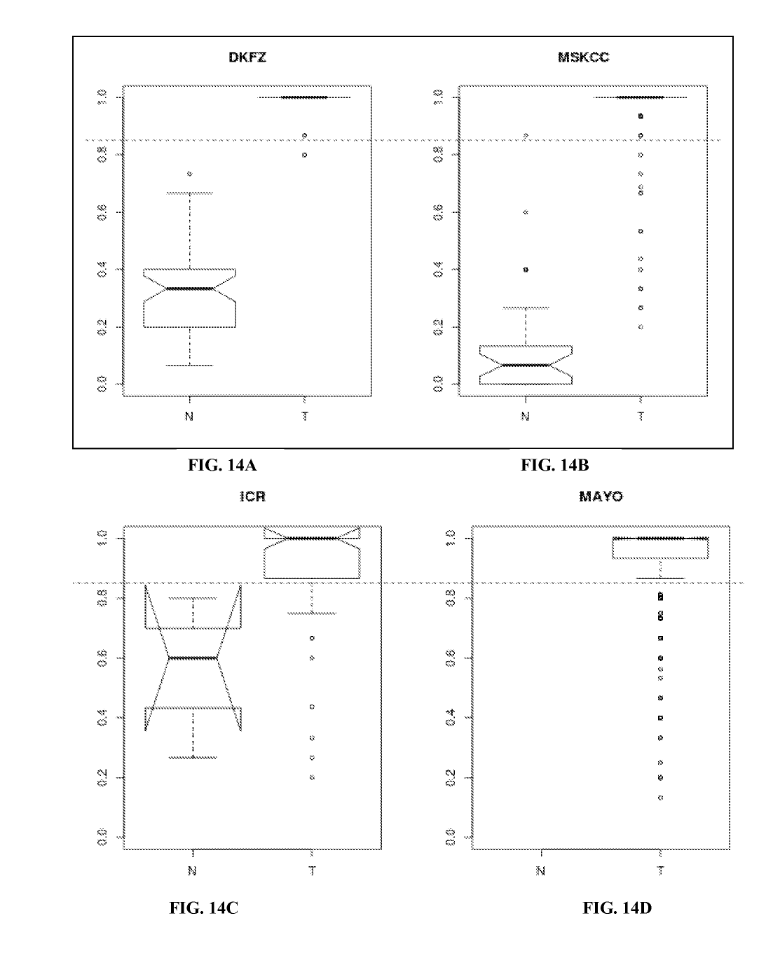

Further details on these sequences are provided in Table 1. Performance of KNN34 is shown in Table 4. In all the validation sets DKFZ, ICR, Norris, and Erasmus the classifier achieved AUCs of 1.0 and 0.87 respectively. Likewise the model's accuracy in the validation sets DKFZ, ICR, and Mayo was 0.98, 0.79, and 0.90 respectively, using a 0.85 classification threshold. These results show the strong ability of KNN34 to predict whether a sample comes from normal tissue or tumor tissue. (FIG. 14)

TABLE 4
The prediction accuracy (cutoff = 0.85) and discrimination of KNN34-NT
in the DKFZ, MKSCC, ICR, and Mayo prostate datasets.

| | | MSKCC | | |
|---|---|---|---|---|
| | DKFZ | (Training) | ICR | Mayo |
| AUC | 1.0 | 0.99 | 0.87 | NA |
| Accuracy | 0.98 | 0.91 | 0.79 | 0.90 |

### Example 10

### A 72-Biomarker Signature that Discriminates Between Patients with High Grade Tumor from Patients with Low Grade Tumor

Methods

The MSKCC and Mayo Training datasets described in Example 1 were used for feature selection and just the Mayo Training and DKFZ datasets, also described in Example 1 were used to train the model. Classifier RF72 is a signature that discriminates between high grade tumors (Gleason 4 or higher) from low grade tumors (Gleason 3 or lower). Top 72 features ranked by AUC as highly differentially expressed between patients with low grade tumor and high grade tumor in the Mayo Training and MSKCC dataset were identified. The 72 features were then z-score standardized and used to generate a classifier from the random forest algorithm tuned for accuracy in the mayo training dataset and DKFZ cohort (tune function in R package e1071_{—}1.6-1 and R package randomForest 4.6-7). The score of the classifier represent the probability an individual would be classified as having high grade tumor based on the expression values of in the training cohort of prostate samples. The probabilities range from 0 to 1 where low probabilities represent a lower chance a patient would have high grade tumor while higher probabilities represent a higher chance a patient would have high grade tumor.

Results

The 72 features that correspond to the generated RF classifier are: SEQ ID NO. 646, SEQ ID NO. 373, SEQ ID NO. 674, SEQ ID NO. 602, SEQ ID NO. 372, SEQ ID NO. 375, SEQ ID NO. 377, SEQ ID NO. 512, SEQ ID NO. 32, SEQ ID NO. 307, SEQ ID NO. 487, SEQ ID NO. 594, SEQ ID NO. 306, SEQ ID NO. 295, SEQ ID NO. 374, SEQ ID NO. 610, SEQ ID NO. 329, SEQ ID NO. 599, SEQ ID NO. 784, SEQ ID NO. 554, SEQ ID NO. 489, SEQ ID NO. 376, SEQ ID NO. 311, SEQ ID NO. 738, SEQ ID NO. 553, SEQ ID NO. 64, SEQ ID NO. 332, SEQ ID NO. 556, SEQ ID NO. 309, SEQ ID NO. 513, SEQ ID NO. 837, SEQ ID NO. 611, SEQ ID NO. 496, SEQ ID NO. 590, SEQ ID NO. 187, SEQ ID NO. 119, SEQ ID NO. 813, SEQ ID NO. 313, SEQ ID NO. 649, SEQ ID NO. 609, SEQ ID NO. 439, SEQ ID NO. 491, SEQ ID NO. 836, SEQ ID NO. 613, SEQ ID NO. 240, SEQ ID NO. 81, SEQ ID NO. 515, SEQ ID NO. 449, SEQ ID NO. 123, SEQ ID NO. 312, SEQ ID NO. 61, SEQ ID NO. 314, SEQ ID NO. 338, SEQ ID NO. 121, SEQ ID NO. 600, SEQ ID NO. 330, SEQ ID NO. 305, SEQ ID NO. 343, SEQ ID NO. 694, SEQ ID NO. 657, SEQ ID NO. 122, SEQ ID NO. 829, SEQ ID NO. 571, SEQ ID NO. 71, SEQ ID NO. 28, SEQ ID NO. 785, SEQ ID NO. 700, SEQ ID NO. 82, SEQ ID NO. 636, SEQ ID NO. 378, SEQ ID NO. 344, SEQ ID NO. 555.

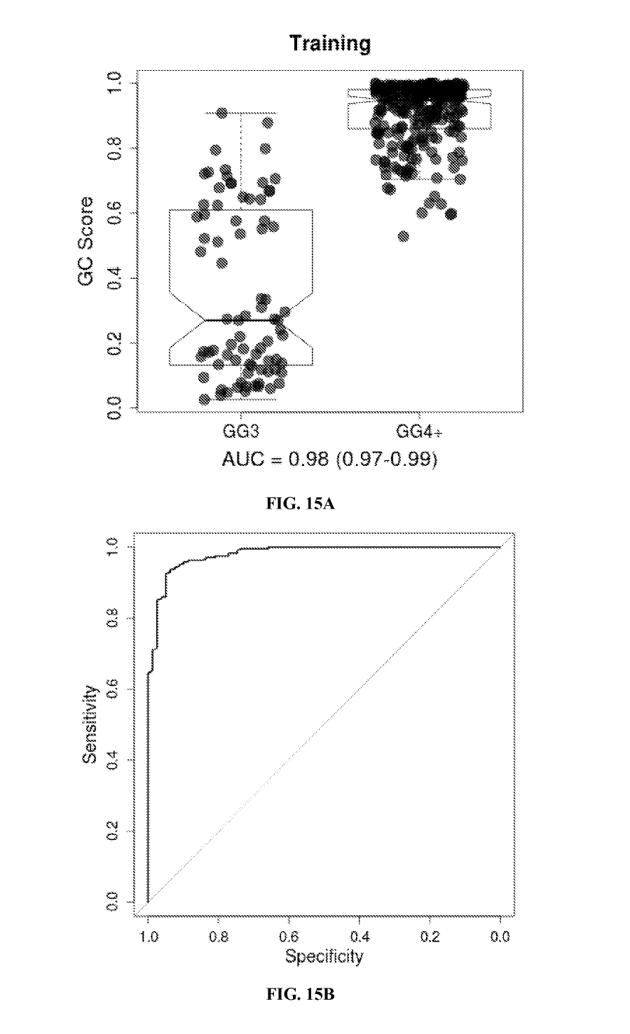

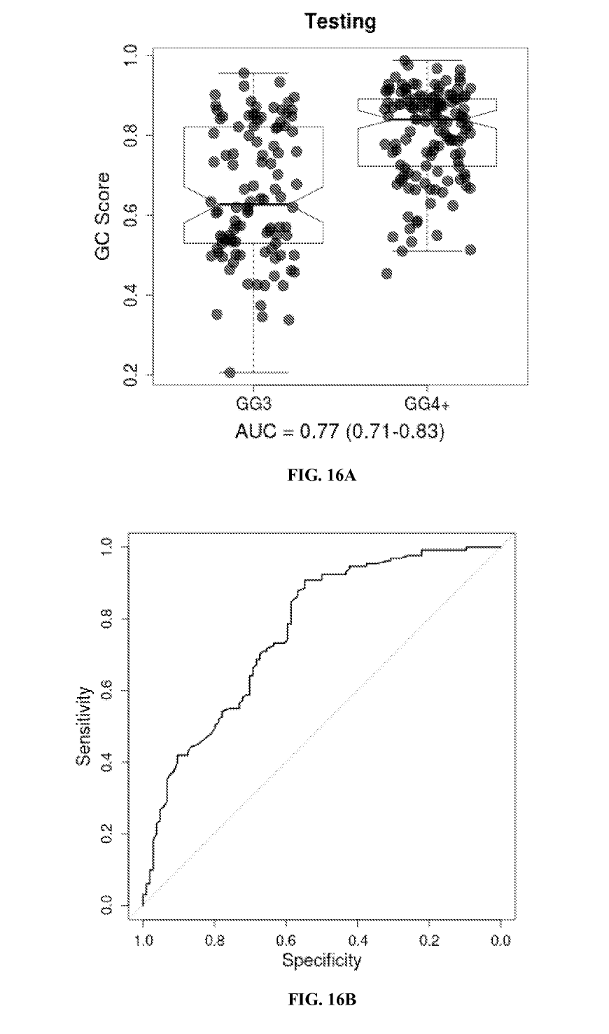

The performance of classifier RF72 is demonstrated by an AUC of 0.98 [95% CI 0.97-0.99] (FIG. 15) and an accuracy of 91% (p<0.01) (in Mayo discovery and DKFZ) and an AUC of 0.77 [95% CI 0.71-0.83] (FIG. 16) and a validation accuracy of 63% (p<0.01) in the Mayo independent validation cohort. The significance is highlighted by a CI that does not span 0.5 which is the performance expected by random chance alone. Furthermore, as judged by a wilcoxon rank sum test, the classifier can significantly discriminate between non-malignant sample and tumor sample in both the training and testing cohort (p<0.001). These results show the strong ability of RF72 to predict whether a patient sample contains Gleason grade 3 or Gleason grade

### Example 11

### A 132-Biomarker Signature that Discriminates Between Patients with High Grade Tumor from Patients with Low Grade Tumor

Methods

The MSKCC and Mayo Training datasets described in Example 1 were used for feature selection and just the Mayo Training and DKFZ datasets, also described in Example 1 were used to train the model. Classifier RF132 is a signature that discriminates between between high grade tumors (Gleason 4 or higher) from low grade tumors (Gleason 3 or lower). Top 132 features ranked by T-test as highly differentially expressed between patients with low grade tumor and high grade tumor in the Mayo Training and MSKCC dataset were identified. The 132 features were then z-score standardized and used to generate a classifier from the random forest algorithm tuned for accuracy in the mayo training dataset and DKFZ cohort (tune function in R package e1071_{—}1.6-1 and R package randomForest 4.6-7). The score of the classifier represent the probability an individual would be classified as having high grade tumor based on the expression values of in the training cohort of prostate samples. The probabilities range from 0 to 1 where low probabilities represent a lower chance a patient would have high grade tumor while higher probabilities represent a higher chance a patient would have high grade tumor. These results show the strong ability of RF132 to predict whether a patient sample contains Gleason grade 3 or Gleason grade 4+.

Results

The 132 features that correspond to the generated RF classifier are: SEQ ID NO. 373, SEQ ID NO. 646, SEQ ID NO. 602, SEQ ID NO. 372, SEQ ID NO. 307, SEQ ID NO. 375, SEQ ID NO. 377, SEQ ID NO. 487, SEQ ID NO. 32, SEQ ID NO. 374, SEQ ID NO. 306, SEQ ID NO. 784, SEQ ID NO. 295, SEQ ID NO. 311, SEQ ID NO. 594, SEQ ID NO. 376, SEQ ID NO. 496, SEQ ID NO. 489, SEQ ID NO. 64, SEQ ID NO. 567, SEQ ID NO. 309, SEQ ID NO. 332, SEQ ID NO. 553, SEQ ID NO. 31, SEQ ID NO. 554, SEQ ID NO. 513, SEQ ID NO. 119, SEQ ID NO. 314, SEQ ID NO. 512, SEQ ID NO. 611, SEQ ID NO. 610, SEQ ID NO. 63, SEQ ID NO. 813, SEQ ID NO. 338, SEQ ID NO. 836, SEQ ID NO. 305, SEQ ID NO. 609, SEQ ID NO. 556, SEQ ID NO. 652, SEQ ID NO. 240, SEQ ID NO. 187, SEQ ID NO. 121, SEQ ID NO. 66, SEQ ID NO. 829, SEQ ID NO. 515, SEQ ID NO. 658, SEQ ID NO. 803, SEQ ID NO. 199, SEQ ID NO. 491, SEQ ID NO. 81, SEQ ID NO. 378, SEQ ID NO. 703, SEQ ID NO. 573, SEQ ID NO. 648, SEQ ID NO. 700, SEQ ID NO. 312, SEQ ID NO. 71, SEQ ID NO. 123, SEQ ID NO. 649, SEQ ID NO. 590, SEQ ID NO. 804, SEQ ID NO. 122, SEQ ID NO. 330, SEQ ID NO. 128, SEQ ID NO. 516, SEQ ID NO. 593, SEQ ID NO. 599, SEQ ID NO. 57, SEQ ID NO. 636, SEQ ID NO. 777, SEQ ID NO. 647, SEQ ID NO. 343, SEQ ID NO. 308, SEQ ID NO. 161, SEQ ID NO. 94, SEQ ID NO. 837, SEQ ID NO. 105, SEQ ID NO. 695, SEQ ID NO. 785, SEQ ID NO. 99, SEQ ID NO. 367, SEQ ID NO. 20, SEQ ID NO. 238, SEQ ID NO. 168, SEQ ID NO. 527, SEQ ID NO. 442, SEQ ID NO. 672, SEQ ID NO. 682, SEQ ID NO. 239, SEQ ID NO. 156, SEQ ID NO. 705, SEQ ID NO. 186, SEQ ID NO. 334, SEQ ID NO. 278, SEQ ID NO. 379, SEQ ID NO. 4, SEQ ID NO. 541, SEQ ID NO. 160, SEQ ID NO. 761, SEQ ID NO. 706, SEQ ID NO. 25, SEQ ID NO. 577, SEQ ID NO. 297, SEQ ID NO. 555, SEQ ID NO. 248, SEQ ID NO. 825, SEQ ID NO. 67, SEQ ID NO. 637, SEQ ID NO. 612, SEQ ID NO. 540, SEQ ID NO. 313, SEQ ID NO. 745, SEQ ID NO. 588, SEQ ID NO. 273, SEQ ID NO. 514, SEQ ID NO. 449, SEQ ID NO. 645, SEQ ID NO. 207, SEQ ID NO. 490, SEQ ID NO. 591, SEQ ID NO. 805, SEQ ID NO. 760, SEQ ID NO. 23, SEQ ID NO. 576, SEQ ID NO. 244, SEQ ID NO. 310, SEQ ID NO. 846, SEQ ID NO. 759, SEQ ID NO. 131, SEQ ID NO. 120, SEQ ID NO. 109, SEQ ID NO. 237.

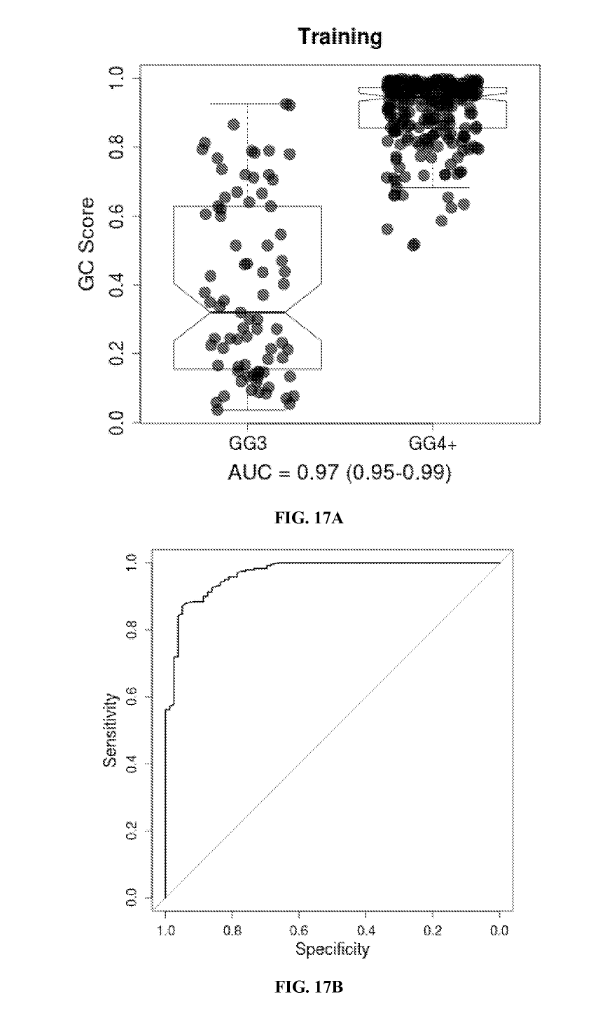

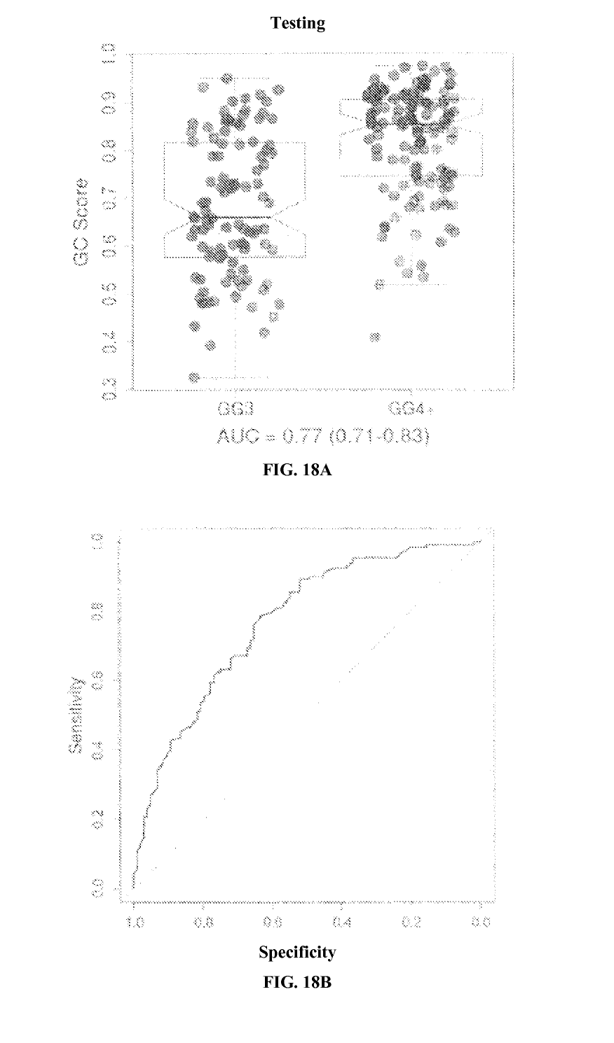

The good performance of classifier RF132 is demonstrated by an AUC of 0.97 [95% CI 0.95-0.99] (FIG. 17) and an accuracy of 92% (p<0.01) in the Mayo discovery and DKFZ cohort, and an AUC of 0.77 [95% CI 0.71-0.83] (FIG. 18) and an accuracy of 61% (p<0.01) in the Mayo independent validation cohort. The significance is highlighted by a CI that does not span 0.5 which is the performance expected by random chance alone. Furthermore, as judged by a wilcoxon rank sum test, the classifier can significantly discriminate between non-malignant sample and tumor sample in both the training and testing cohort (p<0.001).

TABLE 1

| SEQ | | | | |
|---|---|---|---|---|
| ID | AFFYMETRIX | | | |
| NO | ID | GENE | TYPE | CDS |
| 1 | 2316587 | RER1 | exonic | FALSE |
| 2 | 2317282 | ARHGEF16 | exonic | FALSE |
| 3 | 2319378 | | nonunique | FALSE |
| 4 | 2319379 | SLC25A33 | exonic | FALSE |
| 5 | 2320631 | | nonunique | FALSE |
| 6 | 2324040 | CAMK2N1 | antisense | FALSE |
| 7 | 2328706 | KPNA6, RP4-622L5.2 | exonic | FALSE |
| 8 | 2329993 | RP11-435D7.3 | exonic | FALSE |
| 9 | 2333722 | CCDC24 | exonic | TRUE |
| 10 | 2334955 | CYP4B1 | exonic | FALSE |
| 11 | 2342796 | ST6GALNAC3 | intronic | FALSE |
| 12 | 2350042 | VAV3 | antisense | FALSE |
| 13 | 2350396 | RP11-475E11.5 | exonic | FALSE |
| 14 | 2354133 | SPAG17 | antisense | FALSE |
| 15 | 2357650 | | nonunique | FALSE |
| 16 | 2357792 | chr1+: 149273533-149273557 | intergenic | FALSE |
| 17 | 2358921 | PSMB4 | exonic | TRUE |
| 18 | 2360078 | C1orf43 | antisense | FALSE |
| 19 | 2363765 | FCGR2A | exonic | FALSE |
| 20 | 2364004 | OLFML2B | antisense | FALSE |
| 21 | 2364118 | C1orf226 | exonic | FALSE |
| 22 | 2368224 | | nonunique | FALSE |
| 23 | 2369169 | RASAL2 | intronic | FALSE |
| 24 | 2370319 | MR1 | exonic | TRUE |
| 25 | 2371121 | LAMC1 | exonic | TRUE |
| 26 | 2372800 | RGS1 | exonic | FALSE |
| 27 | 2375423 | RP11-480I12.3 | exonic | FALSE |
| 28 | 2376638 | AC119673.1 | intronic | FALSE |
| 29 | 2378767 | chr1+: 211700719-211700853 | intergenic | FALSE |
| 30 | 2381048 | IARS2 | exonic | FALSE |
| 31 | 2382372 | DEGS1 | exonic | TRUE |
| 32 | 2382373 | DEGS1 | intronic | FALSE |
| 33 | 2382379 | DEGS1 | exonic | FALSE |
| 34 | 2382380 | DEGS1 | exonic | FALSE |
| 35 | 2384422 | RHOU | exonic | FALSE |
| 36 | 2387132 | RYR2 | intronic | FALSE |
| 37 | 2389288 | KIF26B | intronic | FALSE |
| 38 | 2393573 | WDR8 | intronic | FALSE |
| 39 | 2395788 | chr1−: 9488721-9488846 | intergenic | FALSE |
| 40 | 2395827 | SLC25A33 | antisense | FALSE |
| 41 | 2400178 | CAMK2N1 | exonic | FALSE |
| 42 | 2400181 | CAMK2N1 | exonic | TRUE |
| 43 | 2402462 | STMN1 | exonic | FALSE |
| 44 | 2403251 | RP1-159A19.3 | antisense | FALSE |
| 45 | 2409349 | MED8 | exonic | FALSE |
| 46 | 2423624 | GCLM | exonic | FALSE |
| 47 | 2424687 | DPYD | intronic | FALSE |
| 48 | 2428763 | RSBN1 | exonic | TRUE |
| 49 | 2432001 | PDE4DIP | exonic | FALSE |
| 50 | 2432137 | | nonunique | FALSE |
| 51 | 2432161 | | nonunique | FALSE |
| 52 | 2432228 | | nonunique | FALSE |
| 53 | 2432306 | | nonunique | FALSE |
| 54 | 2434721 | LASS2 | exonic | FALSE |
| 55 | 2435126 | TUFT1, RP11-74C1.4_AS | antisense | FALSE |
| 56 | 2438284 | IQGAP3 | exonic | FALSE |
| 57 | 2438300 | IQGAP3 | exonic | FALSE |
| 58 | 2438346 | GPATCH4 | exonic | FALSE |
| 59 | 2438915 | FCRL5 | exonic | TRUE |
| 60 | 2440479 | F11R | exonic | FALSE |
| 61 | 2440953 | FCGR3A | exonic | FALSE |
| 62 | 2441248 | UHMK1 | antisense | FALSE |
| 63 | 2441392 | RGS5 | exonic | TRUE |
| 64 | 2441394 | RGS5 | exonic | FALSE |
| 65 | 2442144 | TMCO1 | exonic | FALSE |
| 66 | 2442908 | DCAF6 | antisense | FALSE |
| 67 | 2443144 | DPT | exonic | FALSE |
| 68 | 2445997 | ANGPTL1 | exonic | TRUE |
| 69 | 2447849 | EDEM3 | exonic | TRUE |
| 70 | 2449562 | ASPM | exonic | TRUE |
| 71 | 2450024 | RP11-31E23.1 | exonic | FALSE |
| 72 | 2450389 | KIF14 | exonic | TRUE |
| 73 | 2451070 | LMOD1 | intronic | FALSE |
| 74 | 2455740 | USH2A | exonic | TRUE |
| 75 | 2456850 | IARS2 | antisense | FALSE |
| 76 | 2457596 | | nonunique | FALSE |
| 77 | 2457622 | BROX | antisense | FALSE |
| 78 | 2458063 | NVL | exonic | TRUE |
| 79 | 2458075 | PARP1 | intronic | FALSE |
| 80 | 2459655 | RHOU | antisense | FALSE |
| 81 | 2465564 | ZNF124 | exonic | FALSE |
| 82 | 2465590 | ZNF124 | intronic | FALSE |
| 83 | 2466644 | AC144450.1 | antisense | FALSE |
| 84 | 2467153 | AC144450.1 | exonic | FALSE |
| 85 | 2468976 | IAH1 | exonic | FALSE |
| 86 | 2469277 | RRM2 | exonic | FALSE |
| 87 | 2475153 | PLB1 | exonic | TRUE |
| 88 | 2475696 | LBH, AC104698.1 | exonic | FALSE |
| 89 | 2478939 | MTA3 | intronic | FALSE |
| 90 | 2480977 | EPCAM | exonic | TRUE |
| 91 | 2487116 | ANTXR1 | exonic | TRUE |
| 92 | 2491297 | TMSB10 | exonic | FALSE |
| 93 | 2492206 | RMND5A | exonic | FALSE |
| 94 | 2495652 | chr2+: 99360165-99360384 | intergenic | FALSE |
| 95 | 2504315 | YWHAZP2 | antisense | FALSE |
| 96 | 2506357 | C2orf27A | intronic | FALSE |
| 97 | 2507963 | chr2+: 138992734-138993169 | intergenic | FALSE |
| 98 | 2514940 | AC007405.4 | antisense | FALSE |
| 99 | 2515105 | TLK1 | antisense | FALSE |
| 100 | 2518103 | chr2+: 181343569-181343698 | intergenic | FALSE |
| 101 | 2518112 | AC009478.1 | antisense | FALSE |
| 102 | 2518113 | AC009478.1 | antisense | FALSE |
| 103 | 2518123 | chr2+: 181623018-181623217 | intergenic | FALSE |
| 104 | 2518126 | chr2+: 181653946-181654097 | intergenic | FALSE |
| 105 | 2518128 | chr2+: 181684971-181685155 | intergenic | FALSE |
| 106 | 2518146 | chr2+: 181738756-181739243 | intergenic | FALSE |
| 107 | 2518154 | chr2+: 181750728-181750881 | intergenic | FALSE |
| 108 | 2518161 | chr2+: 181818605-181818727 | intergenic | FALSE |
| 109 | 2518181 | UBE2E3 | intronic | FALSE |
| 110 | 2518196 | | nonunique | FALSE |
| 111 | 2519637 | COL3A1 | exonic | TRUE |
| 112 | 2519657 | COL3A1 | exonic | FALSE |
| 113 | 2521466 | | nonunique | FALSE |
| 114 | 2521494 | HSPE1 | exonic | FALSE |
| 115 | 2525080 | CREB1 | exonic | TRUE |
| 116 | 2529793 | MRPL44 | exonic | FALSE |
| 117 | 2532135 | DIS3L2 | intronic | FALSE |
| 118 | 2533283 | TRPM8 | exonic | FALSE |
| 119 | 2536223 | ANO7 | exonic | FALSE |
| 120 | 2536226 | ANO7 | exonic | FALSE |
| 121 | 2536240 | ANO7 | exonic | TRUE |
| 122 | 2536258 | ANO7 | exonic | FALSE |
| 123 | 2536262 | ANO7 | exonic | FALSE |
| 124 | 2537722 | chr2+: 2669744-2669886 | intergenic | FALSE |
| 125 | 2545278 | OTOF | intronic | FALSE |
| 126 | 2546680 | LBH | antisense | FALSE |
| 127 | 2546780 | LCLAT1 | antisense | FALSE |
| 128 | 2553908 | CCDC104 | antisense | FALSE |
| 129 | 2555014 | BCL11A | intronic | FALSE |
| 130 | 2555017 | BCL11A | intronic | FALSE |
| 131 | 2555050 | BCL11A | intronic | FALSE |
| 132 | 2564601 | MRPS5 | exonic | FALSE |
| 133 | 2568115 | AC108051.3 | antisense | FALSE |
| 134 | 2574517 | | nonunique | FALSE |
| 135 | 2578171 | | nonunique | FALSE |
| 136 | 2584810 | COBLL1 | intronic | FALSE |
| 137 | 2585986 | ABCB11 | intronic | FALSE |
| 138 | 2590289 | chr2−: 181288712-181288835 | intergenic | FALSE |
| 139 | 2590310 | AC009478.1 | intronic | FALSE |
| 140 | 2590313 | AC009478.1 | intronic | FALSE |
| 141 | 2590320 | | nonunique | FALSE |
| 142 | 2590322 | AC009478.1 | intronic | FALSE |
| 143 | 2590342 | AC009478.1 | intronic | FALSE |
| 144 | 2590344 | AC009478.1 | intronic | FALSE |
| 145 | 2590349 | chr2−: 181643108-181643138 | intergenic | FALSE |
| 146 | 2590353 | chr2−: 181673067-181673179 | intergenic | FALSE |
| 147 | 2590359 | chr2−: 181724901-181725200 | intergenic | FALSE |
| 148 | 2590395 | UBE2E3 | antisense | FALSE |
| 149 | 2590916 | | nonunique | FALSE |
| 150 | 2591635 | COL3A1 | antisense | FALSE |
| 151 | 2591638 | COL3A1 | antisense | FALSE |
| 152 | 2591646 | COL5A2 | exonic | FALSE |
| 153 | 2593741 | | nonunique | FALSE |
| 154 | 2595375 | FAM117B | antisense | FALSE |
| 155 | 2598328 | FN1 | exonic | TRUE |
| 156 | 2601027 | FARSB | exonic | FALSE |
| 157 | 2604258 | HJURP | exonic | FALSE |
| 158 | 2604598 | chr2−: 236300744-236300769 | intergenic | FALSE |
| 159 | 2606962 | C2orf54 | intronic | FALSE |
| 160 | 2608319 | LRRN1 | intronic | FALSE |
| 161 | 2608325 | LRRN1 | exonic | FALSE |
| 162 | 2610353 | chr3+: 10195215-10195245 | intergenic | FALSE |
| 163 | 2611934 | SLC6A6 | exonic | TRUE |
| 164 | 2619930 | chr3+: 44155660-44155694 | intergenic | FALSE |
| 165 | 2620374 | TGM4 | exonic | TRUE |
| 166 | 2620381 | TGM4 | exonic | TRUE |
| 167 | 2620388 | TGM4 | exonic | TRUE |
| 168 | 2623152 | MANF | exonic | FALSE |
| 169 | 2625067 | WNT5A | antisense | FALSE |
| 170 | 2630641 | ROBO2 | intronic | FALSE |
| 171 | 2631342 | RP11-260O18.1 | intronic | FALSE |
| 172 | 2633447 | COL8A1 | exonic | FALSE |
| 173 | 2634575 | ALCAM | exonic | TRUE |
| 174 | 2634580 | ALCAM | exonic | FALSE |
| 175 | 2636073 | C3orf52 | exonic | TRUE |
| 176 | 2638451 | NDUFB4 | exonic | TRUE |
| 177 | 2641061 | SEC61A1 | exonic | TRUE |
| 178 | 2647816 | RP11-392O18.1 | exonic | FALSE |
| 179 | 2650228 | SMC4 | exonic | TRUE |
| 180 | 2650232 | SMC4 | exonic | TRUE |
| 181 | 2650237 | SMC4 | exonic | TRUE |
| 182 | 2650245 | SMC4 | exonic | TRUE |
| 183 | 2650247 | SMC4 | exonic | TRUE |
| 184 | 2651875 | GPR160 | exonic | FALSE |
| 185 | 2653214 | NAALADL2 | intronic | FALSE |
| 186 | 2653216 | NAALADL2 | exonic | TRUE |
| 187 | 2653248 | chr3+: 175527761-175528254 | intergenic | FALSE |
| 188 | 2662603 | chr3−: 10195138-10195267 | intergenic | FALSE |
| 189 | 2677192 | RP11-674P14.1 | exonic | FALSE |
| 190 | 2677923 | ASB14 | exonic | FALSE |
| 191 | 2681851 | FOXP1 | intronic | FALSE |
| 192 | 2682663 | PPP4R2 | antisense | FALSE |
| 193 | 2687242 | ALCAM | antisense | FALSE |
| 194 | 2689215 | NAA50 | exonic | FALSE |
| 195 | 2690262 | LSAMP | intronic | FALSE |
| 196 | 2695559 | CPNE4 | exonic | TRUE |
| 197 | 2697930 | NMNAT3 | intronic | FALSE |
| 198 | 2700221 | HLTF | exonic | TRUE |
| 199 | 2701587 | ARHGEF26 | antisense | FALSE |
| 200 | 2701589 | ARHGEF26 | antisense | FALSE |
| 201 | 2703212 | RP11-432B6.3 | intronic | FALSE |
| 202 | 2706143 | NAALADL2 | antisense | FALSE |
| 203 | 2706171 | chr3−: 175524544-175524898 | intergenic | FALSE |
| 204 | 2709360 | RP11-78H24.1 | antisense | FALSE |
| 205 | 2720286 | NCAPG | exonic | FALSE |
| 206 | 2724392 | UGDH | antisense | FALSE |
| 207 | 2725077 | LIMCH1 | intronic | FALSE |
| 208 | 2725416 | SLC30A9 | exonic | TRUE |
| 209 | 2727579 | chr4+: 55366532-55366734 | intergenic | FALSE |
| 210 | 2730538 | UTP3 | exonic | FALSE |
| 211 | 2732312 | 11-Sep | exonic | TRUE |
| 212 | 2733210 | RP11-610O8.1 | exonic | FALSE |
| 213 | 2737932 | CENPE | antisense | FALSE |
| 214 | 2739770 | AP1AR | exonic | TRUE |
| 215 | 2744749 | | nonunique | FALSE |
| 216 | 2749469 | | nonunique | FALSE |
| 217 | 2754760 | SORBS2 | antisense | FALSE |
| 218 | 2757601 | C4orf48 | antisense | FALSE |
| 219 | 2764274 | SEL1L3 | intronic | FALSE |
| 220 | 2768574 | FRYL | exonic | TRUE |
| 221 | 2771431 | EPHA5 | intronic | FALSE |
| 222 | 2772627 | GRSF1 | exonic | FALSE |
| 223 | 2775054 | ANTXR2 | intronic | FALSE |
| 224 | 2777055 | HSD17B13, RP11-529H2.2 | exonic | TRUE |
| 225 | 2779642 | PPP3CA | exonic | FALSE |
| 226 | 2787004 | SCOC | antisense | FALSE |
| 227 | 2789315 | LRBA | intronic | FALSE |
| 228 | 2793953 | HMGB2 | exonic | FALSE |
| 229 | 2803194 | FAM134B | antisense | FALSE |
| 230 | 2805610 | SUB1 | exonic | FALSE |
| 231 | 2805826 | TARS | exonic | FALSE |
| 232 | 2807394 | OSMR | exonic | TRUE |
| 233 | 2808101 | SEPP1 | antisense | FALSE |
| 234 | 2817338 | chr5+: 78664964-78665863 | intergenic | FALSE |
| 235 | 2817622 | THBS4 | exonic | FALSE |
| 236 | 2818565 | VCAN | exonic | TRUE |
| 237 | 2825917 | PRR16 | intronic | FALSE |
| 238 | 2825925 | PRR16 | intronic | FALSE |
| 239 | 2825928 | PRR16 | intronic | FALSE |
| 240 | 2825941 | PRR16 | exonic | FALSE |
| 241 | 2827569 | SLC12A2 | exonic | TRUE |
| 242 | 2828896 | HSPA4 | exonic | TRUE |
| 243 | 2829806 | CTC-321K16.1 | intronic | FALSE |
| 244 | 2833961 | SH3RF2 | intronic | FALSE |
| 245 | 2835934 | SPARC | antisense | FALSE |
| 246 | 2838213 | PTTG1 | exonic | FALSE |
| 247 | 2841541 | BNIP1 | intronic | FALSE |
| 248 | 2844255 | CANX | intronic | FALSE |
| 249 | 2847418 | PAPD7 | antisense | FALSE |
| 250 | 2848429 | ANKRD33B, RP11- | antisense | FALSE |
| | | 215G15.2_AS | | |
| 251 | 2849085 | DNAH5 | exonic | TRUE |
| 252 | 2849097 | DNAH5 | exonic | TRUE |
| 253 | 2849101 | DNAH5 | exonic | TRUE |
| 254 | 2849111 | DNAH5 | exonic | TRUE |
| 255 | 2849128 | DNAH5 | exonic | TRUE |
| 256 | 2849152 | DNAH5 | exonic | TRUE |
| 257 | 2849171 | DNAH5 | exonic | TRUE |
| 258 | 2849993 | FAM134B | exonic | FALSE |
| 259 | 2850078 | chr5−: 16663523-16663973 | intergenic | FALSE |
| 260 | 2852749 | AMACR, RP11-1084J3.3 | exonic | FALSE |
| 261 | 2853003 | RAD1 | exonic | FALSE |
| 262 | 2853095 | AGXT2 | exonic | TRUE |
| 263 | 2855504 | HMGCS1 | exonic | FALSE |
| 264 | 2858556 | PDE4D | intronic | FALSE |
| 265 | 2858567 | PDE4D | intronic | FALSE |
| 266 | 2860474 | chr5−: 67878837-67878884 | intergenic | FALSE |
| 267 | 2863638 | | nonunique | FALSE |
| 268 | 2865309 | CTC-348L14.1 | exonic | FALSE |
| 269 | 2867861 | | nonunique | FALSE |
| 270 | 2872731 | PRR16 | antisense | FALSE |
| 271 | 2872735 | PRR16 | antisense | FALSE |
| 272 | 2873224 | CEP120 | intronic | FALSE |
| 273 | 2874688 | HINT1 | exonic | FALSE |
| 274 | 2875402 | AC004041.2 | intronic | FALSE |
| 275 | 2875667 | HSPA4 | antisense | FALSE |
| 276 | 2876625 | CXCL14 | exonic | FALSE |
| 277 | 2877630 | chr5−: 138271234-138271305 | intergenic | FALSE |
| 278 | 2879111 | SPRY4 | intronic | FALSE |
| 279 | 2879885 | SH3RF2 | antisense | FALSE |
| 280 | 2882121 | SPARC | exonic | FALSE |
| 281 | 2882122 | SPARC | exonic | FALSE |
| 282 | 2882125 | SPARC | exonic | FALSE |
| 283 | 2882868 | C5orf4 | exonic | TRUE |
| 284 | 2893447 | LY86 | exonic | FALSE |
| 285 | 2893942 | TXNDC5, MUTED_AS | antisense | FALSE |
| 286 | 2895783 | CCDC90A | antisense | FALSE |
| 287 | 2897918 | SOX4 | exonic | FALSE |
| 288 | 2898585 | C6orf62 | antisense | FALSE |
| 289 | 2898613 | GMNN | intronic | FALSE |
| 290 | 2898626 | GMNN | exonic | FALSE |
| 291 | 2898627 | GMNN | exonic | FALSE |
| 292 | 2898891 | LRRC16A | exonic | TRUE |
| 293 | 2903184 | | nonunique | FALSE |
| 294 | 2903668 | KIFC1 | exonic | FALSE |
| 295 | 2905908 | GLO1 | antisense | FALSE |
| 296 | 2908456 | chr6+: 44202685-44202903 | intergenic | FALSE |
| 297 | 2910568 | ELOVL5 | antisense | FALSE |
| 298 | 2910834 | | nonunique | FALSE |
| 299 | 2922229 | MARCKS | exonic | FALSE |
| 300 | 2922230 | MARCKS | exonic | FALSE |
| 301 | 2922233 | MARCKS | exonic | FALSE |
| 302 | 2927747 | HEBP2 | exonic | TRUE |
| 303 | 2929419 | chr6+: 145359286-145359591 | intergenic | FALSE |
| 304 | 2931975 | | nonunique | FALSE |
| 305 | 2934526 | SLC22A3 | intronic | FALSE |
| 306 | 2934538 | SLC22A3 | exonic | TRUE |
| 307 | 2934543 | SLC22A3 | intronic | FALSE |
| 308 | 2934546 | SLC22A3 | intronic | FALSE |
| 309 | 2934551 | SLC22A3 | intronic | FALSE |
| 310 | 2934556 | SLC22A3 | intronic | FALSE |
| 311 | 2934557 | SLC22A3 | intronic | FALSE |
| 312 | 2934568 | SLC22A3 | intronic | FALSE |
| 313 | 2934569 | SLC22A3 | intronic | FALSE |
| 314 | 2934571 | SLC22A3 | intronic | FALSE |
| 315 | 2934731 | | nonunique | FALSE |
| 316 | 2937410 | XXyac-YX65C7_A.2 | intronic | FALSE |
| 317 | 2937411 | XXyac-YX65C7_A.2 | intronic | FALSE |
| 318 | 2938797 | GMDS | intronic | FALSE |
| 319 | 2944090 | DEK | exonic | TRUE |
| 320 | 2944282 | chr6−: 19135505-19135580 | intergenic | FALSE |
| 321 | 2944959 | SOX4 | antisense | FALSE |
| 322 | 2944963 | SOX4 | antisense | FALSE |
| 323 | 2946859 | ZNF204P | exonic | FALSE |
| 324 | 2948972 | | nonunique | FALSE |
| 325 | 2949847 | AGER | exonic | TRUE |
| 326 | 2951060 | C6orf1 | exonic | FALSE |
| 327 | 2951708 | SRPK1 | intronic | FALSE |
| 328 | 2952506 | BTBD9 | exonic | FALSE |
| 329 | 2952680 | GLO1 | exonic | TRUE |
| 330 | 2952682 | GLO1 | exonic | TRUE |
| 331 | 2952683 | GLO1 | exonic | TRUE |
| 332 | 2952684 | GLO1 | exonic | TRUE |
| 333 | 2952686 | GLO1 | exonic | TRUE |
| 334 | 2952695 | GLO1 | intronic | FALSE |
| 335 | 2953502 | TREM2 | exonic | FALSE |
| 336 | 2961323 | TMEM30A | exonic | FALSE |
| 337 | 2971087 | | nonunique | FALSE |
| 338 | 2982619 | SLC22A3 | antisense | FALSE |
| 339 | 2985810 | THBS2 | exonic | FALSE |
| 340 | 2985811 | THBS2 | exonic | FALSE |
| 341 | 2985813 | THBS2 | exonic | FALSE |
| 342 | 2987581 | IQCE | exonic | FALSE |
| 343 | 2987678 | TTYH3 | exonic | FALSE |
| 344 | 2988898 | EIF2AK1 | antisense | FALSE |
| 345 | 2992848 | GPNMB | exonic | FALSE |
| 346 | 2993649 | CBX3 | exonic | TRUE |
| 347 | 2993657 | | nonunique | FALSE |
| 348 | 2995379 | GGCT | antisense | FALSE |
| 349 | 2997929 | SFRP4 | antisense | FALSE |
| 350 | 2998432 | RALA | exonic | TRUE |
| 351 | 2998957 | INHBA, AC005027.3_AS | antisense | FALSE |
| 352 | 3000124 | H2AFV | antisense | FALSE |
| 353 | 3002872 | chr7+: 55419044-55419189 | intergenic | FALSE |
| 354 | 3003598 | | nonunique | FALSE |
| 355 | 3006337 | RP5-945F2.3 | antisense | FALSE |
| 356 | 3008101 | ELN | exonic | FALSE |
| 357 | 3009423 | YWHAG | antisense | FALSE |
| 358 | 3009425 | YWHAG | antisense | FALSE |
| 359 | 3017037 | LRRC17 | intronic | FALSE |
| 360 | 3021691 | NDUFA5 | antisense | FALSE |
| 361 | 3025519 | BPGM | exonic | FALSE |
| 362 | 3031189 | ATP6V0E2 | intronic | FALSE |
| 363 | 3034986 | SUN1, GET4_AS | antisense | FALSE |
| 364 | 3037195 | EIF2AK1 | exonic | FALSE |
| 365 | 3037287 | CYTH3 | intronic | FALSE |
| 366 | 3038619 | | nonunique | FALSE |
| 367 | 3039818 | AGR2 | exonic | FALSE |
| 368 | 3039819 | AGR2 | exonic | FALSE |
| 369 | 3042003 | | nonunique | FALSE |
| 370 | 3044132 | | nonunique | FALSE |
| 371 | 3044138 | GGCT | exonic | TRUE |
| 372 | 3046448 | SFRP4 | exonic | FALSE |
| 373 | 3046449 | SFRP4 | exonic | FALSE |
| 374 | 3046450 | SFRP4 | exonic | FALSE |
| 375 | 3046453 | SFRP4 | exonic | TRUE |
| 376 | 3046457 | SFRP4 | exonic | TRUE |
| 377 | 3046459 | SFRP4 | exonic | TRUE |
| 378 | 3046460 | SFRP4 | exonic | TRUE |
| 379 | 3046461 | SFRP4 | exonic | TRUE |
| 380 | 3047596 | INHBA | exonic | TRUE |
| 381 | 3047600 | INHBA | exonic | FALSE |
| 382 | 3049294 | IGFBP3 | exonic | TRUE |
| 383 | 3051867 | GBAS | antisense | FALSE |
| 384 | 3052975 | | nonunique | FALSE |
| 385 | 3054243 | PMS2P4 | intronic | FALSE |
| 386 | 3061759 | COL1A2 | antisense | FALSE |
| 387 | 3063309 | ATP5J2 | exonic | TRUE |
| 388 | 3070716 | WASL | exonic | FALSE |
| 389 | 3074191 | C7orf49 | exonic | FALSE |
| 390 | 3074661 | MTPN | exonic | FALSE |
| 391 | 3076359 | chr7−: 140424479-140424913 | intergenic | FALSE |
| 392 | 3091131 | DPYSL2 | exonic | TRUE |
| 393 | 3092394 | TUBB4Q | exonic | FALSE |
| 394 | 3097077 | KIAA0146 | intronic | FALSE |
| 395 | 3099650 | FAM110B | intronic | FALSE |
| 396 | 3102585 | chr8+: 70984173-70984278 | intergenic | FALSE |
| 397 | 3102708 | AC120194.1 | exonic | FALSE |
| 398 | 3102724 | RP11-382J12.1 | intronic | FALSE |
| 399 | 3104305 | PKIA | exonic | FALSE |
| 400 | 3104626 | TPD52 | antisense | FALSE |
| 401 | 3105911 | CPNE3 | exonic | TRUE |
| 402 | 3107563 | ESRP1 | exonic | TRUE |
| 403 | 3107565 | ESRP1 | exonic | TRUE |
| 404 | 3107711 | INTS8 | exonic | FALSE |
| 405 | 3108061 | UQCRB | antisense | FALSE |
| 406 | 3108479 | MTDH | exonic | FALSE |
| 407 | 3108933 | VPS13B | exonic | TRUE |
| 408 | 3109077 | VPS13B | exonic | TRUE |
| 409 | 3109200 | POLR2K | exonic | FALSE |
| 410 | 3109252 | SPAG1 | exonic | FALSE |
| 411 | 3109448 | YWHAZ | antisense | FALSE |
| 412 | 3110070 | AZIN1 | antisense | FALSE |
| 413 | 3110196 | ATP6V1C1 | exonic | FALSE |
| 414 | 3110496 | RIMS2 | intronic | FALSE |
| 415 | 3112517 | EIF3H | antisense | FALSE |
| 416 | 3112570 | UTP23 | intronic | FALSE |
| 417 | 3114046 | RP11-557C18.3 | exonic | FALSE |
| 418 | 3114390 | FAM91A1 | exonic | TRUE |
| 419 | 3114858 | SQLE | exonic | TRUE |
| 420 | 3118388 | TRAPPC9 | antisense | FALSE |
| 421 | 3126713 | SLC18A1 | intronic | FALSE |
| 422 | 3128632 | chr8−: 26120364-26120507 | intergenic | FALSE |
| 423 | 3130284 | chr8−: 30794711-30794762 | intergenic | FALSE |
| 424 | 3131845 | LSM1 | exonic | FALSE |
| 425 | 3134070 | PRKDC | exonic | TRUE |
| 426 | 3134081 | PRKDC | exonic | TRUE |
| 427 | 3134228 | UBE2V2 | antisense | FALSE |
| 428 | 3138429 | ARMC1 | exonic | TRUE |
| 429 | 3138457 | MTFR1 | antisense | FALSE |
| 430 | 3138883 | SNHG6 | exonic | FALSE |
| 431 | 3138885 | SNHG6 | exonic | FALSE |
| 432 | 3139108 | ARFGEF1 | exonic | TRUE |
| 433 | 3139153 | AC011037.1 | antisense | FALSE |
| 434 | 3139158 | CPA6 | exonic | TRUE |
| 435 | 3139175 | CPA6 | exonic | TRUE |
| 436 | 3139176 | CPA6 | exonic | TRUE |
| 437 | 3139195 | CPA6 | intronic | FALSE |
| 438 | 3139216 | CPA6 | exonic | TRUE |
| 439 | 3139562 | SULF1 | antisense | FALSE |
| 440 | 3139724 | NCOA2 | exonic | FALSE |
| 441 | 3139906 | TRAM1 | exonic | FALSE |
| 442 | 3140115 | EYA1 | exonic | TRUE |
| 443 | 3140723 | STAU2 | intronic | FALSE |
| 444 | 3140840 | TCEB1 | exonic | TRUE |
| 445 | 3141597 | IL7 | exonic | FALSE |
| 446 | 3141598 | IL7 | intronic | FALSE |
| 447 | 3141866 | TPD52 | exonic | FALSE |
| 448 | 3143408 | CNGB3 | intronic | FALSE |
| 449 | 3145085 | ESRP1 | antisense | FALSE |
| 450 | 3145576 | | nonunique | FALSE |
| 451 | 3146436 | COX6C | exonic | FALSE |
| 452 | 3146538 | POLR2K | antisense | FALSE |
| 453 | 3146675 | ANKRD46 | exonic | FALSE |
| 454 | 3146809 | PABPC1 | exonic | TRUE |
| 455 | 3146901 | | nonunique | FALSE |
| 456 | 3146906 | | nonunique | FALSE |
| 457 | 3147325 | UBR5 | exonic | FALSE |
| 458 | 3147479 | KB-1980E6.3 | antisense | FALSE |
| 459 | 3149768 | EIF3H | exonic | FALSE |
| 460 | 3150536 | RP11-4K16.2 | exonic | FALSE |
| 461 | 3150537 | RP11-4K16.2 | intronic | FALSE |
| 462 | 3150804 | MRPL13 | exonic | FALSE |
| 463 | 3152560 | FAM84B | exonic | FALSE |
| 464 | 3153341 | FAM49B | exonic | TRUE |
| 465 | 3157723 | FAM83H | exonic | FALSE |
| 466 | 3159349 | DOCK8 | exonic | FALSE |
| 467 | 3159383 | DOCK8 | exonic | TRUE |
| 468 | 3164986 | MTAP, CDKN2B-AS1 | intronic | FALSE |
| 469 | 3165566 | TUSC1 | antisense | FALSE |
| 470 | 3166461 | chr9+: 32204125-32204151 | intergenic | FALSE |
| 471 | 3173527 | PGM5 | intronic | FALSE |
| 472 | 3175540 | PCA3 | exonic | FALSE |
| 473 | 3178505 | NXNL2 | intronic | FALSE |
| 474 | 3179420 | CENPP | intronic | FALSE |
| 475 | 3180211 | chr9+: 96886673-96886768 | intergenic | FALSE |
| 476 | 3180289 | HIATL1 | exonic | FALSE |
| 477 | 3181440 | ANP32B | exonic | FALSE |
| 478 | 3183802 | RAD23B | exonic | FALSE |
| 479 | 3184980 | DNAJC25- | exonic | FALSE |
| | | GNG10, GNG10, DNAJC25 | | |
| 480 | 3190133 | RP11-203J24.8 | intronic | FALSE |
| 481 | 3191313 | GPR107 | intronic | FALSE |
| 482 | 3191953 | NUP214 | exonic | TRUE |
| 483 | 3202822 | | nonunique | FALSE |
| 484 | 3203313 | APTX | exonic | FALSE |
| 485 | 3204131 | UNC13B | antisense | FALSE |
| 486 | 3205546 | TOMM5, RP11- | exonic | FALSE |
| | | 613M10.8, RP11- | | |
| | | 613M10.9, FBXO10 | | |
| 487 | 3210661 | chr9−: 79534636-79534676 | intergenic | FALSE |
| 488 | 3212374 | RMI1 | antisense | FALSE |
| 489 | 3214846 | ASPN | exonic | FALSE |
| 490 | 3214859 | ASPN | exonic | TRUE |
| 491 | 3214862 | ASPN | exonic | TRUE |
| 492 | 3217118 | ANP32B | antisense | FALSE |
| 493 | 3219845 | EPB41L4B | exonic | TRUE |
| 494 | 3220159 | TXN | exonic | FALSE |
| 495 | 3221146 | C9orf80 | intronic | FALSE |
| 496 | 3241852 | RP11-342D11.2 | exonic | FALSE |
| 497 | 3242831 | | nonunique | FALSE |
| 498 | 3245562 | | nonunique | FALSE |
| 499 | 3255737 | GRID1 | antisense | FALSE |
| 500 | 3261642 | GBF1 | intronic | FALSE |
| 501 | 3265186 | TDRD1 | exonic | TRUE |
| 502 | 3265201 | TDRD1 | exonic | TRUE |
| 503 | 3265206 | TDRD1 | exonic | TRUE |
| 504 | 3265207 | TDRD1 | exonic | TRUE |
| 505 | 3265208 | TDRD1 | exonic | TRUE |
| 506 | 3265210 | TDRD1 | exonic | TRUE |
| 507 | 3265211 | TDRD1 | exonic | TRUE |
| 508 | 3265212 | TDRD1 | exonic | TRUE |
| 509 | 3265217 | TDRD1 | exonic | FALSE |
| 510 | 3265218 | TDRD1 | intronic | FALSE |
| 511 | 3268465 | RP11-107C16.2 | intronic | FALSE |
| 512 | 3284324 | NRP1 | exonic | TRUE |
| 513 | 3284346 | NRP1 | exonic | TRUE |
| 514 | 3284351 | NRP1 | exonic | TRUE |
| 515 | 3284391 | NRP1 | intronic | FALSE |
| 516 | 3284420 | NRP1 | intronic | FALSE |
| 517 | 3286210 | CSGALNACT2 | antisense | FALSE |
| 518 | 3286634 | CXCL12 | intronic | FALSE |
| 519 | 3290532 | BICC1 | antisense | FALSE |
| 520 | 3292624 | HNRNPH3 | antisense | FALSE |
| 521 | 3294585 | USP54 | exonic | TRUE |
| 522 | 3294926 | CAMK2G | exonic | TRUE |
| 523 | 3299263 | ATAD1 | intronic | FALSE |
| 524 | 3300132 | PPP1R3C | exonic | TRUE |
| 525 | 3300608 | MYOF | exonic | TRUE |
| 526 | 3300669 | MYOF | intronic | FALSE |
| 527 | 3301916 | PIK3AP1 | exonic | FALSE |
| 528 | 3302849 | HPS1 | exonic | FALSE |
| 529 | 3305263 | WDR96 | intronic | FALSE |
| 530 | 3307444 | TCF7L2 | antisense | FALSE |
| 531 | 3310123 | FGFR2 | exonic | TRUE |
| 532 | 3310134 | FGFR2 | intronic | FALSE |
| 533 | 3310163 | FGFR2 | intronic | FALSE |
| 534 | 3317547 | SLC22A18 | exonic | TRUE |
| 535 | 3318045 | RRM1 | exonic | FALSE |
| 536 | 3318585 | AC111177.1 | exonic | TRUE |
| 537 | 3323243 | NAV2 | exonic | FALSE |
| 538 | 3332088 | OSBP | antisense | FALSE |
| 539 | 3334113 | NAA40 | exonic | FALSE |
| 540 | 3335233 | NEAT1 | exonic | FALSE |
| 541 | 3335235 | NEAT1 | exonic | FALSE |
| 542 | 3335635 | SNX32 | intronic | FALSE |
| 543 | 3337192 | GSTP1 | exonic | TRUE |
| 544 | 3343904 | | nonunique | FALSE |
| 545 | 3343907 | | nonunique | FALSE |
| 546 | 3343913 | FOLH1B | exonic | TRUE |
| 547 | 3343916 | | nonunique | FALSE |
| 548 | 3345480 | RP11-712B9.2 | intronic | FALSE |
| 549 | 3345483 | RP11-712B9.2 | intronic | FALSE |
| 550 | 3345484 | RP11-712B9.2 | intronic | FALSE |
| 551 | 3354757 | EI24 | exonic | FALSE |
| 552 | 3357277 | RP11-700F16.3 | intronic | FALSE |
| 553 | 3357343 | GLB1L3 | exonic | FALSE |
| 554 | 3357369 | GLB1L3 | exonic | TRUE |
| 555 | 3357382 | GLB1L3 | exonic | TRUE |
| 556 | 3357386 | GLB1L3 | exonic | TRUE |
| 557 | 3360223 | OR51E2 | exonic | FALSE |
| 558 | 3361499 | OR5P2 | exonic | TRUE |
| 559 | 3362160 | NRIP3 | exonic | FALSE |
| 560 | 3362745 | EIF4G2 | exonic | TRUE |
| 561 | 3372905 | FOLH1 | intronic | FALSE |
| 562 | 3372910 | | nonunique | FALSE |
| 563 | 3372912 | FOLH1 | exonic | FALSE |
| 564 | 3372921 | FOLH1 | exonic | TRUE |
| 565 | 3372923 | FOLH1 | exonic | FALSE |
| 566 | 3372927 | | nonunique | FALSE |
| 567 | 3372952 | | nonunique | FALSE |
| 568 | 3372960 | FOLH1 | intronic | FALSE |
| 569 | 3374858 | MRPL16 | exonic | FALSE |
| 570 | 3375519 | C11orf10 | exonic | FALSE |
| 571 | 3377632 | NEAT1 | antisense | FALSE |
| 572 | 3377633 | NEAT1 | antisense | FALSE |
| 573 | 3377641 | NEAT1 | antisense | FALSE |
| 574 | 3377670 | LTBP3 | exonic | FALSE |
| 575 | 3377893 | CFL1 | exonic | FALSE |
| 576 | 3379572 | PPP6R3 | antisense | FALSE |
| 577 | 3382801 | ACER3 | antisense | FALSE |
| 578 | 3383149 | NDUFC2 | exonic | FALSE |
| 579 | 3385956 | NOX4 | exonic | FALSE |
| 580 | 3387255 | SESN3 | exonic | FALSE |
| 581 | 3387257 | SESN3 | exonic | FALSE |
| 582 | 3387260 | SESN3 | exonic | FALSE |
| 583 | 3387273 | SESN3 | exonic | TRUE |
| 584 | 3387283 | SESN3 | intronic | FALSE |
| 585 | 3388797 | MMP10 | exonic | TRUE |
| 586 | 3388925 | RP11-690D19.1 | antisense | FALSE |
| 587 | 3389256 | chr11−: 104748668-104748860 | intergenic | FALSE |
| 588 | 3389668 | chr11−: 106550724-106550914 | intergenic | FALSE |
| 589 | 3393872 | UBE4A | antisense | FALSE |
| 590 | 3394416 | THY1 | exonic | FALSE |
| 591 | 3399563 | NCAPD3 | exonic | TRUE |
| 592 | 3399573 | NCAPD3 | exonic | TRUE |
| 593 | 3399586 | NCAPD3 | intronic | FALSE |
| 594 | 3399591 | NCAPD3 | exonic | TRUE |
| 595 | 3400101 | WNK1 | exonic | TRUE |
| 596 | 3404616 | OLR1 | antisense | FALSE |
| 597 | 3405395 | GPR19 | antisense | FALSE |
| 598 | 3411926 | chr12+: 42075852-42075977 | intergenic | FALSE |
| 599 | 3413681 | AC073610.5, ARF3_AS | antisense | FALSE |
| 600 | 3413826 | TUBA1C | exonic | TRUE |
| 601 | 3416319 | HOXC6 | exonic | TRUE |
| 602 | 3416325 | HOXC6 | exonic | FALSE |
| 603 | 3417063 | | nonunique | FALSE |
| 604 | 3418183 | MARS | exonic | TRUE |
| 605 | 3419453 | PPM1H | antisense | FALSE |
| 606 | 3419620 | RP11-415I12.6 | exonic | FALSE |
| 607 | 3420977 | GS1-410F4.2 | intronic | FALSE |
| 608 | 3424287 | PPFIA2 | antisense | FALSE |
| 609 | 3428610 | MYBPC1 | exonic | TRUE |
| 610 | 3428626 | MYBPC1 | exonic | TRUE |
| 611 | 3428627 | MYBPC1 | intronic | FALSE |
| 612 | 3428641 | MYBPC1 | exonic | TRUE |
| 613 | 3428651 | MYBPC1 | exonic | TRUE |
| 614 | 3428655 | MYBPC1 | exonic | TRUE |
| 615 | 3430967 | ACACB | exonic | TRUE |
| 616 | 3430986 | ACACB | exonic | TRUE |
| 617 | 3433378 | MED13L | antisense | FALSE |
| 618 | 3433778 | RFC5 | exonic | FALSE |
| 619 | 3434307 | | nonunique | FALSE |
| 620 | 3435781 | CDK2AP1, RP11- | antisense | FALSE |
| | | 282O18.3_AS | | |
| 621 | 3436782 | chr12+: 126375306- | intergenic | FALSE |
| | | 126375361 | | |
| 622 | 3439813 | WNK1 | antisense | FALSE |
| 623 | 3440112 | CACNA2D4 | intronic | FALSE |
| 624 | 3447097 | ST8SIA1 | intronic | FALSE |
| 625 | 3449291 | | nonunique | FALSE |
| 626 | 3453875 | TUBA1C | antisense | FALSE |
| 627 | 3454581 | SLC11A2 | exonic | FALSE |
| 628 | 3456527 | HOXC6, HOXC5_AS, | antisense | FALSE |
| | | AC012531.1 AS | | |
| 629 | 3460062 | XPOT | antisense | FALSE |
| 630 | 3462868 | NAP1L1 | exonic | TRUE |
| 631 | 3462969 | OSBPL8 | exonic | TRUE |
| 632 | 3463873 | PPFIA2 | exonic | TRUE |
| 633 | 3465666 | EEA1 | exonic | FALSE |
| 634 | 3466310 | NDUFA12 | intronic | FALSE |
| 635 | 3468077 | chr12−: 102090490-102090744 | intergenic | FALSE |
| 636 | 3468110 | GNPTAB | exonic | TRUE |
| 637 | 3473731 | WSB2 | exonic | TRUE |
| 638 | 3474576 | DYNLL1 | antisense | FALSE |
| 639 | 3475478 | MLXIP | antisense | FALSE |
| 640 | 3477561 | chr12−: 128230446-128230598 | intergenic | FALSE |
| 641 | 3481253 | chr13+: 23510032-23510056 | intergenic | FALSE |
| 642 | 3482132 | PABPC3 | exonic | TRUE |
| 643 | 3485957 | POSTN | antisense | FALSE |
| 644 | 3490910 | OLFM4 | exonic | TRUE |
| 645 | 3498806 | ZIC2 | exonic | FALSE |
| 646 | 3499158 | ITGBL1 | exonic | TRUE |
| 647 | 3499164 | ITGBL1 | exonic | TRUE |
| 648 | 3499166 | ITGBL1 | exonic | TRUE |
| 649 | 3499183 | ITGBL1 | exonic | TRUE |
| 650 | 3499188 | ITGBL1 | exonic | TRUE |
| 651 | 3499195 | ITGBL1 | exonic | TRUE |
| 652 | 3499197 | ITGBL1 | exonic | FALSE |
| 653 | 3499202 | ITGBL1 | exonic | FALSE |
| 654 | 3499216 | FGF14 | antisense | FALSE |
| 655 | 3504994 | chr13−: 22572519-22572642 | intergenic | FALSE |
| 656 | 3505255 | chr13−: 23575190-23575214 | intergenic | FALSE |
| 657 | 3510070 | POSTN | exonic | TRUE |
| 658 | 3510096 | POSTN | exonic | TRUE |
| 659 | 3513056 | LRCH1 | antisense | FALSE |
| 660 | 3513641 | chr13−: 49365707-49365740 | intergenic | FALSE |
| 661 | 3522423 | | nonunique | FALSE |
| 662 | 3523503 | ITGBL1 | antisense | FALSE |
| 663 | 3531094 | SCFD1 | exonic | TRUE |
| 664 | 3536992 | KTN1 | exonic | TRUE |
| 665 | 3537014 | KTN1 | exonic | TRUE |
| 666 | 3544154 | LTBP2 | antisense | FALSE |
| 667 | 3545640 | chr14+: 78455940-78456046 | intergenic | FALSE |
| 668 | 3547899 | FOXN3 | antisense | FALSE |
| 669 | 3552812 | | nonunique | FALSE |
| 670 | 3564236 | PYGL | exonic | TRUE |
| 671 | 3580172 | chr14−: 102518970-102519398 | intergenic | FALSE |
| 672 | 3583749 | NIPA1 | antisense | FALSE |
| 673 | 3588740 | C15orf41 | intronic | FALSE |
| 674 | 3590407 | NUSAP1 | exonic | FALSE |
| 675 | 3590517 | TYRO3 | exonic | TRUE |
| 676 | 3592280 | DUOX1 | exonic | TRUE |
| 677 | 3595452 | AC090651.1, GCOM1, | exonic | TRUE |
| | | GRINL1A | | |
| 678 | 3596817 | chr15+: 62011046-62011085 | intergenic | FALSE |
| 679 | 3601593 | CCDC33 | exonic | FALSE |
| 680 | 3608380 | chr15+: 91379904-91379944 | intergenic | FALSE |
| 681 | 3608543 | UNC45A | exonic | FALSE |
| 682 | 3613341 | NIPA1 | exonic | FALSE |
| 683 | 3617429 | LPCAT4 | exonic | TRUE |
| 684 | 3618346 | MEIS2 | exonic | TRUE |
| 685 | 3618445 | MEIS2 | exonic | TRUE |
| 686 | 3618459 | MEIS2 | exonic | TRUE |
| 687 | 3618462 | MEIS2 | intronic | FALSE |
| 688 | 3618464 | MEIS2 | exonic | TRUE |
| 689 | 3618467 | MEIS2 | exonic | FALSE |
| 690 | 3620836 | TTBK2 | exonic | TRUE |
| 691 | 3628924 | FAM96A | exonic | FALSE |
| 692 | 3630746 | ITGA11 | exonic | FALSE |
| 693 | 3632489 | C15orf60 | antisense | FALSE |
| 694 | 3645018 | PDPK1 | exonic | FALSE |
| 695 | 3650722 | ARL6IP1 | antisense | FALSE |
| 696 | 3661429 | chr16+: 54437159-54437183 | intergenic | FALSE |
| 697 | 3665331 | ELMO3 | exonic | TRUE |
| 698 | 3669724 | WWOX | exonic | TRUE |
| 699 | 3674530 | | nonunique | FALSE |
| 700 | 3675021 | RGS11 | exonic | FALSE |
| 701 | 3678446 | UBN1 | antisense | FALSE |
| 702 | 3680620 | GSPT1 | exonic | FALSE |
| 703 | 3682131 | MYH11 | exonic | FALSE |
| 704 | 3683768 | ACSM1 | exonic | TRUE |
| 705 | 3686386 | XPO6 | exonic | TRUE |
| 706 | 3687415 | FAM57B | intronic | FALSE |
| 707 | 3687792 | DCTPP1 | exonic | FALSE |
| 708 | 3695156 | CMTM3 | antisense | FALSE |
| 709 | 3697019 | AARS | exonic | TRUE |
| 710 | 3699648 | CHST5 | exonic | FALSE |
| 711 | 3699716 | chr16−: 75627719-75628026 | intergenic | FALSE |
| 712 | 3701328 | CDYL2 | intronic | FALSE |
| 713 | 3701921 | chr16−: 82441414-82441529 | intergenic | FALSE |
| 714 | 3714621 | AC090774.1 | exonic | FALSE |
| 715 | 3714889 | | nonunique | FALSE |
| 716 | 3717823 | MYO1D | antisense | FALSE |
| 717 | 3720986 | TOP2A | antisense | FALSE |
| 718 | 3720990 | TOP2A | antisense | FALSE |
| 719 | 3720992 | TOP2A | antisense | FALSE |
| 720 | 3722902 | AC003043.2 | exonic | FALSE |
| 721 | 3726287 | COL1A1 | antisense | FALSE |
| 722 | 3732637 | KPNA2 | exonic | TRUE |
| 723 | 3734666 | SLC16A5 | exonic | TRUE |
| 724 | 3734671 | SLC16A5 | intronic | FALSE |
| 725 | 3736308 | BIRC5 | exonic | FALSE |
| 726 | 3737983 | ACTG1 | antisense | FALSE |
| 727 | 3740674 | C17orf91, MIR22 | exonic | FALSE |
| 728 | 3740957 | | nonunique | FALSE |
| 729 | 3741609 | ITGAE | intronic | FALSE |
| 730 | 3748519 | | nonunique | FALSE |
| 731 | 3750786 | SPAG5 | exonic | FALSE |
| 732 | 3751043 | TLCD1 | exonic | FALSE |
| 733 | 3754010 | CCL3 | exonic | FALSE |
| 734 | 3754568 | ACACA | exonic | TRUE |
| 735 | 3755080 | MRPL45 | antisense | FALSE |
| 736 | 3756203 | TOP2A | exonic | TRUE |
| 737 | 3756204 | TOP2A | exonic | TRUE |
| 738 | 3756211 | TOP2A | exonic | TRUE |
| 739 | 3756230 | TOP2A | exonic | TRUE |
| 740 | 3756233 | TOP2A | exonic | TRUE |
| 741 | 3756460 | KRT25 | exonic | TRUE |
| 742 | 3756592 | KRT23 | exonic | FALSE |
| 743 | 3757083 | KRT15 | exonic | FALSE |
| 744 | 3757509 | | nonunique | FALSE |
| 745 | 3758022 | TUBG1 | antisense | FALSE |
| 746 | 3759078 | SLC25A39 | exonic | FALSE |
| 747 | 3759259 | GPATCH8 | exonic | FALSE |
| 748 | 3762200 | COL1A1 | exonic | FALSE |
| 749 | 3762203 | COL1A1 | exonic | FALSE |
| 750 | 3762204 | COL1A1 | exonic | TRUE |
| 751 | 3762207 | COL1A1 | exonic | TRUE |
| 752 | 3762226 | COL1A1 | exonic | TRUE |
| 753 | 3762244 | COL1A1 | exonic | TRUE |
| 754 | 3766365 | DDX42 | antisense | FALSE |
| 755 | 3768105 | PSMD12 | exonic | FALSE |
| 756 | 3769780 | SLC39A11 | exonic | FALSE |
| 757 | 3772191 | BIRC5 | antisense | FALSE |
| 758 | 3778629 | VAPA | exonic | FALSE |
| 759 | 3780241 | C18orf1 | intronic | FALSE |
| 760 | 3780242 | C18orf1 | intronic | FALSE |
| 761 | 3780263 | C18orf1 | intronic | FALSE |
| 762 | 3784894 | FHOD3 | exonic | FALSE |
| 763 | 3786886 | SLC14A1 | exonic | FALSE |
| 764 | 3786890 | SLC14A1 | exonic | TRUE |
| 765 | 3791878 | SERPINB11 | exonic | TRUE |
| 766 | 3791884 | SERPINB11 | exonic | TRUE |
| 767 | 3795981 | YES1 | intronic | FALSE |
| 768 | 3796566 | | nonunique | FALSE |
| 769 | 3797425 | L3MBTL4 | exonic | FALSE |
| 770 | 3797601 | LAMA1 | intronic | FALSE |
| 771 | 3798470 | VAPA | antisense | FALSE |
| 772 | 3803380 | | nonunique | FALSE |
| 773 | 3804030 | INO80C | exonic | TRUE |
| 774 | 3809609 | ONECUT2 | antisense | FALSE |
| 775 | 3816378 | AMH | exonic | FALSE |
| 776 | 3817086 | GIPC3, AC116968.1 | exonic | FALSE |
| 777 | 3831280 | ZNF146 | exonic | FALSE |
| 778 | 3834142 | HNRNPUL1 | exonic | FALSE |
| 779 | 3835890 | APOE | exonic | FALSE |
| 780 | 3835902 | APOC1 | exonic | FALSE |
| 781 | 3836861 | CALM3 | exonic | FALSE |
| 782 | 3837377 | GLTSCR1 | intronic | FALSE |
| 783 | 3842372 | U2AF2 | exonic | FALSE |
| 784 | 3855230 | COMP | exonic | TRUE |
| 785 | 3855231 | COMP | exonic | TRUE |
| 786 | 3857934 | chr19−: 30609054-30609095 | intergenic | FALSE |
| 787 | 3859338 | UBA2 | antisense | FALSE |
| 788 | 3873715 | STK35 | exonic | TRUE |
| 789 | 3876109 | C20orf103 | exonic | FALSE |
| 790 | 3877802 | SNRPB2 | exonic | FALSE |
| 791 | 3878568 | DTD1 | intronic | FALSE |
| 792 | 3880275 | CST8 | exonic | FALSE |
| 793 | 3881492 | TPX2 | exonic | FALSE |
| 794 | 3881493 | TPX2 | exonic | FALSE |
| 795 | 3883508 | ROMO1 | exonic | FALSE |
| 796 | 3883669 | | nonunique | FALSE |
| 797 | 3884904 | FAM83D | exonic | TRUE |
| 798 | 3887068 | UBE2C | exonic | FALSE |
| 799 | 3891257 | GNAS | exonic | TRUE |
| 800 | 3892784 | C20orf166 | antisense | FALSE |
| 801 | 3894317 | AL121758.1, SRXN1 | exonic | FALSE |
| 802 | 3895596 | ADAM33 | exonic | TRUE |
| 803 | 3897434 | MKKS | exonic | FALSE |
| 804 | 3897507 | JAG1 | exonic | FALSE |
| 805 | 3900116 | RALGAPA2 | intronic | FALSE |
| 806 | 3903114 | NECAB3 | exonic | FALSE |
| 807 | 3907455 | UBE2C | antisense | FALSE |
| 808 | 3908040 | SLC13A3 | intronic | FALSE |
| 809 | 3908589 | RP1-66N13.1 | exonic | TRUE |
| 810 | 3909286 | FAM65C | exonic | TRUE |
| 811 | 3910773 | | nonunique | FALSE |
| 812 | 3910788 | AURKA | exonic | FALSE |
| 813 | 3911474 | VAPB | antisense | FALSE |
| 814 | 3911798 | | nonunique | FALSE |
| 815 | 3912525 | CDH4 | antisense | FALSE |
| 816 | 3915239 | C21orf34 | intronic | FALSE |
| 817 | 3917904 | AP000251.2 | exonic | FALSE |
| 818 | 3930414 | RUNX1 | intronic | FALSE |
| 819 | 3931331 | TTC3 | antisense | FALSE |
| 820 | 3936946 | CDC45 | exonic | FALSE |
| 821 | 3945249 | TMEM184B | antisense | FALSE |
| 822 | 3954253 | MAPK1 | intronic | FALSE |
| 823 | 3955487 | TMEM211 | exonic | TRUE |
| 824 | 3958008 | PRR14L | exonic | FALSE |
| 825 | 3959614 | FOXRED2 | exonic | FALSE |
| 826 | 3963890 | RP11-398F12.1 | intronic | FALSE |
| 827 | 3970262 | REPS2 | exonic | TRUE |
| 828 | 3974802 | USP9X | exonic | FALSE |
| 829 | 3975238 | MAOA | exonic | TRUE |
| 830 | 3976556 | RBM3 | exonic | TRUE |
| 831 | 3979980 | AR | exonic | FALSE |
| 832 | 3985031 | TCEAL2 | exonic | FALSE |
| 833 | 3988994 | NDUFA1 | exonic | FALSE |
| 834 | 3989958 | chrX+: 124339283-124339382 | intergenic | FALSE |
| 835 | 3993168 | | nonunique | FALSE |
| 836 | 3995663 | BGN | exonic | FALSE |
| 837 | 3995664 | BGN | exonic | FALSE |
| 838 | 3999161 | GPR143 | exonic | FALSE |
| 839 | 4002408 | chrX−: 21709519-21709613 | intergenic | FALSE |
| 840 | 4004389 | DMD | exonic | TRUE |
| 841 | 4012185 | CITED1 | exonic | TRUE |
| 842 | 4019610 | NDUFA1 | antisense | FALSE |
| 843 | 4019862 | LAMP2 | exonic | FALSE |
| 844 | 4021473 | AIFM1 | exonic | FALSE |
| 845 | 4025833 | chrX−: 150081082-150081152 | intergenic | FALSE |
| 846 | 4030075 | TTTY15 | exonic | FALSE |
| 847 | 4040797 | | nonunique | FALSE |
| 848 | 4042910 | BROX | exonic | TRUE |
| 849 | 4043134 | AIDA | antisense | FALSE |
| 850 | 4044946 | BROX | exonic | FALSE |
| 851 | 4045341 | | nonunique | FALSE |
| 852 | 4050531 | TPRN | exonic | FALSE |
| 853 | 4054706 | HES4 | exonic | FALSE |

What is claimed is:

1. A method of diagnosing, prognosing, determining progression the cancer, or predicting benefit from therapy in a subject, comprising:
(a) assaying an expression level in a sample from the subject for a plurality of targets, wherein the plurality of targets comprises one or more targets selected from Table 1; and
(b) diagnosing, prognosing, determining progression the cancer, or predicting benefit from therapy in a subject based on the expression levels of the plurality of targets.

2. A method of determining a treatment for a cancer in a subject, comprising:
(a) assaying an expression level in a sample from the subject for a plurality of targets, wherein the plurality of targets comprises one or more targets selected from Table 1; and
(b) determining the treatment for the cancer based on the expression level of the plurality of targets.

3. The method of any of claims 1-2, wherein the cancer is selected from the group consisting of a carcinoma, sarcoma, leukemia, lymphoma, myeloma, and a CNS tumor.

4. The method of any of claims 1-2, wherein the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

5. The method of any of claims 1-2, wherein the cancer is a prostate cancer.

6. The method of any of claims 1-2, wherein the cancer is a pancreatic cancer.

7. The method of any of claims 1-2, wherein the cancer is a thyroid cancer.

8. The method of any of claims 1-2, wherein the plurality of targets comprises a coding target.

9. The method of claim 8, wherein the coding target is an exonic sequence.

10. The method of any of claims 1-2, wherein the plurality of targets comprises a non-coding target.

11. The method of claim 10, wherein the non-coding target comprises an intronic sequence or partially overlaps an intronic sequence.

12. The method of claim 10, wherein the non-coding target comprises a sequence within the UTR or partially overlaps with a UTR sequence.

13. The method of any of claims 1-2, wherein the target comprises a nucleic acid sequence.

14. The method of claim 13, wherein the nucleic acid sequence is a DNA sequence.

15. The method of claim 13, wherein the nucleic acid sequence is an RNA sequence.

16. The method of any of claims 1-2, wherein the plurality of targets comprises at least 5 targets selected from Table 1.

17. The method of any of claims 1-2, wherein the plurality of targets comprises at least 10 targets selected from Table 1.

18. The method of any of claims 1-2, wherein the plurality of targets comprises at least 15 targets selected from Table 1.

19. The method of any of claims 1-2, wherein the plurality of targets comprises at least 20 targets selected from Table 1.

20. The method of claim 1, wherein the diagnosing, prognosing, determining progression the cancer, or predicting benefit from therapy includes determining the malignancy of the cancer.

21. The method of claim 1, wherein the diagnosing, prognosing, determining progression the cancer, or predicting benefit from therapy includes determining the stage of the cancer.

22. The method of claim 1, wherein the diagnosing, prognosing, determining progression the cancer, or predicting benefit from therapy includes assessing the risk of cancer recurrence.

23. The method of claim 2, wherein determining the treatment for the cancer includes determining the efficacy of treatment.

24. The method of any of claims 1-2, further comprising sequencing the plurality of targets.

25. The method of any of claims 1-2, further comprising hybridizing the plurality of targets to a solid support.

26. The method of claim 25, wherein the solid support is a bead or array.

27. A probe set for assessing a cancer status of a subject comprising a plurality of probes, wherein the probes in the set are capable of detecting an expression level of one or more targets selected from Table 1, wherein the expression level determines the cancer status of the subject with at least 40% specificity.

28. The probe set of claim 27, wherein the cancer is selected from the group consisting of a carcinoma, sarcoma, leukemia, lymphoma, myeloma, and a CNS tumor.

29. The probe set of claim 27, wherein the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

30. The probe set of claim 27, wherein the cancer is a prostate cancer.

31. The probe set of claim 27, wherein the cancer is a pancreatic cancer.

32. The probe set of claim 27, wherein the cancer is a thyroid cancer.

33. The probe set of claim 27, wherein the probe set further comprises a probe capable of detecting an expression level of at least one coding target.

34. The probe set of claim 33, wherein the coding target is an exonic sequence.

35. The probe set of claim 27, wherein the probe set further comprises a probe capable of detecting an expression level of at least one non-coding target.

36. The probe set of claim 35, wherein the non-coding target is an intronic sequence or partially overlaps with an intronic sequence.

37. The probe set of claim 35, wherein the non-coding target is a UTR sequence or partially overlaps with a UTR sequence.

38. The probe set of claim 27, wherein assessing the cancer status includes assessing cancer recurrence risk.

39. The probe set of claim 27, wherein assessing the cancer status includes determining a treatment modality.

40. The probe set of claim 27, wherein assessing the cancer status includes determining the efficacy of treatment.

41. The probe set of claim 27, wherein the target is a nucleic acid sequence.

42. The probe set of claim 41, wherein the nucleic acid sequence is a DNA sequence.

43. The probe set of claim 41, wherein the nucleic acid sequence is an RNA sequence.

44. The probe set of claim 27, wherein the probes are between about 15 nucleotides and about 500 nucleotides in length.

45. The probe set of claim 27, wherein the probes are between about 15 nucleotides and about 450 nucleotides in length.

46. The probe set of claim 27, wherein the probes are between about 15 nucleotides and about 400 nucleotides in length.

47. The probe set of claim 27, wherein the probes are between about 15 nucleotides and about 350 nucleotides in length.

48. The probe set of claim 27, wherein the probes are between about 15 nucleotides and about 300 nucleotides in length.

49. The probe set of claim 27, wherein the probes are between about 15 nucleotides and about 250 nucleotides in length.

50. The probe set of claim 27, wherein the probes are between about 15 nucleotides and about 200 nucleotides in length.

51. The probe set of claim 27, wherein the probes are at least 15 nucleotides in length.

52. The probe set of claim 27, wherein the probes are at least 25 nucleotides in length.

53. The probe set of claim 27, wherein the expression level determines the cancer status of the subject with at least 50% specificity.

54. The probe set of claim 27, wherein the expression level determines the cancer status of the subject with at least 60% specificity.

55. The probe set of claim 27, wherein the expression level determines the cancer status of the subject with at least 65% specificity.

56. The probe set of claim 27, wherein the expression level determines the cancer status of the subject with at least 70% specificity.

57. The probe set of claim 27, wherein the expression level determines the cancer status of the subject with at least 75% specificity.

58. The probe set of claim 27, wherein the expression level determines the cancer status of the subject with at least 80% specificity.

59. The probe set of claim 27, wherein the expression level determines the cancer status of the subject with at least 85% specificity.

60. The probe set of claim 27, wherein the non-coding target is a non-coding RNA transcript and the non-coding RNA transcript is non-polyadenylated.

61. A system for analyzing a cancer, comprising:
(a) a probe set comprising a plurality of target sequences, wherein
(i) the plurality of target sequences hybridizes to one or more targets selected from Table 1; or
(ii) the plurality of target sequences comprises one or more target sequences selected from Table 1; and
(b) a computer model or algorithm for analyzing an expression level and/or expression profile of the target hybridized to the probe in a sample from a subject suffering from a cancer.

62. The system of claim 61, further comprising an electronic memory for capturing and storing an expression profile.

63. The system of claim 61 or claim 62, further comprising a computer-processing device, optionally connected to a computer network.

64. The system of claim 63, further comprising a software module executed by the computer-processing device to analyze an expression profile.

65. The system of claim 63, further comprising a software module executed by the computer-processing device to compare the expression profile to a standard or control.

66. The system of claim 63, further comprising a software module executed by the computer-processing device to determine the expression level of the target.

67. The system of any claims 61-66, further comprising a machine to isolate the target or the probe from the sample.

68. The system of any claims 61-67, further comprising a machine to sequence the target or the probe.

69. The system of any claims 61-68, further comprising a machine to amplify the target or the probe.

70. The system of any claims 61-69, further comprising a label that specifically binds to the target, the probe, or a combination thereof.

71. The system of claim 63, further comprising a software module executed by the computer-processing device to transmit an analysis of the expression profile to the individual or a medical professional treating the individual.

72. The system of any claims 61-71, further comprising a software module executed by the computer-processing device to transmit a diagnosis or prognosis to the individual or a medical professional treating the individual.

73. The system of any claims 61-72, wherein the plurality of target sequences comprises at least 5 target sequences selected from Table 1.

74. The system of any claims 61-72, wherein the plurality of target sequences comprises at least 10 target sequences selected from Table 1.

75. The system of any claims 61-72, wherein the plurality of target sequences comprises at least 15 target sequences selected from Table 1.

76. The system of any claims 61-72, wherein the plurality of target sequences comprises at least 20 target sequences selected from Table 1.

77. The system of any claims 61-76, wherein the cancer is selected from the group consisting of a carcinoma, sarcoma, leukemia, lymphoma, myeloma, and a CNS tumor.

78. The system of any claims 61-76, wherein the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

79. A method of analyzing a cancer in an individual in need thereof, comprising:
(a) obtaining an expression profile from a sample obtained from the individual, wherein the expression profile comprises one or more targets selected from Table 1; and
(b) comparing the expression profile from the sample to an expression profile of a control or standard.

80. The method of claim 79, wherein the plurality of targets comprises at least 5 targets selected from Table 1.

81. The method of claim 79, wherein the plurality of targets comprises at least 10 targets selected from Table 1.

82. The method of claim 79, wherein the plurality of targets comprises at least 15 targets selected from Table 1.

83. The method of claim 79, wherein the plurality of targets comprises at least 20 targets selected from Table 1.

84. The method of any of claims 79-83, wherein the cancer is selected from the group consisting of a carcinoma, sarcoma, leukemia, lymphoma, myeloma, and a CNS tumor.

85. The method of any of claims 79-83, wherein the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

86. The method of any of claims 79-85, further comprising a software module executed by a computer-processing device to compare the expression profiles.

87. The method of any of claims 79-86, further comprising providing diagnostic or prognostic information to the individual about the cardiovascular disorder based on the comparison.

88. The method of any of claims 79-87, further comprising diagnosing the individual with a cancer if the expression profile of the sample (a) deviates from the control or standard from a healthy individual or population of healthy individuals, or (b) matches the control or standard from an individual or population of individuals who have or have had the cancer.

89. The method of any of claims 79-88, further comprising predicting the susceptibility of the individual for developing a cancer based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

90. The method of any of claims 79-89, further comprising prescribing a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

91. The method of any of claims 79-90, further comprising altering a treatment regimen prescribed or administered to the individual based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

92. The method of any of claims 79-91, further comprising predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

93. The method of any of claims 89-92, wherein the deviation is the expression level of one or more targets from the sample is greater than the expression level of one or more targets from a control or standard derived from a healthy individual or population of healthy individuals.

94. The method of any of claims 89-92, wherein the deviation is the expression level of one or more targets from the sample is at least about 30% greater than the expression level of one or more targets from a control or standard derived from a healthy individual or population of healthy individuals.

95. The method of any of claims 89-90, wherein the deviation is the expression level of one or more targets from the sample is less than the expression level of one or more targets from a control or standard derived from a healthy individual or population of healthy individuals.

96. The method of any of claims 89-92, wherein the deviation is the expression level of one or more targets from the sample is at least about 30% less than the expression level of one or more targets from a control or standard derived from a healthy individual or population of healthy individuals.

97. The method of any of claims 79-96, further comprising using a machine to isolate the target or the probe from the sample.

98. The method of any of claims 79-97, further comprising contacting the sample with a label that specifically binds to the target, the probe, or a combination thereof.

99. The method of any of claims 79-98, further comprising contacting the sample with a label that specifically binds to a target selected from Table 1.

100. The method of any of claims 79-99, further comprising amplifying the target, the probe, or any combination thereof.

101. The method of any of claims 79-100, further comprising sequencing the target, the probe, or any combination thereof.

102. A method of diagnosing cancer in an individual in need thereof, comprising:
(a) obtaining an expression profile from a sample obtained from the individual, wherein the expression profile comprises one or more targets selected from Table 1;
(b) comparing the expression profile from the sample to an expression profile of a control or standard; and
(c) diagnosing a cancer in the individual if the expression profile of the sample (i) deviates from the control or standard from a healthy individual or population of healthy individuals, or (ii) matches the control or standard from an individual or population of individuals who have or have had the cancer.

103. The method of claim 102, wherein the plurality of targets comprises at least 5 targets selected from Table 1.

104. The method of claim 102, wherein the plurality of targets comprises at least 10 targets selected from Table 1.

105. The method of claim 102, wherein the plurality of targets comprises at least 15 targets selected from Table 1.

106. The method of claim 102, wherein the plurality of targets comprises at least 20 targets selected from Table 1.

107. The method of any of claims 102-106, wherein the cancer is selected from the group consisting of a carcinoma, sarcoma, leukemia, lymphoma, myeloma, and a CNS tumor.

108. The method of any of claims 102-106, wherein the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

109. The method of any of claims 102-108, further comprising a software module executed by a computer-processing device to compare the expression profiles.

110. The method of any of claims 102-109, wherein the deviation is the expression level of one or more targets from the sample is greater than the expression level of one or more targets from a control or standard derived from a healthy individual or population of healthy individuals.

111. The method of any of claims 102-109, wherein the deviation is the expression level of one or more targets from the sample is at least about 30% greater than the expression level of one or more targets from a control or standard derived from a healthy individual or population of healthy individuals.

112. The method of any of claims 102-109, wherein the deviation is the expression level of one or more targets from the sample is less than the expression level of one or more targets from a control or standard derived from a healthy individual or population of healthy individuals.

113. The method of any of claims 102-109, wherein the deviation is the expression level of one or more targets from the sample is at least about 30% less than the expression level of one or more targets from a control or standard derived from a healthy individual or population of healthy individuals.

114. The method of any of claims 102-113, further comprising using a machine to isolate the target or the probe from the sample.

115. The method of any of claims 102-114, further comprising contacting the sample with a label that specifically binds to the target, the probe, or a combination thereof.

116. The method of any of claims 102-115, further comprising contacting the sample with a label that specifically binds to a target selected from Table 1.

117. The method of any of claims 102-116, further comprising amplifying the target, the probe, or any combination thereof.

118. The method of any of claims 102-117, further comprising sequencing the target, the probe, or any combination thereof.

119. A method of predicting whether an individual is susceptible to developing a cancer, comprising:
(a) obtaining an expression profile from a sample obtained from the individual, wherein the expression profile comprises one or more targets selected from Table 1;
(b) comparing the expression profile from the sample to an expression profile of a control or standard; and
(c) predicting the susceptibility of the individual for developing a cancer based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer.

120. The method of claim 119, wherein the plurality of targets comprises at least 5 targets selected from Table 1.

121. The method of claim 119, wherein the plurality of targets comprises at least 10 targets selected from Table 1.

122. The method of claim 119, wherein the plurality of targets comprises at least 15 targets selected from Table 1.

123. The method of claim 119, wherein the plurality of targets comprises at least 20 targets selected from Table 1.

124. The method of any of claims 119-123, wherein the cancer is selected from the group consisting of a carcinoma, sarcoma, leukemia, lymphoma, myeloma, and a CNS tumor.

125. The method of any of claims 119-123, wherein the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

126. The method of any of claims 119-125, further comprising a software module executed by a computer-processing device to compare the expression profiles.

127. The method of any of claims 119-126, wherein the deviation is the expression level of one or more targets from the sample is greater than the expression level of one or more targets from a control or standard derived from a healthy individual or population of healthy individuals.

128. The method of any of claims 119-126, wherein the deviation is the expression level of one or more targets from the sample is at least about 30% greater than the expression level of one or more targets from a control or standard derived from a healthy individual or population of healthy individuals.

129. The method of any of claims 119-126, wherein the deviation is the expression level of one or more targets from the sample is less than the expression level of one or more targets from a control or standard derived from a healthy individual or population of healthy individuals.

130. The method of any of claims 119-126, wherein the deviation is the expression level of one or more targets from the sample is at least about 30% less than the expression level of one or more targets from a control or standard derived from a healthy individual or population of healthy individuals.

131. The method of any of claims 119-130, further comprising using a machine to isolate the target or the probe from the sample.

132. The method of any of claims 119-131, further comprising contacting the sample with a label that specifically binds to the target, the probe, or a combination thereof.

133. The method of any of claims 119-132, further comprising contacting the sample with a label that specifically binds to a target selected from Table 1.

134. The method of any of claims 119-133, further comprising amplifying the target, the probe, or any combination thereof.

135. The method of any of claims 119-134, further comprising sequencing the target, the probe, or any combination thereof.

136. A method of predicting an individual's response to a treatment regimen for a cancer, comprising:
(a) obtaining an expression profile from a sample obtained from the individual, wherein the expression profile comprises one or more targets selected from Table 1;
(b) comparing the expression profile from the sample to an expression profile of a control or standard; and
(c) predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

137. The method of claim 136, wherein the plurality of targets comprises at least 5 targets selected from Table 1.

138. The method of claim 136, wherein the plurality of targets comprises at least 10 targets selected from Table 1.

139. The method of claim 136, wherein the plurality of targets comprises at least 15 targets selected from Table 1.

140. The method of claim 136, wherein the plurality of targets comprises at least 20 targets selected from Table 1.

141. The method of any of claims 136-140, wherein the cancer is selected from the group consisting of a carcinoma, sarcoma, leukemia, lymphoma, myeloma, and a CNS tumor.

142. The method of any of claims 136-140, wherein the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

143. The method of any of claims 136-142, further comprising a software module executed by a computer-processing device to compare the expression profiles.

144. The method of any of claims 136-143, wherein the deviation is the expression level of one or more targets from the sample is greater than the expression level of one or more targets from a control or standard derived from a healthy individual or population of healthy individuals.

145. The method of any of claims 136-143, wherein the deviation is the expression level of one or more targets from the sample is at least about 30% greater than the expression level of one or more targets from a control or standard derived from a healthy individual or population of healthy individuals.

146. The method of any of claims 136-143, wherein the deviation is the expression level of one or more targets from the sample is less than the expression level of one or more targets from a control or standard derived from a healthy individual or population of healthy individuals.

147. The method of any of claims 136-143, wherein the deviation is the expression level of one or more targets from the sample is at least about 30% less than the expression level of one or more targets from a control or standard derived from a healthy individual or population of healthy individuals.

148. The method of any of claims 136-147, further comprising using a machine to isolate the target or the probe from the sample.

149. The method of any of claims 136-148, further comprising contacting the sample with a label that specifically binds to the target, the probe, or a combination thereof.

150. The method of any of claims 136-149, further comprising contacting the sample with a label that specifically binds to a target selected from Table 1.

151. The method of any of claims 136-150, further comprising amplifying the target, the probe, or any combination thereof.

152. The method of any of claims 136-151, further comprising sequencing the target, the probe, or any combination thereof.

153. A method of prescribing a treatment regimen for a cancer to an individual in need thereof, comprising:
(a) obtaining an expression profile from a sample obtained from the individual, wherein the expression profile comprises one or more targets selected from Table 1;
(b) comparing the expression profile from the sample to an expression profile of a control or standard; and
(c) prescribing a treatment regimen based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer.

154. The method of claim 153, wherein the plurality of targets comprises at least 5 targets selected from Table 1.

155. The method of claim 153, wherein the plurality of targets comprises at least 10 targets selected from Table 1.

156. The method of claim 153, wherein the plurality of targets comprises at least 15 targets selected from Table 1.

157. The method of claim 153, wherein the plurality of targets comprises at least 20 targets selected from Table 1.

158. The method of any of claims 153-157, wherein the cancer is selected from the group consisting of a carcinoma, sarcoma, leukemia, lymphoma, myeloma, and a CNS tumor.

159. The method of any of claims 153-157, wherein the cancer is selected from the group consisting of skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

160. The method of any of claims 153-159, further comprising a software module executed by a computer-processing device to compare the expression profiles.

161. The method of any of claims 153-160, wherein the deviation is the expression level of one or more targets from the sample is greater than the expression level of one or more targets from a control or standard derived from a healthy individual or population of healthy individuals.

162. The method of any of claims 153-160, wherein the deviation is the expression level of one or more targets from the sample is at least about 30% greater than the expression level of one or more targets from a control or standard derived from a healthy individual or population of healthy individuals.

163. The method of any of claims 153-160, wherein the deviation is the expression level of one or more targets from the sample is less than the expression level of one or more targets from a control or standard derived from a healthy individual or population of healthy individuals.

164. The method of any of claims 153-160, wherein the deviation is the expression level of one or more targets from the sample is at least about 30% less than the expression level of one or more targets from a control or standard derived from a healthy individual or population of healthy individuals.

165. The method of any of claims 153-164, further comprising using a machine to isolate the target or the probe from the sample.

166. The method of any of claims 153-165, further comprising contacting the sample with a label that specifically binds to the target, the probe, or a combination thereof.

167. The method of any of claims 153-166, further comprising contacting the sample with a label that specifically binds to a target selected from Table 1.

168. The method of any of claims 153-167, further comprising amplifying the target, the probe, or any combination thereof.

169. The method of any of claims 153-168, further comprising sequencing the target, the probe, or any combination thereof.

170. The method of claim 153-169, further comprising converting the expression levels of the target sequences into a likelihood score that indicates the probability that a biological sample is from a patient who will exhibit no evidence of disease, who will exhibit systemic cancer, or who will exhibit biochemical recurrence.

171. The method of claim 153-170, wherein the target sequences are differentially expressed the cancer.

172. The method of claim 171, wherein the differential expression is dependent on aggressiveness.

173. The method of claim 153-172, wherein the expression profile is determined by a method selected from the group consisting of RT-PCR, Northern blotting, ligase chain reaction, array hybridization, and a combination thereof.

174. A kit for analyzing a cancer, comprising:
(a) a probe set comprising a plurality of target sequences, wherein the plurality of target sequences comprises at least one target sequence listed in Table 1; and
(b) a computer model or algorithm for analyzing an expression level and/or expression profile of the target sequences in a sample.

175. The kit of claim 174, further comprising a computer model or algorithm for correlating the expression level or expression profile with disease state or outcome.

176. The kit of claim 174, further comprising a computer model or algorithm for designating a treatment modality for the individual.

177. The kit of claim 174, further comprising a computer model or algorithm for normalizing expression level or expression profile of the target sequences.

178. The kit of claim 174, further comprising a computer model or algorithm comprising a robust multichip average (RMA), probe logarithmic intensity error estimation (PLIER), non-linear fit (NLFIT) quantile-based, nonlinear normalization, or a combination thereof.

179. The kit of claim 174, wherein the cancer is a prostate cancer.

180. The kit of claim 174, wherein the cancer is a lung cancer.

181. The kit of claim 174, wherein the cancer is a breast cancer.

182. The kit of claim 174, wherein the cancer is a thyroid cancer.

183. The kit of claim 174, wherein the cancer is a colon cancer.

184. The kit of claim 174, wherein the cancer is a pancreatic cancer.


SEQUENCE LISTING (853 sequences)

SEQ ID NO: 1 - DNA, 45 bp, Homo sapiens
tctagaagaaactggcgcttaaaccaaatcgcatggatttctttt

SEQ ID NO: 2 - DNA, 95 bp, Homo sapiens
ctgggtctgccggtgatagccacgaggaggactcgaaagccatggcctctgttctgtgggacgcgggaactcttggagcccgtccgtggctgcct

SEQ ID NO: 3 - DNA, 34 bp, Homo sapiens
caggccggaaaattgtgctctagaagaataaaac

SEQ ID NO: 4 - DNA, 164 bp, Homo sapiens
agactgaaacaggaaaggccataaaatatctggttcatatcacctgttggacatttccttttggattcatgctttctggaaggtttaaattcattaacgttaatagttaattataactttttttttaacttaagaggattcagggttaagcaccaactaaatta

SEQ ID NO: 5 - DNA, 31 bp, Homo sapiens
agacattaaacaaagctagccatcatctcaa

SEQ ID NO: 6 - DNA, 1102 bp, Homo sapiens
cagctggtgtgccttaagagaatccctataaataacagaaaagacactccaagcattcctgtacgtggactcagagcacagagaaaagaaactaaaatgccttttggcatttcaagatatttggcactcttgtgattacatttttttacagtccattaaagagaataaactgacataatattagagaaataaacaggctgctcacacaacagactgcaaggggaagttagaaaaagctcaagcatttttttctttgtttttcgtgtgtgtgtgtgtgtgtgtgtgtgtgtgtgtgtgtttttctgacataaaaaatgtgtccatttgcattaacttgggcagatagcttgcagcaacaaagaaacacaagctttacaactcattttaaaataaaatcttttctatgtatcattccttagaaaagttctcttcttgttttaaacacattcctgataacttctaaagatgaccaaaataaaacagaatatctacagagatcattttctgaattttttgtacatccaaggataacaacataaaaaaaataaaactggacagcatttcacatccaagtgcacagaaccatttttgcaagattaaataatgtaaacattgggaacagccaaatcagcgaagaatgccaacacctcaaaacacctggtgttgccgcttcattaagtggttcaaaatccagatctataattgcgcaatattcaccgtatataaaaagaaatggatattaattttgacaaatagctgcaactgagacttctttttatttctttatatgtgtatatagtgaatttttattatttttaaaattttatttatttttttatttttatttttgcagaggagcccagagccttctcctcctcctcctcctcctctcgcctcatctgtctcccggcctgataccagatacaggttgttgatttcatcgtgggtagcaagctagtaataaatttcaaagtgctttctcttttcatgctttttgccaataactgttaccgccgttcttattctctcccttaactcattgtctttgggggagttagacaccaggaggtgccttgtcggtcatatttttcagcacgtcatc

SEQ ID NO: 7 - DNA, 91 bp, Homo sapiens
ccctgggtagacagacacagcttgatttcagagcagacataggcgaagaaaacatggcattgagtgtgctgagtccagacaaatgttattt

SEQ ID NO: 8 - DNA, 77 bp, Homo sapiens
tacttcatcaattacgtcctcttcatattcatcaatttcttccccatcatactcatcttcgcttcccacatcacttc

SEQ ID NO: 9 - DNA, 27 bp, Homo sapiens
agatattccagatgagaggtggtgggc

SEQ ID NO: 10 - DNA, 247 bp, Homo sapiens
ggcattaccatagacgactcctagaggacagtgctatgtaaaaatgtgtgtctataaatgtttatcatgcatgtattctagagctcattcatttattcaacaaacatttggtgagcacctatttcgttcgagaaacttcatttatctcctataattggcaaacttaaaaatgcagcagaaacttacattccaaccttagagactcatagtgagcacaaggaaagttttgccctgagattcatggtta

SEQ ID NO: 11 - DNA, 371 bp, Homo sapiens
ctcctcacggtgaaattgttctttatgatactctgtatctactggtagttcacatccttcacagaatcctctaactatgcaaaacacatagtgaacagttatcatttgaatcttagtacaactttaataagctgtgttttatatttaagtagtttctcccctggtggttgcccgtggcagactttggagtttgatagctcttcactccctattttatacatctctaagtaattataccctgtgcccttaagcctcaaaagcataaaattatttaaaaacatacaattaaacacttcagatgacagtgttaaaacttcagaagaagacactcactttggaagaatggtgcttcgtgctggagcctttatatc

SEQ ID NO: 12 - DNA, 50 bp, Homo sapiens
attggcagtttcgaaaggcacagaagagacgaccatgaaaccgatttaca

SEQ ID NO: 13 - DNA, 78 bp, Homo sapiens
aataatatgtccaaggccacgcccaggcgtcagggattcatgaattttgtatacagtgtatgatgtttctagattaaa

SEQ ID NO: 14 - DNA, 378 bp, Homo sapiens
agtgcatacgcacaacacttgagctcactgcatagcaacaggaggtggctgtgaaattatttcagtaacatactgtgtattacagttcattttatgcagctatgatttaatactgcatctttacattattttacatttctcccaaaactgtgaattgcaccatgtatgatctgtatttgtgtgtgttaatgttgataatagatttgcgtgtattttatggcagtaaatgataaaatagtctagtatctacatatattttgtgcattcatgatataactacttttttcctaatttttttcatctgaaacttttctcattgtttcagatctccaaaaatttttccaatatatttattgaaaatatctgggctgcgtgcag

SEQ ID NO: 15 - DNA, 53 bp, Homo sapiens
caaatgacttgcttccattgtttgtttgttcaattgtctgtttgttaaataaa

SEQ ID NO: 16 - DNA, 25 bp, Homo sapiens
ggctggtaacctacactttcttcta

SEQ ID NO: 17 - DNA, 69 bp, Homo sapiens
ttcaaatcgccactgtcaccgaaaaaggtgttgaaatagagggaccattgtctacagagaccaactggg

SEQ ID NO: 18 - DNA, 554 bp, Homo sapiens
ataaagtcagaccagtgcattgggcttgttaggggaaaccattccttcctactgtcacatagcctggcagattatatcaggtgccaaaacattcccttttattcaatttattaatgagagtgtggaattaaagtagatccttgcatgtcaggaaagctgcaacacatctggtttaagtggagcacaaaggtaagttattccacattcaccaacttccccacttgacctcccaccagttcctgtctgctttttccatcaatcaactctaagatgccctaaacccaaacagttcccattcaactgtgcacaaaggtatagatatggaagacatctcactccaactctcacctttggaattaaataaacctggaacagggaaggtgaactatggctgaaaccaagtgaagatttttaagaagctttgacagaaacattaagaataacacctgaatgcaaatccagaaaaaagcgctactgggggaaaatagtcttagccaatgttctaaaaatgctcataaggaagggttggggaattaccctttagacacaagctc

SEQ ID NO: 19 - DNA, 354 bp, Homo sapiens
gccatgtggtcatactctcagcttgctgagtggatgacaaaaagaggggaattgttaaaggaaaatttaaatggagactggaaaaatcctgagcaaacaaaaccacctggcccttagaaatagctttaactttgcttaaactacaaacacaagcaaaacttcacggggtcatactacatacaagcataagcaaaacttaacttggatcatttctggtaaatgcttatgttagaaataagacaaccccagccaatcacaagcagcctactaacatataattaggtgactagggactttctaagaagatacctacccccaaaaaacaattatgtaattgaaaaccaaccgattgcc

SEQ ID NO: 20 - DNA, 112 bp, Homo sapiens
ttttttctcgccacatagcacttctttcttgcctctttcatttctgctcctggtgttgcctgcctcctgcaagacccagatgaagaaaccttttcaatggtcgagatctgag

SEQ ID NO: 21 - DNA, 204 bp, Homo sapiens
tgtatctgaatgttgtgtatgtggctgatatggaagacatacatgtatgcaatccatcagcgtttaaagaagaagattggctccagttcggaggaggaggaggaagattacagatctattctgagtattttttagagagttaatatttatatttttagtaattttctggtagaaggaaattgcacaataaaatgatttggtttg

SEQ ID NO: 22 - DNA, 111 bp, Homo sapiens
ctgatgtttccagtaagggaatattggtgagctgcatatataaatttgacagatagctatttacatagccttctaagtaaaggcaatgaattctccatttcctactggagg

SEQ ID NO: 23 - DNA, 285 bp, Homo sapiens
gccagacatatgtggtagcagatccccaggtctcctcctcttccccattccactgaacaactgttcttcctacctatacctccaccttagtataagactccaagtatactctttggagggaaaaaaaaaagcccaagtatctttggattgggagatactaggcaaaattgagaacagggcattttaccacatatgagggaattaggtgaatatgtgcacgctgaatgatgggactcctccacctttcttctctcacttgcctttaccctacaggcaggagattag

SEQ ID NO: 24 - DNA, 33 bp, Homo sapiens
atggcgttcctgttacctctcatcattgtgtta

SEQ ID NO: 25 - DNA, 139 bp, Homo sapiens
accagaatttgatggatcgcctacagagagtgaataacactctgtccagccaaattagccgtttacagaatatccggaataccattgaagagactggaaacttggctgaacaagcgcgtgcccatgtagagaacacaga

SEQ ID NO: 26 - DNA, 154 bp, Homo sapiens
ccattgctactattgcttgtcggtgttattttattttattgtttttgactttggaagagatgaactgtgtatttaacttaagctattgctcttaaaaccagggagtcagaatatatttgtaagttaaatcattggtgctaataataaatgtgga

SEQ ID NO: 27 - DNA, 68 bp, Homo sapiens
aacccacaaacgcttgcgatttggcctccttgttttattttgtgtagtcctacaacgtcttgttacta

SEQ ID NO: 28 - DNA, 365 bp, Homo sapiens
catggttaatgggacctgaatgcacatttatagcataaaagaatgtcaattctatttcataaaggaaaaatctcaactctttgtgactgagtttcacattaactggaactttatttgcttaaaacctaaacattgtcagtttgaaaagaaatccactgtgacctgtagactgatcttgttgattaaattctagggtttttttttttttggattcttggtaaaattttatccaaaaaacaggatacatatatatttagagaaggaaatatgaaatcaagagttttggcagcccctgctttttttttttttttagctccctaaagactgtagcaggataaaaggatcactggctccgagtctctttg

SEQ ID NO: 29 - DNA, 135 bp, Homo sapiens
ctcacccaatctttcctggtcctcaccctgcccctctcagtgtctgaccttcaacagtttgagaaacattgtgatttgtattttcagtgtgggaatgcatttcaagccttagaacccttggcgtgttgctggaag

SEQ ID NO: 30 - DNA, 292 bp, Homo sapiens
cctcctgacagtactggctagaagtttggatggattatttacaatataggaaagaaagccaagatttaggtaatgagtggatgagtaaatggtggaggatgggagtcaaaatcagaattatagaagaagtatttcctgtaactatagaaagaattatgtatatatacatgcagaaatatatatgtgtgtgtgtatctgtggatggatatatgtatatctcttcctatatatatccatagtggacttattcagaacatagatatgtattcagcttgtcttcaaatacggccaa

SEQ ID NO: 31 - DNA, 115 bp, Homo sapiens
tctggaagttatcaataccgtggcacaggtcacttttgacattttaatttattactttttgggaattaaatccttagtctacatgttggcagcatctttacttggcctgggtttg

SEQ ID NO: 32 - DNA, 134 bp, Homo sapiens
tggccaggctagtattttgtcagtccaagcagttcattaaaaaaaaaaaaaacaaaaagagcaagaatataaatactgcatcttccagcctacttttacaaagggttcactcttgggtccttaagcttagtggt

SEQ ID NO: 33 - DNA, 171 bp, Homo sapiens
ctatataatatggtaacttgggtaccgggggaactttaaaatttcatctcaaaaataatttttaaaaagcctgaggtatgatatagcataaaagattgagatgaaaatatatttccctgtaagctgaattactcatttaaaaattttaacttctatatgggacccgaatta

SEQ ID NO: 34 - DNA, 34 bp, Homo sapiens
ctgctgaatcctgtacagccttactcataaataa

SEQ ID NO: 35 - DNA, 134 bp, Homo sapiens
gaaacaactccttttactgcgtagaacctatatcgagagtgtgtgtatatgtattataggaggagctctcaattttatgtattctttctgcctttaattttcttgtttgtttgagcttagggatgagatactta

SEQ ID NO: 36 - DNA, 68 bp, Homo sapiens
agaaacccaccgtagattcgacagccctgggccttcacttcaagcctctgtttagacttccctcttgc

SEQ ID NO: 37 - DNA, 129 bp, Homo sapiens
aacaagttaggtggtagtagtggaggtagtgaaggaaaacagggaactttcctgttcgctgcagagcaaaccaggtgccacgtagaccttctcagatgccactactggacgcagccctctagacctgaa

SEQ ID NO: 38 - DNA, 65 bp, Homo sapiens
tggagcatgggagacgtccctgcctcttgcattttaacgtgaaaaggcaagcttgcgcccattag

SEQ ID NO: 39 - DNA, 126 bp, Homo sapiens
atgctgcagtttcccgctggctttctcggagccaccttgcgggcagatatccgagaatcagagttcattctcttcatcttgcccatctcttacctctgctggcgagttcttcgttgcatcccagtc

SEQ ID NO: 40 - DNA, 25 bp, Homo sapiens
tggtgcttaaccctgaatcctctta

SEQ ID NO: 41 - DNA, 700 bp, Homo sapiens
tgttggcattcttcgctgatttggctgttcccaatgtttacattatttaatcttgcaaaaatggttctgtgcacttggatgtgaaatgctgtccagttttattttttttatgttgttatccttggatgtacaaaaaattcagaaaatgatctctgtagatattctgttttattttggtcatctttagaagttatcaggaatgtgtttaaaacaagaagagaacttttctaaggaatgatacatagaaaagattttattttaaaatgagttgtaaagcttgtgtttctttgttgctgcaagctatctgcccaagttaatgcaaatggacacattttttatgtcagaaaaacacacacacacacacacacacacacacacacacacgaaaaacaaagaaaaaaatgcttgagctttttctaacttccccttgcagtctgttgtgtgagcagcctgtttatttctctaatattatgtcagtttattctctttaatggactgtaaaaaaatgtaatcacaagagtgccaaatatcttgaaatgccaaaaggcattttagtttcttttctctgtgctctgagtccacgtacaggaatgcttggagtgtcttttctgttatttatagggattctcttaaggcacaccagctgcctgttttgcatggtatttgcaaaaatgcctcttgcgtgaggaaatcttttacc

SEQ ID NO: 42 - DNA, 63 bp, Homo sapiens
ttgttattgaagatgataggattgatgacgtgctgaaaaatatgaccgacaaggcacctcctg

SEQ ID NO: 43 - DNA, 55 bp, Homo sapiens
gtcccaatcttaccagatgctactggacttgaatggttaataaaactgcacagtg

SEQ ID NO: 44 - DNA, 118 bp, Homo sapiens
tattccagctatacggggcaaaagatgttatggcagggaatagagaagtttaaatacggatgaaataaagggtcaccatctcctcaggcacaaggaacagcttgcttttcgccagatt

SEQ ID NO: 45 - DNA, 118 bp, Homo sapiens
ctcaaggctcacctagatgggtacaataaaaagaacatgggctttcagcagcagacaaatcccacttccaccactgactagctgtgtgaccttggacaagtgacctaatttttctgag

SEQ ID NO: 46 - DNA, 933 bp, Homo sapiens
tcagcagagctttttggtgatctcctacctgcaccctcaactcttgacaaagaagcaagactatagattcattttctgaaggggatcatgtatggaattttttgatgagtttttacttttacctctctactcttgattttctattattgaatactcttttaaaacactgatttttaaggctttatatatgttttccaggctgatgttcacatctttttttcatgaactatcagaatatagtgaacacttttcaaatatttaaggacttaatgtttaaaaagccataaaatagagagtggtaatactaccaaataattacttaaaactgaaagctaagttatcaatagtttatataagagatgttttctgaggagatgtgcatccagtgagaccaaggtagaaagtttatataattgttttttttccagtaaatatgaaaaaaaaagctgtagcttgtttattacatgtccaaaatacagtggagccttactttaacacaatgtactgtaacttggaatttgttctgttatgagtctatcttgaattcccatccatgaaactgtagtcaccaaaagcaacaagtattttcacatgatgtaaaagaccatactatgatggccattgctagaaattgaatcacaaataatagctaataatttttcatttttcaaaaaagatcatttggatagcagctatgtataaaatggaaaataaaaaattattctattttgcatgaatagttcagactttcccataccacagccaagcagtaactaaaattaggatcttaattttcaatgataaaaggtctaaggttcatttaattatgctcctttaacactgtctttctagatttttcacccagtattttcaaaatttgggaatgtaaacaattgatatatttattgtatgttggctagcagttcatccttc

SEQ ID NO: 47 - DNA, 54 bp, Homo sapiens
atggtattattatcagagtctgcccattctggcatattcatgacaccaaaggaa

SEQ ID NO: 48 - DNA, 387 bp, Homo sapiens
gctagaattcagctttgcgacaatgatatctacttcatccctagaaatgtcattcatcagttcaaaacagtttcggcggtgtgcagcttagcctggcatataaggcttaaacagtaccaccctgttgtggaagccactcaaaacacagaaagcaattctaacatggactgtggtttaactggaaagcgagaattagaagttgactcccaatgtgtgaggataaaaactgaatctgaagaagcatgcacagagattcagctgttaacaactgcttcatcatctttcccacctgcatcagaacttaatctacagcaagatcagaagactcagcctattccagttttaaaagtggaaagtagactggactctgaccagcaacacaatctg

SEQ ID NO: 49 - DNA, 30 bp, Homo sapiens
ttcagaggctaaagaatctcctcatctcca

SEQ ID NO: 50 - DNA, 199 bp, Homo sapiens
taccttcaagtctgtccctgttgtgcttcctcagatgttcttgggggtaattcttttttttttcttctcagtttgttggtgagaattgggggtgattctatgaaaaatattccggtataaatcaacttttctaacaccagcttctactatatacagagcctgaactaaccccaaaagatttggacagtctattgggacc

SEQ ID NO: 51 - DNA, 145 bp, Homo sapiens
gcagtatctgccaccacttgttttggtccaagggcagaaatgagcatcctggaaaccaatcagtacttgcgctctgaactggaaaagtgcaaacagaacttccaagacctcacagagaaattcctgacatccaaagctactgcct

SEQ ID NO: 52 - DNA, 99 bp, Homo sapiens
tcaaaagtggactgtatacttcctctttgatgtgatggtccctttggttttggtgcctcttccatgagcttcagatcagaactagctatgtggttccag

SEQ ID NO: 53 - DNA, 234 bp, Homo sapiens
ccacactggcctagcttctttaatacaaggtatacagttatatttgtttcaatgtgcttatctacagattctctcatctgtgtcctttttattgttttttaattctcacctgatgtgtcctttctaagtcagtttaagtagattgattttttcccccttcttatcatggattacagttttctgtttctctacattcctaacattattttatttggggttagacattgagcattg

SEQ ID NO: 54 - DNA, 75 bp, Homo sapiens
taaagccagaattacggctagcacctagcatttcagcagagggaccattttagaccaaaatgtactgttaatggg

SEQ ID NO: 55 - DNA, 25 bp, Homo sapiens
aaaaaggagcagtactaatcacaag

SEQ ID NO: 56 - DNA, 593 bp, Homo sapiens
ccttgacctgggaaagccattactcttgtgtctgctactgccctcccacagtcaccccaatattacaagcactgccccagcggcttgatttcccctctgccttccttctctctgcactcccacaaagccagggccaggctccccatccctacctcccactgcatcagcagtgggtgttcctgcccttcctgagtctaggcagctctgctgctgtgatctgcacaccctccaacctgggcagggactggggggatgcagtgtgtgttagtgcccatgtggcattgtggcactgttgccccccatggcggcatgggcaagatgaccttccattagcttcaagtcttgttctcttgtctgtggtctgtttaatatgtgggtcactagggtatttattctttctcccatccttacactctggatcattgtgcagacttaatcagggttttaacgctttcatttttttttttttttttttttttttgagctcaaagagagttctcattttccctattcaaactaatacccatgccgtgttttttaccttggatttaaagtcaccttaggttggggcaacagattctcactcatgttta

SEQ ID NO: 57 - DNA, 37 bp, Homo sapiens
ccagcctacgcatgggatttcaagagggccaggccac

SEQ ID NO: 58 - DNA, 315 bp, Homo sapiens
cttagttgagggagtcagcacagtcctttctgcagcttctaacccaggaccatgaactcaggtgcctagagaagccaggcagctaaaggacaaggaatgctgggggctgtgggaacaggaatgcagataccctttgaaggagcattcctgctaaaagaagctgaaaatgtagacctatgtgaagtgctctgatttctaaatattgtgaaggttaagaaaaacataaatttaggtctatgggctagatttagcccacagttgccagtttctagcgctaccaaatgaatgaataaacatgagcttgcgctcctagcc

SEQ ID NO: 59 - DNA, 29 bp, Homo sapiens
ggaggagaagcttctttcaacctctctct

SEQ ID NO: 60 - DNA, 137 bp, Homo sapiens
ttctgtggaggatatactaagtgcgactttgccctatcctatttggaaatccctaacagaattgagttttctattaaggatccaaaaagaaaaacaaaatgctaatgaagccatcagtcaagggtcacatgccaata

SEQ ID NO: 61 - DNA, 384 bp, Homo sapiens
ttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacaga

SEQ ID NO: 62 - DNA, 568 bp, Homo sapiens
ttgtgtccatgaactcacgcatagcagcatcctctactcagggacacattatgttgctatgaatccttttaagaattctttgagtttttcttcctttctacttctacctcattatatctacacctttcttaactaccttgcagtcaaaagccaaactacctgggatgctgctcctctagtttgtcttcagctaaggatagaggcaaagaatgagggaaggggaggaaaaagggagcagggatacgagaaacagaaaggaagaaaaaagaatccttttattttccaccctctcttcgcccattttttttcttttcccttcactgtacatctaattcttttgacccacttcaattttcatcatagtaggtagagccacatcttgagaaaccttaaaactatagatttactacaaaaaagatgagctaatacaaagtctaaagtaaactaaaagaggggttgatttacatctggcacaacttagtctttgttccaatcattttagaagtctcaatttaactccaggctagactccagtttgttctgggcctctcaggaattatgttttgtc

SEQ ID NO: 63 - DNA, 150 bp, Homo sapiens
atgctttaagtagaattcagtgccaaggagaacttggtgaaataaattattttaattttttttttatcctttacaaagccatggattttatttggttgatgtgtgctctgtacacaagccatttcaataggatggagctgttaattattt

SEQ ID NO: 64 - DNA, 667 bp, Homo sapiens
ccttggaccaccttcatgttagttgggtattataaataagagatacaaccatgaatatattatgtttatacaaaatcaatctgaacacaattcataaagatttctcttttataccttcctcactggccccctccacctgcccatagtcaccaaattctgttttaaatcaatgacctaagatcaacaatgaagtattttataaatgtatttatgctgctagactgtgggtcaaatgtttccattttcaaattatttagaattcttatgagtttaaaatttgtaaatttctaaatccaatcatgtaaaatgaaactgttgctccattggagtagtctcccacctaaatatcaagatggctatatgctaaaaagagaaaatatggtcaagtctaaaatggctaattgtcctatgatgctattatcatagactaatgacatttatcttcaaaacaccaaattgtctttagaaaaattaatgtgattacaggtagaggccttctaggtgagacacttttaaggtacactgcattttgcaaaaaaaaaaaaaaaaagtaatcttttagcaaccccagtattccttcactatttcgcttcctgcattagcaaattttacttacagtcaaaagtgcagatttatactcctgacgtgtctcattcacagctaaata

SEQ ID NO: 65 - DNA, 137 bp, Homo sapiens
ctttttgccatacttgactcatatactcaaaatacagagttgtcttggtgtaggaattggaaagcaagagtggaaaggaacgtaataactcgttggagtttaaaaatgagggtcttggcaagtaacactgatgtctg

SEQ ID NO: 66 - DNA, 466 bp, Homo sapiens
tacgggcaacagcagagcgtctatatgaattacatattaaaaaaacaaaaataaataaaaatattcacaaagaataatgagaaacttatgaaaatatagtaagtctaggaaatacataaacacactatctcaataaggcacccccaaccaaaagcataaaaacgcagacacaacagtgagaagtcagactagttgagttactacaacaaagtattttagttaagagatcacttagaaatttggtactggatgaaaagtaaaggcaatagcatgcggcaaactggtttttaaagggctattttataaaaacataatacagacaaaattcatgtgcaattttactacaaatataacaaattttatccaatgttctttcccctcttctccatattaagaaaacacataaagagaaatgaaaagaaactaaaggtaatatataatagcagtggatagacttctattttga

SEQ ID NO: 67 - DNA, 218 bp, Homo sapiens
acccgtgaggatcactctcaaatgagattaaaaacaaggaagcagagaatggtcagagaatgggattcagattgggaacttgtggggatgagagtgaccaggttgaactgggaagtggaaaaaggagtttgagtcactggcacctagaagcctgcccacgattcctaggaaggctggcagacaccctggaaccctggggagctactggcaaactctcc

SEQ ID NO: 68 - DNA, 620 bp, Homo sapiens
ctggaccctaggtgtgctattcttcctactagtggacactggacattgcagaggtggacaattcaaaattaaaaaaataaaccagagaagataccctcgtgccacagatggtaaagaggaagcaaagaaatgtgcatacacattcctggtacctgaacaaagaataacagggccaatctgtgtcaacaccaaggggcaagatgcaagtaccattaaagacatgatcaccaggatggaccttgaaaacctgaaggatgtgctctccaggcagaagcgggagatagatgttctgcaactggtggtggatgtagatggaaacattgtgaatgaggtaaagctgctgagaaaggaaagccgtaacatgaactctcgtgttactcaactctatatgcaattattacatgagattatccgtaagagggataattcacttgaactttcccaactggaaaacaaaatcctcaatgtcaccacagaaatgttgaagatggcaacaagatacagggaactagaggtgaaatacgcttccttgactgatcttgtcaataaccaatctgtgatgatcactttgttggaagaacagtgcttgaggatattttcccgacaagacacccatgtgt

SEQ ID NO: 69 - DNA, 105 bp, Homo sapiens
taatgaggtatattagccagccacctcttctacttgatgtgcatatccacaaaccaatgctgaatgctcggacttggatggatgctttgcttgccttcttcccag

SEQ ID NO: 70 - DNA, 27 bp, Homo sapiens
gtgatggatacgcttggcattccttat

SEQ ID NO: 71 - DNA, 118 bp, Homo sapiens
catcacttaggcgaaccagcagacagggcaccaaaatacacattagaggagactggcttccatgagacgcttcgactgtctcatcggggcacttgtaataagcatcttggtgccactg

SEQ ID NO: 72 - DNA, 62 bp, Homo sapiens
tgtggaaagaaaagtttgaacaagctgaaaaaagaaaacttcaagaaacaaaagagttacag

SEQ ID NO: 73 - DNA, 49 bp, Homo sapiens
ttcttggctgccattgcgccactccgacccctggagatcatcttgggag

SEQ ID NO: 74 - DNA, 68 bp, Homo sapiens
tgaggacattgcaagcacctccagaaggtctctctccacctgtgatatcctatgtttctatgaatccc

SEQ ID NO: 75 - DNA, 329 bp, Homo sapiens
ttctgcttggccgtatttgaagacaagctgaatacatatctatgttctgaataagtccactatggatatatataggaagagatatacatatatccatccacagatacacacacacatatatatttctgcatgtatatatacataattctttctatagttacaggaaatacttcttctataattctgattttgactcccatcctccaccatttactcatccactcattacctaaatcttggctttctttcctatattgtaaataatccatccaaacttctagccagtactgtcaggagggttcttgctcgagtgagctgttaatactatt

SEQ ID NO: 76 - DNA, 28 bp, Homo sapiens
aaaccataggcaaaattgcaacatgctt

SEQ ID NO: 77 - DNA, 36 bp, Homo sapiens
atgagtcgtgactctaaatctcttcctttttccgca

SEQ ID NO: 78 - DNA, 48 bp, Homo sapiens
agacctgcagggttcgtggataataaactcaagcagcgagtcatccag

SEQ ID NO: 79 - DNA, 98 bp, Homo sapiens
ctgctatggtcacggttctaaggatgatttaagaagtctctggctcctcaactgtgcactgtagtgtaggctgacttcaaccacggcttctccagctt

SEQ ID NO: 80 - DNA, 42 bp, Homo sapiens
attttcaagtacaacattctgctcaacatcatttacacttga

SEQ ID NO: 81 - DNA, 314 bp, Homo sapiens
taacaatcggatctttcaggaactaatagagcgagaagtcactcattaccacaacagtgccacttatgtggaatctgctcccatgacccaagcacctcacaccaggtcatacctccaacatgaggatcaaatttcagcatgaaatttgaagggagaaaatacccaaactgtattcaatactaagaaactgcatatgagaatattactgtattgttaatagctataggggagggagccatgttgtagactaatcaatccatttatgttcaatttgtttatgttagaaaacctgcactttctctgatattggtagc

SEQ ID NO: 82 - DNA, 584 bp, Homo sapiens
ggacacatgttcatccgtcaccttccggggtcaaattctcggttaagcctgtgtggtagggctgtagagcagtgtgtgcctgggaaagcctctgatcacacatttcctcagggattttttaggcctgatgtccctttgaatttactttaacaatccttttgtaattttattttttacatcaattgtaacttagtattattatatatataggcatttgggtgtgtcttgaaggcatgtcctggtttcaaactgatttgtggtatattattgaattaggcccatttactggaattataaactgctatccaactacacaaagagctgaggattctggggaggtcaggatggagatgaaatagaagtgaggagaaaatagctctctctctcacatgcagattctttatggataaaatgcataattacctaaaatttaaaatactcaaaaaattatctaatcagtatgtaatggaaattatagagcaaagaacaaaacaaacaccaaaataattttcttttttctacataaaaattaaataatttacataatgaaatattccatgatcccagtgtaaaatgccaataga

SEQ ID NO: 83 - DNA, 25 bp, Homo sapiens
gagcccacggtcaggccacatcctg

SEQ ID NO: 84 - DNA, 259 bp, Homo sapiens
atcaggctggacttggacattctgggtgggaagtgtctggaacctggacgctggcttggagaggagcctcaggaggaatcgccaggattctcagctgtcgagaatgtcacccgctgctgtactcccaacagcaagggaggcacctggcacgtcgtgagcaactctcgtgtatttgttgcatgatctagtccaagaatggccaatgagtttcacctgccagttttcacaggcctgtgtgccgagagtgttccttaccatt

SEQ ID NO: 85 - DNA, 29 bp, Homo sapiens
tttcctcaggcttaaacctttgccactga

SEQ ID NO: 86 - DNA, 168 bp, Homo sapiens
tgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggta

SEQ ID NO: 87 - DNA, 45 bp, Homo sapiens
aggtggccggagggaagatcctccaatgagcctgcgcactgtggc

SEQ ID NO: 88 - DNA, 27 bp, Homo sapiens
tgtgacagttgtaaataaagtttgaaa

SEQ ID NO: 89 - DNA, 27 bp, Homo sapiens
gagccgacacagtgccgctgcattcca

SEQ ID NO: 90 - DNA, 28 bp, Homo sapiens
aaccttatgatagtaaaagtttgcggac

SEQ ID NO: 91 - DNA, 99 bp, Homo sapiens
agtcatttcaagttgtcgtgagaggaaacggcttccgacatgcccgcaacgtggacagggtcctctgcagcttcaagatcaatgactcggtcacactca

SEQ ID NO: 92 - DNA, 38 bp, Homo sapiens
ccccccaatcggactgccaaattctccggtttgccccg

SEQ ID NO: 93 - DNA, 267 bp, Homo sapiens
tctcttggtcttggtagccctattatatcattgcagacacagtgttgtgcatgtgtgtattgtatatgtgcaccagcatcaaacattgtgtatctggaaggaaagcagcatgttccagttctaaaatgcagttaccagacccatcactttaaatccttaaagttagaagctagcaaattttctgtagtgcagaacgtttattgctgtttttgtgtttgaatagtataatgtttgatgcctctcttctgcaaagcgtatcatctcatt

SEQ ID NO: 94 - DNA, 220 bp, Homo sapiens
actgagcatccctttgtcttggccagcttagtattcctaactactctcgacacctctgctatttttctataattatagataagactgaaataatagtaaaagcagctagtattcccaaagcatgtatactctaccaggcatcacactaagaatattaatacatattaattaatttaatcttccctacagccctgagaagttgggactctcattttgcctg

SEQ ID NO: 95 - DNA, 40 bp, Homo sapiens
tccctatcttgtcatcaccagcagtaacctcagtcaagta

SEQ ID NO: 96 - DNA, 105 bp, Homo sapiens
tggggttgtctgtcaggaaagtataacttaaatgtttataaagtttcacatacttctctttatattctataggtaatgtagatttgttgacactgctttgattta

SEQ ID NO: 97 - DNA, 436 bp, Homo sapiens
tggatggccagataattgcctgttaaatagtgaatgaaaattgtcagggggatgagggaaaatgcaagtcatttacattcctcagcagaatttgcaaactagttctacaactcaaagtagtttaaaggaaagtgggttgaagggtcattgaaaagagatacaactgataaaggttgattggaatttcttgtcatattagccagttaatgatgtatttaaattatcctgtaggcgctttcattataccttaatgttcagaatttaactcttttagtttgaaagtcctagataaatttttcacatttaaagatgaataattcagatacaggacagagagtactgcttagagctacatacaagatgaagagcaaagggtgatctgttgacaagctgaggaagtcggcaaaggtaggccctttctcacagcggcatttga

SEQ ID NO: 98 - DNA, 48 bp, Homo sapiens
atggacaccgacattccaaccttctttccttcttcggcgctcctgaag

SEQ ID NO: 99 - DNA, 785 bp, Homo sapiens
agcagcaattagagtgccttcaagaagacttaaaaaaaatacaatatccaattagaaaagccatattttaaacatttgtacaagaataagctgctgaaacttagtaattgaaatatgacatctgtacaacaatttacaatagagctagaagggaatttatcattatcctgcatagaactggtctgcatttggttactcactgtcacctgttttgatgaacaaggcctggtaacaaaagaaaaatacctgttatcaattctaacgtgttgaaaacactggcaatattataatttagtgaattcaactgatttcaacacaagcagctcattttgtcaaaggtgtaaagtatttagaaataatagctctccctttaatataaccagtgaaagaaaacggacatgtgatctcgaggtacaacttggtaaaagtctgaataggcaaatgacaaagcctaactttgtccaaagattctaactctcacattctattactataaaacacaactgtcactgtcattcagttcttactttggtttcagcagattaactgccaaatgctgaagaatgtatccaggcactatagttcgtatgttagaaatatgtctcatatttttccatgtgtttttaaataatgaaaaactacccttcatattagaactctctagtaacaaccaaatgtatttaagtattataaacgttatttacagtgttcccccaaataaacaaaatttttttcctctatcttaacatggtattcctgtttctgtgtaacaggcagtcatcctt

SEQ ID NO: 100 - DNA, 130 bp, Homo sapiens
acctggcatgtagagttcaaaagtttaattaaagctcttggcatgaaaaatagaggtgcacagacccttaaatgacagccattaacagttacaggacttggtctgtgtggggccaagagtttcaaacatt

SEQ ID NO: 101 - DNA, 26 bp, Homo sapiens
agaagcaaaaactaggcagacaagaa

SEQ ID NO: 102 - DNA, 34 bp, Homo sapiens
aataaatctgtgaggtctcccatcaacctgaaag

SEQ ID NO: 103 - DNA, 200 bp, Homo sapiens
tgcacctgtttagtttgtgacaatctgagcccagtacatggttctctgattcctaagccaggagtctctctgtaaccaaactgctattatgtgagcatagaacagctctcaaagtaaatgtcccacttctatttctggcaggttatgtttagctacctttccaaaagagtcccaatcctagtatgcctttcaacagtgtc

SEQ ID NO: 104 - DNA, 152 bp, Homo sapiens
cttagtggggtttggaactgcctgagaatattcctatagaaactgggtcatcttgccttctgtgccactagaacctcctgtctctccaatagctgcttctctctaattcttcaccatagttttctttctgtggtcttttgaggttctctcct

SEQ ID NO: 105 - DNA, 185 bp, Homo sapiens
catagctaggcagtgttggagatcagcaggaactagacacaatgaatggatatggcatcaatactcatgaacatgccattcttccagcagtgcttggcaactcaggttgaggaacagagaaggtggatggcttaggtaatggaattggatgctttttaaatgtcagtggctgtcaaaactgtata

SEQ ID NO: 106 - DNA, 488 bp, Homo sapiens
tggcatgacatagctaaagcactgaaggaaaaagtattttatcctagaatagtatatccagtgaaaatatcctttaaaaatgtgggagaaataaagacttctccagacaaactaaaataagggatttcatcaataccagatctgtcctataagaaatgctgaaagaagttcttcagtctgaaataaaaggatgttaatgaattagaaatcatttgaaggtgaaaaactcactaataataggaagtacacagaaagagaacaaaaaaacactgcaattttggtgtgttaactactcatatcttgagtagaaagataaaaaagatgaaccaatcagaaataaccacaacttcttaagacatagacagtacaataaaatttaaatgcaaacaacaaaaagtttaaaagctgggggatgaagtcaaagtgtacagtttttattagttttctttctgagtgtttgtttatgcagttagtgataagttatcatc

SEQ ID NO: 107 - DNA, 154 bp, Homo sapiens
acagcattgataaacctgtagctagactaaccaagagagaagacccaaataaagaaaaacagaaataaaaaaggagacattacagctgataaccacagaaatacaaaagattatcaggcattattataaactacaatacactaaccaactggaa

SEQ ID NO: 108 - DNA, 123 bp, Homo sapiens
tgtccaacaggtgacacacatgttaagtggcagaaatggagtttgaaccatgtgttatggctctagggctcaagctcttaacactatcccaagtagggtgggaaaggacaatttgcctcactc

SEQ ID NO: 109 - DNA, 554 bp, Homo sapiens
tgtactccagagttcctttaaagctctagtgttttagaaacatgagttactacatttaatgtaaattagagtgaggaaatgaaatgaggtaaaatatgtcacttaatcaggaaagagaggtgggagagaaatacagagtagcctccagaaagttgagagcttggatttcgaaagtagaattttcgagcttcttaaatggctggggaaaagaaacatacaattatgttttgccttgagtcccagaagtcctccaccccccacacccccaactgtgaatgtagagggtgggacatcaagaagtggaaatacaacaaaagagacaatttggagaagggatggattgatacagcaccagataagtaggtttgcttaagcataagagaaggaataggcagaaaagtgaagaaaaaaataaaggagattaggagcctgaaatggcctaagagccctgtcaaaactgaaggtaaggattcttacatgtaaaggccataggaaatctttacagggtattgaaaagaggagctgggatttcgaaggtgacagattagcatata

SEQ ID NO: 110 - DNA, 129 bp, Homo sapiens
gctgggcctaaaggagataacatttatgaatggagatcaactatacttggtccaccgggttctgtatatgaaggtggtgtgttttttctggatatcacattttcatcagattatccatttaagccacca

SEQ ID NO: 111 - DNA, 71 bp, Homo sapiens
gtgacaaaggtgaaacaggtgaacgtggagctgctggcatcaaaggacatcgaggattccctggtaatcca

SEQ ID NO: 112 - DNA, 204 bp, Homo sapiens
ctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaa

SEQ ID NO: 113 - DNA, 375 bp, Homo sapiens
tctcatctggtggtggcaagcactaaaatcctgattttaacagaatagtagtaaaaatgcctcagtgatttaagttgaaagcagtacactggtacatggctcttgtacccagtatcaggaatgtacaaatgttttttattcaaaaatacaaaataaattatctgtaggcatggacaatgacagcagtaaaccattatatattttgtcaactgaaaccagtaactgatggttatagtgattttcagccagcctttttcttcattttctccaactgacttctctgaagttattggtgaggaacactgccttgggcttcctgtcacagttcattaataaaggtaaagcactagtctaggagttagaacatgccacctc

SEQ ID NO: 114 - DNA, 71 bp, Homo sapiens
tgaaatggcatcaacatgatgctgcccattccactgaagttctgaaatctttcgtcatgtaaataatttcc

SEQ ID NO: 115 - DNA, 36 bp, Homo sapiens
gaagcagcacgaaagagagaggtccgtctaatgaag

SEQ ID NO: 116 - DNA, 62 bp, Homo sapiens
gcagcctgaaacttgagagcgaaagtgagataaatgtcaaaggtgtttcaagccagacattt

SEQ ID NO: 117 - DNA, 256 bp, Homo sapiens
gttcaacctgtgtatcagtgccctagctgtagcttcactcacatgagctcttgcaggaagggccctgcgggaatctttttggagattgctggcctggccagtagaacttttttgtagtattaaagagagggagatatgtctgtttctaacttcccattgacagatgaatggacaaatgaatgtatgaatctgacatgagtcaaaacaagcaaaaaagaaaattttattaaatatactcagggactgggaaccaagt

SEQ ID NO: 118 - DNA, 59 bp, Homo sapiens
caaataggaattattaacttgagcataagatatgagatacatgaacctgaactattaaa

SEQ ID NO: 119 - DNA, 213 bp, Homo sapiens
tgacccatgaccttgccgcatgaggcctgagggcatggtgtccagagtcccagagcagatcaggccccaaagtcctgctggaccccccagccaccgtgagctcctccgtgtggctagggagctgctgtccagaggcggaggtaaacattgatccctcctgcacactcagctctctcatggaagtcggagccctcagggtcacctgaaaactct

SEQ ID NO: 120 - DNA, 87 bp, Homo sapiens
aggacgggaacaccacagtgcactacgccctcctcagcgcctcctgggctgtgctctgctactacgccgaagacctgcgcctgaagc

SEQ ID NO: 121 - DNA, 96 bp, Homo sapiens
aaatgcaccgcacccagaccaagttcgaggacgccttcaccctcaaggtgttcatcttccagttcgtcaacttctactcctcacccgtctacattg

SEQ ID NO: 122 - DNA, 28 bp, Homo sapiens
acggaacaaaggatgagcagcccgaggg

SEQ ID NO: 123 - DNA, 620 bp, Homo sapiens
ttcgaatgtttcagagcgcagggccgttctccctcgtgtcctctggacccacccgccccttcctgccctgtttgcgcagggacatcacccacatgccccagctctcggaccctgcagctctgtgtcccaggccacagcaaaggtctgttgaacccctccctccattcccagttatctgggtcctctggattcttctgtttcttgaatcaggctctgctttccccctagccactacaggcagcctctgacagtgccgctttacttgcattctgcagcaattacatgtgtccttttgatccttgcccaacttccctccctctcccagctcctggcccctggcccagggcccctcttgctgtttttacctctgttccttggggcctagtacccagcaagcacccaaatgggggaggttttgggatgagaggaggaaacgtgtatacctgtaacatctggtggctcttcccccagaagtttgtgttcatacataattgttttccacgctggatcataatgtgacgtgcagttctgccctgtgctggggagccacatgaagcttcccctggctaacttgctaccccgcagcaatcccagtgtggccgtctgcttgctaaaaaatg

SEQ ID NO: 124 - DNA, 143 bp, Homo sapiens
atggctaccacgctgaagtcggccccttcgtcggtcttcctgctgcacccttcctctccacaccatgctctccattccgggatctcacacgtgcatttgataagttacaagtgcttgttgagaaatgccggtgtcctgtccca

SEQ ID NO: 125 - DNA, 100 bp, Homo sapiens
ccggtgttggtagaggaatttcacatgagcagtctcctcaccactgtccggtggaagtcggagggctatagcagaaggagccaaaaccacactgctgcca

SEQ ID NO: 126 - DNA, 277 bp, Homo sapiens
ctgagcgcatcctggctgcagttctctgctcaatgttttcttaaaaacatgtattttttcttttgcctcatgtttgttttcttaccccaaatgttccttgcttgctttgcacatgccccacctcccacccaccaagacccattggaatcccaccactgcctctactaggctgcctgagttcagataatgatctcaaaaacagcactcgctcccctcacccactaaccccctggctctaagcagcttcctttctcagagggccttgcaaattgtcaga

SEQ ID NO: 127 - DNA, 174 bp, Homo sapiens
ctaaggccattctgtgagttatttttaaaacttggtgttttgcacataatgatcttaaaaaaaaatgaattaccaaaaccaagattctcttctaaaatgaaaatttaatgcaggtacaggataactttagggctatatctaatctgaagcttatcaggtagcaaaaccattttc

SEQ ID NO: 128 - DNA, 44 bp, Homo sapiens
ttgagtttctctgcaagcaatctcctctttagtaatgtttgctt

SEQ ID NO: 129 - DNA, 479 bp, Homo sapiens
taatgcggtcagctgtgtcacaatgtgatatatagattatatttaccatggcatattttgtttgcgaaatgggagcggatgataaatgaagataccctccagttttcacactagttcctgtggtccggagtctctcaaacaataaagcacccctgataatggagaggtatttatgggaacataattgacttcaaagttttagatctctggctgaagtttaagatgggatagtccattacattaatgtctgtgcttaaagctcctatttggcttaaataaattatttagggtttactgcttaaaccttggtcaattcttgaacgtttgggctagttaagtaattttccagtgactttctgtgccttggtgattcatttacttgattgagctcctgtgtgctcgtatgatttctaaatgtatttctcaagttttgcctggcaatgaatgattttgcttactggagtcttgtgtggtacacc

SEQ ID NO: 130 - DNA, 252 bp, Homo sapiens
cataatctgcttcgaggaggttttcatttctggctgaacaaggctgtggtaaggcaagtccggaaggcatgctggaaacttgagggaagttttgaatggaaactgcagtcaacagctccatatgatccgcatgtggcttccccagaggcaagttttcagctgcgtggtggcctctcccagtcactccacaggctgccctgacgctattaatatttgctgaagcaagacctgaggttcgttgcagatggatta

SEQ ID NO: 131 - DNA, 156 bp, Homo sapiens
cctgaacagggcaaaggactgcaaggggcaggagcttgggctgacatgcaaggtggctttacacaaggccctttttagagagtgtgattctctgaagcttttcttggcagcttcagtcttgaacctcactggaagggatcctccaaaacatgaccc

SEQ ID NO: 132 - DNA, 30 bp, Homo sapiens
cacagcttgggatgttaccttgccttttgt

SEQ ID NO: 133 - DNA, 106 bp, Homo sapiens
tgagttcaaatcatccgcactgtcccatgctcacttgttacccagtcactggccacggggaaggaaatttgggtaacaaagtgatatcatcaggaatcctcccaga

SEQ ID NO: 134 - DNA, 84 bp, Homo sapiens
tacaaagacagcatgctaataatgcaattactgagagacaacttgacattgtggacatcggatacccaaggagacgaagctgaa

SEQ ID NO: 135 - DNA, 28 bp, Homo sapiens
tcagtaatagctgaacctgttcaaaatg

SEQ ID NO: 136 - DNA, 84 bp, Homo sapiens
gaagttaaaatgtggtaactgttgaaaattgaaaattagacagctatcccattttaaattgatgggtgcaaagtgtgtttcttt

SEQ ID NO: 137 - DNA, 261 bp, Homo sapiens
ccctaagcagttcccgatttgacttttctctttagcttagtgattttgggggcccatattttcctttcacaaatgtaaatggattaaatgtccactattaaatacatattggaagaatggattaaaaaaataatccaactaaatgctgactataagaaactcaccttacctataaagacacatatagactgagagtaatggggcgtaaaaagatattccatgcaaatggaaaccaaagttgagcaaaagtagccacactta

SEQ ID NO: 138 - DNA, 124 bp, Homo sapiens
ctcaactgtgcctgcaatgaaagttaaagcattttttttttttttttgccatcacaaaaaaaaccaatggccagtcagggttcactagaggagcagcaacatccaagagaggatcacaatgaaa

SEQ ID NO: 139 - DNA, 264 bp, Homo sapiens
gacctgattcattatcttggcattgatttataactacagttctttggaaaatatataaggtctgaagtcaggaaaacatctcagaagcattcactctaccatgtaaccattaatttaccctgatttggaaaaatagcctgaaaactagtgaatgcttccatctcaggagaaacttcagtgtcactagatatggtaaactgtagtatgctattttaacaaaatccagagcaaaaaatggatgaatccaggggaactacctatagc

SEQ ID NO: 140 - DNA, 199 bp, Homo sapiens
tggcctcttttctcactacagcttgtctttcagttccacaatctaaggaggataatagatagccagattttattggtcccaaccacatcgtcttccccacaaaccagcagctcactcacttttctattttttacgttggccagattagaaacttgtgtcatcatcatcattttcccctaccgtgtctcaaagatctagg

SEQ ID NO: 141 - DNA, 281 bp, Homo sapiens
acccagattctcagtccatgacatagcatgcaaatgtcatgctaacactcagatacgaacacatagccaatcctacatcaatgctatcctattcctggatagcatcgtgccataaggaaccagtttgcaggactcatcattgaagtttggcatttcagaagagaaagattagtgccttccttttttatataaaacagccttatagatttccatctaaaaataatttcttcatcaaatatattctaaaaaaaattttgcttactgcttatagctccctcttg

SEQ ID NO: 142 - DNA, 41 bp, Homo sapiens
ctagaattaagatatgctgatgagttgcttctgcagttcat

SEQ ID NO: 143 - DNA, 70 bp, Homo sapiens
ttagcatttctgtgcagacagcttttttgctgttcttggtcctcagcaatgacaatggctccgaggagat

SEQ ID NO: 144 - DNA, 54 bp, Homo sapiens
tcttggaaaatggactcagtgtagacacttcatggtttgtgttctgctcttcag

SEQ ID NO: 145 - DNA, 31 bp, Homo sapiens
atggaatatggagcaaaaggggtcctgtgct

SEQ ID NO: 146 - DNA, 113 bp, Homo sapiens
tgagtgtagtattggtaggatccttcagcaccctgcttctgttatggaagctcaatgggaaaattcctctctccccagcccttggcagacagagctcatgatggtagagtttt

SEQ ID NO: 147 - DNA, 300 bp, Homo sapiens
gtgacttggtccaaaagacctgggcacttggtctaacttttcaaacattatctaacctctgaatctggaataaccaaactgtaagttgacttaattcacagaagtgcagtgatggtaaaatgaaatagcatgagtagagtgataagtgtgatgcaaatgaaagtcatatcttcattactaggctttatttattaaatatagctaaagtactctaaacgtatatgtctacacttttttgaacatggatagtttttacataactgtactgaaagaaagggcactaattactatgcgctctaa

SEQ ID NO: 148 - DNA, 314 bp, Homo sapiens
ttgagatgatacatcaggtgctctctgagctacaaagggtaaaaggaaatcctctcattacaaactgacaaaggtaaaaggtaaaacatttttcacaacagcatgaaccacatactcagaaatagatctgggtttaatacaaatgacataattcctagccatctacagctacttttataacttcatacagataatgaattaggctaaaatgcactataatatttggtagacttcaggaccagtatccttagtacatcacaaaatacattttgatatcccaaagaagcaagacttgcaagtaggcgttttaaaca

SEQ ID NO: 149 - DNA, 590 bp, Homo sapiens
gttgctcccattacaacgggctatacggtgaaaatcagtaattatggatgggatcagtcagataagtttgtgaaaatctacattaccttaactggagttcatcaagttcccactgagaatgtgcaggtgcatttcacagagaggtcatttgatcttttggtaaagaatttaaatgggaagagttactccatgattgtgaacaatctcttgaaacccatctctgtggaaggcagttcaaaaaaagtcaagactgatacagttcttatattgtgtagaaagaaagtggaaaacacaaggtgggattacctgacccaggttgaaaaagagtgcaaagaaaaagagaagccctcctatgacactgaaacagatcctagtgagggattgatgaatgttctaaagaaaatttatgaagatggagatgatgatatgaagcaaaccattaataaagcctgggtgaaatcaagagagaagcaagccaaaggagacacggaattttgagactttaaagtccttttgggaactgtgatgtgatgtggaaatactgatgtttccagtaagggaatattggtgagctgcatatataaatttga

SEQ ID NO: 150 - DNA, 115 bp, Homo sapiens
tctgcagtttctagcggggtttttacgagaaccatcaggactaatgaggctttctatttgtccattaacagacttgagtgaagtcataatctcatcggtgttgattttgaaatcc

SEQ ID NO: 151 - DNA, 731 bp, Homo sapiens
cctgtggcatgccaatgaatctttctgatgggagacatgtacagattttgtgcatttatgttctgaatgcaagtcaacaattctgatctagagtttaaaagtgaaagtacattagcaccataacatgcgtctttaaagccttcccaaatattagtaatcttgaccagcaatgacaagaaaaaagaggagcacctttacaagcagttgatatccaatattaaaataattgtggctttaaaaatatttctttaaattcttgcattacacttttctttttaaaccaatcttccaggagattaatcaatgaaatttataagttttatcaacgtataaaatttttttcatcttctgggactcatagaatacaatctgtgtttctgaccagttgaggtagttaaaatagggagggcttttctaatttcgtatttgactatttcagaaagaaaggttatcttttactggtgagcacagtcattgctctgcagatgggctaggattcaaagaatataacacagtgttgttatcataaagagtgttgaagtttatttattatagcaccattgagacattttgaaattggaattggtaaaaaaataaaacaaaaagcatttgaattgtatttggtggaacagcaaaaaaagagaagtatcatttttctttgtcaaattatactgtttccaaacattttggaaataaataactggaattttgtcggtcacttgcactggttg

SEQ ID NO: 152 - DNA, 31 bp, Homo sapiens
tgcatacaactttggtcatgtatctctgcaa

SEQ ID NO: 153 - DNA, 26 bp, Homo sapiens
ccaccaccagatgagaagttaagcag

SEQ ID NO: 154 - DNA, 85 bp, Homo sapiens
tcttgtgaggcagcaacaagtgttcaggtacagggaatacataagtacagcgtaacaataccgattaccattggaaatgctgttt

SEQ ID NO: 155 - DNA, 26 bp, Homo sapiens
gcaaaccctgacactggagtgctcac

SEQ ID NO: 156 - DNA, 99 bp, Homo sapiens
gattggtctctgtggtgtgattctcttcccaggtgtccctttctcctcccctagtgtccttaagtcctcctccacagggaacatctatttgggctttga

SEQ ID NO: 157 - DNA, 37 bp, Homo sapiens
cttggtgtagaaattatgtgaataaagttgctcaatt

SEQ ID NO: 158 - DNA, 26 bp, Homo sapiens
ggatgttgctaattgtctgccttcct

SEQ ID NO: 159 - DNA, 273 bp, Homo sapiens
ggcaaaggccatgctctgtgctctcagcagacctgtgtgggcagggtgcggtgagggtggggtggtcaccggaggcagccttcaagcagcaccgtgtggggcctcctcctcagtgtaggcccatgccagggacgccgtgtgtgaggaggggctcagggagcagcagaggctggggccgctccgcctctaggatgcctgtccaggcagatacccggggcaggaaggcccggcatggcccagggagggccagttgttccacgctgtgctctcggc

SEQ ID NO: 160 - DNA, 274 bp, Homo sapiens
gaccagtgtggcttagaatagaataaaagggtactttcttttctgcctacttccaaagtgcacttggagttcagagaaggcagaacttgtacaaagttcagcgaaggaagagactgcatcctataccgaagagatatgtcaccatgacagaaggcttgaattggtggagagggaaattgtgtgagtaattctattgagtcttggctggttaggtggcatgtcattgtcttacatgtgtggactggcatatgccaagtcgggcacaccaggaaga

SEQ ID NO: 161 - DNA, 293 bp, Homo sapiens
tggtagtaaggagcacaaagacgtttttgctttattctgcaaaagtgaacaagttgaagacttttgtatttttgactttgctagtttgtggcagagtggagaggacgggtggatatttcaaatttttttagtatagcgtatcgcaagggtttgacacggctgccagcgactctaggcttccagtctgtgtttggtttttattcttatcattattatgattgttattatattattattttattttagttgttgtgctaaactcaataatgctgttctaactacagtgctcaata

SEQ ID NO: 162 - DNA, 31 bp, Homo sapiens
tttttcatttcgaatcttgtgaatgtattaa

SEQ ID NO: 163 - DNA, 31 bp, Homo sapiens
gctatgacctcgctggggagctacaacaagt

SEQ ID NO: 164 - DNA, 35 bp, Homo sapiens
agcacaaaggcatctctacgcccatcggcctgccc

SEQ ID NO: 165 - DNA, 25 bp, Homo sapiens
tatgacaccagattcgtcttctcag

SEQ ID NO: 166 - DNA, 105 bp, Homo sapiens
tgagagggagcacagacgacctgtaaaagagaactttcttcacatgtcggtacaatcagatgatgtgctgctgggaaactctgttaatttcaccgtgattcttaa

SEQ ID NO: 167 - DNA, 113 bp, Homo sapiens
agcctggtgagaccatccaatcccaaataaaatgcaccccaataaaaactggacccaagaaatttatcgtcaagttaagttccaaacaagtgaaagagattaatgctcagaag

SEQ ID NO: 168 - DNA, 163 bp, Homo sapiens
ctgcttactcccaaaacagcctttttgtaatttattttttaagtgggctcctgacaatactgtatcagatgtgaagcctggagctttcctgatgatgctggccctacagtacccccatgaggggattcccttccttctgttgctggtgtactctaggacttca

SEQ ID NO: 169 - DNA, 765 bp, Homo sapiens
gtggactgatgctgctaacgatctcttggagttagctagtaccggtctcttattcagagtatttactctgctatagcgtcatatttgataattctagtggcttgaataatgttttgtaaaaacaaattgaaacttggtattggcaaaattgtacgagaaaagagctagggtaggcaactaaaacttacacagtgccagtctcaggaggtcagtagctcacagaactcaacagataaactggattaaaacttaaaagtcttctttctatttgagcccataatgactattttgaacatggctcttttgctgctgcctatatataaattttttattaattttcttgtattgggaagatcttgaatacgctccaggatgagaagaaaaaatacgctgacactgctaaatcgggtatatgtttttgcaataaagaacactggtcaatatacaactgaggaaaaactgaaacagatgtgagtcctagaaccacaagagtttgaatttgcccagaaatgctattttaaacactctatatgttggtctgctgtttttttgtggaataatgcattcttggcatccttaaaaggtttcaatatgttacaaggttatccggaaagagaaaaagcaaaggctaatgtatcaatctgtggagcactgtccagatttccgtgtacatttttgaaaagcacaataacttgtattaattgcacttaacacaatgaacctttagtttccaaccagttttcattctctgcagacccgggcttt

SEQ ID NO: 170 - DNA, 412 bp, Homo sapiens
ttaagggagaggataggtcacagcaggaaggtgctgagatgcggattcaggaaatatggtgaaggcttatctatcatgagtgctagtgatgcagatatgagtaggacagataattttctacttaggaaaaataatttcaaaacatttaatactaaaaaacaatgaaagtactaaaaggattcataggaaagtttaatataattttagattgaagaaaaacttattcatcaaaacaaaacataacaaaacgtggagaaaaattacaatatgaaaaagttaaatttgcataaggtgaaggacaccataatcaaaactgtaaaagtagtgtcagccagggagaaacattaatagcatatataaccaagtattgatgtacacactgtataatgagcagctatcaatctctgagaca

SEQ ID NO: 171 - DNA, 76 bp, Homo sapiens
aattacagtacttggaattgtgttctttatggttgtagtgttggtaaagcactaatatgccgaaaataaaggaatt

SEQ ID NO: 172 - DNA, 1287 bp, Homo sapiens
tggggaccctggtaatgctttagtcaaagggatatctctcttgtatcagaggctgtgtcttttagtaacaggagtcctcgtcagaattgcgtgtctgttgtctctaaaagaatgggtgaaccaatcggcctttgtgaatttattcagtgccttctctgtaccaagcactgggtaaggcacttttgtggagcattagacagtaaccctcaaggagctagagaaccggatgggagacatgagcagtaattaactcacttgttccccagagtttctatttgttttgattttctttttctgtgacttattttcctattttctttcctccatgtaattttcactatggcccaactaatataaacacctggaaattacaaggaaaaaaaattcttcctctaataactttccaaatttgtggaatatttatttgtaatagcagttatcagttatgcttatatagcattaaaaattctcctcctttgactacacacacaaccacagtgtggttctaatcatggagatatcagtaatttttagtaactgaattttgaggacatttctctgtttagcatgtatgcaaactgatatgtaatctgaggttccaaagtcaatttttttcttttttttttgagatggagtcttactctgtcacccaggctggagtgcagtagcacgatcttggcttactgcaacctctacctcctaggttcaagcaattgtcctgtctcagcctcccgagtactgggactactgtcttgcgccaccatgcctggctaatttttgtatatttagtaaagatgggttttcgccatgttggccaggctggtctcaaactcctggcctcaagtgatgcacctgcctcagccttccaaagtgctgggattacaggcatgagccaccgtgcccggccaaagtcagctttcaaaatccaagccataattggtgaggggggagtttcagaattacatagaaaaattaatatttgaaaaaataattctgaaatttcgaatttaaaaacagatgtgctgcttctgggtgtaggtagtaaaagtataggaaaagaactgtttccttagaagcggactgtggaagggctatgtagaatgtcaaagggcaacaagagcctgtgtttttaatgtcatcctgtactcggcacaaatcaaaggccaatacaagtctgaaaagcagaaataaatatttttccaggtttttgcttgggcacatactaactgctttgggcattctaatctggtctccaaacaccaaagacccatttcgagcctgctatta

SEQ ID NO: 173 - DNA, 33 bp, Homo sapiens
catgtaaacaaggacctcggtaatatggaagaa

SEQ ID NO: 174 - DNA, 516 bp, Homo sapiens
tgggcaatagtgactccgtttaataaaagcttccgtagtgcattggtatggattaaatgcataaaatattcttagactcgatgctgtataaaatattatgggaaaaaaagaaaatacgttattttgcctctaaacttttattgaagttttatttggcaggaaaaaaaattgaatcttggtcaacatttaaaccaaagtaaaaggggaaaaaccaaagttatttgttttgcatggctaagccattctgttatctctgtaaatactgtgatttcttttttattttctctttagaattttgttaaagaaattctaaaatttttaaacacctgctctccacaataaatcacaaacactaaaataaaattacttccatataaatattattttctcttttggtgtgggagatcaaaggtttaaagtctaacttctaagatatatttgcagaaagaagcaacatgacaatagagagagttatgctacaattatttcttggtttccacttgcaatggttaatta

SEQ ID NO: 175 - DNA, 51 bp, Homo sapiens
ctcacagatgtgtacagtacatcgccctctctgggtcgttattttacttca

SEQ ID NO: 176 - DNA, 34 bp, Homo sapiens
aaaattggatcgaacatttcacctctcatattaa

SEQ ID NO: 177 - DNA, 48 bp, Homo sapiens
atggtgatgagaggccaccgagagacctccatggtccatgaactcaac

SEQ ID NO: 178 - DNA, 358 bp, Homo sapiens
cctcagtgtataagatgtgcaagacaaatatgcttatttccttttctagaatataagtgatattatttgcttatgacactaacactattaatgacaggagtcaatcagcctttacagctatcaaaatataatgagatcccaatgatgattcttttttactttgaatgttaattagtttgggactttgattggctggcaaacattttatcattgtcagaatttaatttagatttcaaaaatagcttacaggattttaaacatggtgtggtattctaaagccttttttttaaaaaaagagatctttttgagagaaacaaatgaggattgtaaagtttggggacttacctctgtagcattg

SEQ ID NO: 179 - DNA, 58 bp, Homo sapiens
agatataattggttgtggacggctaaatgaacctattaaagtcttgtgtcggagagtt

SEQ ID NO: 180 - DNA, 77 bp, Homo sapiens
ttatgagttgcagaaacgaattgctgaaatggaaactcaaaaggaaaaaattcatgaagataccaaagaaattaatg

SEQ ID NO: 181 - DNA, 115 bp, Homo sapiens
gccaagagtaacaatatcattaatgaaacaacaaccagaaacaatgccctcgagaaggaaaaagagaaagaagaaaaaaaattaaaggaagttatggatagccttaaacaggaaa

SEQ ID NO: 182 - DNA, 212 bp, Homo sapiens
ggctgtatgggcgaaaaagatgaccgaaattcaaactcctgaaaatactcctcgtttatttgatttagtaaaagtaaaagatgagaaaattcgccaagctttttattttgctttacgagataccttagtagctgacaacttggatcaagccacaagagtagcatatcaaaaagatagaagatggagagtggtaactttacagggacaaatca

SEQ ID NO: 183 - DNA, 38 bp, Homo sapiens
cagttgcaaaacgactctaaaaaagcaatgcaaatcca

SEQ ID NO: 184 - DNA, 256 bp, Homo sapiens
ccctgactgatagcatttcagaatgtgtcttttgaagggctatgataccagttattaaatagtgttttattttaaaaacaaaataattccaagaagtttttatagttattcagggacactatattacaaatattactttgttattaacacaaaaagtgataagagttaacatttggctatactgatgtttgtgttactcaaaaaaactactggatgcaaactgttatgtaaatctgagatttcactgacaacttta

SEQ ID NO: 185 - DNA, 139 bp, Homo sapiens
tgtaacactgtccttcgggcgactgagggagtttcatattttctttagacatcgttaggcgccgaagctcttgcaggacaactttgatgctatatgaattctgccattttgctagcactgatatgctcttgggtccacc

SEQ ID NO: 186 - DNA, 67 bp, Homo sapiens
tttgcaaattgccaacgaacctgttctgccctttaatgcacttgatatagctttagaagttcaaaac

SEQ ID NO: 187 - DNA, 494 bp, Homo sapiens
ctctcaagaggtgtcccaattactaaattatgttgaactgttgtggtgtttagccttgttcaaaatgatttgattatcatctggattcagaaatcttttcattgtacagagatttcattgtcattgtcgttgttgttaactgatttttaatgatattgatttagaggcatttgtcggtagtttttaaattactatatagggttgaatgtctaatttctataatgtgtggatgcccatcagatatttagtgcattctatatttagcccaagtgtgatcacaatttattttataactttttatgtgttacttacatgactcaatatgcatcttacttaagaaattatgttgtgatctggagtgacacaaatggattgagataccacatccattaaaaatataagtgaatattttatatttaagttgtttaaatatttgcacattattgaaaacatagtactaggaaatgaaggcaccatttaacaaacgtgttctg

SEQ ID NO: 188 - DNA, 130 bp, Homo sapiens
ccgtctcttaagagcgatatatttaatacattcacaagattcgaaatgaaaaacaaacattattcatgctacaggtttttaattttccttggacaacaccaaaaacacagtactcaaacggcatgttttt

SEQ ID NO: 189 - DNA, 62 bp, Homo sapiens
actaaatggattttgtgacctctcctccaactccccacattgaaaactgctgctcttaactg

SEQ ID NO: 190 - DNA, 1197 bp, Homo sapiens
tttggtctactggaagcagctatcaaccgactttgttcttccaagtcctagtggaaaagacaaaaaagaaactaagttacttgtctgcagtcaaatggcaaattatgcagttaagatacattcttgagagccacttcttctgtcataaatatgtatgcatacttctgacaacttgataaaaagaaaagctacacccaaacaatacaacaccaaaatgaattttggcaccatcctataaaagccagcattcaaactaaaatctaatattttatctttctttttttatgaccaacaaccaaataaagcccatcatcaatcatctctcactatatattatagtaaaaccctcgaaagggatcacccccacactcctttcccttcccctctccagcccttttctccatccctgtccaattcatctttgctaaatcaggtgtcaccttccctcagcctcgcctatcaagaaagcctcacttcaatgtcatctttgccacttccctcttctttgtccagagagccagcccacagccaatgcttattgatctgttctaagcatcccagctgtctcctcttttctattcctactgtcaatctacaacccataacacgctcttacaatttcatggatcatttaatgcattcttcaagttttgctctgaaatttgtcttttaaaattattctgttggatggacaatgatttgacactcctggcaatattaaaaatatacatttcattatatatttcatataacatatatatttatttatttcccccccatagaaggcaaaatggctttcagcaatggcagttaagtctacacagcctctcaactgctttgacagagacaacctcctctgaaatattaaagttctggtatatttttgaaacagtcatattacatgtcaacgtaactataatgtaaacatcatatcaaaaaacagatgtgacccttttctgctatggtatgaatcatctgtttatgaagtggctgaacaagcagaaacagcccttcatttggaaattcaaatctgaaagtcatagtgctgaattaagtcttcacaaacaacacataaagatccagacattaggactgtataccttttcaatctgtttttgtttattgagttctttctgttgcttctcttttactctctctatttctttttgtttttctgatgccctacgatcctaaaga

SEQ ID NO: 191 - DNA, 35 bp, Homo sapiens
cttgaatggtgtggactgtcccgaggaaatccaag

SEQ ID NO: 192 - DNA, 252 bp, Homo sapiens
tatataccgcttcacaaaggcagtgaacacattacattctacaaaatctactttacagaaatttaaaaactttaaatatcaaaaggtacagctgaagaaacaggtataaatttggcagccagtaattttgacagggaagttacagcttgcatgactttaaatatgtaaatttgaaaatactgaatttcgagtaatcattgtgctttgtgttgatctgaaaaatataacactggctgtcgaagaagcatgttc

SEQ ID NO: 193 - DNA, 1368 bp, Homo sapiens
tgtggctgccattaaacaagtaagctttcctctttatccaggcactgaatcgatggtaataatgttgtctttttttttcccggcaaactttctgctttcggtccagagctctgagtttctcatgttctgctctcgaggttctgacagctgtttttggacttaattaaccattgcaagtggaaaccaagaaataattgtagcataactctctctattgtcatgttgcttctttctgcaaatatatcttagaagttagactttaaacctttgatctcccacaccaaaagagaaaataatatttatatggaagtaattttattttagtgtttgtgatttattgtggagagcaggtgtttaaaaattttagaatttctttaacaaaattctaaagagaaaataaaaaagaaatcacagtatttacagagataacagaatggcttagccatgcaaaacaaataactttggtttttccccttttactttggtttaaatgttgaccaagattcaattttttttcctgccaaataaaacttcaataaaagtttagaggcaaaataacgtattttctttttttcccataatattttatacagcatcgagtctaagaatattttatgcatttaatccataccaatgcactacggaagcttttattaaacggagtcactattgcccagacagtaaatggtttacaaatgtacaataacgtatgtttctaaataatgcagaagtggcaaagtattattcctattctcatctgactttaattttcaatttatttttcattgccttcataattttttatttcatgagtctaagaaacaaacaccagttttccttttcctttcaataacatagttctatacctagaaatctgatgttcatttatcttccctttaaagttgtgtaaatcctgggaaagaaatcgctgtgcagtgttctcccagaaaccaggcttctgcttgaaagagcaaagtttaaaagtctagtcacaagtagcccatatctggtctacttcaaaaagtttccatttatactaccaccttaaaaatttcctcaagcataaaacatgtttataaagttacaaatttgaatgtaactaaagatacatagacattttattattacactttcatgtactgcttgataaattatgtatcagttaaaaacactaaattatctcaaggtgcattcaatatctgtagggtagttaacaaaaacacacacgaaaaaaatatatattctatattctgtggaaaataacacagctttgataatgaccactgttaaaaactccaattctgattgatatctttttaaacatgaaataggtataattttaaggcagtggataacccaaaggattta

SEQ ID NO: 194 - DNA, 804 bp, Homo sapiens
tctctcgggagttggaacattgttatccttgtaagaaatactaagcttatgttgatttttaagtaattatatcttctcttcttgctggtgggtggggcagtttggtttagtgttatactttggtctaagtatttgagttaaactgcttttttgctaatgagtgggctggttgttagcaggtttgtttttcctgctgttgattgttactagtggcattaacttttagaatttgggctggtgagattaattttttttaatatcccagctagagatatggcctttaactgacctaaagaggtgtgttgtgatttaattttttcccgttcctttttcttcagtaaacccaacaatagtctaaccttaaaaattgagttgatgtccttataggtcactacccctaaataaacctgaagcaggtgttttctcttggacatactaaaaaatacctaaaaggaagcttagatgggctgtgacacaaaaaattcaattactgtcatctaatgccagctgttaaaagtgtggccactgagcatttgattttataggaaaaaatagtatttttgagaataacatagctgtgctattgcacatctgttggaggacatcccagatttgcttatactcagtgcctgtgatattgagtttaaggatttgaggcaggggtaattattaaacatattgcttctattcttggaaaaatagaagtgtaaaatgttaataatacaaatgtcactgtgacctcctccactgagaggactggtttatgccagatcattttccggcacacacggagtggctttgaca

SEQ ID NO: 195 - DNA, 252 bp, Homo sapiens
agttcattagaagagctctcccagatgggctttcatttcaaactcatcagctcttgcttttattcccatttttctaaagttctttttgatcatccggctccaactcccatttacgacctgattgttgatctgttcaagagaaagagacaggcctccagcaaaagggaggaaaaaaaaatgctttggtgagagaaagaagaaaaacacacacacagggtgttacacaaggatgaagtcctcctcacaccattg

SEQ ID NO: 196 - DNA, 33 bp, Homo sapiens
agcagctcagaatccattaaaaggagagctggg

SEQ ID NO: 197 - DNA, 87 bp, Homo sapiens
agcagttggcttctttgctaaacagaccataaagagaaaccatgcaaagagcattcagaaaacccaggatgcctcagataaaacaga

SEQ ID NO: 198 - DNA, 72 bp, Homo sapiens
tataatgttaacgatgactctatgaaacttggaggaaacaataccagtgaaaaggcagatggactaagcaaa

SEQ ID NO: 199 - DNA, 142 bp, Homo sapiens
gtgaactgctcagctattctgtgtacctcaggaggtctgcaagtgtgtggttaggtaaaaactgagctgtgcaaactcactgtatccaagctcttctcatgagagagcagaacaacctggcaagcttaaaggcaagtgtttt

SEQ ID NO: 200 - DNA, 106 bp, Homo sapiens
agaggtgacggaatgcttgttttgcaaaccccaacacatctttcactgtggaccattgctattacttcattaacacagttgctcagatctcaaggtactcacggtc

SEQ ID NO: 201 - DNA, 27 bp, Homo sapiens
tttccatttcagcaattcgtttctgca

SEQ ID NO: 202 - DNA, 111 bp, Homo sapiens
agacacttatgggacagttaagagcttggcaagtatcagaaggccaggaaagagtctttcttcattgaaggtctctgataatgccagtggagtcttggaatatggaacttc

SEQ ID NO: 203 - DNA, 355 bp, Homo sapiens
agcctactcgtcatgatattccacaatggtgcacttgccttttaatgctcttatagatatcttcaaacttgcctacatatatacgcctttgttggagtgggctaccatcatcaggaatgatgtcatttgtttcttcaaactcctttattataccaaaaaagtgacagactccacagtctgatcagttttggagaaatatgttaacattttcaattatctcactttctagaatcaaaatagtctgattttttttttcggcactcagtgtaaagaacaaagaactgaatacagtgggcccagaagagaaatatgcctcatcatttttattagctttggaactgtggacaagtcactc

SEQ ID NO: 204 - DNA, 91 bp, Homo sapiens
gggactctggctgaatccttcttcaagaattaccctcaacccaagatagcagttcttgatccaccccaggatcatgacctcttcctggaag

SEQ ID NO: 205 - DNA, 31 bp, Homo sapiens
tggaggtggaatcctttaagattatgtccag

SEQ ID NO: 206 - DNA, 574 bp, Homo sapiens
gtctccctcttctcattgaactgcaaaattcctgaaggatgagacctgggatgtttaatgcaaactgtacattctcagcagagcacaagtatcaaagggacattggatatattttaataatgatctaacacaagcaaaaataaccactgaaaatataaaactcaacaagagacataagaaaaaagcagacagaaaacaaaaaaaattcttattttagaatgatgctatatgtaacttgtaaaatatttaagtttttatacatgagattatattggtttccttatttaaagaaaaaaattacaattaagaatggaaattaaaatgtaaaaccaagataaatatttttggcttttggactaaaaaaaaaaagattagtgaggaaaaccaataaataatggggagagaaagaaatgctactaattttctcttcatgattaaaaaaaaaatcttttaaccatatttggggtgactggaaattaacagtgatgattttgagctaaaaaatcagtgacacattttattaaaacaaaaggaacaaaggaactttaaccagaccatacatgtaaaggctgtt

SEQ ID NO: 207 - DNA, 424 bp, Homo sapiens
tataggacagacagcctggaatgtgtgggtgccagtgaagacctcatgaggagcgaagtgatgaaggggttggatgtggtgtgggaggagttaaagccagggatctgtgggctttgtggctctggattaagagaaaactgcacatctgaggaaaagaagaaaattcaacatcattttaaagggtcttgtaaactatgatcagagctgtgttgggaaaaatctttgattgattttcaatcctggctgcacaacagaaccattgtatggcgcttttaatagtagagggaactgggccccactccaagcttgtttaattactatgaccagagtgtgtggctcagatatatatctatatctatagatctatctatatatatgtctatatgtatgtgtatgtatatatttaaggctccataggtgattc

SEQ ID NO: 208 - DNA, 88 bp, Homo sapiens
gaagaactagagacctttatgcttaaacatggagaaaatattattgatactttaggagctgaagtagatagacttgagaaggaactga

SEQ ID NO: 209 - DNA, 203 bp, Homo sapiens
ctcctctgcccacggaatgagcatgaaacccaaaatggacaggtagactgccctgggattttaaacactggaggaagagaagtgagggctttgtctccagtgccaaagaagatgtggggccctggaactttgagcaaccagcgctccagtcttgtggagaaatcaggtttgagaaaaagagccgcttataggaaaaacagctg

SEQ ID NO: 210 - DNA, 48 bp, Homo sapiens
ttttgcttagcataaggtttttggcagttttggatcaataaattttta

SEQ ID NO: 211 - DNA, 51 bp, Homo sapiens
gagacatatgaagcaaaaaggaatgaattcctgggagaactgcagaagaaa

SEQ ID NO: 212 - DNA, 38 bp, Homo sapiens
aatgggacgacatgaactcaaggaggctatttatgacc

SEQ ID NO: 213 - DNA, 160 bp, Homo sapiens
tctctctatctgaagggcctcctgtactcttttcatttcctctttttccttaatcataatttgtatttcttcttgactttcttgaagtctgttggtcaactcgagcatcttactttctatactttgtagtgctgaatccttggctttgcgatgctccttg

SEQ ID NO: 214 - DNA, 34 bp, Homo sapiens
agaaaggcattatgattccattgccgaaaaacaa

SEQ ID NO: 215 - DNA, 247 bp, Homo sapiens
gcatgtaggacaactcagttagaaaagtatagtgaatggatggaatctactgtatgataaaaatgctacaagcaccatttagttgccatcaataagaaatttacttgttttaaaaaaatccaaatgctggcattgtccagaaaaatttaacaggtttatttataattattataaagttgaaccgctgaaacttgttcactgaaacattttaacttgcattaatgctttacgtctccgcatttatatt

SEQ ID NO: 216 - DNA, 195 bp, Homo sapiens
atagacttaactcctgtgcaagatactcctgtggcttcaagaaaagaagatacatatgttcattttaatgtggacattgagctccagaagcatgttgaaaaattaatcaaaggtgcagctatcttctttgaattcaaacactacaagcctaaaaaaaggtttaccagcaccaagtgttttgctttcatggagatg

SEQ ID NO: 217 - DNA, 25 bp, Homo sapiens
tgggaaccgctgagccctctgacag

SEQ ID NO: 218 - DNA, 32 bp, Homo sapiens
attcacacggagcagcgtggtcctcagtgccc

SEQ ID NO: 219 - DNA, 81 bp, Homo sapiens
cttcatcacacgacatacgagcattttcgcttaaagttaagcattgctataaagccggggagcaacaaaatctgcaaaagc

SEQ ID NO: 220 - DNA, 66 bp, Homo sapiens
tattcaggaaccaacactgggaatgtgcatattattgctgatttatatgcagaggtgataggggtt

SEQ ID NO: 221 - DNA, 542 bp, Homo sapiens
ctgccaaaaccagtcagagtgaagaaggtatatagtacatgctaaaatacgggataaggagggttacacgtcaagggctgtggagaaaaagtacttattaattgtccaactgggagatgattgcacagcgaggaaaatgtagtgaaaatacccaattcaagaaagagccttgattcagaaacagtacagttacagcagggagtagaatgtcgatcattcgtacttctggtgcatatttatgattgaagcaaaattggctggaaatatattaggtatattaggtgtccatgcaattatggattagagagcctagttatttttaattagggacattaccaaaaaacatcaaaggggttgaacttatcgttctatttagtatgttattatatgtaacaaaaggaacctattttaatttcaaaatttgaacaaatgatggcaattatggaagtacagaatatcaatggtgcctgtgtgtgttgtgttggtgtgtgtgtgtgtgtgtgtgtgtgttctgcttttgtgaggcacaaccaaggcaatta

SEQ ID NO: 222 - DNA, 84 bp, Homo sapiens
tcttggacttgggatatacagttccagtttattagcagcaactgctagggaaatgattttggtgttttgggttaattgcttcta

SEQ ID NO: 223 - DNA, 695 bp, Homo sapiens
ttggagataggtagacttggtcctcggtttgaatcctaattctgtgttttctctttgtatatatttgggcaagttattgaagttacagctttcctctccttatatttaaaatgaggataaacatagtgattgcacagagttattatgaaaataaaagagctatagagacagagcatgtggcatctggcagcaccaataaagggtggttcctgttaatagtgttaacaacacacagatgagccttagctctggttcagtttctgaaggtactattatttgttgtgatggtggttgtgtgccttaatttaccagatcaaaatgtgatttagatagagctttgagggcttcgttttcattattaacaatgtagaaaatatacttgtcaggactttaattcattttaaatgtataataagtcatgaattattcacacaaaagaataatggcatgctattgaaaagaactctaatgttcttttctggaataaggtgaatataattgcatatagacatatggccatatttatccatggaaaggcatagaagcaaaaacctggtggaaacattgtaatatgtattttttatcattttataagaattgacattttgacaaggacctacagttaatccaatttatttcccagatgaataaacttagtatacttctatctcctcttctacaattgggttatctga

SEQ ID NO: 224 - DNA, 87 bp, Homo sapiens
gggacagtatatccagccgatcttctcagcaccaaggatgaagagattaccaagacatttgaggtcaacatcctaggacatttttgg

SEQ ID NO: 225 - DNA, 355 bp, Homo sapiens
atcagcagttggatgttcttgcctctgacagtagcttatttgctctgggggccaggaattggattcagtttacactatcattaaaaaagagggagagagataataaactatattttggtggggatggtgattaaacacctcttttgggtatgccttttaaaaatgcttatagagaaaaaaaattttaaaaagaaagctaatgctagtatatactgcaatgttaggggaatgaacatgttttcctactgcattggggacttctagataggttaatgaaaggccttttattctgttactggacatgaaaactttgtctaatttcttactctattgtacgtttacagtcgcagcacta

SEQ ID NO: 226 - DNA, 27 bp, Homo sapiens
cacacagctaaagtcattcatcaactc

SEQ ID NO: 227 - DNA, 206 bp, Homo sapiens
tgctacttcaaccaggtaattaaggctaatatcagtgacaaaacacgttgatagcatgtagctatgatatgatatgatgtgatgcggatggcactatacctctgtggacttccttcctaaaacctgtaaccccattctaaccatgagaacatcagacaaatccaaattgaggggaattctacaaatgccctggtcagtactcttta

SEQ ID NO: 228 - DNA, 213 bp, Homo sapiens
gtgcagctcaatactagcttcagtataaaaactgtacagatttttgtatagctgataagattctctgtagagaaaatacttttaaaaaatgcaggttgtagctttttgatgggctactcatacagttagattttacagcttctgatgttgaatgttcctaaatatttaatggtttttttaatttcttgtgtatggtagcacagcaaacttgta

SEQ ID NO: 229 - DNA, 98 bp, Homo sapiens
ttcaggtatattcctccaaaacccacacagttcagagattttcaaacaccaggtttccatttgtattaaaatgggcaagataatgaaggcacaggctc

SEQ ID NO: 230 - DNA, 25 bp, Homo sapiens
tctaaatgttctccaagctattgta

SEQ ID NO: 231 - DNA, 113 bp, Homo sapiens
gaaacatgaggaatgctttagtgtaatgtgggagaacttttttgtaaatttaatgcaattgaaaaagttttcaaattcaattaagataactagaattggattatggtgtaaaa

SEQ ID NO: 232 - DNA, 83 bp, Homo sapiens
gagcccaaggacttttcttgtgaaaccgaggacttcaagactttgcactgtacttgggatcctgggacggacactgccttggg

SEQ ID NO: 233 - DNA, 147 bp, Homo sapiens
ggacagagcaaccactgtcaccctcaaagaagagaaaaaggaatatgagcttgccacccagaatgggcttcacaaagccacaaccatcatttttagcaacatgctgtcttccacactaattggacttttagctgaattggttaatta

SEQ ID NO: 234 - DNA, 900 bp, Homo sapiens
acaggggaatcgacccaagtatccatcaacagatgaattaataaagtatcatatatccacacaatggaaaattattcagccttgaaaagtaatgaaattctgatatatgccacaacatggatcttgaaagtattatgctaagtgaaacaagccagacacaaacaaaacaaatattatatgattccacttatatgaggtaccttgaataggccaatttataaagggataaagtagaacagaggttgccacaggctgcaggggcaggtggggagggtggagagggtagaaggaatagggagctattattaaatgggtacagagtttctatttgggatgatgaaaagttctggaaagggatacgtggtgatggttacacaacgttgtgaatgtgcttaatgttgctgaatcatacacttaaaaaactgtgaaaacggcaaattttgttatgtacattttattacaataagaaaggttaacaactcaacacttgaggctgggcacagtggctcacccctgtaatcccagtactttgggaggctgaggtgggtggatcacctgaggtcaagagttcaagaccagcctggctaacatggtgaaaccccatctctactaaaaatgcaaaaattagccaggtgtggtggcgtgcgcctgtaatcccagctactcaggaggctgggacatgagaatcgcttggactttggtagcagaggttgcagcgagctgagattgtgccactgcactccagcctgggcaacaagggcaaaactcagaccaaaaaaaaaacaaaacaaaaaaaaccgaccccaaaaagcaaaacaaaacaaaacaaaaaaaccgaagtgtcttcaccctgatctttgttaccatctctctgaagatcctagagccatgagttatttc

SEQ ID NO: 235 - DNA, 101 bp, Homo sapiens
tgcttttcggaacactaaaaccatatatattttaacttcaattttctttagcttttaccaacccaaatatatcaaaacgttttatgtgaatgtggcaataa

SEQ ID NO: 236 - DNA, 1546 bp, Homo sapiens
ggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaaatactatcatagatattgatcatactaaacctgtgtatgaagacattcttggaatgcaaacagatatagatacagaggtaccatcagaaccacatgacagtaatgatgaaagtaatgatgacagcactcaagttcaagagatctatgaggcagctgtcaacctttctttaactgaggaaacatttgagggctctgctgatgttctggctagctacactcaggcaacacatgatgaatcaatgacttatgaagatagaagccaactagatcacatgggctttcacttcacaactgggatccctgctcctagcacagaaacagaattagacgttttacttcccacggcaacatccctgccaattcctcgtaagtctgccacagttattccagagattgaaggaataaaagctgaagcaaaagccctggatgacatgtttgaatcaagcactttgtctgatggtcaagctattgcagaccaaagtgaaataataccaacattgggccaatttgaaaggactcaggaggagtatgaagacaaaaaacatgctggtccttcttttcagccagaattctcttcaggagctgaggaggcattagtagaccatactccctatctaagtattgctactacccaccttatggatcagagtgtaacagaggtgcctgatgtgatggaaggatccaatcccccatattacactgatacaacattagcagtttcaacatttgcgaagttgtcttctcagacaccatcatctcccctcactatctactcaggcagtgaagcctctggacacacagagatcccccagcccagtgctctgccaggaatagacgtcggctcatctgtaatgtccccacaggattcttttaaggaaattcatgtaaatattgaagcgactttcaaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaaccgatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatc

SEQ ID NO: 237 - DNA, 403 bp, Homo sapiens
atgggccattcgtcaccagggttaagtttgccagtggtaaatgtcgttcctgtaggttactccacagacagaacataattagtactgaggtcctgagagtggctgttagtatacatagattcagggataaaacttgttcgtgttttcagggtttctgtaggattattgcttcacaactggttgacagatggtggggttatgcattgaaattcaattctaaaaatgctgtccctttctttgttattcttttatatgacatctttggtagactgtgttttcatctgaaacatggcgttttaaaaaatgaaattgggcatgttcctttcggcagaattgtgcttaatagttggtgaggtttctatgggcgatgtggaaaacctacagcggcacagaaacgtattta

SEQ ID NO: 238 - DNA, 25 bp, Homo sapiens
atggaatatgagaaggaacctgctg

SEQ ID NO: 239 - DNA, 95 bp, Homo sapiens
ttggctctttcactttgccatgaggaggaacatataggcaccagctatagcagcttgctctgctgctgtattgcccggagaatatctgacactga

SEQ ID NO: 240 - DNA, 288 bp, Homo sapiens
tgcaattgacctatgacaaactgtgaacctgcagatttcacctattttgatttactataagagctgggatttgattcattttatttatgcctaagtcatctatgcattaacatgtcatattcttaactttgatctaatgctttttactaggaaattttaatactgaaggactattttattatttttttctaaagatgtttgtcactagtttttcattattaaatgctgaggccaataccaagaagtttattttctatattatacaattatgaattacatgctcagcta

SEQ ID NO: 241 - DNA, 86 bp, Homo sapiens
ttccatacttatgatagatgaaatcaatgatatccgaattattggagccattacagtcgtgattcttttaggtatctcagtagctg

SEQ ID NO: 242 - DNA, 46 bp, Homo sapiens
tgccttcggattcagacaagaagcttcctgaaatggacattgattg

SEQ ID NO: 243 - DNA, 182 bp, Homo sapiens
gcgcatggtcatcttagctttcgaaagaggactgcactgtttaacattgaagaattacatggggaatcacaaatatattgctttagtactgcatgttctgttgtggtgagggaaagaaacatgctttgaaggttttcccttgtcaacagaatgtgtgtctgtagctgtgtattgcgcatgta

SEQ ID NO: 244 - DNA, 198 bp, Homo sapiens
tggggaaccaacattctcaggaagaacaagtgcctattagaggatacgctcctgagggttcttaggtggcagaggatcaatgctttcactgtgtttatactttttccttgatgatctctctaatgcaaaaaaaggctcccatccccactaatctcccacatgaatgatacatacccagtccatctatctgtgtgcagg

SEQ ID NO: 245 - DNA, 467 bp, Homo sapiens
cctgtgagatccgaccatcccattaactttgaagtttctcttgattaatagaagaaaaaaggggagggtgaagaaaaggaggaacatgctaaaaaccttatgacaatcatccaaatgtgaggaaagaacaaccgattcaccaactccactttttctattttacaactttctacatctcactcttgattttggccttcctggctgaaacagcctggcagtccctagagcccctgagaagagccctggttctccaaaagacagaggaggagaagccctgcaggatgcgctgaccacttcccagagaactgacagtccgtgctcccaaaagtttgaaccaacagcctaatgtgaaaagaaactgcactgaaaggtaaaggaggaaatggtgatgaactgggcttatgtgagaatgtctatattttcataacacagccccagaatctttctctcagttacagcctcaggca

SEQ ID NO: 246 - DNA, 31 bp, Homo sapiens
tcttagtgcttcagagtttgtgtgtatttgt

SEQ ID NO: 247 - DNA, 186 bp, Homo sapiens
tggggcccattttgtacaagcgataactattgaagaaaagagaagaatacagttcttaaccctttccttatgcctcacaaattcagcttgatttaaaagcctactaggaccataattttaaaacaaaaagcttattaggaatatggtgtctggaatttgctccacaattgcaggagtagttgcagt

SEQ ID NO: 248 - DNA, 98 bp, Homo sapiens
tacccgatggatttgtgaggattaaattaagatatgtatacaataagacagggaagtccgacatttagtaactgctcgatgatcattgcggtacgtta

SEQ ID NO: 249 - DNA, 107 bp, Homo sapiens
gcttccatattgttgtccacacacaaaaaaatcgaaacatgatatctttttgtcaaacctgtagcacgcgaccacagtggtagccaggccaattcatttgcttcacc

SEQ ID NO: 250 - DNA, 148 bp, Homo sapiens
tcacagggcacactcgctagagatagggatccatgtttggtgtttcctctctgcttaacaacatctgctcctgcagctcttgagttacttgccataggaaaagcccaggactggccctgctgggtcccaccaatcacccggggagtga

SEQ ID NO: 251 - DNA, 31 bp, Homo sapiens
tctgatggaagatgtaactgcaaacaaaaga

SEQ ID NO: 252 - DNA, 35 bp, Homo sapiens
attgtggacagcatctctaaagacaaagccattgc

SEQ ID NO: 253 - DNA, 113 bp, Homo sapiens
tcctacaacacatcaaatctgatggaagatctgaaggttttgtatcgaacagctggtcagcaaggcaaaggaatcacttttattttcacagacaatgagattaaagatgagtc

SEQ ID NO: 254 - DNA, 103 bp, Homo sapiens
gtgctattaattggtgaacaaggaacagccaaaacagtaataattaaaggatttatgtcaaaatatgatcctgaatgtcacatgatcaagagtctgaattttt

SEQ ID NO: 255 - DNA, 36 bp, Homo sapiens
tttgatgaatttaaccgtattgatctaccagttctc

SEQ ID NO: 256 - DNA, 85 bp, Homo sapiens
gaagaagcccgcgagttactctctcatttcaaccatcagaacatggatgctcttctgaaagttacaaggaatacactagaggcca

SEQ ID NO: 257 - DNA, 123 bp, Homo sapiens
gtgaaccttcgaaagtgtgacatacttgaactgaaaaccctaaaggaacctacggactacttgactctagcaaataaccctgagactttgggaaaaatagaggattgcatgaaagtatggatc

SEQ ID NO: 258 - DNA, 131 bp, Homo sapiens
cattagtgcctcatgactgtgtttgatgtcctttattgatacaaagtgagcctgtgccttcattatcttgcccattttaatacaaatggaaacctggtgtttgaaaatctctgaactgtgtgggttttgga

SEQ ID NO: 259 - DNA, 451 bp, Homo sapiens
caagtccctgctatacagtggtataatatttgcatagaacctgcacacatctttccttaccctttaaatcatctccaggttacagatacctagcacaacataaatgctgcataaatagctgttataccgtgtgtgtctgtgttttaaacattgtttatttgagacagggtctcactctgtcaccaggctacagtgcagaggcacagtcacagctcattgcagcctcaacctcctgggctcaagcagtcctccacctcagcctcccaaagtgctaggatgacaggtgtgagccactgtgccccaccaaaaaaagttttttcaaatattttctatctgtggttggattcagaccacatgaacactgagggctgactgtagttttgaatgtctgttactgaggaggcaccagcataaagtattttatcacttcagacgctgacaatctagtatt

SEQ ID NO: 260 - DNA, 33 bp, Homo sapiens
gctaatgaattagtcgctaatgctggtgacagc

SEQ ID NO: 261 - DNA, 68 bp, Homo sapiens
caaggtcatgtaccctaatagttactatgttttgtaaatccattttgtagagggcatgtaaataaatg

SEQ ID NO: 262 - DNA, 78 bp, Homo sapiens
tcccttggctacaaccgtgtcctggaaatccacaaggaacatctttctcctgtggtgacggcatatttccagaaaccc

SEQ ID NO: 263 - DNA, 350 bp, Homo sapiens
tgacccaagtttttacctagtctgactagaagtattccacttcaaggtctgaagtaggacttttaccttaaaaaacaacaacaaacaaaactatcacacaggatagataagaagattggttaaacagttttgtgtagatctttttggtgctgaactatgacatgagccttatagattgtaaaatagggatagttggaactaatgtacagaactaaattttttaaactttatttgctgttaaattctgtgaagtttcagttatctaaaataaatatacacaaatatgaaatataatgtttcagattgcaaggtaatatgtaatagtagtgtttgtaagatactcttgtcta

SEQ ID NO: 264 - DNA, 127 bp, Homo sapiens
atcagatgatcttataatagggagattatcctggattatcctggtgggcccaattcaatattttaaaaggtaggcaggaaaagagagggaattgtgactaaggaaactaaggggcagtagtcagagg

SEQ ID NO: 265 - DNA, 69 bp, Homo sapiens
gaatccctagatccaactttctttggtgagtttgcaggggtttctgatggttctcttgtttcacgggtt

SEQ ID NO: 266 - DNA, 48 bp, Homo sapiens
gtcttgagtaccacttcagctcaagcccttgctcatattctgaaacag

SEQ ID NO: 267 - DNA, 107 bp, Homo sapiens
tttgcattaaatcctggggtccattttacaatccattatttttgaccactgctatgtgttcaagtagtatgagaatgtgattgtttttatctggttacatatatatt

SEQ ID NO: 268 - DNA, 176 bp, Homo sapiens
ccagagccctaggtctatatccgtatgaaatgcattgatcggtaaccttatgagttagtgttatgaaagccattaaaaagtagacctggttgagttaattattagcactttgacccctaaaaaatgttcctggttacagaaaagtcctccattcaggcctttcttgtaagtgaaga

SEQ ID NO: 269 - DNA, 59 bp, Homo sapiens
aatggtttgccgctggagtatcaagagaagttaaaagcaatagaaccaaatgactatac

SEQ ID NO: 270 - DNA, 117 bp, Homo sapiens
cctccagggaacaaactcacgatgcaattgctagtcagcagccaacactttctcccagcagtagccctaagcaggctgcacatgctcaacctactccaggcccaactataactactg

SEQ ID NO: 271 - DNA, 75 bp, Homo sapiens
aagcagagaaataatggccttgagctgaaggattgtgatcacggaaattatcaacgtaaaagtcgacatgggctc

SEQ ID NO: 272 - DNA, 112 bp, Homo sapiens
catctggtgagggacttcgtcctacatcctcacatattggaaggctgaagagcaagagaggaactctctccaccaagcctttttataagggcacctgatctcattcattagg

SEQ ID NO: 273 - DNA, 27 bp, Homo sapiens
ttctcttctttaggcaatgattaagtt

SEQ ID NO: 274 - DNA, 265 bp, Homo sapiens
tcctgctggcacataaaatagtggtatgtcttataactgatgagacagtgaccttattctgataaggagtgccatgaaaactctaacgggtcttcagcttcttgttctacatttagcctatcctgtgagaatgcttcaggcccttcttttaaaagtctacataatgttgcaggaaatgttggttagcttcaggagagtgtaataatagtagctgagcctgattcattttatatagcagcaaagagcttcccaccattcaggtgta

SEQ ID NO: 275 - DNA, 60 bp, Homo sapiens
ggaatcaatcaatgtccatttcaggaagcttcttgtctgaatccgaaggcacagctgtgt

SEQ ID NO: 276 - DNA, 429 bp, Homo sapiens
tcctgtgatggcgagacaaatgatccttaaagaaggtgtggggtctttcccaacctgaggatttctgaaaggttcacaggttcaatatttaatgcttcagaagcatgtgaggttcccaacactgtcagcaaaaaccttaggagaaaacttaaaaatatatgaatacatgcgcaatacacagctacagacacacattctgttgacaagggaaaaccttcaaagcatgtttctttccctcaccacaacagaacatgcagtactaaagcaatatatttgtgattccccatgtaattcttcaatgttaaacagtgcagtcctctttcgaaagctaagatgaccatgcgccctttcctctgtacatatacccttaagaacgccccctccacacactgccccccagtatatgccgcattgtactgctgtgttata

SEQ ID NO: 277 - DNA, 72 bp, Homo sapiens
acagtgaggtctcaagacaattgccagtgtggctggcagtggaaggcagtcaccttgacgaggggaggtgga

SEQ ID NO: 278 - DNA, 27 bp, Homo sapiens
ctgagcattgcccatttcttttgcttt

SEQ ID NO: 279 - DNA, 100 bp, Homo sapiens
gtacccttcaacctaaatagccacatgctgctagaaatacttcagcttctggattgccacgaccacccctacgcgtcttcagttggcaacacagtgacca

SEQ ID NO: 280 - DNA, 219 bp, Homo sapiens
tggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtg

SEQ ID NO: 281 - DNA, 103 bp, Homo sapiens
gctgttggttcaaacttttgggagcacggactgtcagttctctgggaagtggtcagcgcatcctgcagggcttctcctcctctgtcttttggagaaccagggc

SEQ ID NO: 282 - DNA, 265 bp, Homo sapiens
ccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgg

SEQ ID NO: 283 - DNA, 27 bp, Homo sapiens
ctggacatatgctacacaatgaaaagt

SEQ ID NO: 284 - DNA, 33 bp, Homo sapiens
ggagtgcttcctcacagttaccaagaataaaga

SEQ ID NO: 285 - DNA, 125 bp, Homo sapiens
cttggaatacaagactcgtgatgcaaagctgaagttgtgtgtacaagactcttgacagttgtgcttctctaggaggttgggtttttttaaaaaaagaattatctgtgaaccatacgtgattaata

SEQ ID NO: 286 - DNA, 97 bp, Homo sapiens
cgcacccagccaacaatctggtattcttaaggcaaaacagtagaaatcactttaaatagacactttattagatccacaggcgataaaatcccagagc

SEQ ID NO: 287 - DNA, 1872 bp, Homo sapiens
ctccacgcaggcgaattcccgtttggggcttttttttcctccctcttttccccttgccccctctgcagccggaggaggagatgttgaggggaggaggccagccagtgtgaccggcgctaggaaatgacccgagaaccccgttggaagcgcagcagcgggagctaggggcgggggcggaggaggacacgaactggaagggggttcacggtcaaactgaaatggatttgcacgttggggagctggcggcggcggctgctgggcctccgccttcttttctacgtgaaatcagtgaggtgagacttcccagaccccggaggcgtggaggagaggagactgtttgatgtggtacaggggcagtcagtggagggcgagtggtttcggaaaaaaaaaaagaaaaaaagaaaaaaaaagaaaaaaaaaagatttttttcttctcttaatcggaatcgtgatggtgttggattatttcaatggtggggttaatatagcatgttatcctgtctatcttttaaagatttctgtataagactgttgagcagtttttaaaatagtgtaggataatataaaaagcagatagatggcgctatgtttgattcctacaacgaaattatcaccagctttttttcattcttaactctttaaaggattcaaacgcaactcaaatctgtgctggactttaaaaaaacaattcaggaccaaattttttctcagtgtgtgtgtttattccttataggtgtaaatgagaagacgtgtttttttccttcaccgatgctccatcctcgtatttctttttccttgtaaatgtaatcagatgccattttatatgtggacgtatttatactggccaaacatattttttcttttgtccctttttttctttcctttctttttacttcctttatttctttattccttccttttcctttttttcttttttttttctttttttttttttttttttggtagttgttgttacccacgccattttacgtctccttcactgaagggctagagttttaacttttaattttttatatttaaatgtagacttttgacacttttaaaaaacaaaaaaagacaagagagatgaaaacgtttgattattttctcagtgtatttttgtaaaaaatatataaagggggtgttaatcggtgtaaatcgctgtttggatttcctgattttataacagggcggctggttaatatctcacacagtttaaaaaatcagcccctaatttctccatgtttacacttcaatctgcaggcttcttaaagtgacagtatcccttaacctgccaccagtgtccaccctccggcccccgtcttgtaaaaaggggaggagaattagccaaacactgtaagcttttaagaaaaacaaagttttaaacgaaatactgctctgtccagaggctttaaaactggtgcaattacagcaaaaagggattctgtagctttaacttgtaaaccacatcttttttgcactttttttataagcaaaaacgtgccgtttaaaccactggatctatctaaatgccgatttgagttcgcgacactatgtactgcgtttttcattcttgtatttgactatttaatcctttctacttgtcgctaaatataattgttttagtcttatggcatgatgatagcatatgtgttcaggtttatagctgttgtgtttaaaaattgaaaaaagtggaaaacatctttgtacatttaagtctgtattataataagcaaaaagattgtgtgtatgtatgtttaatataacatgacaggcactaggacgtctgcctttttaaggcagttccgttaagggtttttgtttttaaacttttttttgccatccatcctgtgcaatatgccgtgtagaata

SEQ ID NO: 288 - DNA, 294 bp, Homo sapiens
tctaagctgagcacgtagtctgttcagagcttgtttcttccgggagtttgggtcccccattttgaaatacaggtggtactaaagcctttggaaattgtcactaaactatgggcactttttcttaagactcaagtacaacagaaacaagtcattttttttcctgctaatatgattgattagcgaaaatcacgactataacccaaaaactgcaccttctgtcaatattagcagactgtcatattacagggtcaagaaacaaaagctgctgtccagtcatgtttggacaataacgtt

SEQ ID NO: 289 - DNA, 647 bp, Homo sapiens
gcactgacattttagccaccttctaatgaatgggcttgaagcagaactgcttctactatcaggtaatggttgaggggggatgtctattacacatgtactttgttttgtgtaaagtatgttctggaaagttacattcttttggtgagtacatgtaagtatttgagggatattcatgatttaaggaggcctgaaatgaatctttttgattagggaaagaaatattttgcagtgtaaatgattaaccatttcttccagtttacctgtttggtgagttcataccataaatgttacgatgtttggttttataaaagagtacctgtaagtttttagtttctgtgtggaagattttttcagagagaatacagatagccttcttgtattagaattttgtcatatgtggtttgaaactgttcattggagctgaggaattttgttacgtttgttttaactagatatgattaacaaaatgaccatagcaagagcatgcattgaaatgagagaagttacgtgtgtttgtgtctagtttgggtaatatgattcattcttaagaattcttcaaactttattataatttttggaaataacaagtttgtcagttgattaaaccagtgtcttgttcataggcgaccttacgcaattatatagca

SEQ ID NO: 290 - DNA, 111 bp, Homo sapiens
tgccgaagtttacctccactagttctttgtagcagagtacataactacataatgccaactctggaatcaaatttccttgtttgaatcctgggaccctattgcattaaagta

SEQ ID NO: 291 - DNA, 50 bp, Homo sapiens
gtgaataggattttctcagttgtcagccatgacttatgtttattactaaa

SEQ ID NO: 292 - DNA, 149 bp, Homo sapiens
gccagcaattatcccatgggccctacttgaatttatctgaggcagctacagattgccctgcaagatgagtttttggagataaatgaaataactggacacacactcacacaagtaacaccacagcagacctcggagtactgctaagtgta

SEQ ID NO: 293 - DNA, 79 bp, Homo sapiens
atgggcaaaaaactcgaggactgtatttgtgactaattgtataacaggttattttagtttctgttctgtggaaagtgta

SEQ ID NO: 294 - DNA, 74 bp, Homo sapiens
gtgtgtccctatgtctatgtatcgggtgaggggtgggagggttgctggagggtgctttattgggtggagggcac

SEQ ID NO: 295 - DNA, 304 bp, Homo sapiens
gcacggttggcatggcctttccaaaggtcttccactagagtctagagaaatctaaatatagtcatccacaaactggatgtttttattttctgagccattagagattttcaaaatcactttgatttttaaaaactcatcaaatgtgaatcatggcggggaagaccactgagctgatttctgataactaagttatcactgaacataatttatcatatatggctactggcatcatgaagaccttgggatagggaagactctttatgagaaatataaacatcacttgtgtaggaatcaccaggtgtcc

SEQ ID NO: 296 - DNA, 219 bp, Homo sapiens
ggcacctttgtcagcgcttcctttctcactggagtttaatttaattgagaacttcaggcctcattaagggctgttagcgcatttttaaggctctttgatgagctctgaagccctaatggggtagctaagtataccagggcactgggaactgaagttcatgtgctattaatcatttgtcagaccagagtatttatctgcggtgctggaaaacaaggtatt

SEQ ID NO: 297 - DNA, 761 bp, Homo sapiens
ggtgagctaagcacactttgacagcacactattgaatgttatagtttctgtattgaaatatgtaaagacatctgcaaattagtacctagcaatgaagacatacatttataaatatacacattctaggtttgataaggtaaatgtaaacagatgccatgactccttttcaaacagaaaacccacaagactaatagagaaccaataggctccctatagtacgaatgtgcaaaattaaagcatggtaaactgatatttacataaatatcaaaccaacaattagtttatacattgtcaatgaccttctaagatatgtcatgagtggttccaagaatatctttcccccaatggagaaggtattcagaggctaaattccgacactttaaaatgacacacatcataggctttacctgtttgaccactgcctcaaatgtgtgagatgtgattttatgataagcagtctattatttttaaacaagaggttctaaacacatctttagattctaagcagaaagaaattacaagtactattagatttacattaaacaagttacctctatcaaatttctcgattctcttatgctgtagctatatttgattttaccttaaaaagttctaaggtcctttccccccctttgttaattttggtaaactaggcacattttacaaggaaatctgtgtcaaaacagataacagtgcataaatcaatccaaatttagaccctggcaaccagttccccattgctcatctacgggactctgtca

SEQ ID NO: 298 - DNA, 308 bp, Homo sapiens
ttcaataattgtgactggcctcaggaattctcccttccacacctgcccacctcacctcaccgcaccgcaccgcaccgcaccgcaccagagccagagcagctgcttgtctgcagcaggacacagttcctacatacgtttcagttctttcatggtaagctcaatggactttgaattgtttacagtgctgtatgtccaattgttaaatgtaccattctgaacgatgttaaagcaagtgtggtttatttatggcatgaaccatgtaacttgaaatatgaacttacaaggaggggcactcattaggtaacaag

SEQ ID NO: 299 - DNA, 143 bp, Homo sapiens
ctttgtcaatctatggacatgcccatatatgaaggagatgggtgggtcaaaaagggatatcaaatgaagtgataggggtcacaatggggaaattgaagtggtgcataacattgccaaaatagtgtgccactagaaatggtgta

SEQ ID NO: 300 - DNA, 438 bp, Homo sapiens
ctcatgttagctgtaccagtcagtgattaagtagaactacaagttgtataggctttattgtttattgctggtttatgaccttaataaagtgtaattatgtattaccagcagggtgtttttaactgtgactattgtataaaaacaaatcttgatatccagaagcacatgaagtttgcaactttccaccctgcccatttttgtaaaactgcagtcatcttggaccttttaaaacacaaattttaaactcaaccaagctgtgataagtggaatggttactgtttatactgtggtatgtttttgattacagcagataatgctttcttttccagtcgtctttgagaataaaggaaaaaaaaatcttcagatgcaatggttttgtgtagcatcttgtctatcatgttttgtaaatactggagaagctttgaccaatttgactta

SEQ ID NO: 301 - DNA, 611 bp, Homo sapiens
tgatgtcagttcatgggcctttagcatagttttaagcatcatttttttttttttttttgaaagtgtgttagcatcttgttactcaaaggataagacagacaataatacttcactgaatattaataatctttactagtttacctcctctgctctttgccacccgataactggatatcttttccttcaaaggaccctaaactgattgaaatttaagatatgtatcaaaaacattatttcatttaatgcacatctgttttgctgtttttgagcagtgtgcagtttagggttcatgataaatcattgaaccacatgtgtaacaactgaatgccaaatcttaaactcattagaaaaataacaaattaggttttgacacgcattcttaattggaataatggatcaaaaatagtggttcatgaccttaccaaacacccttgctactaataaaatcaaataacacttagaagggtatgtatttttagttagggtttcttgatcttggaggatgtttgaaagttaaaaattgaatttggtaaccaaaggactgatttatgggtctttcctatcttaaccaacgttttcttagttacctagatggccaagtacagtgcctg

SEQ ID NO: 302 - DNA, 40 bp, Homo sapiens
cactgcaggctacaacagtcctgtcaaattgcttaataga

SEQ ID NO: 303 - DNA, 306 bp, Homo sapiens
gtccttggccttctagccctgttcagtaagaatcctgccaagccagatgagcagaagccccgtcacccccaaccactgatatctaaccaagttcctttagtaatttttcattcactcccttacccttcccattggctataaacccccagctgcttcgtctgtacccagaattgtgttccctctggcaacagacttgacccctattgtaatcgtcttaaataaagtcttccttgtttgtttaactctgtgcagtgcagtttttctttgccatagcccaaacccacaccatatcggaaagaggtcttc

SEQ ID NO: 304 - DNA, 26 bp, Homo sapiens
ccaccaccagatgagaagttaagcag

SEQ ID NO: 305 - DNA, 112 bp, Homo sapiens
ttccggattcgcttatggtctccagggtcaaaggaaagggcgaaccaacgtttgaaggcagcagcagcttttttccagttgcccaaatggttactcaaaatgcaaggggaca

SEQ ID NO: 306 - DNA, 141 bp, Homo sapiens
tgacagaaatagtaggttcgaaacaaaggaggattgtgggaatcgtgattcaaatgttctttacccttggaatcataattctccctggaattgcctacttcatccccaactggcaaggaatccagttagccatcacgctgc

SEQ ID NO: 307 - DNA, 203 bp, Homo sapiens
aggctctctgaacatacaaacagtataactgttgttcactaaatggaaaaatcccaaaatcaaaaaccaatgcaaaacagtgaagtggcttgagctcctaggaggttaggtagaaattaaagagaatcagtggatgggtagaattttaagcagtaggtagttacccaatgtagaacgaggattagcttagacacctagtctgg

SEQ ID NO: 308 - DNA, 143 bp, Homo sapiens
tgtcattcaacaccgaaagatattgaggggaggatgttctcctacacctttaattgaaatggaaattcaatttaaaggtgctttaaagaatgagattgcagccttggagcaggctaggcagacttgtgggatgcctcatcaga

SEQ ID NO: 309 - DNA, 39 bp, Homo sapiens
aaatggaggaaattccgagatgacagtggttttggttct

SEQ ID NO: 310 - DNA, 48 bp, Homo sapiens
actaacctctgcagtttaaccttgagcgataccttttcccatgaatag

SEQ ID NO: 311 - DNA, 120 bp, Homo sapiens
agagtgtcgatactaggcaacaagcctctgaacagatagtgttacccggaacatcacccttttctccctttgcttcaaatcaaaaccagcatcccccatttagacagcataaaaggtatg

SEQ ID NO: 312 - DNA, 101 bp, Homo sapiens
actggaggaagggttgatccctacagtgaggagcccaaagagacaggcctaggagaaggctggtcccggagtagacaccaggacaagctttccactgtaag

SEQ ID NO: 313 - DNA, 49 bp, Homo sapiens
aggggcaggtaagtgagagtgtcagtgagcgggcgcagaggggactccc

SEQ ID NO: 314 - DNA, 202 bp, Homo sapiens
tatacaggggccgtggtgctactcagggtttgaggaagggagagaacctttgaagctgtggtaagggagagctggggcattgatctgggatgcagaggttgctgtggttgagagctactccagtgagcaacatgatggcttcagagtgagcaggccccatgggagagggcccagctgtgtcttcctggagcggtaacacctt

SEQ ID NO: 315 - DNA, 38 bp, Homo sapiens
ttgttacttggattgagggagtgatgagaaataattaa

SEQ ID NO: 316 - DNA, 285 bp, Homo sapiens
acgcttcgttggtctcgggaatacagctccacacgcaaaaaagtaaaaagtgcagcaaaacaacaacacaacgatcaacctcaaaggaaacaacaaaattaattttatcaaaatgcaatgtgtacattaagactaaagttatggattgttcctgtttggcatagaaatgtgatgactattaacagaaaggggaaaaagatttgccccctatccatcatcagacagacagacttctcttactaaaccccttatgtggagtgggatgagtgactatttcctgcagaa

SEQ ID NO: 317 - DNA, 362 bp, Homo sapiens
tggcttatgcacagtattcccttcaactccatatacacataaatacaataagtaattacattttatatgtaaacgtcattctttttaaacaaacaagaaaaagcaatgtaatggcatgcccaattttcactccacataaagtctcatatatcaccaaactggtttcctctagtgggttagatgttcatctctgagttccattgatatttatcctctgaaggcacttgggtttggggttgctggcaggaggtgaagaaccatcagagttaaggtcaagggacaggaggtgctgctagagagagaagccacaaggaccacaatgaactgaggtgtctagggaccatggcatgcacagggcattg

SEQ ID NO: 318 - DNA, 64 bp, Homo sapiens
catgtcgtgcttcgcagctcctagtgccggaaagggaaaagaaccggtgatcaatgaacttgcg

SEQ ID NO: 319 - DNA, 75 bp, Homo sapiens
aagagcatctgtgaggttcttgatttggagagatcaggtgtaaatagtgaactagtgaagaggatcttgaatttc

SEQ ID NO: 320 - DNA, 76 bp, Homo sapiens
cacaggcacaacaggcaaagccctacatgaagcccatactacatacctcatggttcctgttcagggacttctctga

SEQ ID NO: 321 - DNA, 319 bp, Homo sapiens
gtaggaatcaaacatagcgccatctatctgctttttatattatcctacactattttaaaaactgctcaacagtcttatacagaaatctttaaaagatagacaggataacatgctatattaaccccaccattgaaataatccaacaccatcacgattccgattaagagaagaaaaaaatcttttttttttctttttttttctttttttctttttttttttccgaaaccactcgccctccactgactgcccctgtaccacatcaaacagtctcctctcctccacgcctccggggtctgggaagtctcacctcactgatttc

SEQ ID NO: 322 - DNA, 217 bp, Homo sapiens
tcaacaatctgacaggcagtgaacttgacatgattagctggcatgattttttcttttttttcccccaaacattgtttttgtggccttgaattttaagacaaatattctacacggcatattgcacaggatggatggcaaaaaaaagtttaaaaacaaaaacccttaacggaactgccttaaaaaggcagacgtcctagtgcctgtcatgttatattaa

SEQ ID NO: 323 - DNA, 28 bp, Homo sapiens
tggggccaagacatcaagagtagagcag

SEQ ID NO: 324 - DNA, 41 bp, Homo sapiens
gacattgctaaaagagttaaaagtcattgctctggagaatt

SEQ ID NO: 325 - DNA, 34 bp, Homo sapiens
tgcctctgaactcacggctggtgttcccaataag

SEQ ID NO: 326 - DNA, 80 bp, Homo sapiens
gggcttgggcacaaatccccaggcaggctttggagttgtttccatggtgatggggccagatgtatagtattcagtatata

SEQ ID NO: 327 - DNA, 31 bp, Homo sapiens
gtattttgtgtttgcaagtctgccactctat

SEQ ID NO: 328 - DNA, 115 bp, Homo sapiens
tgattttagttagaagcacaagccatgatggatgtgggactcttctcaattgctctttaaattgtcactttttaaatcctattaaatgaagtgtcgattcctgacaaatacatta

SEQ ID NO: 329 - DNA, 79 bp, Homo sapiens
gccatgccaaccgtgcttgtactgctagaagcactttatgtttcctttttgggtgaaatggatttatgtgagtgcttta

SEQ ID NO: 330 - DNA, 402 bp, Homo sapiens
gacactgggctaggctgcaactttatctcatttaatactcccagctgtcatgtgagaaagaaagcaggctaggcatgtgaaatcactttcatggattattaatggatttaagagggcatcaatcagctcaactcaagatttcataatcatttttagtatttagattgtgcctcaaagttgtagtacctcacaatacctccactggtttcctgttgtaaaaaccttcagtgagtttgaccattgtgctcttggctcttgggctggagtaccgtggtgagggagtaaacactagaagtctttagtacaaaactgctctagggacacctggtgattcctacacaagtgatgtttatatttctcataaagagtcttccctatcccaaggtcttcatgatgccagta

SEQ ID NO: 331 - DNA, 61 bp, Homo sapiens
agatcctgatggctactggattgaaattttgaatcctaacaaaatggcaaccttaatgtag

SEQ ID NO: 332 - DNA, 37 bp, Homo sapiens
gaagaactgggagtcaaatttgtgaagaaacctgatg

SEQ ID NO: 333 - DNA, 51 bp, Homo sapiens
gatgatgagacccagagttaccacaatggcaattcagaccctcgaggattc

SEQ ID NO: 334 - DNA, 673 bp, Homo sapiens
tgtttttcaaggtgcgccgatttcatattgttcaaacacagttttacaatcaatttgtacagttaacacaattatctcagtggtcctgaggtgatgtacatcctcagcttacgaagataacaggattaagagattaaagacaggcataagaaattataaaagtattatttgggaactgataaatgtccatattaaaatgaaatcttcacaatttatgttcctctgccacagctccagctggtccctccattcggggtccctgacttcccacaacagttttaggatggtagaattgcaatgcagggcaggtactggagagttagttaaatgttattggttgcagttcaggttatctaagcagtggtggaagaggctaacatggtaggttggggccagattgtggagacagctttgagtgtcacatctaaggaagttagattttatatacatagtggggaatgagtcagggttttgaagtattctataaagtagattggagggatccaagtctggaggtgattaggccacttaaaaagctcttccagaaggtgagataagtgatatgagggtctcgtttgtaaagaagacagcagcaatggcacaaagtcgacttgttttttgatcatttgcatcttcattataaaggaagtccagagaatgtatggctatgtca

SEQ ID NO: 335 - DNA, 76 bp, Homo sapiens
gggaggtggtaagaacacctgacaacttctgaatattggacattttaaacacttacaaataaatccaagactgtca

SEQ ID NO: 336 - DNA, 466 bp, Homo sapiens
ttgcttcttccctacggatgacttctaaaaatatatgacgggtataaaaaaattagctatattgatcatatcaacactgtaactgctgaaatggcattctaatgtttgctttttattcggacaggccacatgatgcatagagcctctttcatgtgacttgtgtctactgcttaaatctttatgctgtgttgatgatattatattgacatatgaagctgtatatgtgtatgtattttgtggagaaagggattacaagatgtatgagtataatgacttgctaacctttcaggattcagagaaagatgaagaaagaccatatctaaataatacacttcatcattttcatgtgtataaatgcttaaagtaccatctttgttgaggtggttcatgtatccagtttatccagtacagttatttgtcaagcttagctttgatttcaaaggacacgcttaccttgtctggcata

SEQ ID NO: 337 - DNA, 111 bp, Homo sapiens
aaataactacatcctaatgaaaatcaagtttgatatgttcattttgaaagtagcgttggaagagttgctggggggttttttgaatccatagcactggttactttgaacaaa

SEQ ID NO: 338 - DNA, 312 bp, Homo sapiens
gtggtacggaccctttccatgatccccaaaagtttccttgtgtctcagtgaagtcagtcccctccccccactctgggctctggaaacccagatctgctttctgtcatgacagttttgctttttctagaatttcatagaaacagaatcaacagtgtctcacttggcgtcccgttttgtcataccttttatgctgtctaaatgggggatgctggttttgatttgaagcaaagggagaaaagggtgatgttccgggtaacactatctgttcagaggcttgttgcctagtatcgacactctctattcatgggaaaa

SEQ ID NO: 339 - DNA, 223 bp, Homo sapiens
gtgttgtggggtcaaccgtacaatggtgtgggagtgacgatgatgtgagtatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacctcattttta

SEQ ID NO: 340 - DNA, 564 bp, Homo sapiens
tcacgatgaagcatgctagaagctgtaacagaatacatagagaataatgaggagtttatgatggaaccttaaatatataatgttgccagcgattttagttcaatatttgttactgttatctatctgctgtatatggaattcttttaattcaaacgctgaaaagaatcagcatttagtcttgccaggcacacccaataatcagtcatgtgtaatatgcacaagtttgtttttgtttttgttttttttgttggttggtttgtttttttgctttaagttgcatgatctttctgcaggaaatagtcactcatcccactccacataaggggtttagtaagagaagtctgtctgtctgatgatggatagggggcaaatctttttcccctttctgttaatagtcatcacatttctatgccaaacaggaacaatccataactttagtcttaatgtacacattgcattttgataaaattaattttgttgtttcctttgaggttgatcgttgtgttgttgttttgctgcactttttacttttttgcgtgtggagctgtattcccgagaccaacg

SEQ ID NO: 341 - DNA, 206 bp, Homo sapiens
tgggcatgccattacattgctttttcttgtttgtttaaaaagaatgacgtttacatataaaatgtaattacttattgtatttatgtgtatatggagttgaagggaatactgtgcataagccattatgataaattaagcatgaaaaatattgctgaactacttttggtgcttaaagttgtcactattcttgaattagagttgctcta

SEQ ID NO: 342 - DNA, 48 bp, Homo sapiens
cctctgggcgcctgcgagtctgcggcccgcaggtgacggagctcacgg

SEQ ID NO: 343 - DNA, 148 bp, Homo sapiens
catggctgtatgtactgtcgctgtgtttttttgtttttttagaactgggtttgggggctgatttttatttctttgggggctttttttcttggcaaatactaaaaatctcgtcaatgtaatttctgtggtttctattcagcttgggttt

SEQ ID NO: 344 - DNA, 164 bp, Homo sapiens
gtttcagagactaggcatatggttaatatttaggtaggaaattcaggaaaaggagcttgtggggcaggaagggaaggaacggcagcttggggcactctgacatctttaacaagtcttgtaaaggcttactaaatacaacgaagcattgtaccaactatacccta

SEQ ID NO: 345 - DNA, 427 bp, Homo sapiens
gaggcgggatactttcagctttccatgtaactgtatgcataaagccaatgtagtccagtttctaagatcatgttccaagctaactgaatcccacttcaatacacactcatgaactcctgatggaacaataacaggcccaagcctgtggtatgatgtgcacacttgctagactcagaaaaaatactactctcataaatgggtgggagtattttggtgacaacctactttgcttggctgagtgaaggaatgatattcatatattcatttattccatggacatttagttagtgctttttatataccaggcatgatgctgagtgacactcttgtgtatatttccaaatttttgtacagtcgctgcacatatttgaaatcatatattaagactttccaaagatgaggtccctggtttttcatggcaacttga

SEQ ID NO: 346 - DNA, 40 bp, Homo sapiens
agattgtccagaattgattgaagcgtttcttaactctcag

SEQ ID NO: 347 - DNA, 110 bp, Homo sapiens
aaataactacatcctaatgaaaatcaagtttgatatgtttgttttgaaagtagcgttggaagagttgttgggggttttttgcatccatagcactggttactttgaacaaa

SEQ ID NO: 348 - DNA, 96 bp, Homo sapiens
tgtctgcagattactctcattaagctgatttttaaaaatctcagacagagcagagcaattcaccagcaccatcatcaagtgagctacaaatctatc

SEQ ID NO: 349 - DNA, 54 bp, Homo sapiens
ttcccgccaaattaaatttgacataatttgaaaacaaaacgctcacctcttgct

SEQ ID NO: 350 - DNA, 83 bp, Homo sapiens
aaattcgagcgagaaagatggaagacagcaaagaaaagaatggaaaaaagaagaggaaaagtttagccaagagaatcagagaa

SEQ ID NO: 351 - DNA, 116 bp, Homo sapiens
ggcgttgctgaatactgtccactaactgtacaaaatattgactgcatgcctcgcaaacaccaaaatatccgctggaatgccatagaaataaataacttctgctataaacacatgaa

SEQ ID NO: 352 - DNA, 80 bp, Homo sapiens
aacctagccaatcaacacacaactgtcaccacatgaaaaggtacttttatcaaacttcgagtctaagaacatacaaatgt

SEQ ID NO: 353 - DNA, 146 bp, Homo sapiens
gggctgctagccatggcctagcacagtgcagcacagggagcaccacacacatcctcagctgtgctcccagggatgcctcctgcacagtccagtgtgaacagctgcccagagccaagaagggcagtgttgctccacacgggcagttg

SEQ ID NO: 354 - DNA, 61 bp, Homo sapiens
ggagggagaggtgtcgcagacacccctggctccagtcttgcctcacgatccaacccctgca

SEQ ID NO: 355 - DNA, 27 bp, Homo sapiens
aagatggaaactgcgggaatgatcaga

SEQ ID NO: 356 - DNA, 195 bp, Homo sapiens
tgattgttgatattctcttttggttttattgttgtggttcattgaaaaaaaaagataatttttttttctgatccggggagctgtatccccagtagaaaaaacattttaatcactctaatataactctggatgaaacacacctttttttttaataagaaaagagaattaactgcttcagaaatgactaataaatga

SEQ ID NO: 357 - DNA, 540 bp, Homo sapiens
aactgctaaagatggtccgtgtgtgaaataattccttagagaaacacggagctggaaaaataatcactgattagaccttaaaaatagttcactgcataacatgacaaaaagcacaaaggctcattcagagaacatatttgttgttctccaacactgtaatagttataatttcaccatgacaaacaccagacattaagattaagctaacactggtgtttcttttgctcccccccttttaaaaacaaaatatataactgcatgtcactatagcaacatccaaaacagatcaatttgttacaatcactatttggtagagcaaactttacccccaaaaggaaaaattaaattaaaaaaaaaaaacctttaaaaaatttgaagacttaattttttttgtcataggaatacataacacctacagtataagttaatcaatttcaagctactgtatagaaatacaacactagcatgcacaatattgtatcaatagagtaatggaggagtaatcatttcactggagagacagtatcataggcaagtgca

SEQ ID NO: 358 - DNA, 879 bp, Homo sapiens
ctgtggtcatcgtggttcttctatcttcactgtcacctgtatcctgttacacatactcagttcctaattgtaagctcaattttggtattagcaaaagcatctgtcagtttttcctcaattactcacacctcttcttgcctaaataaaacaaagaaacaaagaaaacaagtgtggtgtcattacacgtctcgggagttcctcgtcactgactttatatatataaaaaaaagaatgcacatgcgggccacgttcacagatagacagattcacccgaaatgagaatgagggccttaaggctgccgaaaacaaatgggtggaaatagcaacgttgtttccgtcaattccaaatgtgcactggctgcgtgagacaagccaatctccaattcctatgcttttttcattaaaaagaaaaaactatctcgagaggaaaaagttctggtcaaataggagttcagtgtttgttatctggtaacagtttttttatcttcctacaattacttggggaaggagtgtcttcggggaggaaaaactgacacactgacaaggctcccgaaattggacagaatggttaaggatatagctcttccttttgggtttttctaccagtaggcttctagcacctgaattttcacacactgcaccacagaccttctccaaacgcctctccgaccccacagcttctgcacccagctgcgtccgccagcacgaggcgcagtaggaaacgccccagtgcagagtgggtgacggcagtgtgatccctgaggagggagctgcagcagcttcagaagcgggatgttctctgaaaataccaacacgatccatgacatacagacctgaattttctctatttttgtggcgtttcacgtttctctcaagagg

SEQ ID NO: 359 - DNA, 34 bp, Homo sapiens
gaatgtaagggccagaaagcttgaatgcccagaa

SEQ ID NO: 360 - DNA, 25 bp, Homo sapiens
tggtcgtatgactaagatgttttga

SEQ ID NO: 361 - DNA, 32 bp, Homo sapiens
ataaagttcctgataataaagtgactgaaaat

SEQ ID NO: 362 - DNA, 62 bp, Homo sapiens
aggtgattctgacgtgctgcccgccaggcctgccctgttcgctccctggtgcatggagccgg

SEQ ID NO: 363 - DNA, 757 bp, Homo sapiens
tgcgcaacggttcacacaagccttcctgaaattccactttacagtaaataaagctgtgcgtttccccttcccatgcacaactgcgtatcaatctacaactgtcatttaactgtgaaaaaatagagcgtctccccttttgtcatcgttctggtaacatttggagtagcatctgacagaacggagctgctcactcctggaccggttatttggttaaaacccaaaatgttaggtcgaaagaatcaatcatcacccaatacaaataaatattgcgttatgaaagagacgggcagagtcccacggtatccctttttaaagcggcatttccagcacagcagcgtggcgctcacagagaccagcagggcgcagctctgggatgccacatgggacacagctgcaagtacccgtaggcaccgtcccgccgcacgcgtccctccaccgagcgtgaggatgtggcgcagggcacgtgcactccgtctgcgacggggaccggttcgtcctgactgcagaatgaccccgtgttcaaaaacagccgctcccatttgcagaccacatgggccctggctgggctctcaggtccgagggccagcacctgctttggtgaccgcagctgtgccggacgcccggcaaggagcgtgatgtgcgcagccccccgaggacgggcctcctgtgctgcccatcagccctcccgaaaggacccagaacaaaggagtgggagagggtgacgccgcggtaaggcctgtgctttattgtgggtcaa

SEQ ID NO: 364 - DNA, 27 bp, Homo sapiens
tattgacacttgacaactggcctaaat

SEQ ID NO: 365 - DNA, 69 bp, Homo sapiens
gaatgagtttaagggtggttatgcttatcaaggattggggattggcaatgggtctagtggaagggtaaa

SEQ ID NO: 366 - DNA, 102 bp, Homo sapiens
ccagaagccacatccgcacaattttccacttaaccaggaaatatttctcctctaaatgcatgaaatcatgttggagatctctattgtaatctctattggaga

SEQ ID NO: 367 - DNA, 71 bp, Homo sapiens
gctgacctattgctgaggactatgagaaaaaagttattacagaatgagtcatatggaaaacacttgcaaac

SEQ ID NO: 368 - DNA, 63 bp, Homo sapiens
gccacgatgaattaggagaacaagatgtcaaattacactgatgctgagtcaagcttctcaaag

SEQ ID NO: 369 - DNA, 30 bp, Homo sapiens
tatgtgtgatcacaagactaaagataatta

SEQ ID NO: 370 - DNA, 31 bp, Homo sapiens
aatggtttgccgctggagtatcaagagaagt

SEQ ID NO: 371 - DNA, 85 bp, Homo sapiens
tagaagttaaagttgcaactcaagaaggaaaagaaataacctgtcgaagttatctgatgacaaattacgaaagtgctcccccatc

SEQ ID NO: 372 - DNA, 28 bp, Homo sapiens
tgttgttgcaatgttagtgatgttttaa

SEQ ID NO: 373 - DNA, 46 bp, Homo sapiens
aaataatgcttgttacaattcgacctaatatgtgcattgtaaaata

SEQ ID NO: 374 - DNA, 88 bp, Homo sapiens
gtttgccctttggtacagaaggtgagttaaagctggtggaaaaggcttattgcattgcattcagagtaacctgtgtgcatactctaga

SEQ ID NO: 375 - DNA, 121 bp, Homo sapiens
caggaacagcggagaacagttcaggacaagaagaaaacagccgggcgcaccagtcgtagtaatccccccaaaccaaagggaaagcctcctgctcccaaaccagccagtcccaagaagaaca

SEQ ID NO: 376 - DNA, 36 bp, Homo sapiens
ttagttgaaaaatggagagatcagcttagtaaaaga

SEQ ID NO: 377 - DNA, 150 bp, Homo sapiens
gtcacaacggtggtggatgtaaaagagatcttcaagtcctcatcacccatccctcgaactcaagtcccgctcattacaaattcttcttgccagtgtccacacatcctgccccatcaagatgttctcatcatgtgttacgagtggcgctca

SEQ ID NO: 378 - DNA, 54 bp, Homo sapiens
cggtgcaagtgtaaaaaggtgaagccaactttggcaacgtatctcagcaaaaac

SEQ ID NO: 379 - DNA, 41 bp, Homo sapiens
caggaaaggcctcttgatgttgactgtaaacgcctaagccc

SEQ ID NO: 380 - DNA, 254 bp, Homo sapiens
aggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtca

SEQ ID NO: 381 - DNA, 70 bp, Homo sapiens
gtgccaataccatgaagaggagctcagacagctcttaccacatgatacaagagccggctggtggaagagt

SEQ ID NO: 382 - DNA, 26 bp, Homo sapiens
gagaagtttgtcttgcaatgtattta

SEQ ID NO: 383 - DNA, 89 bp, Homo sapiens
tgatatgagatttattcacactttacacatacattcttgattgtggggcagacaattgttcatttcaacaggactttcacatggtaaca

SEQ ID NO: 384 - DNA, 61 bp, Homo sapiens
ggagggagaggtgtcgcagacacccctggcttcagtcttgcctcacgatccaacccctgca

SEQ ID NO: 385 - DNA, 60 bp, Homo sapiens
cagccatccagtgctccttggttgcgccttaaggccgaggcaggctccttcggggtcttt

SEQ ID NO: 386 - DNA, 805 bp, Homo sapiens
aacaaagcttctgtggaaccatggaagaagatgaaaatgagactggcaaagaacaaatgctgaatctgaagaagatttgggcaaataatctgcatacttttaattgggaataagatggaaaatatgaatgctaaatcaaattttttaaaaaatacaccacacgatacaactcaatacaggagtatttcttctcaaattcttctagcaccatcaacattcttcaagtatctgaaatactattaattagcacctttgtattatgaacaaaacaaaacaaggacctcagttcatctctgtctaggtcagcacctaacaatgtggatcacactcatgggaaagtgttttgaggtagtttaaacctttggaagtttgggttttaaacttccctctgtggaagatattcaaaagccacaagtggtgcaaatgtttatggtttttatttttcaatttttattttggttttcttacaaaggttgacattttccataacaggtgtaagagtgttgaaaaaaaaattcaaatttttgggggagcgggggaaggagttaatgaaactgtattgcacaatgctctgatcaatccttctttttctcttttgcccacaatttaagcaagtagatgtgcagaagaaatggaaggattcagctttcagttaaaaaagaagaagaagaaatggcaaagagaaagttttttcaaatttctttcttttttaatttagattgagttcatttatttgaaacagactgggccaatgtccacaaagaattcctggtcagcaccaccgatgtccaaaggtgcaatatca

SEQ ID NO: 387 - DNA, 29 bp, Homo sapiens
agcacgagcggctccgcaaataccactga

SEQ ID NO: 388 - DNA, 1249 bp, Homo sapiens
tacaccaggatgctcacttgatttgtgaatatttttcattccatcaacaagggagtttaaatactatatgtgagactgacaaaaaccttaatgtaatttacttataatgccagaaggaaaacactattttcataccctactttttctgtacctaaattttcttaaaaaaaaatctagtatagcactacattcttttttaagtgatgcagaccttagtttctttagcccctttattttgaatacaatgctacatatgaatgttgaagctgatacattgcacagttctgtagacatcactacaccgatgtagtttctcaaattttagcaatatgctctacataaaatcactacagagatactagtggggaagacgattaacacacctcttacagtaatactgcctgttattggtatagcagtggtatttgcagactgggatcataaggagcccttaaatacttgttattgactggggttatttttatgctgtagcaaatgtgacaggctctttttagcaaaatttttgaaaatttttttggtattactctgaaacaaaatttaagttggagtttcagggatttagggagtagttttcattctacatgaactgaggtaatattatggtaactccaatatttggttaaaaaaactatacaaatcagaatagtactaaaatactgtagaattttagcatttttattttgcactttgtgtggattgaggtgttcagaaataccaaccataaaaatgtaatctagttggcaaaggtgtgcgctaaaacacggaaccgaacatgcattgatttggataacttttgagggtttttgtcaaatagcatgtgaagagttacatttttcttaaaagattggtggtcccaatgtcagagttcttggaacagataactgaatgatagattttttttttttaaagataaaactttacaacctgcacatttgttatgcatactaaatggtgtgttaaaattagggtttctttgcctctctacactacactaatctgcctaaaggtggttgtttcatatttataatgctaattatcatacctacctactttaaattttaggtagaaaattatctgatttaaatacaaacatatttttctcacattgagtaatatgcataatgtagttccaaatgtatttcattactatagtcacaatatccaactaaaaattacgctatctagaattgtaccaaccaaaatctcgtattggcagatcttgacaggc

SEQ ID NO: 389 - DNA, 34 bp, Homo sapiens
gtgtgacgagagaacgagatttaccttcctgaat

SEQ ID NO: 390 - DNA, 131 bp, Homo sapiens
tctaagcggtaatggaggaagactagtgctttgtgcattttgatatatttgagttcattttttccacaatgtcatacttttgacgcagttgggtttctcataagtatcctagttcatgtacatccgaatgc

SEQ ID NO: 391 - DNA, 435 bp, Homo sapiens
cctggtcttcaaaaggttgggcaatttggcagctgaattcccagacagagaatagagcaattttagggatattaggactgagggagggtgtgggaaagctgtcatcagttgtttttatagaaagaactggcattcattaagaacctaaatcttatctttgcacaaatggaaaatataacctagttatagcttcctttggcctttattaaagggtaatatcaatcacagtcatagcaaagaaagcggatgtattaatggcaaattaatggaaaacctcccttatcaggaatctagactcagaatttaggaacacaaatcaaatcagaccaaccaagctatagccaaggacttgaaagaaattaaacaagacccagaataaatcaaggaattagaaattgttatttaaaaatttcagattgtaactccaggccctgc

SEQ ID NO: 392 - DNA, 110 bp, Homo sapiens
tgggacaagcctgctcgctgcctttgaccagtggagggaatgggccgacagcaagtcctgctgtgactactctctgcatgtggacatcagtgagtggcataagggcatcc

SEQ ID NO: 393 - DNA, 92 bp, Homo sapiens
atctagggcatattccaaccttccagcctgcgacctgcagagaaaaaaaaattacttattttcttgccccgtacataccttgaagtgagcaa

SEQ ID NO: 394 - DNA, 32 bp, Homo sapiens
tggaggacacaggaactctgacgcagaaagaa

SEQ ID NO: 395 - DNA, 896 bp, Homo sapiens
agtctggctacagcggctttctctggctgcagtgggctccacccagttcgaacttcctggcagctttgtttatgctgtgaggggaaaactgcttattcacgcttcagtaatgacagatgcccctctcccaaccaagctcgagtgtcccgggtggacttcagactgctgtgctggctgcaagaatttcaagccagtggatcttagcttgctgggttccttgggggtgggatccattgaggtagaccacttggctccctggcttcagccccgtttccaggggagtgaacggttctgtcttgctggcattccaggcaccactggggtatgaaaagaaacccctgaagctagctcgatgtctgcccaaacggctgcccagttttgtgcaggaaaccagggccctggtggcataggcaccagaaggaatctcctgttctgagggttgtgaagactgtgggaaaagtgtagtatctcggctggaatgcatagcccctcacagcttcccttggctaggggagggagttccctggctccttgtacttcccggatgaggcgacgcccaaccctgcttctgctcgccctccgtggtctgcacccactgtctaaccagtcctaatgagatgagctaggtacctcagttggagatgcagaaatcacccaccttctgcattgatcttgctgggagctgcagaccagagctgttcctattcagccatcttgccagccctttgacttgctatatttttcatgccgactgtccctgtgcagacaggatacattcttgttcactcttccctgttgataggaggttttgtaggttttgtgtgcagtgtttctattcctgctgatttcttagttgaaaactggtgtaatacctcaagggatgtagacaaatactc

SEQ ID NO: 396 - DNA, 106 bp, Homo sapiens
cttcgggaaaggctagcgaccttggacgcgcaggctctgaaaaccaaggtccagacccataccgagcgcccaaagaccgcatttgaattctcctcgccggccattg

SEQ ID NO: 397 - DNA, 335 bp, Homo sapiens
tggttccactgaaactccataatgctacagagagctactactttttccaggaagtaggttaacagctagaaagaaaaaggacaatttcctagcagcatggcaacttaaactgcagatctaataggtctgcaacttttacactaaaaatggcacaaacagctggtgacacaagtgagaaatggggaacaagatgtgaacactgaaaagaacaatatatatactgtaaatatgatgaataaaccaaatgtagctataagaatcttaaaggatgattatagaaaagggaaccaaatattttcctcaaatgctttttagagcctttctgtagagataca

SEQ ID NO: 398 - DNA, 260 bp, Homo sapiens
tttggttccagcttgtggtccactggccctgtgctactgaccttcctagcgcctgtctctccaacttaaagtgatattcatgggagaactctgatgggtattatttttcctcaaaggaaactaaggcatctgtaaaaatattttaaatgctggtaatagaagaaaatttggaagcatactcacatatttttccttctttttctagttttttcaaatgaagtaagagattatgtttagccatttcatgtaaattctcagga

SEQ ID NO: 399 - DNA, 237 bp, Homo sapiens
catgtcttttttcagccctctcagatccaaatgttattatgcactttttaatgtttgtaaacttttactaataattagtgtgaattgcattctgatacaataatgattatcattagaagctaacaaaattctcattaatactgtgtttgatggcctctgctgtgttttaacatcgtgcttcttatatggaaagtttttgtgagctgtgtaatccctctggtcagtattatgaaatca

SEQ ID NO: 400 - DNA, 181 bp, Homo sapiens
taggaggttccactctcaagtcacctagaagtttgattacatattgttacttacaaaactataataaattggatgcacagctgtttacttcagtctggtgtcttcaaccaaaatatgtaccttataccaaaacaatgcttattccaaaatattttttgtagctagtagttctttccttgga

SEQ ID NO: 401 - DNA, 82 bp, Homo sapiens
ttggggtttatgacatcgacaacaaaactattgagctgagtgatgatgacttcttaggggaatgtgaatgtacccttggaca

SEQ ID NO: 402 - DNA, 52 bp, Homo sapiens
agaagagtagttcagtctctcgatatggagcctctcaagttgaagatatggg

SEQ ID NO: 403 - DNA, 66 bp, Homo sapiens
tattgatgataacaccgtagtcagggcacgaggtttaccatggcagtcttcagatcaagatattgc

SEQ ID NO: 404 - DNA, 26 bp, Homo sapiens
acaatgggctaaaaataaacagtatt

SEQ ID NO: 405 - DNA, 68 bp, Homo sapiens
cttcaagttcaggtccagtgccctcttaatgcgaaacatcctgtcattataaaggttctcaggaagtc

SEQ ID NO: 406 - DNA, 520 bp, Homo sapiens
gccttaacctgtagtgcgtagaatatgcatcaatttcttgaaggagattcatgtttttataagaattttcatgtaattattgcaattgtggtcaaataaggaacgtttcctgcttgaaattatattgatttaaatgatgtgtgagatgtttcaccattttcaggcactgtgtaattctattgtaataaactggcaggtatctttgtaactataaatagtgcatgctcagccatgtacactgtaaatagcctttaccaaacgtgtttgacaaggaccataattaacatcacttagtgaattgtgataaagaaaaaaaagccatgatttattcgatgtgattggcttgtttttatgtggcgccaagaacgaacctgtttaacagctgtaaccaatggtactgatctatccatccaatgttgtcattatatttgactgtggttcaacagtattgcgttgtcagactaggaaagctaaacgaacaaaatggttttagttttgctgaagactggccttattaatg

SEQ ID NO: 407 - DNA, 101 bp, Homo sapiens
ttgagtacgaaaggtttcacataccttacaaattcattgtttgattaccgaagcccagaaaataatggtactcgcgcagaatttatcttggattcaactca

SEQ ID NO: 408 - DNA, 35 bp, Homo sapiens
gttccttgtagcttggaatactgggatgaactcca

SEQ ID NO: 409 - DNA, 300 bp, Homo sapiens
ttgtacagtactcacctattttagaatgtggttgactacaggtaaccaaaaccacagaaagggaaactttggatgaggggggcactactgtacttaggaatacaactatatacatatgattttatttttaagaccatattatatttgggtatctactaatattttgtataaagcaattttttgttccattacgtgactttttgttttattgtatatgtaatttaacacacaataaagggtaaagttgcttccccaaaccacacttttaatcaaaacctagaatcatctgcagtccttgtt

SEQ ID NO: 410 - DNA, 145 bp, Homo sapiens
tgttgcttctttggaccacttggtggcactagatttaccttcaggcaagccactggatgaaccgccagcatgacttcgggtggcactgccatcatccacactactgcttccactgttgtgcctgaatttggatggctttgactca

SEQ ID NO: 411 - DNA, 25 bp, Homo sapiens
cccgccaggacaaaccagtatgtag

SEQ ID NO: 412 - DNA, 89 bp, Homo sapiens
tcatcagctaggttcccaaggtggctcagcgttgaagtcgaactgctctctatacagcacttctcctaggccctctgggtagttgtata

SEQ ID NO: 413 - DNA, 450 bp, Homo sapiens
tgcagtatggtcgtacttttaactctagtatcctttgcttgcttcttacgccctttcctaggtgaattctcccacagtggtctgtatctcaacattttctttttaaaggaaaaaatatatatatatagtttctttttattgatcaggtctgtaaatgtgtactaaaaaaatcagagtttatttataaacaaaatagtttatttaaagagaaggtctcttccttattgatatcatggtatgcattaattccatttgttactattgtgcacaaaagccctgttcacaggggaatggtgtaaacatttatactgttttgttcactgtatttagtagacataactgttgaatagttactgaatcatgatgtaaagaatatgtgaccatcttcaggtatgggatttctgaacgtttcaaatttcaatcaatgagcactgtcaacacccacaggag

SEQ ID NO: 414 - DNA, 81 bp, Homo sapiens
agcatttcgtggatatgttaaagtatattgttgtagcaggtgcagtcagaaggatagttgaagctagaggggtgactcata

SEQ ID NO: 415 - DNA, 161 bp, Homo sapiens
actgttatagccaaaatcggcaatgacactaaagaaatcctctgtgcttttcaatatgcaaatatatttcttccaagagttgccctggtgtgacttcaagagttcatgttaacttcttttctggaaacttccttttcttagttgttgtattcttgaagagc

SEQ ID NO: 416 - DNA, 532 bp, Homo sapiens
ccaggtgcataatttcccatcagtctgtccttgtagtaggcagggcaatttctgttttcatgatcggaatactcaaatatatccaaacatctttttaaaactttgatttatagctcctagaaagttatgttttttaatagtcactctactctaatcaggcctagctttgctcattttggagcctcactaaaataacagatttcagtatagccaagttcatcagaaagactcaaatggaatgatttacaaaatagaacactttaaaccaggtcagtcctatctttttgtagctgaaggctatcagtcataacacaatttcgcgtacacctctgctcattatggaattacacttaaaacgaatctcaagagggtgaccattgttgtttcagataccatccctaaggagagtggttaacaggaagattgccagtgttactgatggaaagaagtgtttgtttgtttttttttcttgtcaaagacttacaccatagttttaaattaaactgtcaggcattttctcagacaggttttc

SEQ ID NO: 417 - DNA, 380 bp, Homo sapiens
gcccaattctgcactgaaggacaaggactgctgcaaaagaaatgagtgagaccctcaaaataaatgaaaggcacacagctcagccactaaggagatggacaagccatgtcaacctctgatctgctactttttgcatctataaaatggtaacactgctacttatacctcaggatgttgtgaggattaaaaattgagtaagctaagtgcctggtgcagtcctggcacagtcactaagcatttactatctttatgcagtttctttttgtaatgctaactgccctccaaattcctccaaagtaaacacatggattagtaaatgagaaagaaacaatgcttaaaacaactatgggtaacatacctgtttcaagtcgcgtagaata

SEQ ID NO: 418 - DNA, 38 bp, Homo sapiens
aaatggtcattgaggaagcaactatagattcagcaaca

SEQ ID NO: 419 - DNA, 37 bp, Homo sapiens
ttcaaacttggtggcgaatgtgttgcgggtcctgttg

SEQ ID NO: 420 - DNA, 25 bp, Homo sapiens
ggtggctctctgtatatcatgcata

SEQ ID NO: 421 - DNA, 149 bp, Homo sapiens
ctgagttatggcttctgtgcttgcttttgggattcggtgctctgcctgtggttctcccaggacaaatattagtaccagggagatctagtggaaaccaggagtccagacatctattgtccaaccctgaccttgctaccaatccactctga

SEQ ID NO: 422 - DNA, 144 bp, Homo sapiens
tgggacttagaagcgcacattcctaaccaggcctccagaggcagaggagcctcctagggatcccgctgagatgcgaattctgactcagcagatgtggggcgtggcctgagagtctgcatttgtaaccagctcccacatgatgtg

SEQ ID NO: 423 - DNA, 52 bp, Homo sapiens
ccctggatgtctatgtgagcactgagcagcagcctgcccatgtcccttgccc

SEQ ID NO: 424 - DNA, 227 bp, Homo sapiens
cacgcatttgatcacagactgtagagttttgaaaagtcacttttatttttaattattttacatatgcaacatgaagaaatcgtgtaggtgggttttttttttaataacaaaatcactgtttaaagaaacagtggcatagactccttcacacatcactgtggcaccagcaactacttctttatattgttcttcatatcccaaattagagtttacagggacagtcttca

SEQ ID NO: 425 - DNA, 45 bp, Homo sapiens
gattggagcaatgatgtaagagctgaactagcaaaaacccctgta

SEQ ID NO: 426 - DNA, 152 bp, Homo sapiens
cctcttccagaagataatagtatgaatgtggatcaagatggagaccccagtgacaggatggaagtgcaagagcaggaagaagatatcagctccctgatcaggagttgcaagttttccatgaaaatgaagatgatagacagtgcccggaagca

SEQ ID NO: 427 - DNA, 236 bp, Homo sapiens
cacacaagaacagcttttaacaggaattcatcaactccccccaacattttttattataaatacaggtatcaaaacaatcctgaacatatttttgatactactagtagtcagcacccacattgcttcaaaaattaacacatctgtgacaatattcattgtacctgctactttttaaacaatttcaactgcagctctctttcactaagtaagatgggtaaagcatgccatttctgttt

SEQ ID NO: 428 - DNA, 27 bp, Homo sapiens
cagcaatagcatcaaccaaggttatga

SEQ ID NO: 429 - DNA, 95 bp, Homo sapiens
gtaacttccttatatgggggcactgtccaaaattcaatactgatacaaaaatatgtttcttttaggtcaaaatacgtttcaggtgaattttctga

SEQ ID NO: 430 - DNA, 31 bp, Homo sapiens
aggaaaattgaagacgtgttcaagaaaacat

SEQ ID NO: 431 - DNA, 60 bp, Homo sapiens
tgctgtagatgaatgttcttagctgtcatgtttaaaaatacttctgcttcgttacctcaa

SEQ ID NO: 432 - DNA, 101 bp, Homo sapiens
ctatgactgtgacttaaatgcagccaatatatttgaaagactagtaaatgatctatcaaaaattgctcaaggaaggggcagtcaagaacttggtatgagta

SEQ ID NO: 433 - DNA, 350 bp, Homo sapiens
tccaggccagccaggtattgattgaagaaatctagaaaggcaaatggaccactgttatactgacagtgtttgtctaaccagctgagtgtgggcattttgaggaatggggccagagagccaagcccagggctactgcaagttgggaagtctaatagattctacttctaccagaattctgggattccaaagaatgataccttcagtgtaagggtaaattagaaataagcctccatagtactcataatgggccacaagaaaaactgaccatttcaaattttggcaagagtggagaagagagaaattgccactgagaatttggaaccatgaggcagcctcacacaagtttgtgg

SEQ ID NO: 434 - DNA, 108 bp, Homo sapiens
tggaataccttatgcatttgctttcgaactacgtgacactggatattttggatttttactcccagagatgctcatcaaacccacctgtacagaaactatgctggctgt

SEQ ID NO: 435 - DNA, 53 bp, Homo sapiens
ggtcaaggaactcaaggtttcgctgccgtggagtggatgccaatagaaactgg

SEQ ID NO: 436 - DNA, 60 bp, Homo sapiens
ctatatttctatatcatgcctgtgtttaacgtcgatggataccattttagttggaccaat

SEQ ID NO: 437 - DNA, 192 bp, Homo sapiens
tgctagtcatgcacctcagacagtgcaaggtgcttcctttgatctatcatgtcagcagtgggagaggtccttagcctaacagaggtctgactaaaagaacagccttcaaagtgagtgtcattttcagaaataaccatgctctgccagatctgtatggggttttttaatcgcatgctgctgacagaacgtttc

SEQ ID NO: 438 - DNA, 44 bp, Homo sapiens
tttgaagattctgcaaccggggcacagccacctttataacaacc

SEQ ID NO: 439 - DNA, 354 bp, Homo sapiens
ttagcaagtcgaggtaaaacacatgcaacattttctggcaaaagcttaatgtcaaacaatatgtgatccatactgtgtgtcgtccttgggggtttatttgactttgtcacaatgacagccaacagtgagactgataagcctgtaaaaataaaaaaataagactaatcaaatagacatggcattttaatctcaaagtgcaaaatcatctaactgaaaatgacggcattgaaaaattccagtggttaaaaatgaatcaaaacttcattacgcaggcagtggaagtgtgttgaaagatttaccaggggtgtcaagttttagacactcagaaaggcaccattctagccatcttgattg

SEQ ID NO: 440 - DNA, 185 bp, Homo sapiens
tcttcaggagaaagcttacagtgtttcaggttgtgatttattttcaaaatggagttgactctgaacatcactttgttcacctgcattgatgtttgtatttctagtgtgagcagtttatgcaaaggtagatatattcagagaaattttaaatacatttatgcaagttaatattgctttgctgatgc

SEQ ID NO: 441 - DNA, 173 bp, Homo sapiens
cacttgtgtcaccagctgtttgtgccatttttagtgtaaaagttgcagacctattagatctgcagtttaagttgccatgctgctaggaaattgtcctttttctttctagctgttaacctacttcctggaaaaagtagtagctctctgtagcattatggagtttcagtggaacc

SEQ ID NO: 442 - DNA, 94 bp, Homo sapiens
gcccgcatagccgtctgagtggtagtagtgaatcccccagtggccccaaactcggtaactctcatataaatagtaattccatgactcccaatgg

SEQ ID NO: 443 - DNA, 414 bp, Homo sapiens
cctctgcagtgtagtggcacctttgttataaacctggtgactgtaactcagtattgagaagtaataggtctgtggaggaaagatccagtcttgactccacagtgtgtagatgggtcttaaaaagaagttatatcctaatgaggagttaagttcatttgcagcatggttgatctgttagtaattgggaatatattatagaactgttttgctttctcccttttatttttttttaaactatggaagtaatttcagatttatatacaatttgtggaaatagtacatagacttcccatgtatccttcacctttattccccattgatagcatttctgaaccatctgggaataagttgtagacatgatgtcccattaccccttaatatttcaatgtatatttcttaaggacacggacaccg

SEQ ID NO: 444 - DNA, 32 bp, Homo sapiens
gcacctgaaattgcactggaactgctgatggc

SEQ ID NO: 445 - DNA, 119 bp, Homo sapiens
ttgttgacatgctctacaggagtgaccttaacatacctaatggtaactaaaactgttctctttaattacaaaattcccagcatctatcctactatgatactatctgaagataggcacca

SEQ ID NO: 446 - DNA, 239 bp, Homo sapiens
aagctctcttaggtttgattgaatagaaattactacagaattactgccatcaaggcaatctactactaactactatgatatactgaaagtgacaatgattaagagctgtgtcgtgatcaggttaaagagaaaaaaataattaaatttgtgtgtatgtttaattaggtaagtcaattatgcattaaaacaatacattttcagtgggtccttaatgtgacgtgaatggtgtttcaacatta

SEQ ID NO: 447 - DNA, 890 bp, Homo sapiens
ctgccagatgctgcaagcgaggtccaagcacatcttgtcaacatgcattgccatgaatttctaccagatgtgcttttatttagctttacatattcctttgaccaaatagtttgtgggttaaacaaaatgaaaatatcttcacctctattcttgggaaacaccctttagtgtacatttatgttcctttatttaggaaacaccattataaaaacacttatagtaaatggggacattcactataatgatctaagaagctacagattgtcatagttgttttcctgctttacaaaattgctccagatctggaatgccagtttgacctttgtcttctataatatttcctttttttcccctctttgaatctctgtatatttgattcttaactaaaattgttctcttaaatattctgaatcctggtaattaaaagtttgggtgtattttctttacctccaaggaaagaactactagctacaaaaaatattttggaataagcattgttttggtataaggtacatattttggttgaagacaccagactgaagtaaacagctgtgcatccaatttattatagttttgtaagtaacaatatgtaatcaaacttctaggtgacttgagagtggaacctcctatatcattatttagcaccgtttgtgacagtaaccatttcagtgtattgtttattataccacttatatcaacttatttttcaccaggttaaaattttaatttctacaaaataacattctgaatcaagcacactgtatgttcagtaggttgaactatgaacactgtcatcaatgttcagttcaaaagcctgaaagtttagatctagaagctggtaaaaatgacaatatcaatcacattaggggaaccattgttgtcttcactta

SEQ ID NO: 448 - DNA, 136 bp, Homo sapiens
tgtaagatatatttagcatcaattcatatattagtaacttacacatgtattgtcaagtatgacatccataaaagcacaaaacagtttctaacacgttccatcaaacttctggcgtactttatgtgtcatttccatg

SEQ ID NO: 449 - DNA, 447 bp, Homo sapiens
ctttgggcagctttgggcatcttaaggcatcaagtatacagaaatttcttttcgatcttaagtgccagttatcaccaattttcacacaaacctttttttttttcttcctattgcagttaaagggccattgccagtcagctgaagaaggaaatgtttgcttctccctttaaggtgttaaagtaatgcacagaaaataaaaatagcagcctcataaatctgcacggcattgcattcaagcaaaggacaatatgagtaacttagagaaatatccacattcaatgcacttaatgaaatcctgttttctttggagttacatgaggcagcagtactagctagtgtctaatattgcacttttatagcataaacacagctaaacatagtgttaaacactgacagcatcagtacctgttctaattgcatcagtgtttacctctcagtctagcatgc

SEQ ID NO: 450 - DNA, 105 bp, Homo sapiens
tcctgagaacctttataatgacaggatgtttcgcattaagagggcactggacctgaacttgaagcatcagatcttgcctaaagagcagtggaccaaatatgaaga

SEQ ID NO: 451 - DNA, 35 bp, Homo sapiens
ttgtgttcctgaactatgacacatgaatatgtggg

SEQ ID NO: 452 - DNA, 70 bp, Homo sapiens
gactgcagatgattctaggttttgattaaaagtgtggtttggggaagcaactttaccctttattgtgtgt

SEQ ID NO: 453 - DNA, 156 bp, Homo sapiens
tttcttagcgcaaagcagtgagggcagtacatgttctttttgcatttttaattattgtaatccttttagataatgatgtgttcatttgaactaactacatactatgatcaagtatattgcatcctaacgctacctctgactcaacctgactttgta

SEQ ID NO: 454 - DNA, 73 bp, Homo sapiens
tttgttggacgatttaagtctcgtaaagaacgagaagctgaacttggagctagggcaaaagaattcaccaatg

SEQ ID NO: 455 - DNA, 32 bp, Homo sapiens
ccttttgaataattgtcccaaatattacattc

SEQ ID NO: 456 - DNA, 36 bp, Homo sapiens
ttgtggacatcggatacccaaggagacgaagctgaa

SEQ ID NO: 457 - DNA, 227 bp, Homo sapiens
gctgagtttatagcttaaaggcctaaggagcactagcaacatttggctatattggtttgctagtcaccaacttctgggtctaaccccagccaaagatgacagcagaacaacataatttacactgtgatttatctttttgctgagggggaaaaaatgtaaatgttctgaaaattcactgctgcctttgtggaaactgtttcagcaaaggttcttgtatagagggaata

SEQ ID NO: 458 - DNA, 71 bp, Homo sapiens
tgactgtgacagacatccggtctgggacagaagataacaaagagaaagccttaccagtccatcctcccaca

SEQ ID NO: 459 - DNA, 135 bp, Homo sapiens
agttaacatgaactcttgaagtcacaccagggcaactcttggaagaaatatatttgcatattgaaaagcacagaggatttctttagtgtcattgccgattttggctataacagtgtctttctagccataataaaa

SEQ ID NO: 460 - DNA, 217 bp, Homo sapiens
gccactcattcatggttgttctatgttccatgaactctaatagcccaacttatacatggcactccaaggggatgcttcagccagaaagtaaagggctgaaaaagtagaacaatacaaaagccctcgtgtggtgggaactgtggcctcactcttacttgtccttccattcaaaacagtttggcacctttccatgacgaggatctctacaggtaggtta

SEQ ID NO: 461 - DNA, 168 bp, Homo sapiens
ttgtcattcaactcacaagtctagaatgtgattaagctacaaatctaagtattcacagatgtgtcttaggcttggtttgtaacaatctagaagcaatctgtttacaaaagtgccaccaaagcattttaaagaaaccaatttaatgccaccaaacataagcctgctata

SEQ ID NO: 462 - DNA, 35 bp, Homo sapiens
agcacctgttttatgtgccgaatcactgtggggaa

SEQ ID NO: 463 - DNA, 166 bp, Homo sapiens
ttggctataatacttgtgactcatgaagaattatgttgacaaacaggataaattccacatgcattttatttcccagtgagttgtataaactttatttttgttgaaggttgtatgttaaatcaatgttacattcttatatcacttcttgagaaggaagttccgattt

SEQ ID NO: 464 - DNA, 79 bp, Homo sapiens
tgaattggcaaatcgaatgtctttgttttatgctgaggcaactccaatgctgaaaaccttgagtgatgccacaacaaaa

SEQ ID NO: 465 - DNA, 79 bp, Homo sapiens
cctgggccttcggagcctcctgcctgcaccctccacctcttctaaaccatgatgtggcacattttggtgttaataaaac

SEQ ID NO: 466 - DNA, 153 bp, Homo sapiens
gacccttgtaaggactatagtcctctcttcagctgaccttcaggtccccagtcagaaccaagggaaccgactctgtcccttctcatagcacctgaaaaccccgggaaacgaggggaatgctttgctctcagctactgaggttgtcctgtcacc

SEQ ID NO: 467 - DNA, 101 bp, Homo sapiens
agctgaatccttctgccagcgtttggggaaataccggatgccctttgcctgggcacccataagcttatcaagcttcttcaatgtctccacccttgagaggg

SEQ ID NO: 468 - DNA, 407 bp, Homo sapiens
ccaccttcaggttatactcagtacaaattaaatgccattttattctctaaacgtgcagagacaagaaagttgatggtaaagtgatgatcatcattatggaaaaacaaatcttgatttccattggaacatgggaatctattttgttaaatgatttaggggcagagttaaatttattcggcttttaaagttttaaattatttgccttgctgacccctcctccataatccaggtctacaaatatttattaggtagtcaactactgtttgttagaagttgggagtaatggtttaggggagaaaataaacaactaagtttttttctttcttttttttttaatttatttagttctcatagcaaatcccgtgcggaaggcttttgtttgtcatgtgtctgagctcataactggc

SEQ ID NO: 469 - DNA, 281 bp, Homo sapiens
gggttcctgtagaggctgctcctccttgtccccgctcggacgtggtccccgctgctcgaggtgcagctgcagcagggcccggcggtacatggcttccagcttctcaagtcgggccctcagggccctggggctgcccggctcgtcctctcggccgctgtctctagccctccggttcgtgctggcctcttcagggacccggcgtgcctccgcgggcgtcccgttgccgccttcgccggggagccgcaaagcctgacggaagaggctgcggttctcgcgcttca

SEQ ID NO: 470 - DNA, 27 bp, Homo sapiens
ttctggacaatttctcctcagtcagat

SEQ ID NO: 471 - DNA, 99 bp, Homo sapiens
tttctcaccttgctgcggcctgctgtttggcaggacgacttgactggctgcgctgtggtttctgcgcctgtgatggctccttctgaatgccctctgagc

SEQ ID NO: 472 - DNA, 98 bp, Homo sapiens
ataaataagtgaagagctagtccgctgtgagtctcctcagtgacacagggctggatcaccatcgacggcactttctgagtactcagtgcagcaaagaa

SEQ ID NO: 473 - DNA, 300 bp, Homo sapiens
agggtgcagaccagattcgaaaacccagggcatcggcaccccaagggggctcgtggagcatggacgcgggcgcgtttctgcctggggtctggagctccgggaatctgcattgcgaaggagctcccagtggtggtgatgctgtgagttcgcgggctcggctccgggaaccgccggcccaatcagccctggactcccggcgccgctgcgtcatcctcccagttagtgggcaggaagcacagggacatgacccggtgtctggatgcggggatgagggatgacccgcgaggacatcagctctgt

SEQ ID NO: 474 - DNA, 272 bp, Homo sapiens
gcatttatatagagctttcggtttctgtctgtttaatttgatgctctgcttattcagttatactagatgtgtttctcagagttatccagtccatacgtatttgaagagacaatttggtgtagaattgttagtgtccaggctcttccaagcaaggtcttccaaagggatatctcaaaaatattccttagagttgaagtggcaatgttatatagcctaacaattttcatgctattaaaagcttataatagcggatcattaaaatgcgagttaca

SEQ ID NO: 475 - DNA, 96 bp, Homo sapiens
caccaacagtgcactagggtttcagtttctccacatccttgccaacacttgttattttctgggtatttttgataatagccttcctcatggatatga

SEQ ID NO: 476 - DNA, 1125 bp, Homo sapiens
acgctagcggcatccttcagggccaagtttgataaataccaccgccatcattctgctcatcctcctcctgttttttttttttctcttacattctttttttttttcctgtttatacattagaacaagataagatttgaaatacttccttgcaaataatgtgcaactcccaaggtgaaactcaaatagaaaaagtcatctctctggtagaaaggatggctttcctgtaatgactatagagtaagagtggcagcaatctttccatgcccttttcagcagaaggcacagaacagtagcgggactgccatctctggcaagatttcaggtaaagaatctcttcttaatttctaccttcctgtttctctgaatcagcccataggtgttgatgagtggccactcttaaagagtcactcagtatcagggatctactgtctttgttcaaaggtcaaataaaaacctagtctccttttattctactttctattcttagctagaatgaaactcagcatatatacacttctggacataataatattgaatagtaattacctttactagatgaaagaaattttcattacaaacttaaatcatgtaaaactcaacaactcagattcctggacctggtgtcctggttgggtccaaggtgattttacagaagaaaaaaacaactcaagcattctggtggcaacatagagattgtaggctgcttctaagaaagttattaacaatttggaaattcctaagtaggatgagagttagtaactggatacgagtgaagtttatatccaagttcagactcaaaggcattattatgatttgcttcttcccatgtcttccatgtcctgcttctcaaagtttttctcatccatcacactcctgccttaactgctctgagtatgcatttgttttcaattcatctttatttcaatctgtttaacttttgaatcgcatgggaatacgcacattaagttcctttctaaaataaggttttatgaagctgagtttcacgataagtgtcttgctattttttgagatgttttatggacaaagaaaactttacagatttatatgtattttgctgcaccagtaaatggaccattaactagggcccacctttaacagagcacc

SEQ ID NO: 477 - DNA, 86 bp, Homo sapiens
ttaggtttttgtgtaagattcttgctgtagcgtggatagctgtgattggtgagtcaaccgtctgtggctaccagttacactgagat

SEQ ID NO: 478 - DNA, 737 bp, Homo sapiens
atgccgcaactccacacagtgtgtaaaatatatacaaccaaaaatcagcttttgcaggtctttatttcttctgtaaaacagtaggtaacttttcctaggtttcactctttttagtgtactagatccagaaacttagtgtaatgccctgctttatatttctttgacttaacattggtttcagaaagaatcttagctacctagaatttacagtctctgtttcatggcaacactggataatggctttgtgaaatttaaaaaatttttgtagcgactgtaaacagaaatgccaaattgatggttaattgttgctgcttcaaaaataagtataaaattaatatgtaaggaagcccattctttcatgttaaatacttggggtgggaggggagaaagggaaccttttcttaaaatgaaaataattactgctattttaaaatttcttgatcattgaatgtgagacccttctaacatgatttgagaagctgtacaagtataggcagagttattttcctgtttacattttttttttgttttggggaaaaaattggtaggtgtctaattactgtttacttcattgttatattgcagtaaaagttttaaaacaaccattgcatgtttgcttttgatgtatccctttgtgaaattagcacttttggggccaatggagaaatgcagcattcactctccctgtcttttccccttccctcagcagaaacgtgtttatcagcaagtcgtgagtc

SEQ ID NO: 479 - DNA, 100 bp, Homo sapiens
cttcacaagtgttttacttcgacgatgtgcctttgatttaatttgggacacttttttagaaggatacattattcgtgtttgcaacggtctttgaagagct

SEQ ID NO: 480 - DNA, 37 bp, Homo sapiens
aaaccaagacgctgacactagcctgagttcccctaac

SEQ ID NO: 481 - DNA, 224 bp, Homo sapiens
tggctgctcactgcatcatgctggtttcaccctgctcagagcgggctccacccctgctcggcatcctgactcatcctatgcatatggcagcgctgcttgcaggagagcccaggcgccctggcagcccccaggtccccatttgttccagttttactcactggattcttgccagaggtggcagccgctgtgacacagtatatgctacatttggtattgggctaatc

SEQ ID NO: 482 - DNA, 61 bp, Homo sapiens
ttcccttaagccatctgggcctacaccagcatccggtcagttatcatctggtgacaaagct

SEQ ID NO: 483 - DNA, 116 bp, Homo sapiens
gcccatatgattctcatgcatttgatatttatgtttaaaagtgtttatatatgtatgtaaaaagggaaccatatgttttgagaatttgtaaagtgagagacatgatcctattaaaa

SEQ ID NO: 484 - DNA, 94 bp, Homo sapiens
ttgaagttcagctaacaatcacagcataggttctgatgcatggaaaggtggttggtgaatgaaaaagttgcgtagagccactactttctttttc

SEQ ID NO: 485 - DNA, 199 bp, Homo sapiens
gactagcctataatctcctgtaacaatggcacatataataattaacaacagcaaagatgcttggtttcttgtttcatgtaatggccagtacatctgtggacaatgtcgagtcctcaggaagtccaggaggctgctacagaggaaatccaagaaccatgtcacatctctcaacaagtcttgggaagtccatctgactctc

SEQ ID NO: 486 - DNA, 81 bp, Homo sapiens
agacaggacatcacatatgaatgcacgatatgaagagcctggttacagtttcgactcctctctgcaagtgaataggcccag

SEQ ID NO: 487 - DNA, 41 bp, Homo sapiens
caggggcactgttcaatccactgtagcatgggaagccctga

SEQ ID NO: 488 - DNA, 248 bp, Homo sapiens
aatcatgggctaagggtacattacaatgaactgaaaaaatactcctgttttggtctctaacagatggacaaccaaatgatttgtcttcattagtcatttgttcagatacatttttttcactccaattattgtttccatttttgcaagtcaaagaaaagttattcgtagtattaggattatgtgaaaatctctctatacttcgatcggcatttctgttaaaagtcaatggttgcaattccttagtttcc

SEQ ID NO: 489 - DNA, 389 bp, Homo sapiens
tcatgtcttagagcccgtctttatgtttaaaactaatttcttaaaataaagccttcagtaaatgttcattaccaacttgataaatgctactcataagagctggtttggggctatagcatatgcttttttttttttaattattacctgatttaaaaatctctgtaaaaacgtgtagtgtttcataaaatctgtaactcgcattttaatgatccgctattataagcttttaatagcatgaaaattgttaggctatataacattgccacttcaactctaaggaatatttttgagatatccctttggaagaccttgcttggaagagcctggacactaacaattctacaccaaattgtctcttcaaatacgtatggactggataactctgagaa

SEQ ID NO: 490 - DNA, 28 bp, Homo sapiens
attccatttgatactcgaatgcttgatc

SEQ ID NO: 491 - DNA, 60 bp, Homo sapiens
tttgatctgtttccaatgtgtccatttggatgtcagtgctattcacgagttgtacattgc

SEQ ID NO: 492 - DNA, 223 bp, Homo sapiens
actggtagccacagacggttgactcaccaatcacagctatccacgctacagcaagaatcttacacaaaaacctaacaacgcttacaattttgttggggtgaagtccacaagcttggtggtagattaccaagagggactacatggaaggaaggcggaatgtcacaggaactattctttggctctttgggtgggtgggtgtcctggggtttgtatcacagctacc

SEQ ID NO: 493 - DNA, 87 bp, Homo sapiens
cacatggctccaggttacgaagaaccagcacctttgagaggaagcctagtaaacgttatccatcccggagacattcaacgttcaaag

SEQ ID NO: 494 - DNA, 28 bp, Homo sapiens
tatgaagacataaacccagttgccatct

SEQ ID NO: 495 - DNA, 262 bp, Homo sapiens
agtaaggcttgttggcagagtcctctgctgctgtccctaggctaagagtctagagttgtctccagtagaggagaagttctatcctatctttaggcagaaagtggggaggggagagagagagcttttcctcagtttgctgctgccttttatttttatttttaagcaaccctattaactgagctgcttcttaattgctttcagctcaaaaataatttttatatcaaagaagcatattttaggtgacatattctagttcccttca

SEQ ID NO: 496 - DNA, 516 bp, Homo sapiens
cacctcctctaatgtcatggctggaaggaaataagaagcccttcctcgggagaatgccagaggcccttgattgaagtcaaggacataagggatgacctcctcccagtccccagccctcaccttcgccttgcgtttgctgtcatccatgatcatcttccagtccgagccgttgttgctgtacccgatcttgaacttcctcatgaacaccttgttctctcggtgcttcccaccctgaatgatgatgcccctcacgatcttctcctcccccaggtctatttggagccactcattgatgtaggaatgaggtgcgggtggaagtgcccagccagagcgactggttaccaggcggatgttttcaggcatccagtttctgtccccttggttggatgatgtgatctgggagtcagaaataagtccagacaccatacccaacattccagagcaaggataatctgggaagtgaaatgaaacagataatgtaaaatgatttcctgggcgactccatgctaagaaata

SEQ ID NO: 497 - DNA, 29 bp, Homo sapiens
aataagcctgtcttcctatctggattttt

SEQ ID NO: 498 - DNA, 44 bp, Homo sapiens
ccagggctgacagccccgatggaaagctgatgccctgcctttgg

SEQ ID NO: 499 - DNA, 112 bp, Homo sapiens
agctgataagcaggaaggctctccaccgcccacttggtgaaaaaggagtggacggggacttccagccttacagagcagatgaggatcgactgaaacacacacttgtgaccca

SEQ ID NO: 500 - DNA, 285 bp, Homo sapiens
cacacacgttcttctctaggttttttctgtaaattttacatctctaggtgggggtagtaggggaagagaagcaggttttttttacagctgatgtgatcactgtacagacaagcttggaaaatgctacccaatctgccaattaattaaagcgttaatctgaggagcagccgggcccagggaatgtttacccagggaggagtgggggtggtaggaactgagtaggcagctccctatatttgcatgaaaatgaaggtgcttaggaaaccctgcttccttttgtctcta

SEQ ID NO: 501 - DNA, 34 bp, Homo sapiens
gaattccttgtccataagtaatccagggctcttc

SEQ ID NO: 502 - DNA, 80 bp, Homo sapiens
aggatgatcagtggtaccgtgcctctgttttggcttacgcttctgaagaatctgtactggtcggatatgtagattatgga

SEQ ID NO: 503 - DNA, 81 bp, Homo sapiens
aaactcaatgatttgaacaagtcattagcagaacactgccagcagaagttacctaatggtttcaaggcagagataggacaa

SEQ ID NO: 504 - DNA, 165 bp, Homo sapiens
ggtagttggtatcgtgctttagtcaaggaaatcttaccaaatggacatgttaaagtacattttgtggattatggaaacatcgaagaagttactgcagatgaactccgaatgatatcatcaacatttttaaaccttccctttcagggaatacggtgccagttagca

SEQ ID NO: 505 - DNA, 105 bp, Homo sapiens
atgtgtgttgctgggataaaattgcaagccagagtggttgaagtcactgaaaatgggataggagttgaactcaccgatctctccacttgttatcccagaataatt

SEQ ID NO: 506 - DNA, 108 bp, Homo sapiens
ctgagcaatggaagacgatagaattgccagtggataaaactatacaagcaaatgtattagaaatcataagcccaaacttgttttatgctctaccaaaagggatgccag

SEQ ID NO: 507 - DNA, 89 bp, Homo sapiens
gacagctgaattattagaatactgcaatgctccgaaaagtcgaccaccctatagaccaagaattggagacgcatgctgtgccaaataca

SEQ ID NO: 508 - DNA, 144 bp, Homo sapiens
tcgtgcagttgttctggggacatcagacactgatgtggaagtgctctatgcagactatggaaacattgaaaccctgcctctttgcagagtgcaaccaatcacctctagccacctggcgcttcctttccaaattattagatgttc

SEQ ID NO: 509 - DNA, 294 bp, Homo sapiens
ctgtgatcaccaataggacatcttcaggcatattggcaggatagagctaatggagtgaaacctattgtaaggctgtactttcgtgatttaatgacctgaggtttggtcataatgcttctgctgtttttgtaggtttatctgatcgttttcctttgctactgctaatggaactgaacccccaggggtattccagttgtaatagcctttccttactgttgtttggttctgtgaatgcctatgttattgatatgtggagggacttgtaaaacttgttgtgacataaagcttagcctc

SEQ ID NO: 510 - DNA, 100 bp, Homo sapiens
actcagagctcacttatgcacattgctgtaaatatgtttagcactttttgcgaggggtatctaaagggccaagaacggaaagtggatctaaagggccaag

SEQ ID NO: 511 - DNA, 25 bp, Homo sapiens
tagattccttctgatctttcaccca

SEQ ID NO: 512 - DNA, 35 bp, Homo sapiens
aaagcccacggtcatagacagcaccatacaatcag

SEQ ID NO: 513 - DNA, 215 bp, Homo sapiens
gatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatga

SEQ ID NO: 514 - DNA, 118 bp, Homo sapiens
acttgggccttctgcgctttgtcacggctgtcgggacacagggcgccatttcaaaagaaaccaagaagaaatattatgtcaagacttacaagatcgacgttagctccaacggggaaga

SEQ ID NO: 515 - DNA, 429 bp, Homo sapiens
gacgggttattgcaatcttttctgaaattgaatttcagtttgcttcaatagcatttcccagtaaataggagtgactctggtatctcgtttgtgaatcttgatgtaataatcggaggtaattgaagcgtgcacaaaccacagagacttatgacatgtttttaaacaaggattgactgatacgaaactaattgaacaaaagctggaaatcagattcctccaagaaaccttgcacagaatacagggtctttcataatggcactgagtttgactttaaaagtggcgtgttcaagtttccttattttcctgaaggagtttaggagtaagttactgacaggcaaggttgtgacccaatgaccagagttaatcatctggcgactggaattaatcttttgtctgtcctgtggtttggattaaccagggtcggtcatc

SEQ ID NO: 516 - DNA, 48 bp, Homo sapiens
catagtttatcagttgctaggctccaagcagaggtaataattgctcag

SEQ ID NO: 517 - DNA, 27 bp, Homo sapiens
atgtcaaaaacgtatagcatttccatt

SEQ ID NO: 518 - DNA, 103 bp, Homo sapiens
atctctggaccaaatattcttagacatatatttaatctctgatgaaaggatccacaagttcaaataatttggggtattaagtggggcttggataaaatcttca

SEQ ID NO: 519 - DNA, 77 bp, Homo sapiens
gttctgatggcattccgaagaatcagccccagacagacagtaggattgttcaggaaggcccaaatcttgcaattgtg

SEQ ID NO: 520 - DNA, 376 bp, Homo sapiens
cccaatgggcttgctctttacaaaaacaaaaaactagacacacccctaatagcttaaggaggtaaggtctctaaaatgttagttcactagaaccaggtttttatctactgcagttcagtcagtttaccaaaacaaggaaatcaaagataacattattttaaaaaggcaattagatggatttccagataacggcacaaatgaaaagaggacagaaaaaagatgtgtagtccaatccaattagtaaagctatttgttatatcatatataactttacaggacagtctggactatctcttcaaagggtcactcaaacctactgtttcaaggtaagattttcgtaaacttttccttggcaatcttgagaaagcatcactga

SEQ ID NO: 521 - DNA, 762 bp, Homo sapiens
ctctgcattccacaggcaaggtttacctaaagcaccagggtggactgagaagaattctcatcatagttgggagccattggatgccccagagggtaagctgcaaggctctaggtgtgacaacagcagttgcagcaagctccctccacaagaaggaagaggcattgctcaagaacagctgttccaagaaaagaaggatcctgctaacccctccccggtgatgcctggaatagccacctctgagaggggtgatgaacacagcctaggctgtagtccttcaaattcatcagctcagcccagccttcccctgtatagaacctgccaccccataatgcctgttgcttcttcatttgtgcttcactgtcctgatcctgtgcagaaaactaaccaatgcctccaaggccaaagcctcaaaacttcattgactttaaaagtggacagaggcagtgaggagacctataggccagagtttcccagcacaaaggggcttgtccgttctctggctgagcagttccagaggatgcagggtgtctccatgagggatagtacaggtttcaaggatagaagtttgtcaggtagtctaaggaagaactcttccccttctgattctaagcctcctttctcacagggtcaagagaaaggccactggccatgggcaaagcaacaatcctctctggagggtggggatagaccactttcctgggaagagtccactgaacattcttctcttgccttaaactctgggctgcctaatggtgaaacttc

SEQ ID NO: 522 - DNA, 28 bp, Homo sapiens
ttaccggcggggagctgtttgaagacat

SEQ ID NO: 523 - DNA, 458 bp, Homo sapiens
tcctggctctgaccgtgtttacctgtccaggtctcatgattcttatctgaaaatgaaattgatagtttctgagaagattaaatgagaataatacatgcaaagtgcttaccccagtgaatgagacataagaatcatttactagatggtagttatagatttttttcttgcttattttctcttcttaatctgtacataagtttagtatgattagactatctcaagtctattatgtggctaggacagtcacttaattggcttgactataagcttagcttgaggagcaaaagtataggtgctaaattcatgttgagataatagaataccaagcattacaattaaaaggaatgttgagtgttttgtttttttttttttgtaatggactcttgatgtaggttatttgccaagtactgtatgttgaatgtattacagaaaaatgtcaccttggtaccttcttgaga

SEQ ID NO: 524 - DNA, 824 bp, Homo sapiens
ggttttagatccacgtcctttgacaagttcggtcatgcccgtggatgtggccatgaggctttgcttggcacattcaccacctgtgaagagtttcctgggcccgtacgatgaatttcaacgacgacattttgtgaataaattaaagcccctgaaatcatgtctcaatataaaacacaaagccaaatcacagaatgactggaagtgctcacacaaccaagccaagaagcgcgttgtgtttgctgactccaagggcctctctctcactgcgatccatgtcttctccgacctcccagaagaaccagcgtgggatctgcagtttgatctcttggaccttaatgatatctcctctgccttaaaacaccacgaggagaaaaacttgattttagatttccctcagccttcaaccgattacttaagtttccggagccactttcagaagaactttgtctgtctggagaactgctcgttgcaagagcgaacagtgacagggactgttaaagtcaaaaatgtgagttttgagaagaaagttcagatccgtatcactttcgattcttggaaaaactacactgacgtagactgtgtctatatgaaaaatgtgtatggtggcacagatagtgataccttctcatttgccattgacttaccccctgtcattccaactgagcagaaaattgagttctgcatttcttaccatgctaatgggcaagtcttttgggacaacaatgatggtcagaattatagaattgttcatgttcaatggaagcctgatggggtgcagacacagatggcaccccaggactgtgcattccaccagacgtctccta

SEQ ID NO: 525 - DNA, 81 bp, Homo sapiens
gttcaccaacccatgcaagaccatgaagttcatcgtgtggcgccgctttaagtgggtcatcatcggcttgctgttcctgct

SEQ ID NO: 526 - DNA, 29 bp, Homo sapiens
cccagaaggcagccgtatcaggaggttag

SEQ ID NO: 527 - DNA, 116 bp, Homo sapiens
ccatgctcagtaagtccatttttgcatggaatatggagccttaaaacatgtcatgaatttggagtccctggcacataaatctaccttcaaatcagaggtccttaatgatgcctaaa

SEQ ID NO: 528 - DNA, 166 bp, Homo sapiens
gacatctgtcttaggctccagtggacccccgtgcctcctaaggcttgagtgcaggtacccgtgttcctagagcacaggacgctgtctgcggctccccatcttccctgccagctccagcctgaactcaaggattgttaagaccactccactgatccctaaagctgtt

SEQ ID NO: 529 - DNA, 26 bp, Homo sapiens
cttttatgactcttgtcctcttcaag

SEQ ID NO: 530 - DNA, 56 bp, Homo sapiens
tctgatttaattcacggagcaggggggatacacagccatgattccactcctgattt

SEQ ID NO: 531 - DNA, 122 bp, Homo sapiens
cggggtgttaatgtgggagatcttcactttagggggctcgccctacccagggattcccgtggaggaactttttaagctgctgaaggaaggacacagaatggataagccagccaactgcacca

SEQ ID NO: 532 - DNA, 192 bp, Homo sapiens
gctgaccaccgcactagcaaggataccttccctaaagaaaataaaccgagaataccaatgtgaaatttaatagccgttccaccagtaattgacattctctaaaacgtcactaggaaaatactcaggcgcgtgtgtaccctaagtctcattagttccatatgataagcactccatgctttagtaagccgctga

SEQ ID NO: 533 - DNA, 258 bp, Homo sapiens
ttccaagcactgactgataagctcgaatgtggagttgacacctggaagggagttttgggagcttttggcagcccatccccagtggaagaaggaggaatggctgaaattgttgactgcttggggcagcctctgtcccaggaagacaaaggctccagtgtgatagctgagctggggccaggggacggaggcacggcggagtccctcaaaccacagctggagtgtgaagtttggactgtctcctgggttgggaacaaagta

SEQ ID NO: 534 - DNA, 109 bp, Homo sapiens
ggatgtccagcgtcttccacttctgcctcctggtgcccggcctggtgttcagcctctgcaccctcaacgtggtcaccgacagcatgctgatcaaggctgtctccacctc

SEQ ID NO: 535 - DNA, 393 bp, Homo sapiens
tttgcttgaggtggtaaggctttgctggaccctgttgcaggcaaaaggagtaattgatttaaagtactgttaatgatgataatgattttttttttaaactcatatattgggattttcaccaaaataatgcttttgaaaaaaagaaaaaaaaaacggatatattgagaatcaaagtagaagttttaggaatgcaaaataagtcatcttgcatacagggagtggttaagtaaggtttcatcacccctttagcactgcttttctgaagacttcagttttgttaaggagatttagttttactgctttgactggtgggtctctagaagcaaaactgagtgataactcatgagaagtactgataggacctttatctggatatggtcctataggttattc

SEQ ID NO: 536 - DNA, 234 bp, Homo sapiens
tgccatccactctttcatgtgcatggagtcaggcatcttcctctgcatggcagtggatagatatatggccatttgttatccccttcagtacacttccatagttactgaagcttttgtcatcaaagccacactgtcagtagtgctcaggaatggcctgttgaccatcccagtgccagtattggctgcccagcgacactactgctccaggaatgagattgatcagtgcctctgctc

SEQ ID NO: 537 - DNA, 304 bp, Homo sapiens
ctcgggaaaggatcatcgccgttgaaatgaaaagagagacagagagaaaaaaaaaaagagaacccacatgaagctctgaaaccaaacagcatcctgccatgagcttcccagagacagaagagactggagcaaagtcggaaacacagagaagcacggcttcccctcagcacagaccctccagactgggtctcagagccgtgccacccaccctcccacacagccggccacagggagaactggtgctaaccagggtgcttgctttggtcacgttcaacgcactacagagctacgacacagggaaacc

SEQ ID NO: 538 - DNA, 258 bp, Homo sapiens
agtggtctccagttgctattaaagatgatggagaacaacagaggggtgtttgatgcaagtaaaaacagaaacttccaagaatgtaactctgaatattccagccctatagaacaatctctctcacaggtgttccaatatcgcagttatctccgcgtggacactccattaaaccgaccccgcgtgtgaacacacttgatgatgttatacctaagctccactgcaaccattcacaatgcctccattggtaagtcttatgtg

SEQ ID NO: 539 - DNA, 41 bp, Homo sapiens
tgatctcttcttgcattggttgtatttgtatgtcacaaata

SEQ ID NO: 540 - DNA, 2150 bp, Homo sapiens
tgcctgtccataccggaatcgagtataacacggtgcctggcttagcacaaaacagtagtgggtcctgcaggccccagagtctaattcctggtattctttcccctacacagattaaataaaccaaaaacaaactattctaggaaagcgtctgtgacatttgtaaaaagtggtatttaatgatcttttattcacttgtctgtttagtttgttgaaatcttaagtggcatcctggtctgggaaggagtgctgtctgcgcctgccctccgctgggcacagcgtggctgcttcaggggctaagcacacactttctgtcttctaaagggccgccacatgccaggagctcaggtgtgagcccggctctggctcttacctcatagggtcactcataggggcacagggagcagaacattgtacacagcgaggcaccacccggcttggcatctgcctcggtggacttactacctctagaaggaaatacctgagttcctctggcctcagctcctagagtgactggtgtgctgtccctgttactcttctgtcaaggtgacaactgtgtgacccatcatctgtgtgtcaaagcaaggccctgcctgggcctctgctcctgtgctgaccccaaaggcaaatgctttgctagtttccttccagttaatttcacctatgaatagatgtgtgaaaactgttcaaagccatacctgcacatgtttgaacttcaaaccctgtgggtgattcagtggcatctttctctaacccccagcctcccttcccacagaggccaccgtcatggccagttgctgcagtttctttccagagaacctgtgtatgtgtaaagctgtacaggcgtgggtacaccacacagcctgtcttgcactgtggactgttgagttactagtacatctaggtaagcaccgcatatctgtattcatgtctgccttggtcttttcaacatctgtgtggtagccgtgtttgaattacccattccctttttggggaaccattaagttgtttcagcaatttttactgtagataaggctataccgcatatctgtgtacatgggtttttatgtacatgggcaagtatatctgtgagagaaaagtttcctcaggaggaattctgggcacagcatgtgtaaatttctaaatatgatggacacccccagcttccacctcaaggaggttggtcccattgacatttccccacaccttcacccaggctgtgcccttaaacttggttatttgtcaatgtgagaagtggaaaatagtatttaattgtagtttggatttgtatttctattgggttgtatacttactgattaataataagagctctttacatattaaggaaattaacccttttcaaatacattcctatttctcactaatctttaagttttattgtaatattttgctctttagtttatatatatatgtatatatatatatatgtatatatatatatatacatatatatatacatatatatatactaattttcttttatggttcctggattttgtgagtagtttgaaaaggctaatccagctgaagattttgttgttgttgttaaaccccatgttttctcctaactctttttatttttattttggaggactctatctagacttaattttagcataacaagtgacagggttagttagcctgttgtccttacaccattttctggctaatacagctattaactattgatctgtctattcacgtgccagttcctaatggttttacatagtgtaatctgcacttcaaaatagcgaagggaagccctacctcattattctacttttccagaattctcctggctattccaggctgcatgtttaccttaaccttccctgtgatgtcttcatgccgttgtcttcttatgcaagaataaggtacgtctttccatccactcacgtctatttaatttgactttgcattacacagaaagctggtcttggtctgtctacctcggcatctagttgtcctcactgccccctagccgaccccaccccatctgactgactaccccatcacagagtacttttatttacgttttgctctgcctaatggttacttgatactgtcacgccgacagtgtccagttcagtggtctttgcagttgaaatgctcccgtacacactgtcttgtta

SEQ ID NO: 541 - DNA, 176 bp, Homo sapiens
aggctcgcattgtgtgtctggttcacttatgatcacgcttgcctacttttaagaatggaagaggggaggtggagggtggctgcacagtcgagggtgtgaggcagtcttgctctagccccaccatgccctcagcccgctgtggccacgctggttcctcaattgctggggcgtgcagt

SEQ ID NO: 542 - DNA, 148 bp, Homo sapiens
aggtcccttactggtcctgcttccatgagtagccgtgaccaggggaaaagggagaggaaccagccggcacagggaggggtcatctccacaacattccatttatacacagaactaaacagacaagcacagagtcactattgcggttaga

SEQ ID NO: 543 - DNA, 81 bp, Homo sapiens
cctgctctcagcatatgtggggcgcctcagtgcccggcccaagctcaaggccttcctggcctcccctgagtacgtgaacct

SEQ ID NO: 544 - DNA, 52 bp, Homo sapiens
tacacagataccacatttagcaggaacagaacaaaactttcagcttgcaaag

SEQ ID NO: 545 - DNA, 57 bp, Homo sapiens
tgaaaatgtttcggatattgtaccatctttcagtgctttctctcctcaaggaatgcc

SEQ ID NO: 546 - DNA, 35 bp, Homo sapiens
agtctcaaagtgtcctacaatgttggacctggctt

SEQ ID NO: 547 - DNA, 29 bp, Homo sapiens
tggtcttcttggttctactgagtgggcag

SEQ ID NO: 548 - DNA, 420 bp, Homo sapiens
gatccatttaggtcagctttagtcagaactgtaaaatcagcaaacataagaaaaacaaaacctagtaatacatacaaaagctttcatgggttctagaaccttcttaactgctgattcatgtggagggcattaagagttgaaaaggcttatatggttaactaccttagactatatctacagcagggtctggtttgccagaacaagtttaaagtggctgtttattaagtttgctattttcagaattgaaactataagaccgccatttgacactgaaacttgcgtgaatcctaaattgcatcaattatctatttgataaaagcttattctaatttaaaaccttatagagtaagagactgatatatatagcagtcttaaagatcacgtcatctgccttacattagtccagtcacgtgcttcgta

SEQ ID NO: 549 - DNA, 268 bp, Homo sapiens
tcagtcttgtgtgggtacaggtttatatgtgccaactaaatgaactgtaagcagcactgtgagcactgattatttaaggtgactgaggattactgtcagtgataggattgtgttataattccacttatatacatctgatatggaaaactgaaacttccattttagaagagaaagaaaatagctgaatttggcatttcagtagcatgctatagaattcatcatgattaatttcacattaaattatgggcaatagctgtatcacatttca

SEQ ID NO: 550 - DNA, 270 bp, Homo sapiens
gtgcctgacaaataatctcctggggacacaaatatgtgtccagtgagcagcttggttcaacagcgcatgcttttcctccaccagatttaggacacaacattaaagttttagaatggccagagaggtcactctttgctgaattccaatatctgaaaaacaattgcttgagcagattgtaagcacttgtccttattcttaaaaaaaaaagggcggggtggggggagaaaaaatacacatacacacaacccaaaggcgctgaagttaagcatt

SEQ ID NO: 551 - DNA, 164 bp, Homo sapiens
aacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggatt

SEQ ID NO: 552 - DNA, 226 bp, Homo sapiens
catgaaacttatatgtagacgttcctaattagtggcgtgtctgataccaaactaggtatcttttaaatttttattattctactatcagttatattcatttttcccctcataaaatattccttaaagtaaagaataaaatttcacaattcatttcagactcttcttatcctcctcccctccaaaatttctacataactgcatggggtcagttacagatacatccaga

SEQ ID NO: 553 - DNA, 56 bp, Homo sapiens
atggacgatctaggaaactttcagggagctaaggtgtcggagagcaaagtatcctt

SEQ ID NO: 554 - DNA, 68 bp, Homo sapiens
ccttctgattatggaatactgggtcggctggttcgacagatggggagataagcaccatgttaaagatg

SEQ ID NO: 555 - DNA, 68 bp, Homo sapiens
gtttttggatgagacaatgatagggattctgaatgagaataataaggacctgcacattcctgaactca

SEQ ID NO: 556 - DNA, 109 bp, Homo sapiens
ttatggatttgtgttcatcaatggacgtaaccttgggcgatattggaatattgggcctcagaaaacactgtaccttcctggagtttggcttcatccagaagacaatgag

SEQ ID NO: 557 - DNA, 841 bp, Homo sapiens
tgcccggcctgtgtacaactttttaaatagggaatatgatagcttcgcatggtggtgtgcacctatagcccccactgcctggaaagctgaggtgggagaatcgcttgagtccaggagtttgaggttacagtgatccacgatcgtaccactacactccagcctgggcaacagagcaagaccctgtctcaaagcataaaatggaataacatatcaaatgaaacagggaaaatgaagctgacaatttatggaagccagggcttgtcacagtctctactgttattatgcattacctgggaatttatataagcccttaataataatgccaatgaacatctcatgtgtgctcacaatgttctggcactattataagtgcttcacaggttttatgtgttcttcgtaactttatggagtaggtaccatttgtgtctctttattataagtgagagaaatgaagtttatattatcaaggggactaaagtcacacggcttgtgggcactgtgccaagatttaaaattaaatttgatggttgaatacagttacttaatgaccatgttatattgcttcctgtgtaacatctgccatttatttcctcagctgtacaaatcctctgttttctctctgttacacactaacatcaatggctttgtacttgtgatgagagataaccttgccctagttgtgggcaacacatgcagaataatcctgttttacagctgcctttcgtgatcttattgcttgcttttttccagattcagggagaatgttgttgtctatttgtctcttacatctccttgatcatgtcttcattttttaatgtgctctgtacctgtcaaaaatttt

SEQ ID NO: 558 - DNA, 134 bp, Homo sapiens
tcctggtggagagaaatacagtctcctaccttggatgtgccatccagcttggttcagcggctttctttgcaacagtcgaatgcgtccttctggctgccatggcctatgaccgctttgtggcaatttgcagtcca

SEQ ID NO: 559 - DNA, 368 bp, Homo sapiens
agggagcactgggtctaatttttgggggctgagaaggtaagaaggtgaggtcagtttttcccaggagtcctaaaaaattctggtaccttacattgagggtgtgggagaaagggtgtcatagttctgaaaataggcagtagcatgaagcaccagacctgtctcattccttattagatgtctatctcaaataacagagtttgaaaaatattggttttatcatttgatatttccatgcctgactcgggaaaataacattttctgacttctttctattttcttgccctgcacagaccctacctggtacaattttctatttcttagctcaaagtgtctatacaatgggttgcctggtatgtcagctgccctca

SEQ ID NO: 560 - DNA, 204 bp, Homo sapiens
gaaactgttgtgactgaatatctaaatagtggaaatgcaaatgaggctgtcaatggtgtaagagaaatgagggctcctaaacactttcttcctgagatgttaagcaaagtaatcatcctgtcactagatagaagcgatgaagataaagaaaaagcaagttctttgatcagtttactcaaacaggaagggatagccacaagtgac

SEQ ID NO: 561 - DNA, 70 bp, Homo sapiens
tctttgtgaatttgattccgaaacccattgcatcttattgaaattatccttcattcgattgtctcaactg

SEQ ID NO: 562 - DNA, 65 bp, Homo sapiens
gattttgaggtgttcttccaacgacttggaattgcttcaggcagagcacggtatactaaaaattg

SEQ ID NO: 563 - DNA, 37 bp, Homo sapiens
tctgaggcaactagcgattggagtcgaccccccagca

SEQ ID NO: 564 - DNA, 40 bp, Homo sapiens
tggagaggaagtctcaaagtgccctacaatgttggacctg

SEQ ID NO: 565 - DNA, 39 bp, Homo sapiens
atccttgaatatcataggaaacttaacatttgaaagaga

SEQ ID NO: 566 - DNA, 57 bp, Homo sapiens
tgaaaatgtttcggatattgtaccacctttcagtgctttctctcctcaaggaatgcc

SEQ ID NO: 567 - DNA, 25 bp, Homo sapiens
tttgtaaagacaatcataccatgtg

SEQ ID NO: 568 - DNA, 25 bp, Homo sapiens
tggtgtacttgaaagagataggaag

SEQ ID NO: 569 - DNA, 121 bp, Homo sapiens
tgagtgtaggagataactgtatataggctactgaaagaaggattctgcatttctattcccctcagcctacccactgaagtctttgggtagctcttaagccataactaaggagcagcatttg

SEQ ID NO: 570 - DNA, 26 bp, Homo sapiens
ttactgttgctggaagtgtcccacct

SEQ ID NO: 571 - DNA, 143 bp, Homo sapiens
taaaaccattaggaactggcacgtgaatagacagatcaatagttaatagctgtattagccagaaaatggtgtaaggacaacaggctaactaaccctgtcacttgttatgctaaaattaagtctagatagagtcctccaaaata

SEQ ID NO: 572 - DNA, 112 bp, Homo sapiens
caaattaaatagacgtgagtggatggaaagacgtaccttattcttgcataagaagacaacggcatgaagacatcacagggaaggttaaggtaaacatgcagcctggaatagc

SEQ ID NO: 573 - DNA, 176 bp, Homo sapiens
ctgccttcgcagacaggaatgctgtccttccagcttctcctacccttcgggtcagagcccaaaggtccccacccctggttagggctgttttcactgggcggggggcgggggactacactccttggtaactgtcacaactgcccttaactaatgatttgtgcattgccacctgaggc

SEQ ID NO: 574 - DNA, 498 bp, Homo sapiens
atcactgagggatttccgcgagctcggcctcacttctgccccgacttgtggctcggacccagggaccttcagggcccgcagaccctcccggcgccttgagacccgaggcgcccctaccggcccccctccccggttagcgggcggttgtaaggtctccggcgggcgctgcctgccttcctcccagagggtgtttcctagaaactgataaatcagatcgtgcctctttacccttggctttcgaagcaaattgatgttcacgtctgacgtgggcgcgggctgcgcagggcggcgccagaccccagccgcctcccaggggctagactgagcccggcacaaggggtgtgaaatagaatttattgtggctctgattatgtacacgtgagatggcctggctgggccggccgggctcacatggtttgtacaataaatacatctgtggggcgggctctccgcagccgggaagggccaccgccacggttcagtccagcttccgggctc

SEQ ID NO: 575 - DNA, 194 bp, Homo sapiens
catcccttgacggttctggccttcccaaactgcttttgatcttttgattcctcttgggctgaagcagaccaagttccccccaggcaccccagttgtgggggagcctgtattttttttaacaacatccccattccccacctggtcctcccccttcccatgctgccaacttctaaccgcaatagtgactctgtgct

SEQ ID NO: 576 - DNA, 489 bp, Homo sapiens
caatgtaatgtttaggcacgctgcttgggatgctacttctaaaaaaattgttggccatttttcagaatatccttttggttttaaatactggtcaggaaaaacaaatgatgtaaaaatacgtgaataattttctattacagaaatgaaaaactgatttgcatctaaaagtgcaagaggtgaagtaatttaaccctttcaccagacgatatggcaatatacaatatattgcttgagctgtttgagaaggctgtgatgtatttttgtattgacatagaaaattataaattacattgaattagtatccataatcactatatatatacacaaaccagttctaaaaaaatacactggtttaaatttatgagtgaaaacctcacaaggtacagtaaacaattagcatgcttcggtgccagattttgatttctacttttaaaatactagcctgtaaaatgaacgcactctaattccattagcagcacgagcattttc

SEQ ID NO: 577 - DNA, 469 bp, Homo sapiens
gctggctcacagctaactatctggatatggggttttgttggctcttagctgactgcattatttaatcaaatattaagaaaggtataattcacatgcaacccttggcaaaaagaaaagaaggttaatttcttataaccagcacaaactgttaacctgttaagaaaagcccaactgtttctgcatttgaggcatacacacacatcatctctgtttctttataaactggagcgtgaaaggaggagggggacaaataagataaagccatgagctaaacacccagcaggccctactccttattctctgtgtgggtggcagcattagtcagaaagtcactcctctgctgtgatggtcatgacatatttgaacttgtagatctttaaggatttccttatttattctgccaggtctatggtaggtgcttgtttgttttcttggtggaatgattcatcaatgcttcctgagaggctca

SEQ ID NO: 578 - DNA, 66 bp, Homo sapiens
gctccagtttcactgatacctgctgtttctgaatttgatggaacatgtttcttatgacagttgaag

SEQ ID NO: 579 - DNA, 68 bp, Homo sapiens
tggtggattcaagaactccctagtgaggagctgaacttgctcaatctaaggctgattgtcgtgttcct

SEQ ID NO: 580 - DNA, 1103 bp, Homo sapiens
tcttctctccagctaggtgcacttgaggttgttcataaatgtaaaattatgtcaggtttctaacatgggacactgcacacagttgtctgacctgatgaaccatcccatttgaaagtatagattattattatttcttgtagtatttggttgttttccatctcattcatgaacaactcaacctgatagtagtatccaataaatgcctttcagggctcaggaatgaattgacatcctagttaagaaatgagacttaataatggagactgaatgaggcggtttgtattaaattatatgccatgaagtgttcattttagctttaacctaattatgactgtaccaccatgaagtacagaatgaaaaattatatatatgggggggaaacagaatgaatatctgattcttttgaatgcttgtggaaatctttgagatcgtgcagggcataccacaaaatagcctttagaacagatacccaattttacagttcataggacaacatcaaacattagtaagtctaaataagatgaatagaatttttgttatgtaaattttgctagaacagtctattttcttgcacccctcaagttaacctcttaaaaaaatgaatgtataatttctaccgaaagaatatcagagagaatctctctggcctatagtgttaaaatattgttcacaaatcctgattagttaagtgcatacattatgaaacttacagaataaaacttattatacatctctttcttaaattaatatctttacacattttcaactggctccccaagtctgataaggaaggattaaaagaaaaaagaaatgtattagttgggtggccaaggagtttcctttgtaatgttgagagacttccgctttctgaatttcgctggttctctaaggtaaaagagttaaatagtacccttgttcaccaaggaaagtgatccaaactatatatctagtgcagatatttcctttgcattatttagtcttctctggagagaaaatacagtttccccttcctctttctcttcacatttactcttttcaacccaaaataagagacatagaaagcaaaccacagccagtttggcatcttctcagtgctactagta

SEQ ID NO: 581 - DNA, 649 bp, Homo sapiens
tggcttagttagcagacctagaatctgcccaggtgagacctagaacaaaaatagctggggtgaaatggataagagaggtagaggtatatgtcaaggcagagccctatgaggaaggaagagttttcaaagaatatgaggaacatagtgctgagagtgtggctgccttcagcaccgtacacctaatctagagaaaatatttcccatgtgggaggtcctgtctgcattcagtccacccttttctgcctgcttcttcctccaagtgcctcaacctctacatgctcactctcctccccttccctcagcccatcttggtctaagcagctttcacaatccaaaccaaacatcaccagccacccgctgataagtcaccagcatttactttcctgagttactttttctccattcattgagactatggattcatcccaactccttctaaatccctcaaccatccagctatattttggctaacctttgccctagacactctaccagatgttaatgcagtatcaagtgtaaattgtgtcaccctattctgttctacccttttccctgctgccgaaatatcttgctctcctctacctcatccccaaagagcctataaattcagagtatccaaccttttcatggattcactcactgttgttca

SEQ ID NO: 582 - DNA, 200 bp, Homo sapiens
ctagtgtcaaagtttagccgtttgtttttttttttttgttttttgcggctgtaatgtgcaatgatgtgttttattttccttgatgcttaacattactaacaattgcaaaaataatactgaggagcactactttgcattgtttgtagttggagttttggatactgatcataaatcatgaatctggcgtattaatgcttaac

SEQ ID NO: 583 - DNA, 126 bp, Homo sapiens
ttttaaagactggaggtattgctgagtggttgaatggtttggaatatgtgccacaaagactgaaaaatcttaatgaaattaataagctgctagcacatcgaccttggctgatcacaaaagagcaca

SEQ ID NO: 584 - DNA, 473 bp, Homo sapiens
tggcgagatgtggttctgctatttattttaagttagaactcttattctgagagttttccatagaaacacagtaagatatgtggttaagttccagagtactgttaatattacaggtatgttcaggcatttgttaattagcctagaaacctaaccactgggtataacgtttccagaggaacatatatcctgaaattcaagtaaccgcttcactagagactgtctttcctaacttgtttactcagcattaaaaaggtaatgattttggcaaaaaaaaaaatatttttcaatatgtttactccaaggatgtgcttgtcggttgtgctgtgtctaataggaaaactgtgttaatgaagttccatccaatatttaggtaggagttgaatgaagaaagagataaagttgggtaataataagcagggtgttgggacaactgatggccaatatcaaactggcttatccttgttcactttgta

SEQ ID NO: 585 - DNA, 47 bp, Homo sapiens
tcctgctttgtccttcgatgccatcagcactctgaggggagaatatc

SEQ ID NO: 586 - DNA, 27 bp, Homo sapiens
cacctgaacccaggtacttctctcttt

SEQ ID NO: 587 - DNA, 193 bp, Homo sapiens
ccctgtttgcacctctctaagtgaattaccttaccccaattcattctccttgttttttgctcagtattttaatcaatttaataagccatgtggtttggtcattttaatttggtctgaaagcctttctgttcctcgacctctgggatagctcaccagttagtgcttccactgcatcaaggcaatttcccacata

SEQ ID NO: 588 - DNA, 191 bp, Homo sapiens
tggcatttacacagcagactcaggacaaagggacaaatggagtgagtgaggtgggcaggagctttagaagagatgaagcaaagagagttagttttttattcaggaagatgtagagactcttaaaggagaggcaatttccgtaacaaacacctctggtttaatggaagccacatgatttatttgactactgg

SEQ ID NO: 589 - DNA, 131 bp, Homo sapiens
cctcttccagaaagctgtccgtttctggcctctggatcctgaagtcaggcatcattggtgtaggcgtggtggtagcaatgaccctgcagcaacttggggaaaagttcaaagctctcttttcagctgtctta

SEQ ID NO: 590 - DNA, 39 bp, Homo sapiens
gtgccaccacagcttatggcatctcattgaggacaaaga

SEQ ID NO: 591 - DNA, 123 bp, Homo sapiens
gcaccattgcaatcctgaattctgtcaagaaagccgtggagtcaaagagcaggcatcggagtcggagcttaggagtgctgcctttcactttaaattctggaagcccagaaaaaacgtgcagtc

SEQ ID NO: 592 - DNA, 25 bp, Homo sapiens
agagaagcggctgttttcattgaag

SEQ ID NO: 593 - DNA, 59 bp, Homo sapiens
cagaagaaaggcctttgttggcagcagtgtttaacttgcttctcccttcatattcctcc

SEQ ID NO: 594 - DNA, 72 bp, Homo sapiens
ggcagacatctaaccgttccgaaccctcaggggagatcaacatagacagcagtggtgaaacagttggatctg

SEQ ID NO: 595 - DNA, 122 bp, Homo sapiens
caaagaccttttgaaccatgccttcttccaagaggaaacaggagtacgggtagaattagcagaagaagatgatggagaaaaaatagccataaaattatggctacgtattgaagatattaaga

SEQ ID NO: 596 - DNA, 354 bp, Homo sapiens
tgaattcatacgagcatcaagatggagacatatgaatctcaaaataattatgcaaagtgaaagaagccagacaaaaaaaaaagacatactatatgattccattcacataaaactacaaaatgcaaaatgaaagccgattggtggttttctggggaggagtcatcaggaggagcattgtgacactttgagggatgatggatatgttcactctcttaattgtagtatgcatgttctccacaaggtgtgaaaacttatcaaatagtacactttaaatatgttcagtttattttatgttaattatatctcaataaagcttttaacaaaagcaaacatctcttgcctacataaggatga

SEQ ID NO: 597 - DNA, 96 bp, Homo sapiens
tgagttgactgtttttaaataactctttggattttaattgtgatgcagaagttatagtaacaaacatttggttttgtacagacattatttccacgc

SEQ ID NO: 598 - DNA, 126 bp, Homo sapiens
tctaagttcaaccagcaggggctccctgaaggccagacctgccagctggcaccaagggcttccaggtggtcatctcagctaattgacaacgccactggagagattgccaactacactgactcatag

SEQ ID NO: 599 - DNA, 64 bp, Homo sapiens
aaccagggtcacagtcatcgcgttatcccacattttgagcaaggatagagaaggtgagttatta

SEQ ID NO: 600 - DNA, 40 bp, Homo sapiens
ctgtacttttacactcctttgtcttggaactgtcttattt

SEQ ID NO: 601 - DNA, 62 bp, Homo sapiens
gaatgtcgtgttcagttccagccgggggccgtatgactatggatctaattccttttaccagg

SEQ ID NO: 602 - DNA, 190 bp, Homo sapiens
tttatgtttagcaccgtcagtgttcctatccaatttcaaaaaaggaaaaaaaagagggaaaattacaaaaagagagaaaaaaagtgaatgacgtttgtttagccagtaggagaaaataaataaataaataaatcccttcgtgttaccctcctgtataaatccaacctctgggtccgttctcgaatattta

SEQ ID NO: 603 - DNA, 26 bp, Homo sapiens
ccaccaccagatgagaagttaagcag

SEQ ID NO: 604 - DNA, 65 bp, Homo sapiens
agcctcagtgtaaagtctgccgatcatgccctgtggtgcagtcgagccagcacctgtttctggac

SEQ ID NO: 605 - DNA, 88 bp, Homo sapiens
caagagtccagctttatggccactgcattcagacattaccagattccaagtttttcccatttgaagtagctggtgcagttcatttctt

SEQ ID NO: 606 - DNA, 158 bp, Homo sapiens
gatgagcgccttaagaatctctttggcaagtttgggcctgccttaaatgtgaaagtaatgactggtgaaagtggaaaatctaaaggatttggatttgtaagctttgaaaggcttgaagatgcacagaaagctgtggatgaaatgaaaggagctcaatg

SEQ ID NO: 607 - DNA, 117 bp, Homo sapiens
gaaaatggttgggtgcacgaggggccagatgctggaaccagttctgaagaagtgttcctggggccaagaggatctgagaggtggccaggtgtgaagactgaacaagctgagcgttaa

SEQ ID NO: 608 - DNA, 30 bp, Homo sapiens
actcctgagacaacatgaccaatctctccc

SEQ ID NO: 609 - DNA, 32 bp, Homo sapiens
agagaaggaggccggaactacaccagcaaaag

SEQ ID NO: 610 - DNA, 31 bp, Homo sapiens
aggatgcaggagaacttgactttagtggtct

SEQ ID NO: 611 - DNA, 348 bp, Homo sapiens
agtgagacattgtggcctggaagtctttcagacacccactcagagaagtcaagtttaaagtggatgttcttcagagaatttttcatttctgaaaatgtgttttgcttatagaatataacagagttgactagaaagagagaaacaactgcatactaatcttttaaagcctttaacagttgcttttaaactttctttttaaatgtttcatgactcttcacctatttttttttaaatggggacgaagagatatgaaaactgagacataagacaaatacctagaaacctctaagactgcacatatgatttggtagaagtctgaaggtatacacattgtaagaggcagaccta

SEQ ID NO: 612 - DNA, 99 bp, Homo sapiens
ctgatcctccagtggcaccgactgtgacagaggtgggagatgactggtgtatcatgaactgggagcctcctgcctacgacggaggctctccaatcctag

SEQ ID NO: 613 - DNA, 102 bp, Homo sapiens
ccaaattgtgaagattgaggatgtctggggagaaaatgtcgctctcacatggactccaccaaaggatgatggaaatgctgctatcacaggctataccattca

SEQ ID NO: 614 - DNA, 81 bp, Homo sapiens
tcagaggcacccatgtttactcagcctttggttaacacctatgccatagctggttacaatgccaccctaaactgcagtgtg

SEQ ID NO: 615 - DNA, 28 bp, Homo sapiens
atgacggactccaagccgatcaccaaga

SEQ ID NO: 616 - DNA, 114 bp, Homo sapiens
tgagatcccaggctcgcccatctttctcatgaagctggcccagcacgcccgtcacctggaagttcagatcctcgctgaccagtatgggaatgctgtgtctctgtttggtcgcga

SEQ ID NO: 617 - DNA, 31 bp, Homo sapiens
ccagtgccctactgccgtcagcttcttttaa

SEQ ID NO: 618 - DNA, 93 bp, Homo sapiens
ttcttcaagcaacaaaggtggtagaggaaattcctcaactttctcaacgagtcatgtaacgttacactggcctccataaagcaccgattaaga

SEQ ID NO: 619 - DNA, 176 bp, Homo sapiens
taagcctgaagaactggttgactacaagtcttgtgctcatgactgggtctatgaataagaggtggacacagcagcagtctccttcagcacggtgtggtgtgtccctggacacagctcttcattctactgacttagaggcaacaggattgatcattcttttatagagcatatttgcc

SEQ ID NO: 620 - DNA, 312 bp, Homo sapiens
tatggcaaatgggaaccggctactaagtaaagcgtgctgtcaatatgcgttcaaaacaaaatccctacagtggtattagcttatgaaaaggaacaaagaacaccatgggtaacaaatgtatacaaaagagaagattaaagggagacaatggtgtcttggaggcaaactacagtttgctgtaagataactttccgtgcatcttttaaatcaatgcttaaaaaacaaaaaaaacctgggcagttcctaactacttaaaatgcaaatcctaattaactgcaaaatcttttctcaatctttgagactgtaggttca

SEQ ID NO: 621 - DNA, 56 bp, Homo sapiens
atgcacgtgccctacgtccctctctctcctccttcaaaaacgtcatttcttaatgc

SEQ ID NO: 622 - DNA, 194 bp, Homo sapiens
cttctgggacatctctctctaaatcaaaagaaaactcaatagcttcattatctttgtattttccctttaatttcttaatatcttcaatacgtagccataattttatggctattttttctccatcatcttcttctgctaattctacccgtactcctgtttcctcttggaagaaggcatggttcaaaaggtctttg

SEQ ID NO: 623 - DNA, 38 bp, Homo sapiens
tgaggcccacggctctccgggccctgtcgctctgggga

SEQ ID NO: 624 - DNA, 83 bp, Homo sapiens
agaggacagggtcaccggcgaggaccgccttgcaccgtctcgcagaggcatccctttcatccgctcttccatgtcctttccct

SEQ ID NO: 625 - DNA, 30 bp, Homo sapiens
ggagaacatttctgcaatctactcatctga

SEQ ID NO: 626 - DNA, 296 bp, Homo sapiens
caatttacggcaatagacatttacagaacaaaaataagacagttccaagacaaaggagtgtaaaagtacagcacacaggttaatactcttcaccctcatcctctccgtcagcactatctgctccaacctcctcataatccttctcaagggcagccatgtcctcacgggcctctgaaaactcgccttcctccatcccctcacccacgtaccagtgaacaaaggcacgcttggcatacatcaggtcaaacttgtggtccaggcgagcccaggcctcggcaacagctgtggtattgctc

SEQ ID NO: 627 - DNA, 120 bp, Homo sapiens
cccaaaggtgttgagtcagcaaatcctgcagcctttgtgtgactttgagcatcactttcccctttcagcattaaatatatgacctctctgccttattttagaacttactacatttcaata

SEQ ID NO: 628 - DNA, 178 bp, Homo sapiens
ttctcctactggctaaacaaacgtcattcactttttttctctctttttgtaattttccctcttttttttccttttttgaaattggataggaacactgacggtgctaaacataaatataaatacagagaccacaaatacagctaaaaatcttcacacagaaacggtcacagtttgtaaa

SEQ ID NO: 629 - DNA, 236 bp, Homo sapiens
tacatattggcgttgaaacagtttaaagaaatgaaatggctatctacaaaaagttagttttgattgctgtcttcccccatactttgtgtcttcacacataaagaaaattttcaaagattttatattcagcaattttttaaaaagtacactgttttccactgctatggtctttataaaggacttgacttaaaatttcaaataaaaaagaattaaggttctaggataactcttgtgtc

SEQ ID NO: 630 - DNA, 25 bp, Homo sapiens
tgctgacaaagacatacaggatgag

SEQ ID NO: 631 - DNA, 67 bp, Homo sapiens
ctggagcttggtggaacagtcaatattacatgtcaaaaaactggatacagtgcaatacttgaattta

SEQ ID NO: 632 - DNA, 47 bp, Homo sapiens
ggaaatattgaagaacgtatgagacatttagagggtcaacttgaaga

SEQ ID NO: 633 - DNA, 173 bp, Homo sapiens
gcgctcgcaggctgcctagtcgttcgctcgctctctctcgcgtgctccctctcgccgcctgtggggaagtcggggccgcccgagcggccgtcgccaccgcctcgccccgacctccttcccggtcccctcccggcccagtccctcccgcccgcgcaccccgagtagtgagtggc

SEQ ID NO: 634 - DNA, 122 bp, Homo sapiens
tatagcctaactgtattgtcagggcttgacttatgaaacctttcccaaggtctcctccatctgcctgcacattcccctgcctcactggattatagtttacttttagttagctattcttaaga

SEQ ID NO: 635 - DNA, 255 bp, Homo sapiens
acacgcattggtaggtcaccggccccttatctcacagtgcactcacttggacaggggacatctgtcataaacacgttctgttgcacaacctgagacaggcctgtcacttgctttgctcttcacttcatggctctgatctgcacgcactttctaaggcctaggacaagccagaaattgcttccaagacacttgtgtggcgcaacctctatcggcaagtgcagtttgcctcacgggcttcgtcaaacacttggcaca

SEQ ID NO: 636 - DNA, 68 bp, Homo sapiens
gagggcttatcgagacaaattgaagttttggacccattgtgtactagcaacattgattatgtttacta

SEQ ID NO: 637 - DNA, 76 bp, Homo sapiens
tggccacgtccagttctggacagctcctagggtcctgtcctcactgaagcacttatgccggaaagcccttcgaagt

SEQ ID NO: 638 - DNA, 82 bp, Homo sapiens
cgacaaactagtacagagaatgccctgtacaaaacacaacaaaggttcaaacatcgagatgttcccttagcaaggctgaaaa

SEQ ID NO: 639 - DNA, 168 bp, Homo sapiens
gcccagatctgggtagattctttgctgtctgatctgagagatgcctagaaacagctctgagggctgaccaccacagagtgctgcctgccaacattgtttccttctccaaacacaagacgggacagacttttgagtgtgggggcatctttggtccgagaactgtcctgg

SEQ ID NO: 640 - DNA, 153 bp, Homo sapiens
agatctattcatcaagcctgtttcaatggtgtgcatgtttccctgctattggcctcttcaaccccagaaaaggtcatgaactatatccctccagctgcccggcttgttacgtcggctgatgggacagatggcagtgccgatgataacacatta

SEQ ID NO: 641 - DNA, 25 bp, Homo sapiens
cccgtgtgctttcatcatcgagccc

SEQ ID NO: 642 - DNA, 140 bp, Homo sapiens
cagaacttctttatatgctcgagtctccagagtcactccgttctaaggttgatgaagctgtagctgtactacaagcccaccaagctaaagaggctacccagaaagcagttaacagtgctaccggtgttccaactgtttaa

SEQ ID NO: 643 - DNA, 312 bp, Homo sapiens
cataacatacatacaaggctcggtcttttcaatgggataacagttcacaactcttcgatttgaattgtaatgaatctggtgacaaggatttttctctaatggattccaaagttagccagaacttttaatgtcaagatgaaaaagggtgtaaggtgttatattttcttcaattcctttaccacaggaggctaactccacaatttccctcatgtttctcattcagaaaaaaaaatattaaatttgtgttcagaattatttgatgattgcttctttgtgctgatgtttcagttcctgaagtcaacttggctctca

SEQ ID NO: 644 - DNA, 100 bp, Homo sapiens
gtgagggaatatgtccaattaattagtgtgtatgaaaagaaactgttaaacctaactgtccgaattgacatcatggagaaggataccatttcttacactg

SEQ ID NO: 645 - DNA, 365 bp, Homo sapiens
tgctaatctccatgcccacgttctttcccaccctgttcccagtcttctgacaaactgtgtacatagcggactcctcctttctcctccgaggtggttttaaaggctttttggtgtatagaagtttgtccatttgtaaaactccggattgcgttcctccccgccttccgccccttcccttccctaaagtgatgggctttctcttttctctttttagtttacccggtttctttttaagtaatgtggaagaaaatggtttattttgtattgtggtattgaatattgtgttcctttttatgaggcaacctgattgtaaacttcatgtaactatagactggaaaaaatgagccgtgccaaagtctcccttc

SEQ ID NO: 646 - DNA, 98 bp, Homo sapiens
gcaggtgtaagtgtgataattcagatggaagtggacttgtgtatggtaaattttgtgagtgtgacgatagagaatgcatagacgatgaaacagaagaa

SEQ ID NO: 647 - DNA, 122 bp, Homo sapiens
gtggtgaatgtacctgtcacgatgttgatccgactggggactggggagatattcatggggacacctgtgaatgtgatgagagggactgtagagctgtctatgaccgatattctgatgacttc

SEQ ID NO: 648 - DNA, 136 bp, Homo sapiens
atggacagtgtaattgcggaagatgtgactgcaaagcaggctggtatgggaagaagtgtgagcacccacagtcctgcacgctgtcagctgaggagagcatcaggaagtgccagggaagctcggatctgccttgctc

SEQ ID NO: 649 - DNA, 70 bp, Homo sapiens
ctatcctccaggagatcgccgggtgtatggcaagacttgtgagtgtgatgatcgccgctgtgaagacctc

SEQ ID NO: 650 - DNA, 51 bp, Homo sapiens
tgtgagagaggatggtttggaaagctctgccaacatccgcggaagtgtaac

SEQ ID NO: 651 - DNA, 112 bp, Homo sapiens
ttcttgtcattgtgggaagtgcatttgttctgctgaagagtggtatatttctggggagttctgtgactgtgatgacagagactgcgacaaacatgatggtctcatttgtaca

SEQ ID NO: 652 - DNA, 342 bp, Homo sapiens
tggttcctaacgagagcaatttttccacccaaaagtcatttggcaacatctacagacaattttgattgtcacactgggtcgggtaggaaggtatgctgcagacatttggtgggtagaggccagggatgctgctgagcatcccgcagtgtacaggacagcccccaaacaaggaattatccagccccaaatgccaatagggctcaaactgagaaacattgagttatatggctattagaaatccacattcttacacaagaaagaccatattagaatctaaggaaaacatgcatattcacattaattaatcgatcagatttttccagaattccgtatcagtcacca

SEQ ID NO: 653 - DNA, 180 bp, Homo sapiens
gtgttggcactaacgctgcttgtttggcaaatcatcatcactgaggtattccacccagagactttttcaaaaaagtcaactaaagtgctaagtcataagaggagagccattatacccgttgtctttctgggctccttgagtttatctggattccaacagcacttggaaagtaccgccctc

SEQ ID NO: 654 - DNA, 117 bp, Homo sapiens
ccctcccagtaacagctcaataatgaccctcatcctggactccttaggagtatctctcctcagaggaggcttgaacttaaaggaggaggaggtctccaagtccaagttcttactttg

SEQ ID NO: 655 - DNA, 124 bp, Homo sapiens
tgcaggtccttcacacaagtctctgctctggaaggctcgagttgcacttcatggactgggtgtctggacccctactggacagctcccagtaggcacagatgccgtctctcacttgtcagggctc

SEQ ID NO: 656 - DNA, 25 bp, Homo sapiens
gtaagactctatccttccaacatcc

SEQ ID NO: 657 - DNA, 36 bp, Homo sapiens
tctagaagacgattaagggaaggtcgttctcagtga

SEQ ID NO: 658 - DNA, 72 bp, Homo sapiens
agggagaaacggtgcgattcacatattccgcgagatcatcaagccagcagagaaatccctccatgaaaagtt

SEQ ID NO: 659 - DNA, 114 bp, Homo sapiens
tggtcacagcaagaaaggcatgggagccagtgaacaaaggcatggtgacttgagcactttggcatttcatcattctgggagagtgttcacagcatgcacagttccaggaagctc

SEQ ID NO: 660 - DNA, 34 bp, Homo sapiens
gcccacaccagggaccatgtttcatcctcctaaa

SEQ ID NO: 661 - DNA, 141 bp, Homo sapiens
gtgagttgtcaaatccctagactgtaagctcttcaaggagcaagaggcgcattttctccgtgtcatgtaatttttctaaggtgcttggcagcactctgtaccctgtggagtactcagtaccttttgtttgatgttgctgac

SEQ ID NO: 662 - DNA, 137 bp, Homo sapiens
gagaatcttctttggtgagtttaaagtgagagtagacaaataacttagtggctaaatgatttaatgtcagatgacttaatgaccagtttttgcccttgatccagccccactctgatattctacctcctgtgtgcttg

SEQ ID NO: 663 - DNA, 31 bp, Homo sapiens
acagtgtcctaacagtgaaaatcagagttat

SEQ ID NO: 664 - DNA, 30 bp, Homo sapiens
gcacacattgttacagctagagtgtgaaaa

SEQ ID NO: 665 - DNA, 102 bp, Homo sapiens
atactgatgttgtacactgttttacttaacattttgggaagtaactgcctctgacttcaactcaagaaaacacttttttgttgctaatgtaatcggtttttg

SEQ ID NO: 666 - DNA, 985 bp, Homo sapiens
cagttacgaaggtgtagtgccagcaggatatatttgggggatggtaagttctccgatggtgaggtataaggcggagtggcaaggatgaggtgaacagacaaccgcttctctttccccattctttagacaaatagtgttatgttacatgctgttatgcaaccctcccttttccccttaatttgttttggatatctttcttaaggaatttagagccaccttttttaagggttccatagcaattaattgtttctaatctttagctattacaaatgatgcagtaataacttcttacgtatgtcattttatatctggtgaaaatatatctgaaggttaagttcctagaagtggaaactactggatcaaagagttagtgcttttataattttgaaggacattgccatattgtcctcttcaatatgtacttctgtcagctgttatgagagtacctatttcccacatgcttgccagctaggagtcttaacaagctttttgatctttgccaatctgacagttttaaaaaggatctcagtatggttttaagttgcatttcttttattatgagtgaaagaatatttttacatgcgtaagagtgacttatttttgtctgctgaaaacatctcaattagtatagggacctagagcaaattatttaagctttctatgtctcagtttcctcacctgtaaattgagaataaacaatagtaagtaggattattggataattaaatgaaatgtatgtatggcactcagcacagtgcctggcacatggcaagcattcaataagttatgtgatggctaacaatcagaggagcaggtaggacagggcagattaaattaagaaagtagcattcacttccatgctccagtcacatggagccttgttcatttacattgccattcatcagagaagtacgaagtcagagctttccttgtcttttaatagtaaagagtcatttcccattggaacccatcataggctatcagc

SEQ ID NO: 667 - DNA, 107 bp, Homo sapiens
gctgtgaaaacatcccacggcagtcctaagggagccatccgagcctccaaatcctgtcccctcttcactgcctgcccccactgccaaagaaagctacagtgttctct

SEQ ID NO: 668 - DNA, 186 bp, Homo sapiens
agacgccattgtatttgcagcaacaccaaaagctcatttggcgaagctcacccagtaatgaggctcataccaaaggcacgagccatgagtcaatgcacagacagcacttgagcatgacacgtgtgtatgtaaaagcaaaggggaaaagtgatgaagcaggatgcggggagaaaacatcaccatctc

SEQ ID NO: 669 - DNA, 28 bp, Homo sapiens
ctcgatttcactaacgttgtatatgaaa

SEQ ID NO: 670 - DNA, 127 bp, Homo sapiens
aagaagcagatgattggctcagatatggaaacccttgggagaagtcccgcccagaattcatgctgcctgtgcacttctatggaaaagtagaacacaccaacaccgggaccaagtggattgacactca

SEQ ID NO: 671 - DNA, 429 bp, Homo sapiens
gttagacttgattccgcctcgcagaactgctcttggactgagaaacacttttgtcctcagcttttagatctttatgatggcgattcggaagacacccccagtcattccaactggagctgcaacgactgttctctgggtgacatgagctgtaaggtggcttcctacacactgaagtcagagtccagaggaagttcatatagtagacgccctgtcacaaaaaacccacattcttgtcatagaagtggccagctgggcccgtgtccctggaagccaaagatgagggcacgtagcccgaggactcgaggatggcggaggaggaccagacccccagggccgccgagcccttcctcactcccatggagccccctgagactggcagacacgtgccaagcagagctcgaggggcatggccaccacctaggtttggga

SEQ ID NO: 672 - DNA, 246 bp, Homo sapiens
tttgagctattgaaaagtcatcctgccagtggaacactacctcatgttcattaactcttctctctctctctatttactattaaaatgtcttgggtgggggtgggggtcgaagagtcaacttgtcagaggattaaaaacaattcaatgtatatgcctcttatttggatgacatacttgaatgttatacatgcttaatgcaaactaacacaactacagacacattcactgtacagtgccagagttcaa

SEQ ID NO: 673 - DNA, 101 bp, Homo sapiens
gggtgacgttgctgatagctcaatacttaacgtacagcaggaaggagcactgaggcagtggcttgagctcagtctgtgggaggagacctgttttgatccag

SEQ ID NO: 674 - DNA, 470 bp, Homo sapiens
cccactttagtcacgagatctttttctgctaactgttcatagtctgtgtagtgtccatgggttcttcatgtgctatgatctctgaaaagacgttatcaccttaaagctcaaattctttgggatggtttttacttaagtccattaacaattcaggtttctaacgagacccatcctaaaattctgtttctagatttttaatgtcaagttcccaagttccccctgctggttctaatattaacagaactgcagtcttctgctagccaatagcatttacctgatggcagctagttatgcaagcttcaggagaatttgaacaataacaagaatagggtaagctgggatagaaaggccacctcttcactctctatagaatatagtaacctttatgaaacggggccatatagtttggttatgacatcaatattttacctaggtgaaattgtttaggcttatgtaccttcgttcaaata

SEQ ID NO: 675 - DNA, 34 bp, Homo sapiens
tcctgtgaagctcacaacctaaaaggcctggcct

SEQ ID NO: 676 - DNA, 34 bp, Homo sapiens
ccagaaggttctgaaccggagtctattcacaggc

SEQ ID NO: 677 - DNA, 126 bp, Homo sapiens
tgacctgagcaatggagaacctaccaggaaacttcctcagggtgttgtttatggtgtggtgcgaagatcagatcaaaatcagcagaaagaaatggtggtgtatgggtggtccaccagtcagctgaa

SEQ ID NO: 678 - DNA, 40 bp, Homo sapiens
atccagtgggggacagacaagctatctcggggataggaag

SEQ ID NO: 679 - DNA, 644 bp, Homo sapiens
tgctccattaccaaccgaaggctcggtcttaattcccccacctgtctaaggggaacagaaaacccctgccaaaactaccctttcccccatggggtgctgtgaaactcaaggagatgactgtggatgaggggtccatgcaggcatctccaggatgccctgacactgtgctttaggactggctgcgctcatgggcagagtagcttgtggccctacccccctgaatccatagagcctaagattctcaaattagcacacccttctcagatggccacgatgtaggctccactttgccaacttcccgagaacaaacaggcctgctcacaactgcccctcaagttcaaccccgggaccgcccagccccgcaaagcaaagaccctgacttctcctgctcttgagccagctgtgctttgcccgtggccagaagctcagcagcggctcctgggaaaggcctggaaaggatgagggaacaggcaaggaacaaaggcaacctttgcacactcacctgagccctggggacccttggagaaattaggtgtcacctgctgtggcaaaatgggtttagaaatctttgccaggtcatggcccactggattctggatactctgaggcctggtttgtcaggcaatatgcccttgagcaggtca

SEQ ID NO: 680 - DNA, 41 bp, Homo sapiens
atgcttgtcccagtcctgatcctaagcccctgcctcgttgg

SEQ ID NO: 681 - DNA, 166 bp, Homo sapiens
gaggctggttacagcttcagtccctttgtctgtctgtccatccattcctccatccacccatctgtccatccgcccatctgcccgtccatccatccatccactcttccaccctctaccccttaccttattctaaaaagaacttaggtaggttatggtgcctcacacc

SEQ ID NO: 682 - DNA, 909 bp, Homo sapiens
gtcctcgatcatggtgttagaattgactggatagtaacaggtggtctggtggatagcggggagcatggctcagcaccagagcagaggcccagccagccctctgcagcccaaacgtccccaacggttgcctggcaccatctctctctgatgagacgaatctcattttcatttccattaacctggaagctttcatgaatattctcttcttttaaaacattttaacattatttaaacagaaaaagatgggctctttctggttagttgttacatgatagcagagatatttttacttagattactttgggaatgagagattgttgtcttgaactctggcactgtacagtgaatgtgtctgtagttgtgttagtttgcattaagcatgtataacattcaagtatgtcatccaaataagaggcatatacattgaattgtttttaatcctctgacaagttgactcttcgacccccacccccacccaagacattttaatagtaaatagagagagagagaagagttaatgaacatgaggtagtgttccactggcaggatgacttttcaatagctcaaatcaatttcagtgcctttatcacttgaattattaacttaatttgactcttaatgtgtatatgttcttagattagaataatgcaacttcgagtatgctttaatatttcaatattcaagttacaaatgtataaggcagttagaaataatacagtcacatgtcacttaatgatagggaaacattctgagaaatgcattgtaaggtgactttattgtgtgaacatcatggagtgcacttatacaaacctagatgggacacctatgacccacccaggccagatggtacagcctgttgctcctgggccacacacctgtacagcatgtgaccgcactgaataccgcagg

SEQ ID NO: 683 - DNA, 75 bp, Homo sapiens
ttccttcctgtgtatcaccccagccctgaggagagcagggaccccaccctctatgccaacaatgttcagagggtc

SEQ ID NO: 684 - DNA, 85 bp, Homo sapiens
ccaaggagcagcatatagtccagagggtcagcccatggggagctttgtgttggatggtcagcaacacatggggatccggcctgca

SEQ ID NO: 685 - DNA, 99 bp, Homo sapiens
acccttcttcttggcgagaccacgatgatgcaacctcaacccactcagcaggcaccccagggccctccagtgggggccatgcttcccagagcggagaca

SEQ ID NO: 686 - DNA, 118 bp, Homo sapiens
tctggtctttgagaagtgcgagctggcgacctgcactccccgggaacctggagtggctggcggagacgtctgctcctccgactccttcaacgaggacatcgcggtcttcgccaagcag

SEQ ID NO: 687 - DNA, 316 bp, Homo sapiens
catcccgaagtgtggctaagccgcccggaggaacacaaagggcatacgcgcacgcacacttaaagttttaaaacacgatttatttatttttgtctgctgcaacgctgggagaaatgtggtctttggaaggaagctctccagtgtgtaaccttcctattattttggcccccacactgtggctttagtagaacaggagcaaacaagtttataaggcaaggaggtggagagattaaaagagcattctcttgcatttatgaagtgtcactccggtgtgtatgtaggtgaagcctttggcctcgtctgaaatgcccattaa

SEQ ID NO: 688 - DNA, 99 bp, Homo sapiens
acgggccgccgctccacgccacacagcactacggcgcgcacgccccgcaccccaatgtcatgccggccagtatgggatccgctgtcaacgacgccttga

SEQ ID NO: 689 - DNA, 266 bp, Homo sapiens
aatcgttacacgctttcgcctcgctccagcttttaccccagcagcgcgattcccccggttctcctgctgccgctgctgcgccgctgccgccgtggctcgcgcggctcggggcgggcacgggctgcgtaggagccgccgccggggccgccgctggggccgcgctgccgcagccgctgcgccgccgcctagactactagcctgggctgcttgttttgtctctgaaattgacaaggacgcagggaatccgctgctaaataatgtttctg

SEQ ID NO: 690 - DNA, 50 bp, Homo sapiens
ccttattctacatgttggtggagtttgtggttggtcagctgccctggaga

SEQ ID NO: 691 - DNA, 293 bp, Homo sapiens
ccactggcctgtaattgtttgatatatttgtttaaactctttgtataatgtcagagactcatgtttaatacataggtgatttgtacctcagagtattttttaaaggattctttccaagcgagatttaattataaggtagtacctaatttgttcaatgtataacattctcaggatttgtaacacttaaatgatcagacagaataatattttctagttattatgtgcaagatgagttgctatttttctgatgctcattctgatacaactatttttcgtgtcaaatatctactgtg

SEQ ID NO: 692 - DNA, 341 bp, Homo sapiens
gactgtaaatacgaacccaatctgcacactccaggcctctagttccagaaggatccaagacaaaacagatctgaattctgcccttttctctcacccatcccacccctccattggctcccaagtcacacccactcccttccccatagataggcccctggggctcccgaagaatgaacccaagagcaagggcttgatggtgacagctgcaagccagggatgaagaaagactctgagatgtggagactgatggccaggcaagtgggaccaggatactggacgctgtcctgagatgagaggtagccgggctctgcacccacgtgcattcacattgaccgcaactc

SEQ ID NO: 693 - DNA, 402 bp, Homo sapiens
gtggacgtcacacaaccatagttttgaatgaagaggttttgcagcagggggcaaggggctgtctgaaagcaagtctgagttaacagctgaatcctcctgggtttcacatccttagctgagtactaacaatctacccatatagaaattgttgtagatctgaatccacattcattttgatatttgtcttaaaatagttcccaaatttcatttccataattacaatggagtcctccatttattcttttttgggaaagtggtgattaaattttctatattggacattcaaccacttttccccgttttatttccttaggagggtagatcttgactatgtgcctgcagccatcatgtctcacctcacaacttactgtaataaaacttaactctgcaaggggtagatga

SEQ ID NO: 694 - DNA, 230 bp, Homo sapiens
tcggggccacgttgttgtatgtattgatgtacagccttgaatgtgaataattattgtaaactatattttacaactttttttctggctttattatataaattttctattgggtcagtgatttaatcatataatttaatgaatctgtttatcctttttttttttccaaatacttgtgctttaggtgtagttaccagatgatgaattttcctcgtatggtcagtagtcttgta

SEQ ID NO: 695 - DNA, 125 bp, Homo sapiens
ccagatagtacagcagaaacggttcccggggcaatgggtgctgcattaatcacactgattaaagcagatgaatcattcgtttttcttttctttttgtttgagaagtttgtttatctccctcttgg

SEQ ID NO: 696 - DNA, 25 bp, Homo sapiens
tgccacatggaccagattgcagagg

SEQ ID NO: 697 - DNA, 143 bp, Homo sapiens
gtgctacgtgaacatgaacctcatggatgcctccgtgcctcccctggcccttgggctgctggagagtgtgaccttgagcagcccagccctgggccagctggtcaagagcgaggtgcccctggataggctgctggtgcacctac

SEQ ID NO: 698 - DNA, 182 bp, Homo sapiens
gggcgatgctggcttataacaggtccaagctctgcaacatcctcttctccaacgagctgcaccgtcgcctctccccacgcggggtcacgtcgaacgcagtgcatcctggaaatatgatgtactccaacattcatcgcagctggtgggtgtacacactgctgtttaccttggcgaggcctttc

SEQ ID NO: 699 - DNA, 25 bp, Homo sapiens
acttaataaatctattgctgtcaga

SEQ ID NO: 700 - DNA, 294 bp, Homo sapiens
ctcgccaaacgcgttccagaatttgtccacaggtgcgccggcacctgctttcccacctcgaggccgcggcctcccccccgatttatagacaactctgacattgtcaccccactgacgaggcccgattccatagggtggatccttgccaggcgtccctgatcctccctgcccaagtcttccttcgtgagctggccttgctccccatcccccaagtgcctcaccagtcccccagactgggtgaaggtacagctggctcctttcgggggtgcagcttcaactctctcggcggtaggg

SEQ ID NO: 701 - DNA, 97 bp, Homo sapiens
tcagtaactacttaccgcctcagagttatcgatgaaggagtcggattcatcataaccataccccatatcgatcaagtcctgtattcggtcttttcta

SEQ ID NO: 702 - DNA, 62 bp, Homo sapiens
cagatcattctgagtgtgcgagtgtgtgtgcacatgttacaaaggcaactaccatgttaata

SEQ ID NO: 703 - DNA, 58 bp, Homo sapiens
gtcagatccgagctcgccatccagtttcctctccactagtccccccagttggagatct

SEQ ID NO: 704 - DNA, 182 bp, Homo sapiens
tggctaatgaggttccggaccctctggggcatccacaaatccttccacaacatccaccctgccccttcacagctgcgctgccggtctttatcagaatttggagccccaagatggaatgactatgaagtaccggaggaatttaactttgcaagttatgtactggactactgggctcaaaagga

SEQ ID NO: 705 - DNA, 82 bp, Homo sapiens
atggaaatccgtagctgtctgcccaaactccttttggctcaccataaaaccttaccttactttatccggaacaagctctgca

SEQ ID NO: 706 - DNA, 311 bp, Homo sapiens
tcatgactggacagctaagcggggcaagaaccggcccaggctcccctcgtggggagaggtccaggccagcagttcactctcagtctcacctcaggacagacaccctgcgggcttgagaagagggagactcagtttccacaaatggggaaggaagaaggtggcaaggagtggggtggactgaggaacagcagccgagcgggccggagagccgtgaggtacctgtggtggggcacacctcaggcagcccagccttgggcgaaggcaggcaaggactggcccgagaagtggctgaacgcaaagcgcagtcgtta

SEQ ID NO: 707 - DNA, 65 bp, Homo sapiens
tgccatctttgacacttgcaagctaaatgacattactcaaattaatcgttctgcacttcagcttc

SEQ ID NO: 708 - DNA, 275 bp, Homo sapiens
ttttcagggtaagagattcacacgcaagagaataaggctacaaatgctccaaacatgggtccagaactctggctcttttgtgaggcctagagtcacagaaaatgccatctgttaagaagctgcttttgcataaaaaaatataaaaagttacaaaattccatcaaagacagatacacagtataaatcgttaggtgaacacagaaggctggtctcatctcctgcagtggcccatagacctgccctgaggtctggaagttcactgtcctgagggtcca

SEQ ID NO: 709 - DNA, 36 bp, Homo sapiens
gctggcaagatcacgtgcctgtgtcaagttccccag

SEQ ID NO: 710 - DNA, 58 bp, Homo sapiens
ggggagaggcgtccctctggctgctgctccagctctggcagacacgccaagctttgag

SEQ ID NO: 711 - DNA, 308 bp, Homo sapiens
tctgggatatccacctgccctaatcaccgcagggccaggtaccagaaagctagggacagcccctaggccccacagcctgctgaaattattcgaactcgccagtctgtttactcggccttgccccttccttcccatggagatcacaaggctcttactccacttgatcccatcgttccctctacctcctcactgaccctgggcttccagtgtggctccctgtggcgtgccatgccccttttggaatctgtaagtacaacaagctttcttgtcaatggcagtcacctgatctattgccctcaccatacttg

SEQ ID NO: 712 - DNA, 91 bp, Homo sapiens
tgtgccttctacagctgggctaaagcttctagaacattcctgaaagcagatgggctccctagaaggaagcagtgggttcttgtagaggcct

SEQ ID NO: 713 - DNA, 116 bp, Homo sapiens
gggttggtgcctcccttacagaggaccggattctgtcactgctgggcaaaccttcagagggttcccctccccacatgaaaacagcccaacttctctgctcagaggcttgcaactga

SEQ ID NO: 714 - DNA, 32 bp, Homo sapiens
attgagtgtttcatgctgcggtggctcctcca

SEQ ID NO: 715 - DNA, 28 bp, Homo sapiens
tcagtaatagctgaacctgttcaaaatg

SEQ ID NO: 716 - DNA, 76 bp, Homo sapiens
tgctgatgctattgccatggaaaccatccctctgcgaggagctctgtctgtctttgtcgaggagcactccagctcc

SEQ ID NO: 717 - DNA, 108 bp, Homo sapiens
ttttggtcttaggtggactagcatctgatgggacaaaatcttcatcatcagttttttcatcaaaatctgagaaatcttcatctgaatccaaatccattgtgaattttg

SEQ ID NO: 718 - DNA, 58 bp, Homo sapiens
ttgttttaggccttctagttccacaccatcttcttgagggcttccttcagtattttca

SEQ ID NO: 719 - DNA, 118 bp, Homo sapiens
catttctatggttattcgtggaatgactctttgaccacgcggagaaggcaaaacttcagccatttgtgtttttttccccttggccttcccccctttcccaggaagtccgacttgttca

SEQ ID NO: 720 - DNA, 111 bp, Homo sapiens
tggagacatgcccagctacagcaaacacagggaaacacgaagggggcagctggaagatttggtcttgaacttggggggtgggtaagtgatgatccccacgactggagcagc

SEQ ID NO: 721 - DNA, 772 bp, Homo sapiens
cggcacaagggattgacacgcgttccccaaatccgatgtttctgctttgtcgtggcccttcctgactctcctccgaacccagtgaggggctggtggctcccccggcatgaccccctcaaaaacgaaggggagatgttgcaagagccatgggagcgccagatggcaaggcttctttggcagtctgagaaccccaggtcccccagggcctgggggtgctgggcgggcaggagcgggctgagggtgggggccacttgggtgtttgagcattgcctttgattgctgggcagacaatacattgtttcctgtgtcttctggggagacagatttgggaaggagtggaggggaggccccaaggggggtgtggagaaaggagcagaaagggcagcattggggtttcataagcccaacgggcagaaagggacttacccccgcatgggtcttcaagcaagtggaccaagcttccttttttaaaaagttatttatttattctttttttttttttttttttggtaaggttgaatgcacttttggtttttggtcatgttcggttggtcaaagataaaaactaagtttgagagatgaatgcaaaggaaaaaaatattttccaaagtccatgtgaaattgtctcccattttttggcttttgagggggttcagtttgggttgcttgtctgtttccgggttggggggaaagttggttgggtgggagggagccaggttgggatggagggagtttacaggaagcagacagggccaacgtcgaagccgaattc

SEQ ID NO: 722 - DNA, 33 bp, Homo sapiens
ctgtgtataaggcttcgttaagcttaattgaga

SEQ ID NO: 723 - DNA, 47 bp, Homo sapiens
ggttgctcctggacgccaccaacaactttagctatgttttctacatg

SEQ ID NO: 724 - DNA, 33 bp, Homo sapiens
cttttccctgccgtgttattggtgtcaaccctg

SEQ ID NO: 725 - DNA, 31 bp, Homo sapiens
tgttcctgagaaataaaaagcctgtcatttc

SEQ ID NO: 726 - DNA, 114 bp, Homo sapiens
ccacggtgttctggccaaagacatcagctaagaaaggaaactgggtcctacggcttggactttccaaccctgacagacccgcaagacaaaacaactggttcttgccagcctcta

SEQ ID NO: 727 - DNA, 27 bp, Homo sapiens
gagcctgttcctctcacgccctcacct

SEQ ID NO: 728 - DNA, 41 bp, Homo sapiens
gtctatatttacgtgcctaacagtagctctgggattttatc

SEQ ID NO: 729 - DNA, 27 bp, Homo sapiens
tttaaaggcagttctttatcagtcaca

SEQ ID NO: 730 - DNA, 54 bp, Homo sapiens
tttggtatagatgtgacacattttcttgctctcctgtgttctgctaaatcacca

SEQ ID NO: 731 - DNA, 30 bp, Homo sapiens
tagagccaactgagataaatgctatttaaa

SEQ ID NO: 732 - DNA, 356 bp, Homo sapiens
caaaaacggacaaggccagaaacagcttcatatggacactgggacttagccccaagcctgggtgtcctctgaggccagcctctccaccttctgagcctgcgcccacactattgaaaacactaatgaaagtactcctctgaagtccttgtcctttattgggcaaggggtgagggaagcagacagaccggttggacatagtagatgggtgtgtgaggacaaaatgctaccagagagccagccaatccacactcccctgaaatggatgctaacacccttcaaaccctgggcattggtttcatacacagcctcttccagggataaaaggggttcttgtacagcttgttactcaaccatgg

SEQ ID NO: 733 - DNA, 25 bp, Homo sapiens
ggaaataataaagatgctcttttaa

SEQ ID NO: 734 - DNA, 94 bp, Homo sapiens
aacatcctagatagccatgcagctacattgaaccggaaatctgaacgggaagtcttctttatgaatactcagagcattgttcagctggtacaga

SEQ ID NO: 735 - DNA, 172 bp, Homo sapiens
ttccataaaatgcaaatgctgattcatcagtgagtcagtatatgaaaaagggcctcttaaatgtcttataaacactaattattcttccccagtcttcatttccttaaagtcacatcgctcacaagtaggctcatcttccacttctgccatctgaaggctggtccatgcccag

SEQ ID NO: 736 - DNA, 63 bp, Homo sapiens
aaaatactgaaggaagccctcaagaagatggtgtggaactagaaggcctaaaacaaagattag

SEQ ID NO: 737 - DNA, 127 bp, Homo sapiens
aacaagatgaacaagtcggacttcctgggaaaggggggaaggccaaggggaaaaaaacacaaatggctgaagttttgccttctccgcgtggtcaaagagtcattccacgaataaccatagaaatgaa

SEQ ID NO: 738 - DNA, 111 bp, Homo sapiens
tcaagggtactattgaagaactggctccaaatcaatatgtgattagtggtgaagtagctattcttaattctacaaccattgaaatctcagagcttcccgtcagaacatgga

SEQ ID NO: 739 - DNA, 63 bp, Homo sapiens
taatgctgcggacaacaaacaaagggacccaaaaatgtcttgtattagagtcacaattgatcc

SEQ ID NO: 740 - DNA, 36 bp, Homo sapiens
cctgtaaatgaaaatatgcaagtcaacaaaataaag

SEQ ID NO: 741 - DNA, 29 bp, Homo sapiens
ttgggcctggctcttgccgtggtcttgat

SEQ ID NO: 742 - DNA, 185 bp, Homo sapiens
ggaaagtccttgcacagacaccagtgagtgagttctaaaagatacccttggaattatcagactcagaaacttttattttttttttctgtaacagtctcaccagacttctcataatgctcttaatatattgcacttttctaatcaaagtgcgagtttatgagggtaaagctctactttcctactgc

SEQ ID NO: 743 - DNA, 243 bp, Homo sapiens
ttgagtaggcttcattcagggcatgtctctcccccaggccccatcttcatacacttccgctaagatgcccacaattcatcccccttcatgtgcagcttgaaaaaccctgacattgttgctccctgccgggcatacctgccattgccactgcccatctctgtgatgaggaattcaacaccagatctctggcagctcttgagggattggccagtggccattctcaggggtgctgtctagttttcc

SEQ ID NO: 744 - DNA, 25 bp, Homo sapiens
ttacataaaagcctacttgagaaga

SEQ ID NO: 745 - DNA, 287 bp, Homo sapiens
gtgaggtccctgatctgtgctctgagggggtggtcagtcaggcagggcttgggccaaccagtaaggcagatgagggtccctgtcctgggggactcactgctcctgggtgccccaggagatgtagtctggccgtgtggccgcatggtactcatcgatgagctgctgcacaatctccctggatgtgtccatctcatcaaagttgtccttgaacatgtcctccttgcggaactgctccaggaaggcctcccgcttacgcagcttgtcatactggcgacaggttctctcga

SEQ ID NO: 746 - DNA, 314 bp, Homo sapiens
cccgtctctcccacggatggggagagggcaggaggagacccagccaagtgccttttcctcagcactgagggagggggcttgtttcccttccctcccggcgacaagctccagggcagggctgtccctctgggcggcccagcacttcctcagacacaacttcttcctgctgctccagtcgtggggatcatcacttacccaccccccaagttcaagaccaaatcttccagctgcccccttcgtgtttccctgtgtttgctgtagctgggcatgtctccaggaaccaagaagccctcagcctggtgtagtctccctga

SEQ ID NO: 747 - DNA, 44 bp, Homo sapiens
atggcggaccgcttctcccgcttcaacgaagaccgagactttca

SEQ ID NO: 748 - DNA, 31 bp, Homo sapiens
attattttgattgctggaataaagcatgtgg

SEQ ID NO: 749 - DNA, 114 bp, Homo sapiens
gacaatttcacatggactttggaaaatatttttttcctttgcattcatctctcaaacttagtttttatctttgaccaaccgaacatgaccaaaaaccaaaagtgcattcaacct

SEQ ID NO: 750 - DNA, 129 bp, Homo sapiens
agtcacaccggagcctggggcaagacagtgattgaatacaaaaccaccaagacctcccgcctgcccatcatcgatgtggcccccttggacgttggtgccccagaccaggaattcggcttcgacgttggc

SEQ ID NO: 751 - DNA, 124 bp, Homo sapiens
agactggtgagacctgcgtgtaccccactcagcccagtgtggcccagaagaactggtacatcagcaagaaccccaaggacaagaggcatgtctggttcggcgagagcatgaccgatggattcca

SEQ ID NO: 752 - DNA, 67 bp, Homo sapiens
tgctggtcccaaaggtgctgatggctctcctggcaaagatggcgtccgtggtctgactggccccatt

SEQ ID NO: 753 - DNA, 26 bp, Homo sapiens
gtttccctggcgcagatggtgttgct

SEQ ID NO: 754 - DNA, 131 bp, Homo sapiens
cccagcagttgcctccagtgcccccaaggactggcggccttgcgtccagaaaccccacgcacaaacctgctgctatggctgcagcggcagctgctacaaccgcgggcgcggagcgaatcccggagcccaaa

SEQ ID NO: 755 - DNA, 419 bp, Homo sapiens
gtaactgtgtcccagtgaccaaattgcactcgactcgatcagctgttcatccatttcgtgttttttcctgtcaaacattaatccagcaaatatatgaggtatttaccaatttattttcttagtattacaaaataattcattagcataaagtacaatagtgaaatatttgagttgttcggaacctcaattaatcctgttttacatttcagacctaaagctggcaatcaggagaagaagcactttgttttaaatgtggagaagataacacttgattccatttcattgtcattagtgtattaaccagcaggagaggtgatgagccatttttcaaatgaaataccttttatttccatataatttttttattttagagttcaatagctgtttctatgattatcctcaatttccatatgttactg

SEQ ID NO: 756 - DNA, 51 bp, Homo sapiens
tggacaagacctcataactgtgattaatatcaataaaaaggggatgttgtg

SEQ ID NO: 757 - DNA, 30 bp, Homo sapiens
caggctttttatttctcaggaacagccgag

SEQ ID NO: 758 - DNA, 80 bp, Homo sapiens
caaactgttcggctactggacaggttgtatattaccagatcatcactagcagatgtcagttgcacattgagtcctttatg

SEQ ID NO: 759 - DNA, 87 bp, Homo sapiens
ttctgattcagtccggccaattaaatctaaatccacccctgaaagccatctggtgtggataacaagcccacaaatgagcagtcagct

SEQ ID NO: 760 - DNA, 212 bp, Homo sapiens
gcctcccgggtactgtcatcctgaattctgagaccatccagcacttcctttagttttgccctggtgctgttgacttttgtttactgaagagtgtgctggaggcaggacaagggacatggaaggctgcaatttaagagtctaaaaggttttagaatcctgaaggaggtttaacaagctgaattgaagaataatacctttctcaactggagaga

SEQ ID NO: 761 - DNA, 82 bp, Homo sapiens
catctgaaattaatgggactaatgtaaggattgatattttcatatttgctcaattgcatcctgggttaagcacattcaacag

SEQ ID NO: 762 - DNA, 65 bp, Homo sapiens
ggaatgaatggagtaataaaccgcaatgaaaccattcagtggctgtacactctcattgggtcaaa

SEQ ID NO: 763 - DNA, 250 bp, Homo sapiens
tccctgtgtaggatggcttcccgttattttttttttaagcaaagtaaatgaacatcaaatttccatagtcagctgctgtctttctgcccactgagagctctttggtgaaggcaaagtcctccttcttcattagcggtctcccatgtggggccacatcttccctcaccaggaacccagtgggcgcgctccagcccccctcagcttgccttttgcgtggtcattagagctagggcacacgtcatgctgattc

SEQ ID NO: 764 - DNA, 25 bp, Homo sapiens
ggttagagtggacagccccactatg

SEQ ID NO: 765 - DNA, 56 bp, Homo sapiens
ttcattttagtcatactgtagactcattaaaaccagggttcaaggactcacctaag

SEQ ID NO: 766 - DNA, 98 bp, Homo sapiens
ttaagctgttctgagaaatggtatcaagccaggttgcaaactgtggattttgaacagtctacagaagaaacgaggaaaacgattaatgcttgggttga

SEQ ID NO: 767 - DNA, 127 bp, Homo sapiens
ctccatgtttgatattgtgacaaagaaggaagtcaacccccacccccaccccatcttctatcttttgagtcctggtgatttgagtaattcctgtgtggagaccagcttcctgccaaattctcattta

SEQ ID NO: 768 - DNA, 106 bp, Homo sapiens
tctgattatacaagtgctaagtggcagaaaggtctggaataaatacatcaaaaagaagaggcaaagctgtgaaactaagttgcatgcaacaggttctatgagggtg

SEQ ID NO: 769 - DNA, 52 bp, Homo sapiens
attgctatgctatgccttgtatgactaagacctcttaccaagaatacgtgtc

SEQ ID NO: 770 - DNA, 30 bp, Homo sapiens
ccttgcccagtggctttgtattccttcttt

SEQ ID NO: 771 - DNA, 292 bp, Homo sapiens
tttacactgtaagaggcaccatttccccaaggaatacctcttggcatttcctgaatgagtgggattagcaatctaaataaatcatatttcaagaggtaacagcaacagataaaatttaaagggattattaaaataacatttacaagactctgaacaattcttgaactcttattaaaaccacaaagaaagaacaattctttatttatgaatttcataaaggactcaatgtgcaactgacatctgctagtgatgatctggtaatatacaacctgtccagtagccgaacagtttg

SEQ ID NO: 772 - DNA, 47 bp, Homo sapiens
gttttagacaaatactcatgtgtatgggcaaaaaactcgaggactgt

SEQ ID NO: 773 - DNA, 50 bp, Homo sapiens
ttcagtattgatgctcctccatcctttaagccagctaagaagtattctga

SEQ ID NO: 774 - DNA, 90 bp, Homo sapiens
gctggttacttccaatcaacctaactccaaaacctccaaggaaagcttgggctctagcaataactgcttcaggctgtcagagtcattttg

SEQ ID NO: 775 - DNA, 61 bp, Homo sapiens
gcgccccttcccatatttattcggaccccaagcatcgccccaataaagaccagcaagcaac

SEQ ID NO: 776 - DNA, 505 bp, Homo sapiens
gaagtcagcttagttcgggaacccagctgatttccggagcccaacccagctccagaactcagactagttctggaaaccagctcagctccgggacccagaccactgcaagaattcagctcagccctggagctcatcttagctccagaacccagtccagtcctgagaccaggctcagctctgaggctcagtccagctctggaacccagttcagttctgggattcagtttggctctgaaacccagaccggctcaagaattcagcccgactctggagcccaacttagttccagaacccagtccagctctgagaccgggctcagctctgaaacccagaccggctcaagaattcagcccagccctggagcccaccttagttctggaacccagcctgtttctggaacccaatccagttccagaactcacaccagttcaagaatctggctgagccctggaaatggaatctgcccacagacccctggccttgaccctagaatccagcttgaggc

SEQ ID NO: 777 - DNA, 55 bp, Homo sapiens
ctaaaattttatcacggatgaatggatggacttgctctctatgtaatatgaaatt

SEQ ID NO: 778 - DNA, 170 bp, Homo sapiens
gtgccagacccaaaagcttttcctacagtgataccctttatttttacttccccttgactcatatgttttaacatgattttaacaaactgcacttattaagaaatgtgtttgccctgttttgtttggtttcgttttgttttctttgaataaatgacatggcacctcctagc

SEQ ID NO: 779 - DNA, 83 bp, Homo sapiens
cgcgcagcctgcagcgggagaccctgtccccgccccagccgtcctcctggggtggaccctagtttaataaagattcaccaagt

SEQ ID NO: 780 - DNA, 58 bp, Homo sapiens
gaggggcctctgaaatttcccacaccccagcgcctgtgctgaggactccctccatgtg

SEQ ID NO: 781 - DNA, 58 bp, Homo sapiens
tctctcttcaacactcccctgcgtaccccggttctagcaaacaccaattgattgactg

SEQ ID NO: 782 - DNA, 185 bp, Homo sapiens
tctctccctagaacgtgcgcggtgatctcctacttttgtgtgtatacttactctttcgtggacagtgtatatatatatgtatatatacatatatatatacacattttttttcccaaccccttttttttttccttttttttgtgtgtgtgtgtttgagatggggtctcgctgtttcgctctgtccc

SEQ ID NO: 783 - DNA, 30 bp, Homo sapiens
tcatatttgctaagacgaatttgctcatta

SEQ ID NO: 784 - DNA, 26 bp, Homo sapiens
gatgcttgtgacagcgatcaagacca

SEQ ID NO: 785 - DNA, 86 bp, Homo sapiens
ggatccgcaaccaggccgacaactgccctagggtacccaactcagaccagaaggacagtgatggcgatggtataggggatgcctgt

SEQ ID NO: 786 - DNA, 42 bp, Homo sapiens
gaaaacacaataaggagaggccaccaggtagcagaatcctca

SEQ ID NO: 787 - DNA, 87 bp, Homo sapiens
gaacgtcatcttgctcttgagcttatttcagaaaaaagggaaaaagaaaatgggtaaaagcgtgggagttacctgtgctcacctgta

SEQ ID NO: 788 - DNA, 31 bp, Homo sapiens
aatgtgtatgaatgactgaggctttgtaaat

SEQ ID NO: 789 - DNA, 49 bp, Homo sapiens
ctgtgccaggtccaagtcgggggacctgaagaatcaatctgtgtgagtc

SEQ ID NO: 790 - DNA, 86 bp, Homo sapiens
catttgggatagtcgtctttaaaagacttggtgttatttacagtgtttgttttgataacatttggctgggtcattttaatagttag

SEQ ID NO: 791 - DNA, 25 bp, Homo sapiens
tgaagaatctgagccctttcatcac

SEQ ID NO: 792 - DNA, 32 bp, Homo sapiens
aggaggagagtcgactttgcctcttgcccaag

SEQ ID NO: 793 - DNA, 28 bp, Homo sapiens
ggaaccgtgcagtgtgcattttaagacc

SEQ ID NO: 794 - DNA, 25 bp, Homo sapiens
tggcctggaataaatacgttttgtc

SEQ ID NO: 795 - DNA, 58 bp, Homo sapiens
ggttgccaactacatctgtcccttcccatcaatcccagcccatgtactaataaaagaa

SEQ ID NO: 796 - DNA, 93 bp, Homo sapiens
gtcattcacaatcacaagcagtttatagtttgacatattcttctagatcctgtgtgtaggcacaacatccaattttatgggactgagactgta

SEQ ID NO: 797 - DNA, 120 bp, Homo sapiens
tgacagttcggactatcacaggaaatatctactatgcaaggtcaggaactaagattattgggaaggttcacgaaaagttcacgttgattgatggcatccgcgtggcaacaggctcctaca

SEQ ID NO: 798 - DNA, 36 bp, Homo sapiens
gtgatttctgtataggactctttatcttgagctgtg

SEQ ID NO: 799 - DNA, 124 bp, Homo sapiens
tcacccaccatagggcatgattaacaaagcaacctttcccttcccccgagtgattttgcgaaacccccttttcccttcagcttgcttagatgttccaaatttagaaagcttaaggcggcctaca

SEQ ID NO: 800 - DNA, 79 bp, Homo sapiens
ccagcatctcgtgtgggctgcgccttccttggaggctacatgcctccgagttcctctgccttgccctacagccgggagg

SEQ ID NO: 801 - DNA, 33 bp, Homo sapiens
ttctctgtaaatctagtatcattccaaaataaa

SEQ ID NO: 802 - DNA, 84 bp, Homo sapiens
aggaatgccagctattatctgcgtccctggccaccccggggctccaaggacttctcaacccacgagatctttcggatggagcag

SEQ ID NO: 803 - DNA, 56 bp, Homo sapiens
gagaatagcatgttcgtattacaagagaaacaaataaactagtctgttggcaattg

SEQ ID NO: 804 - DNA, 300 bp, Homo sapiens
ggaagcacaccaatcttactttgtaaattctgatttcttttcaccattcgtacataatactgaaccacttgtagatttgatttttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttg

SEQ ID NO: 805 - DNA, 156 bp, Homo sapiens
ttgacttcatgacaaatgggccatcaaactaaaacttagcagaactatggcagcctgagagttccagagtggaatttggccttagaacatttcctaagtgcgagtttgggcacagccagccaaagaggctcggtgacttgtcactccagagaatgc

SEQ ID NO: 806 - DNA, 1033 bp, Homo sapiens
gagaccagctccgtctaagtttatgagatcccttccaggccctatgtcaggggctttcagcacctcccacccccgcctggtccagggcttctgctcatttgagagggagacagggaagttctgagcctaggagctgaagagaggacactagggaattccccctgcctctagctccagtagtaccaactagaactgattctccgtgttgggatctccacccgttcgtccagggttgggagagaatcccactcatgctcaccccccggggctctcaagtctcatccactttctggttctggaatctctgccctcccttctgggctttttttgtttaagactgagtcaaacagggtcttgctgtgtcctacaggctggagcagtggtgctgtcaacggctcactgcatccctgacctcctgggctcacgtagtccacctcagtcttctgagtagctgggactgcgggcattcaccaccacaaccagctaagttttaatttttgtagagatggggcctccctatgttgcccaggccaatctcaaactcctgggctgaagttatccacccaccttggcctaccaacgtgttgggattacaggcgtgagccaccttgaaaggacccccatccacacccatatgggctcctgggatcctcatccttgggagagattttcgaattcctccctcaccccctaggacttctgcatacaaggcctagactcccagcatcccttcccacccacttgtccagctttctctgccccccagctgcctccccagccctgctccccatcccccgccctcacagccggcttcttccgagtgaggtggttgaggaacgatttccaccaggccttgtccatgtcagccggcctctccttgcgggcctgcttgggtgctttggtccttttgtgggaactatagtttcccatggcagggctggtcaccactggggacgatggcccacactccgtctgctctgccccctgggccctggcaacattgtgacctcacgggctgacctcaccaattgca

SEQ ID NO: 807 - DNA, 216 bp, Homo sapiens
tatacaagggctcaaccgaggcttaatttaaaagacaaaaacaaaacaaaaataccacagctcaagataaagagtcctatacagaaatcacaaaaaggacagaccatctaaggaaaaattaaaaagacgacacaaggacaggctgggcagcctgggtcagggctcctggctggtgacctgctttgagtaggtttcttgcaggtacttcttaaaagc

SEQ ID NO: 808 - DNA, 79 bp, Homo sapiens
agccctctgcgcagagagaggacttcgatttccactgtactcagctcccctacaagaaagtgccgacacctgggaggct

SEQ ID NO: 809 - DNA, 125 bp, Homo sapiens
caacctctcgctctctggacttgggttgtaaatcgcctcaaggggacagaaaccacccagaaagctgggaggggccccacgagcttacgactgaggtgggataatccgcggagtggtcacccacg

SEQ ID NO: 810 - DNA, 44 bp, Homo sapiens
gcttggtgggctacgcacgcctctgtcccggagaccactatgag

SEQ ID NO: 811 - DNA, 57 bp, Homo sapiens
ttatccttgagatttcactacctttatgttaaaagttgtgtataattgttaaaatct

SEQ ID NO: 812 - DNA, 90 bp, Homo sapiens
gccagggctgccatataacctgacaggaacatgctactgaagtttattttaccattgactgctgccctcaatctagaacgctacacaaga

SEQ ID NO: 813 - DNA, 104 bp, Homo sapiens
tgctacatagtcaaacagaactgggttggtcttttatggcataaaattaattcacagtcaaactctcaactagtactagaaggattcgctgaagagtttctctc

SEQ ID NO: 814 - DNA, 29 bp, Homo sapiens
aatcctgtcttgttcataaattgacaatg

SEQ ID NO: 815 - DNA, 92 bp, Homo sapiens
gaagctgacaaacttcttaacagccactacacatccaaacgcaacacacttggaaggggaagaaaacagcagcaaccccctggcgcggcaga

SEQ ID NO: 816 - DNA, 418 bp, Homo sapiens
cagtgacttctatcagactcttgaatatgcacccttgggaaatggagtaagaaccatagcttaaatcgtttaaactcatatgggacaaaacgatgaaaatatgcaagagggaacaatcttacattggaaaagaaatcaagccaatgacccaatatatctttttattatttcttgggagaacctgtatgtttaattcatggaattcttcactgtttctatgttgaaatacacttatgtgaaaattgcactcctattaataaaataggactttaaaaagcccaaggattttagtatcgaaatgtttctgtgcaatatattaacattcattgactatgtgaatccccacctgtacttcgtgtaaattaccaaaggtgccctttcctctgtgcacatgacacgtgtgcccttgccggtgtaa

SEQ ID NO: 817 - DNA, 300 bp, Homo sapiens
gactatgggcgcacgccgggtatccgcgccgcagccaggctcggagcgcgcggctggaggggcgcgggcggcgcgcgctccagagatgccttcctgcgaggctccgtcagcgcggcccggggagatcgcccgcaggcagcggaggcgcggcgggccgggggacgaccagggagcccctaacaaagcagctttgggagggccggaggcctctgcctccctcggcgataggtccgcgaagctgcctgtcatctcgcataggtggaggtggccaatggcgtgcacagacggagctctagttgg

SEQ ID NO: 818 - DNA, 110 bp, Homo sapiens
tgtccttgtacccaaagctgtaaacaagagccctttgtgagggagaaccacatgactggccaaaacgccccacctactagtgtgtgctcggtggaaatgtcttgtgactg

SEQ ID NO: 819 - DNA, 122 bp, Homo sapiens
gcaaactgatcctcatgcacgttaacacccatagcgctcaattcttatggatatgcagacagtgttttggggaccaccgtggagaatctgtttcaaactaaggcgtgagcttctaagaacta

SEQ ID NO: 820 - DNA, 45 bp, Homo sapiens
attgtaagttatggacatgatttgagatgtagaagccatttttta

SEQ ID NO: 821 - DNA, 297 bp, Homo sapiens
gctcacttctgtcacgcatttaaaatgtcacagagaccaaaatagagtggctttctggtggaactcatggcagtcatacaacaagatacaaaactaggagactctgtcttctcatacatcatacaattaatttttcaagtattctttatgtacaaagagctactctacctggaaagaaaattaaaaaaaaaaaagacaaggtaggttatgcatcctaaggaggaggaggggatggagcggtcgggcacatgttcccgtccccacgaccccacggaccgctgattccctgccactgag

SEQ ID NO: 822 - DNA, 84 bp, Homo sapiens
tcatgcttagctcagttgttttgtgggtaagttacttaaacaggaaattgaagtatttctgtgactacttctgctctcactact

SEQ ID NO: 823 - DNA, 29 bp, Homo sapiens
ggagaagcagtgttccaatgccaggggtg

SEQ ID NO: 824 - DNA, 454 bp, Homo sapiens
ggctggaattccgtctcataatcaatgccatgtacattaagatctgcgaaagaccaacttttaggcagtgatacttttctcccattccctggggtggggggagtatgcagttggtgctttctgtaattcccttgttctgttttgtttctgtaagcttttcccctggtgtcatggaaaggacttcttaaataaccacattgtgggtggctgtatccaaagtttaaataattggccagaagtgcagagtatcctttcctggattcgtgtcagaaaagggctccttgccacaactgaacttactgtataaaaacctggctagggagatttaattttactaaaattacagtttaatgttaccgtctagccacaaatcaagcagcaaaagctattttgatgatgaaagggggtccccgttgagctggccatctagtgcagtgtgctctcagattccatg

SEQ ID NO: 825 - DNA, 511 bp, Homo sapiens
tccagtagccttattctgtcagggacgaattgtttaccccatgataatgtaggatatcataaattcttcttcaaaggttttagcctgtaatttgttaagtacattgagttctgagatcctctccaaagaaccaatgtatcagtatgttcagctcacctgttctttgttcttcattttaacttttaatttcctcattctttatgtctccttgcccctagtttcagtaaacaatcccctcctagcctctgtcacctgctctgacctgagtcacctttagtcacctggtccgtaaccatctttcctgtctaaacttctcaccccaccactctggcttatacccctgctctctttaaaatagccagtcagaattagcttagattgtgcggtccaaccctagcccataggggaacaacacagcagtagggggtacctgcatcagggataagaacccattcccctcccttgttccggtgtgctctcgccattgcaccatccatgagacgcactcttg

SEQ ID NO: 826 - DNA, 384 bp, Homo sapiens
tacacactgtgatagacggtttggagaattcaggaaagaaaaagaaaaagaaaaaaaattcctcctaggccctctgctccagagcctccagccgtcacatgacagtgtatttctttacaattacagaaatagatacgtgactgttacattttgaaactttttcttttaccttgacttatttttaaatagaaacactagtgaggccccacagaactgagcagaactaatggccccccatgtggtggaatagttctgagctggccccactttggatgcagccttccccggcaagtctaaacccagtctctgtggctgcctgttcacagggaccgtggactgtgatgtttacagtacgaaagcactgggagagaacgctacatcaca

SEQ ID NO: 827 - DNA, 92 bp, Homo sapiens
gccatgaaaaggggcgaggaccctcccaccccgccacctcggccacagaaaacccattccagagcctcctccttggatctgaataaagtctt

SEQ ID NO: 828 - DNA, 191 bp, Homo sapiens
tgcacataattaactggttccatcaagactgtgcacccaggccttacagtccaacctttttctgtgtctggctaatatttaaaactagaaaaactattcctaatcaacatggagtggagagtttattcactgtcttatctgcagaaatttgctgtcaatatataacccgcctgcagtggaaagtgtatagt

SEQ ID NO: 829 - DNA, 69 bp, Homo sapiens
ctgctgccaaactcttgactgaatatggcgttagtgttttggttttagaagctcgggacagggttggag

SEQ ID NO: 830 - DNA, 75 bp, Homo sapiens
tctgatccaggatcgtccttccaaatggctgtatttataaaggtttttggagctgcaccgaagcatcttatttta

SEQ ID NO: 831 - DNA, 168 bp, Homo sapiens
tgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaa

SEQ ID NO: 832 - DNA, 116 bp, Homo sapiens
gcaggcatttgtcaggccatatgttttaaccttatggtaatactttgctttagtcgttcctcctgctaccagtagcgttttgacccacctgccagtgtttgcttgctctatgtttc

SEQ ID NO: 833 - DNA, 72 bp, Homo sapiens
agttatggccatctacccctgctagaaggttacagtgtattatgtagcatgcaatgtgttatgtagtgctta

SEQ ID NO: 834 - DNA, 100 bp, Homo sapiens
ccagaaactcgcgtcagaagccttagtagtccgactgggcatgctcgggacttggggcgcttcgcatggaggcactctccagcggctaacatctgtccga

SEQ ID NO: 835 - DNA, 41 bp, Homo sapiens
cttttcattacatcaggtatattgccctgtaaattgtggta

SEQ ID NO: 836 - DNA, 655 bp, Homo sapiens
tccagtcctaggagaacagtccctgggtcagcagccaggaggcggtccataagaatggggacagtgggctctgccagggctgccgcacctgtccagacacacatgttctgttcctcctcctcatgcatttccagcctttcaaccctccccgactctgcggctcccctcagcccccttgcaagttcatggcctgtccctcccagacccctgctccactggcccttcgaccagtcctcccttctgttctctctttccccgtccttcctctctctctctctctctctctctctctctttctgtgtgtgtgtgtgtgtgtgtgtgtgtgtgtgtgtgtgtgtgtgtcttgtgcttcctcagacctttctcgcttctgagcttggtggcctgttccctccatctctccgaacctggcttcgcctgtccctttcactccacaccctctggccttctgccttgagctgggactgctttctgtctgtccggcctgcacccagcccctgcccacaaaaccccagggacagcagtctccccagcctgccctgctcaggccttgcccccaaacctgtactgtcccggaggaggttgggaggtggaggcccagcatcccgcgcagatgacaccatcaaccgccagagtcccagacaccggttttcct

SEQ ID NO: 837 - DNA, 36 bp, Homo sapiens
gggctgcttctgaggtcggtggctgtctttccatta

SEQ ID NO: 838 - DNA, 56 bp, Homo sapiens
gtaagtgtaaggtccacatccttggggaagtagttaaataaaatagttatgactga

SEQ ID NO: 839 - DNA, 95 bp, Homo sapiens
agacctccatccctagttggagatatttgtctctggatggctcttcccacattctctatggaagtgctttgtactttggagcctctgcccactag

SEQ ID NO: 840 - DNA, 29 bp, Homo sapiens
caaaatgggtaaatgcacaattttctaag

SEQ ID NO: 841 - DNA, 34 bp, Homo sapiens
atgccaacaacgtcgaggcctgcacttgatgtca

SEQ ID NO: 842 - DNA, 84 bp, Homo sapiens
ttattaagcactacataacacattgcatgctacataatacactgtaaccttctagcaggggtagatggccataactgagttatt

SEQ ID NO: 843 - DNA, 168 bp, Homo sapiens
taccttgatggtaacgctctatctggttttgggtgtttttcatgttttagcatttgtataaagaaactggtccatgtaaatactttccatgttttttcttcaaatgtttaaaccactagttgatgtatggtatctttagatatttgcctgtctgtttgctcaaaattg

SEQ ID NO: 844 - DNA, 106 bp, Homo sapiens
aggtcgaatgggtaaaggagcatttttttattcagcagactttctctgtgtatgagtgtgaatgatcaagtcctttgtgaatattttcaactatgtaggtaaattc

SEQ ID NO: 845 - DNA, 71 bp, Homo sapiens
ggagcaccaaatattctcctctccacgaagccaacttggacaatgtttattttgcaccaatcatatgcatg

SEQ ID NO: 846 - DNA, 1019 bp, Homo sapiens
gggaatccgtgagttcttaatcttactgagtttagttgtgttctttgattattttggatagctttgtttaacggtgaatgaaatgattaaaatggaaaaattaagtgcagaaaatatgtttaacattatataggatgtcttgttttaggcatttattgctagataacatatgtccattcttgtattgctataaagaaatattggatactggctaatttataaataaaagagatttattggctcatgattctgcaggctgtacaggaaatatgattctggaatccgcttggcttctggaaaggcctcaggaaacttagaatgatgacacaaggcagaggggaagcaggcacatcttacatggcaggagcagggagcaagaaagagtgaagcgggaggtgctacacacttttaataatccagatctgagtcaggcatagcagcttatgcctataatcccagcacttttgggaggccaaggcaggcagatcacctgagggtcaggagttcaaggccagcctggccaacatggtgaaacctcatgtctactaaaagtacaaaaattagctgggtgtggttgcacctgcttataatcccagctgctcaggaggctgaggcaggagaattgcttgaacccgggaggcagatgtttcagtgaaccaagattgcaccactgcactccagcctgggtgaacagagtaagactctgccttaaaagtaaaataaaattaaattaaaattaaaaaccagatcttgcaagaactcactgtcagaagaacagcaccaagggtatggtgctgaaccattcatgaaggagccatcctcaagatccagtcatctcataccaggtccacctccatctaatattgggaattacaattcatcatgagggttggtggagacatggatccaagccatgtcacatattatcctgactgtagtggtttaagataacaatttttatctcacaatttattttgaatacaggcatgatttagctgtgtcctttggttcagtatctc

SEQ ID NO: 847 - DNA, 30 bp, Homo sapiens
ggatcacctaaactgagtccagctggctaa

SEQ ID NO: 848 - DNA, 79 bp, Homo sapiens
aactcattacacctgcggaaaaaggaagagatttagagtcacgactcatagaagcatacgttattcaatgtcaggctga

SEQ ID NO: 849 - DNA, 61 bp, Homo sapiens
cctgtaaagctgcacttcgcaattccaagcatgttgcaattttgcctatggttttctgcag

SEQ ID NO: 850 - DNA, 162 bp, Homo sapiens
aaaggtgacactttgtgactaaagtatcccattatatataatgttttttgaaatgttggaaattttggggaattatcaaatgtatagaagttgcatgaaggttatagagaggtgtaactgtttgttaactattacatggatttcatactaggcagtgacaac

SEQ ID NO: 851 - DNA, 372 bp, Homo sapiens
gttgtagcccttgcacttcaagagatctagtctttactttcagttgtctgttaggtccattctgtttactagacggatgttaataaaaactatgcgagcctgaatgaattctcagccaaatttagtcttgtctctcatcttgattggattaattccaaattctaaaatgattcagtccacaatagctctaggggatgaagaatttgccttactttgcccagttcctaagactgtgagttgtcaaatccctagactgtaagctcttcaaggagcaagaggcgcattttctccgtgtcatgtaatttttctaaggtgcttggcagcactctgtaccctgtggagtactcagtaccttttgtttgatgttgctga

SEQ ID NO: 852 - DNA, 82 bp, Homo sapiens
gctacttgtcctctgcaggaccctaagcccctgcccgcagcccacatgccctctgtgatgagtggcgtctttcctgcctctg

SEQ ID NO: 853 - DNA, 64 bp, Homo sapiens
gagactgcatcggaggcggcgccccgttctagggccgtggcctttgccgagactgtagcagaga